(19) United States
(12) Patent Application Publication
(10) Pub. No.: US 2024/0067651 A1
(43) Pub. Date: Feb. 29, 2024

(54) PRODRUGS OF MYELOPEROXIDASE INHIBITORS

(71) Applicant: Biohaven Therapeutics Ltd., New Haven, CT (US)

(72) Inventors: Gene M. DUBOWCHIK, Middlefield, CT (US); Joseph Theodore LUNDQUIST, IV, Limerick, PA (US); Melody A. MCWHERTER, Boyertown, PA (US); Jeffrey C. PELLETIER, Lafayette Hill, PA (US); Allen B. REITZ, Lansdale, PA (US); Eric STROBEL, Warrington, PA (US); Yan ZHANG, Berwyn, PA (US)

(21) Appl. No.: 17/766,539

(22) PCT Filed: Oct. 9, 2020
(86) PCT No.: PCT/US20/54896

§ 371 Date: Apr. 5, 2022

Related U.S. Application Data
(60) Provisional application No. 62/913,417, filed on Oct. 10, 2019.

(51) Int. Cl. C07D 487/04; C07D 498/18; C07D 515/18; C07F 9/6561

(52) U.S. Cl. CPC C07D 487/04; C07D 498/18; C07D 515/18; C07F 9/6561

(57) ABSTRACT

Disclosed are prodrugs of myeloperoxidase (MPO) inhibitors, methods of treating MPO related disorders, e.g., multiple system atrophy, amyotrophic lateral sclerosis, and Huntington's disease, and methods of neuroprotection, which include administering to a patient in need thereof the prodrugs, pharmaceutical compositions including the prodrugs, and kits including the pharmaceutical compositions and instructions for use.

### CROSS-REFERENCE TO RELATED APPLICATIONS

The application claims priority to U.S. Provisional Patent Application No. 62/913,417 filed on Oct. 10, 2019 in the United States Patent and Trademark Office (USPTO), and all the benefits accruing therefrom under 35 U.S.C. § 119, the content of which is incorporated herein in its entirety by reference.

### FIELD OF THE INVENTION

The present invention relates to prodrugs of myeloperoxidase (MPO) inhibitors and their use in treating MPO-related disorders, such as multiple system atrophy (MSA), amyotrophic lateral sclerosis (ALS) or Huntington disease (HD). The present invention further relates to the use of prodrugs of MPO inhibitors for neuroprotection.

### BACKGROUND OF THE INVENTION

Prodrugs are molecules with little or no pharmacological activity that are converted to the active parent drug in vivo by enzymatic or chemical reactions or by a combination of the two. Prodrugs are often designed to improve bioavailability when a drug itself is poorly absorbed from the gastrointestinal tract. Since 2008, at least 30 prodrugs have been approved by the U.S. Food and Drug Administration (FDA). See, e.g., Rautio, Jarkko; Meanwell, Nicholas A.; Di, Li; Hageman, Michael J., Nature Reviews Drug Discovery, volume 17, pages 559-587 (2018).

Myeloperoxidase (MPO) is a heme-containing enzyme found predominantly in polymorphonuclear leukocytes (PMNs). MPO is one member of a diverse protein family of mammalian peroxidases that also includes eosinophil peroxidase, thyroid peroxidase, salivary peroxidase, lactoperoxidase, prostaglandin H synthase, and others. The mature enzyme is a dimer of identical halves. Each half molecule contains a covalently bound heme that exhibits unusual spectral properties responsible for the characteristic green color of MPO. Cleavage of the disulfide bridge linking the two halves of MPO yields the hemi-enzyme that exhibits spectral and catalytic properties indistinguishable from those of the intact enzyme. The enzyme uses hydrogen peroxide to oxidize chloride to hypochlorous acid. Other halides and pseudohalides (like thiocyanate) are also physiological substrates to MPO.

PMNs are of particular importance for combating infections. These cells contain MPO, with well-documented microbicidal action. PMNs act non-specifically by phagocytosis to engulf microorganisms, incorporate them into vacuoles, termed phagosomes, which fuse with granules containing myeloperoxidase to form phagolysosomes. In phagolysosomes, the enzymatic activity of the myeloperoxidase leads to the formation of hypochlorous acid, a potent bactericidal compound. Hypochlorous acid is oxidizing in itself, and reacts most avidly with thiols and thioethers, but also converts amines into chloramines, and chlorinates aromatic amino acids. Macrophages are large phagocytic cells, which, like PMNs, are capable of phagocytosing microorganisms. Macrophages can generate hydrogen peroxide and upon activation also produce myeloperoxidase. MPO and hydrogen peroxide can also be released to the outside of the cells where the reaction with chloride can induce damage to adjacent tissue.

Linkage of myeloperoxidase activity to disease has been implicated in neurological diseases with a neuroinflammatory response including multiple scleroses such as amyotrophic lateral sclerosis, Alzheimer's disease, and Parkinson's disease.

MPO positive cells are immensely present in the circulation and in tissue undergoing inflammation. More specifically, MPO containing macrophages, microglia, astrocytes and/or neurons have been documented in the CNS during disease; multiple sclerosis (Nagra et al. Journal of Neuroimmunology 1997; 78(1-2):97-107; Marik et al. Brain 2007; 130: 2800-15; Gray et al. Brain Pathology 2008; 18: 86-95), Parkinson's disease (Choi et al. J. Neurosci. 2005; 25(28):6594-600), and Alzheimer's disease (Reynolds et al. Experimental Neurology 1999; 155:31-41; Green et al. Journal of Neurochemistry 2004; 90(3):724-33). It is supposed that some aspects of a chronic ongoing inflammation result in an overwhelming destruction where agents from MPO reactions have an important role.

The enzyme is released both extracellularly as well as into phagolysosomes in the neutrophils (Hampton et al. Blood 1998; 92(9):3007-17). A prerequisite for the MPO activity is the presence of hydrogen peroxide, generated by NADPH oxidase and a subsequent superoxide dismutation. The oxidized enzyme is able to use a plethora of different substrates of which chloride is most recognized. From this reaction the strong non-radical oxidant—hypochlorous acid (HOCl)—is formed. HOCl oxidizes sulfur-containing amino acids like cysteine and methionine very efficiently (Peskin et al. Free Radical Biology and Medicine 2001; 30(5):572-9). It also forms chloramines with amino groups, both in proteins and other biomolecules (Peskin et al. Free Radical Biology and Medicine 2004; 37(10):1622-30). It chlorinates phenols (like tyrosine) (Hazen et al. Mass Free Radical Biology and Medicine 1997; 23(6):909-16) and unsaturated bonds in lipids (Albert et al. J. Biol. Chem. 2001; 276(26):23733-41), oxidizes iron centers (Rosen et al. Journal of Biological Chemistry 1982; 257(22):13731-354) and crosslinks proteins (Fu et al. Biochemistry 2002; 41(4):1293-301). Various compounds that are MPO inhibitors are disclosed in WO 2001/085146 published Nov. 15, 2001, J. Heterocyclic Chemistry, 1992, 29, 343-354, J. Chem. Soc., 1962, 1863, WO 2003/089430 published Oct. 30, 2003, and WO 2006/062465 published Jun. 15, 2006.

Multiple system atrophy (MSA) is a neurodegenerative disorder presenting with autonomic failure and with motor impairment resulting from L-DOPA-unresponsive Parkinsonism, cerebellar ataxia, and pyramidal signs. Histologically, there is neuron loss in the striatum, substantia nigra pars compacta, cerebellum, pons, inferior olives, and intermediolateral column of the spinal cord. Glial pathology includes astrogliosis, microglial activation and α-synuclein containing oligodendroglial cytoplasmic inclusions. The pronounced neuroinflammation with activated microglia contribution as well as cytoplasmic inclusion bodies, containing aggregated and oxidatively modified proteins, makes it plausible to consider a significant contribution of MPO activity in the progressive neurodegeneration characterizing the MSA pathology.

Support for MPO inhibition in an MSA-like pathology can be generated through the use of preclinical disease models for MSA, like transgenic mice with oligodendroglial overexpression of human α-synuclein with or without a toxin addition like 3-nitropropionic acid.

Huntington's disease (HD) is a hereditary progressive neurodegenerative disorder characterized clinically by motor and psychiatric disturbances and pathologically by neuronal loss and gliosis (reactive astrocytosis), particularly, in the striatum and cerebral cortex. HD is a neurodegenerative disorder caused by expansion of a CAG repeat in the HD gene, coding for polyglutamine in the huntingtin protein. Explanations to the pathological mechanisms include oxidative stress, impaired energy metabolism, and abnormal protein-protein interactions. Such mechanisms are possible to link to MPO activity, which might be manifested through its observed overexpression in pathological HD tissue (Choi et al. J. Neurosci. 2005; 25(28):6594-600).

Support for MPO inhibition in an HD-like pathology can be generated through the use of preclinical disease models for HD. Such models might be mice or rats treated with mitochondrial toxins like 3-nitropropionic acid or malonate (Matthews et al J. Neurosci. 1998; 18:156-63). Useful models might also be transgenic mice expressing mutants of the huntingtin protein with or without a toxin addition like 3-nitropropionic acid (Bogdanov et al. J. Neurochem. 1998; 71:2642-44).

There is a large unmet need for medications that can be used for the treatment of Huntington's disease, for the treatment of multiple system atrophy and/or for neuroprotection. Several earlier stage MPO inhibitors are currently in development, such as disclosed, for example, in US 2009/0054468 published Aug. 21, 2008. Among them, BHV-3241 is a first-in-class, brain-penetrant, irreversible myeloperoxidase (MPO) inhibitor that is being developed for the treatment of multiple system atrophy (MSA) and amyotrophic lateral sclerosis (ALS). However, certain MPO inhibitors may have pharmacokinetic properties that render them challenging to prepare in oral and/or parenteral dosage forms. Enhancing the oral bioavailability, modifying the pharmacokinetic profile or increasing the aqueous solubility of such compounds would therefore be desirable.

### SUMMARY OF THE INVENTION

The present invention is directed to the treatment of MPO-related disorders, e.g., multiple system atrophy, amyotrophic lateral sclerosis, and Huntington's disease, and methods of neuroprotection, which include administering to a patient in need thereof the prodrugs, pharmaceutical compositions including the prodrugs, and kits including the pharmaceutical compositions and instructions for use.

In an aspect of the invention, there is provided a compound having one of General Formula (1):

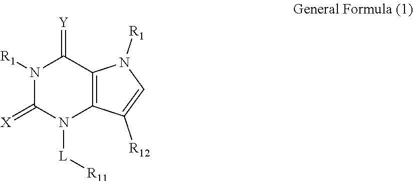

In General Formula (1):
at least one of X and Y may represent S, and the other may represent O or S;
L may represent a direct bond or C1 to C7 alkylene group as described below;
R¹¹ may be hydrogen or an unsubstituted or substituted carbocyclic, heterocyclic, or aromatic ring as described below;
R¹² may be hydrogen, a halogen, or a carbon optionally substituted with one to three halogen atoms;
each R₁ may independently be H,

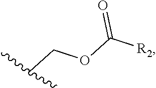

—CH₂OP(═O)(OH)₂, AA₁₋₃, C(═O)(CH₂)ₙ₁X₁AA₁₋₃, or C(═O)AA, provided that at least one R₁ may not be H; wherein
AA₁₋₃ may be a moiety consisting of 1 to 3 natural amino acids linked through a peptide bond,
n1 may be an integer of 1 to 5,
X₁ may be S, O, or NH, and
and R₂ may be —[C(R₃)₂]ₙR₄, —NR₃R₄, or —OR₄, wherein each R₃ may independently be hydrogen or C1-C10 alkyl wherein R₃ may be optionally connected to form a ring, and R₄ may be a substituted or unsubstituted C1-C20 alkyl group, a substituted or unsubstituted C2-C20 alkenyl group, a substituted or unsubstituted C2-C20 alkynyl group, a substituted or unsubstituted C1-C20 heteroalkyl group, a substituted or unsubstituted C2-C20 heteroalkenyl group, a substituted or unsubstituted C2-C20 heteroalkynyl group, a substituted or unsubstituted C3-C20 cycloalkyl group, a substituted or unsubstituted C3-C20 heterocycloalkyl group, a substituted or unsubstituted C6-C20 aryl group, or a substituted or unsubstituted C1-C20 heteroaryl group, and n may be 0 or 1.

In an aspect of the invention, there is provided a pharmaceutical composition including a therapeutically effective amount of the compound in accordance with the invention described herein.

In an aspect of the invention, there is provided a method of treating multiple system atrophy in a patient in need thereof, which includes administering to the patient a pharmaceutical composition including a therapeutically effective amount of a compound in accordance with the invention described herein.

In an aspect of the invention, there is provided a method of treating Huntington's disease in a patient in need thereof, which includes administering to the patient a pharmaceutical composition including a therapeutically effective amount of a compound in accordance with the invention described herein.

In an aspect of the invention, there is provided a method for neuroprotection, which includes administering to the patient a pharmaceutical composition including a therapeutically effective amount of a compound in accordance with the invention described herein.

In an aspect of the invention, there is provided a kit for treating multiple system atrophy, the kit comprising:
(a) a pharmaceutical composition comprising a therapeutically effective amount of a compound in accordance with the invention described herein; and
(b) instructions for administering the pharmaceutical composition.

In an aspect of the invention, there is provided a kit for treating Huntington's disease, the kit comprising:
(a) a pharmaceutical composition comprising a therapeutically effective amount of a compound in accordance with the invention described herein; and
(b) instructions for administering the pharmaceutical composition.

In an aspect of the invention, there is provided a kit for neuroprotection, the kit comprising:
(a) a pharmaceutical composition comprising a therapeutically effective amount of a compound in accordance with the invention described herein; and
(b) instructions for administering the pharmaceutical composition.

### DETAILED DESCRIPTION OF THE INVENTION

The following detailed description is provided to aid those skilled in the art in practicing the present invention. Those of ordinary skill in the art may make modifications and variations in the embodiments described herein without departing from the spirit or scope of the present disclosure. Unless otherwise defined, all technical and scientific terms used herein have the same meaning as commonly understood by one of ordinary skill in the art to which this disclosure belongs. The terminology used in the description is for describing particular embodiments only and is not intended to be limiting. Terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

As used in this application, except as otherwise expressly provided herein, each of the following terms shall have the meaning set forth below. Additional definitions are set forth throughout the application. In instances where a term is not specifically defined herein, that term is given an art-recognized meaning by those of ordinary skill applying that term in context to its use in describing the present invention.

The articles “a” and “an” refer to one or to more than one (i.e., to at least one) of the grammatical object of the article unless the context clearly indicates otherwise. By way of example, “an element” means one element or more than one element.

The term “or” means “and/or.” It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

The term “about” refers to a value or composition that is within an acceptable error range for the particular value or composition as determined by one of ordinary skill in the art, which will depend in part on how the value or composition is measured or determined, i.e., the limitations of the measurement system. For example, “about” can mean within 1 or more than 1 standard deviation per the practice in the art. Alternatively, “about” can mean a range of up to 10% or 20% (i.e., ±10% or ±20%). For example, about 3 mg can include any number between 2.7 mg and 3.3 mg (for 10%) or between 2.4 mg and 3.6 mg (for 20%). Furthermore, particularly with respect to biological systems or processes, the terms can mean up to an order of magnitude or up to 5-fold of a value. When particular values or compositions are provided in the application and claims, unless otherwise stated, the meaning of “about” should be assumed to be within an acceptable error range for that particular value or composition.

The term “administering” refers to the physical introduction of a composition comprising a therapeutic agent to a subject, using any of the various methods and delivery systems known to those skilled in the art. Administering can also be performed, for example, once, a plurality of times, and/or over one or more extended periods and can be a therapeutically effective dose or a subtherapeutic dose.

The term “AUC” (area under the curve) refers to a total amount of drug absorbed or exposed to a subject. Generally, AUC may be obtained from mathematical method in a plot of drug concentration in the subject over time until the concentration is negligible. The term “AUC” (area under the curve) could also refer to partial AUC at specified time intervals.

The term “Cₘₐₓ” refers to a maximum concentration of a drug in blood, serum, a specified compartment or test area of a subject between administration of a first dose and administration of a second dose. The term Cₘₐₓ could also refer to dose normalized ratios if specified.

The term “dosing interval,” refers to the amount of time that elapses between multiple doses of a formulation disclosed herein being administered to a subject. Dosing interval can thus be indicated as ranges.

The term “dosing frequency” refers to the frequency of administering doses of a formulation disclosed herein in a given time. Dosing frequency can be indicated as the number of doses per a given time, e.g., once a week or once in two weeks.

The terms “in combination with” and “in conjunction with” refer to administration of one treatment modality in addition to another treatment modality. As such, “in combination with” or “in conjunction with” refers to administration of one treatment modality before, during, or after administration of the other treatment modality to the subject.

The term “pharmaceutically acceptable salt” refers to a salt form of one or more of the compounds or prodrugs described herein which are presented to increase the solubility of the compound in the gastric or gastroenteric juices of the patient's gastrointestinal tract in order to promote dissolution and the bioavailability of the compounds. Pharmaceutically acceptable salts include those derived from pharmaceutically acceptable inorganic or organic bases and acids, where applicable. Suitable salts include those derived from alkali metals such as potassium and sodium, alkaline earth metals such as calcium, magnesium and ammonium salts, among numerous other acids and bases well known in the pharmaceutical art.

The terms “subject” and “patient” refer any human or non-human animal. The term “non-human animal” includes, but is not limited to, vertebrates such as non-human primates, sheep, dogs, and rodents such as mice, rats and guinea pigs. In some embodiments, the subject is a human. The terms, “subject” and “patient” are used interchangeably herein.

The terms “effective amount”, “therapeutically effective amount”, “therapeutically effective dosage” and “therapeutically effective dose” of an agent (also sometimes referred to herein as a “drug”) refers to any amount of the agent that, when used alone or in combination with another agent, protects a subject against the onset of a disease or promotes disease regression evidenced by a decrease in severity of disease symptoms, an increase in frequency and duration of disease symptom-free periods, or a prevention of impairment or disability due to the disease affliction. The therapeutically effective amount of an agent can be evaluated using a variety of methods known to the skilled practitioner, such as in human subjects during clinical trials, in animal model systems predictive of efficacy in humans, or by assaying the activity of the agent in in vitro assays.

The term “Tₘₐₓ” refers to a time or period after administration of a drug when the maximum concentration (Cₘₐₓ) is reached in blood, serum, a specified compartment or test area of a subject.

The term “treatment” refers to any treatment of a condition or disease in a subject and may include: (i) preventing the disease or condition from occurring in the subject which may be predisposed to the disease but has not yet been diagnosed as having it; (ii) inhibiting the disease or condition, i.e., arresting its development; relieving the disease or condition, i.e., causing regression of the condition; or (iii) ameliorating or relieving the conditions caused by the disease, i.e., symptoms of the disease. Treatment could be used in combination with other standard therapies or alone. Treatment or “therapy” of a subject also includes any type of intervention or process performed on, or the administration of an agent to, the subject with the objective of reversing, alleviating, ameliorating, inhibiting, slowing down or preventing the onset, progression, development, severity or recurrence of a symptom, complication or condition, or biochemical indicia associated with a disease.

With respect to multiple system atrophy and Huntington's disease, “treatment” is an approach for obtaining beneficial or desired clinical results. For purposes of this invention, beneficial or desired clinical results include, but are not limited to, one or more of the following: improvement in any aspect of a major symptom including lessening severity, alleviation of major symptom intensity, and other associated symptoms, reducing frequency of recurrence, increasing the quality of life of those suffering from the symptom, and decreasing dose of other medications required to treat the symptom.

An “alkyl group” as used herein refers to a linear or branched aliphatic hydrocarbon monovalent group having a specified number of carbon atoms. Non-limiting examples of the “alkyl group” are a methyl group, an ethyl group, a propyl group, an iso-butyl group, a sec-butyl group, a tert-butyl group, a pentyl group, an iso-amyl group, and a hexyl group. An “alkylene group” as used herein refers to a divalent group formed by abstraction of hydrogen from the alkyl group.

An “alkenyl group” as used herein refers to a hydrocarbon monovalent group containing at least one carbon-carbon double bond in the middle or at the terminal thereof and having a specified number of carbon atoms. Non-limiting examples thereof are an ethenyl group, a propenyl group, and a butenyl group. An “alkenylene group” as used herein refers to a divalent group formed by abstraction of hydrogen from the alkenyl group.

An “alkynyl group” as used herein refers to a hydrocarbon monovalent group containing at least one carbon-carbon triple bond in the middle or at the terminal thereof and having a specified number of carbon atoms. Non-limiting examples thereof are an ethynyl group and a propynyl group. An “alkynylene group” as used herein refers to a divalent group formed by abstraction of hydrogen from the alkynyl group.

A “heteroalkyl group” as used herein refers to an alkyl group as defined above, in which at least one carbon atom is replaced with a heteroatom selected from N, O, P, and S or in which at least one carbon atom is replaced with a group containing at least one of the above heteroatoms. Non-limiting examples thereof are a methoxyethyl group and a dimethylaminoethyl group. A “heteroalkylene group” as used herein refers to a divalent group formed by abstraction of hydrogen from the heteroalkyl group.

A “heteroalkenyl group” as used herein refers to an alkenyl group as defined above, in which at least one carbon atom is replaced with a heteroatom selected from N, O, P, and S. Non-limiting examples thereof are a methoxybutenyl group and a dimethylaminobutenyl group. A “heteroalkenylene group” as used herein refers to a divalent group formed by abstraction of hydrogen from the heteroalkenyl group.

A “heteroalkynyl group” as used herein refers to an alkynyl group as defined above, in which at least one carbon atom is replaced with a heteroatom selected from N, O, P, and S. Non-limiting examples thereof are a methoxybutynyl group and a dimethylaminobutynyl group. A “heteroalkynylene group” as used herein refers to a divalent group formed by abstraction of hydrogen from the heteroalkynyl group.

A “cycloalkyl group” as used herein refers to a monovalent hydrocarbon monocyclic group having a specified number of carbon atoms. Non-limiting examples thereof are a cyclopropyl group, a cyclobutyl group, a cyclopentyl group, a cyclohexyl group, and a cycloheptyl group. A “cycloalkylene group” as used herein refers to a divalent group formed by abstraction of hydrogen from the cycloalkyl group.

A “heterocycloalkyl group” as used herein refers to a monovalent monocyclic group having at least one heteroatom selected from N, O, P, and S as a ring-forming atom and specified number of carbon atoms. Non-limiting examples thereof are a tetrahydrofuranyl group, and a tetrahydrothiophenyl group. A “heterocycloalkylene group” as used herein refers to a divalent group formed by abstraction of hydrogen from the heterocycloalkyl group.

An “aryl group” as used herein refers to a monovalent group having a carbocyclic aromatic system having a specified number of carbon atoms. Non-limiting examples of the aryl group are a phenyl group, a naphthyl group, an anthracenyl group, a phenanthrenyl group, a pyrenyl group, and a chrysenyl group. When the aryl group includes two or more rings, the rings may be fused to each other. An “arylene group” as used herein refers to a divalent group formed by abstraction of hydrogen from the aryl group.

A “heteroaryl group” as used herein refers to a monovalent carbocyclic aromatic system having at least one heteroatom selected from N, O, P, and S as a ring-forming atom and a specified number of carbon atoms. Non-limiting examples of the heteroaryl group are a pyridinyl group, a pyrimidinyl group, a pyrazinyl group, a pyridazinyl group, a triazinyl group, a quinolinyl group, and an isoquinolinyl group. When the heteroaryl group includes two or more rings, the rings may be fused to each other. A “heteroarylene group” as used herein refers to a divalent group formed by abstraction of hydrogen from the heteroaryl group.

At least one of substituents of the substituted alkyl group, substituted alkenyl group, substituted alkynyl group, substituted cycloalkyl group, substituted heterocycloalkyl group, substituted aryl group, and substituted heteroaryl group may be selected from:

deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C1-C10 alkyl group, a C2-C10 alkenyl group, a C2-C10 alkynyl group, and C1-C10 alkoxy group;
a C1-C10 alkyl group, a C2-C10 alkenyl group, a C2-C10 alkynyl group, and C1-C10 alkoxy group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C3-C10 cycloalkyl group, a C1-C10 heterocycloalkyl group, a C3-C10 cycloalkenyl group, a C1-C10 heterocycloalkenyl group, a C6-C20 aryl group, a C6-C20 aryloxy group, a C6-C20 arylthio group, and a C1-C20 heteroaryl group;
a C3-C10 cycloalkyl group, a C1-C10 heterocycloalkyl group, a C3-C10 cycloalkenyl group, a C1-C10 heterocycloalkenyl group, a C6-C20 aryl group, a C6-C20 aryloxy group, a C6-C20 arylthio group, and a C1-C20 heteroaryl group; and
a C3-C10 cycloalkyl group, a C1-C10 heterocycloalkyl group, a C3-C10 cycloalkenyl group, a C1-C10 heterocycloalkenyl group, a C6-C20 aryl group, a C6-C20 aryloxy group, a C6-C20 arylthio group, and a C1-C20 heteroaryl group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C1-C10 alkyl group, a C2-C10 alkenyl group, a C2-C10 alkynyl group, and C1-C10 alkoxy group, a C3-C10 cycloalkyl group, a C1-C10 heterocycloalkyl group, a C3-C10 cycloalkenyl group, a C1-C10 heterocycloalkenyl group, a C6-C20 aryl group, a C6-C20 aryloxy group, a C6-C20 arylthio group, and a C1-C20 heteroaryl group.

For example, at least one of substituents of the substituted alkyl group, substituted alkenyl group, substituted alkynyl group, substituted alkoxy group, substituted cycloalkyl group, substituted heterocycloalkyl group, substituted aryl group, and substituted C1-C30 heteroaryl group may be selected from:
deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C1-C10 alkyl group, a C2-C10 alkenyl group, a C2-C10 alkynyl group, and C1-C10 alkoxy group;
a C1-C10 alkyl group, a C2-C10 alkenyl group, a C2-C10 alkynyl group, and C1-C10 alkoxy group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C3-C10 cycloalkyl group, a C1-C10 heterocycloalkyl group, a C3-C10 cycloalkenyl group, a C1-C10 heterocycloalkenyl group, a C6-C20 aryl group, a C6-C20 aryloxy group, a C6-C20 arylthio group, and a C1-C20 heteroaryl group;
a phenyl group, a pentalenyl group, an indenyl group, a naphthyl group, an azulenyl group, a heptalenyl group, an indacenyl group, an acenaphthyl group, a fluorenyl group, a spiro-fluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pyrrolyl group, an imidazolyl group, a pyrazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a carbazolyl group, a phenanthridinyl group, an acridinyl group, a phenanthrolinyl group, a phenazinyl group, a benzoxazolyl group, a benzoimidazolyl group, a furanyl group, a benzofuranyl group, a thiophenyl group, a benzothiophenyl group, a thiazolyl group, an isothiazolyl group, a benzothiazolyl group, an isoxazolyl group, an oxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyrimidinyl group and an imidazopyridinyl group, each substituted with at least one selected from a cyclopentyl group, a cyclohexyl group, a cycloheptyl group, a cyclooctyl group, a cyclopentenyl group, a cyclohexenyl group, a cycloheptenyl group, a phenyl group, a naphthyl group, an anthracenyl group, a pyrenyl group, a phenanthrenyl group, a fluorenyl group, a carbazolyl group, a benzocarbazolyl group, a dibenzocarbazolyl group, a pyridinyl group, a pyrimidinyl group, a pyrazinyl group, a pyridazinyl group, a triazinyl group, a quinolinyl group, an isoquinolinyl group, a phthalazinyl group, a quinoxalinyl group, a cinnolinyl group, and a quinazolinyl group; and
a phenyl group, a pentalenyl group, an indenyl group, a naphthyl group, an azulenyl group, a heptalenyl group, an indacenyl group, an acenaphthyl group, a fluorenyl group, a spiro-fluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pyrrolyl group, an imidazolyl group, a pyrazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a carbazolyl group, a phenanthridinyl group, an acridinyl group, a phenanthrolinyl group, a phenazinyl group, a benzoxazolyl group, a benzoimidazolyl group, a furanyl group, a benzofuranyl group, a thiophenyl group, a benzothiophenyl group, a thiazolyl group, an isothiazolyl group, a benzothiazolyl group, an isoxazolyl group, an oxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyrimidinyl group, and an imidazopyridinyl group, each substituted with at least one selected from a cyclopentyl group, a cyclohexyl group, a cycloheptyl group, a cyclooctyl group, a cyclopentenyl group, a cyclohexenyl group, a cycloheptenyl group, a phenyl group, a naphthyl group, an anthracenyl group, a pyrenyl group, a phenanthrenyl group, a fluorenyl group, a carbazolyl group, a benzocarbazolyl group, a dibenzocarbazolyl group, a pyridinyl group, a pyrimidinyl group, a pyrazinyl group, a pyridazinyl group, a triazinyl group, a quinolinyl group, an isoquinolinyl group, a phthalazinyl group, a quinoxalinyl group, a cinnolinyl group, and a quinazolinyl group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C1-C10 alkyl group, a C2-C10 alkenyl group, a C2-C10 alkynyl group, and C1-C10 alkoxy group, a C3-C10 cycloalkyl group, a C1-C10 heterocycloalkyl group, a C3-C10 cycloalkenyl group, a C1-C10 heterocycloalkenyl group, a C6-C20 aryl group, a C6-C20 aryloxy group, a C6-C20 arylthio group, and a C1-C20 heteroaryl group.

When a group containing a specified number of carbon atoms is substituted with any of the groups listed in the preceding paragraph, the number of carbon atoms in the resulting “substituted” group is defined as the sum of the carbon atoms contained in the original (unsubstituted) group and the carbon atoms (if any) contained in the substituent. For example, when the term “substituted C1-C20 alkyl” refers to a C1-C20 alkyl group substituted with C6-C20 aryl group, the total number of carbon atoms in the resulting aryl substituted alkyl group is C7-C40.

The starting materials useful for making the compounds and pharmaceutical compositions of the present invention are readily commercially available or can be prepared by those skilled in the art.

The subject compounds are useful as prodrugs in a method of inhibiting MPO in a patient, such as mammal in need of such inhibition, wherein the method comprises the administration of an effective amount of the compound. Embodiments of the present invention are directed to the use of the compounds disclosed herein as prodrugs of MPO inhibitors. In addition to primate, especially humans, a variety of other mammals can be treated according to the method of the present invention.

An embodiment of the present invention provides a compound having General Formula (1):

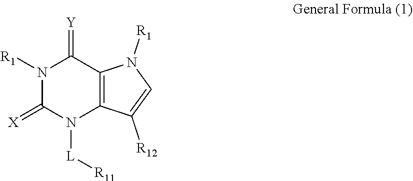

In General Formula (1), at least one of X and Y may represent S, and the other may represent O or S. L may represent a direct bond or C1 to C7 alkylene group, wherein the C1 to C7 alkylene group may optionally incorporate a heteroatom selected from O, S(O)ₙ (wherein n represents an integer 0, 1, or 2), and NR¹⁶. The C1 to C7 alkylene group may optionally incorporate one or two carbon-carbon double bonds, and the C1 to C7 alkylene group may be optionally substituted with one or more substituents selected independently from OH, a halogen, CN and NR¹⁴R¹⁵, a C1 to C6 alkyl group and a C1 to C6 alkoxy group, wherein the C1 to C6 alkoxy group optionally incorporates a carbonyl group adjacent to the oxygen.

In some embodiments, R¹¹ may be hydrogen. In other embodiments, R¹¹ may be a saturated or partially unsaturated 3 to 7 membered ring optionally incorporating one or two heteroatoms selected independently from O, N, and S, and optionally incorporating a carbonyl group. The ring may be optionally substituted with one or more substituents independently selected from a halogen, SO₂R⁹, SO₂NR⁹R¹⁰, OH, a C1 to C7 alkyl group, a C1 to C7 alkoxy group, CN, CONR²²R²³, NR²²COR²³ and COR²³, wherein the C1 to C7 alkoxy group may be further substituted with a C1 to C6 alkoxy group and may optionally incorporate a carbonyl group adjacent to the oxygen. The C1 to C7 alkyl group may be further substituted with hydroxy or a C1 to C6 alkoxy group, and the C1 to C7 alkyl group or the C1 to C6 alkoxy group may optionally incorporate a carbonyl group adjacent to the oxygen or at any position in the C1 to C7 alkyl group.

In some other embodiments, R¹¹ may be an aromatic ring system selected from phenyl, biphenyl, naphthyl or a monocyclic or bicyclic heteroaromatic ring structure containing 1 to 3 heteroatoms independently selected from O, N, and S. The aromatic ring system may be optionally substituted with one or more substituents independently selected from a halogen, SO₂R⁹, SO₂NR⁹R¹⁰, OH, a C1 to C7 alkyl group, a C1 to C7 alkoxy group, CN, CONR²²R²³, NR²²COR²³ and COR²³, wherein the C1 to C7 alkoxy group may optionally be further substituted with a C1 to C6 alkoxy group, and the C1 to C6 alkoxy group may optionally incorporate a carbonyl group adjacent to the oxygen. The C1 to C7 alkyl group may be further substituted with hydroxy or a C1 to C6 alkoxy group, and the C1 to C7 alkyl group and the C1 to C6 alkoxy group may optionally incorporate a carbonyl group adjacent to the oxygen or at any position in the alkyl group.

R¹² may represent hydrogen, a halogen, or a carbon optionally substituted with one to three halogen atoms.

At each occurrence, R⁹, R¹⁰, R¹⁴, R¹⁵, R¹⁶, R²², and R²³ may independently represent hydrogen, a C1 to C6 alkyl group or a C1 to C6 alkoxy group, wherein the alkoxy group may optionally incorporate a carbonyl group adjacent to the oxygen. The C1 to C6 alkyl group may be further substituted with a halogen, a C1 to C6 alkoxy group, CHO, a C2 to C6 alkanoyl group, OH, CONR⁷R⁸, and NR⁷COR⁸. In some embodiments, the groups NR⁹R¹⁰, NR¹⁴R¹⁵, and NR²²R²³ may each independently represent a 5 to 7 membered saturated azacyclic ring optionally incorporating one additional heteroatom selected from O, S and NR¹¹, wherein the azacyclic ring may be optionally substituted with a halogen, a C1 to C6 alkoxy group, CHO, a C2 to C6 alkanoyl group, OH, CONR⁷R⁸ and NR⁷COR⁸. At each occurrence, R⁷, R⁸ and R¹¹ may independently represent hydrogen or a C1 to C6 alkyl group, or the group NR⁷R⁸ may represent a 5- to 7-membered saturated azacyclic ring may optionally incorporate one additional heteroatom selected from O, S, and NR¹¹.

In some embodiments, each R₁ may independently be H or

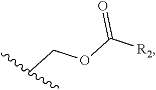

provided that at least one R₁ is

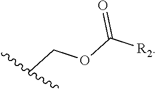

When each R₁ is

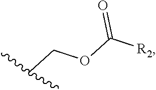

R₂ may be optionally connected to form a ring.

R₂ may be —[C(R₃)₂]ₙR₄, —NR₃R₄, or —OR₄. R₃ may independently be hydrogen or a C1-C10 alkyl group. When each R₃ is a C1-C10 alkyl group, R₃ may be optionally connected with a single bond to form a ring. R₄ may be a substituted or unsubstituted C1-C20 alkyl group, a substituted or unsubstituted C2-C20 alkenyl group, a substituted or unsubstituted C2-C20 alkynyl group, a substituted or unsubstituted C1-C20 heteroalkyl group, a substituted or unsubstituted C2-C20 heteroalkenyl group, a substituted or unsubstituted C2-C20 heteroalkynyl group, a substituted or unsubstituted C3-C20 cycloalkyl group, a substituted or unsubstituted C3-C20 heterocycloalkyl group, a substituted or unsubstituted C6-C20 aryl group, or a substituted or unsubstituted C1-C20 heteroaryl group. For example, R₄ may be a substituted or unsubstituted C1-C15 alkyl group, a substituted or unsubstituted C2-C15 alkenyl group, a substituted or unsubstituted C2-C15 alkynyl group, a substituted or unsubstituted C1-C15 heteroalkyl group, a substituted or unsubstituted C2-C15 heteroalkenyl group, a substituted or unsubstituted C2-C15 heteroalkynyl group, a substituted or unsubstituted C3-C15 cycloalkyl group, a substituted or unsubstituted C3-C15 heterocycloalkyl group, a substituted or unsubstituted C6-C15 aryl group, or a substituted or unsubstituted C1-C15 heteroaryl group. In another example, R₄ may be a substituted or unsubstituted C1-C10 alkyl group, a substituted or unsubstituted C2-C10 alkenyl group, a substituted or unsubstituted C2-C10 alkynyl group, a substituted or unsubstituted C1-C10 heteroalkyl group, a substituted or unsubstituted C2-C10 heteroalkenyl group, a substituted or unsubstituted C2-C10 heteroalkynyl group, a substituted or unsubstituted C3-C10 cycloalkyl group, a substituted or unsubstituted C3-C10 heterocycloalkyl group, a substituted or unsubstituted C6-C10 aryl group, or a substituted or unsubstituted C1-C10 heteroaryl group.

In formula —[C(R₃)₂]ₙR₄, n may be 0 or 1. When n is 0, moiety [C(R₃)₂] is absent, and R₂ may be the same as R₄ described above. When n is 0, examples of R₁ groups may include:

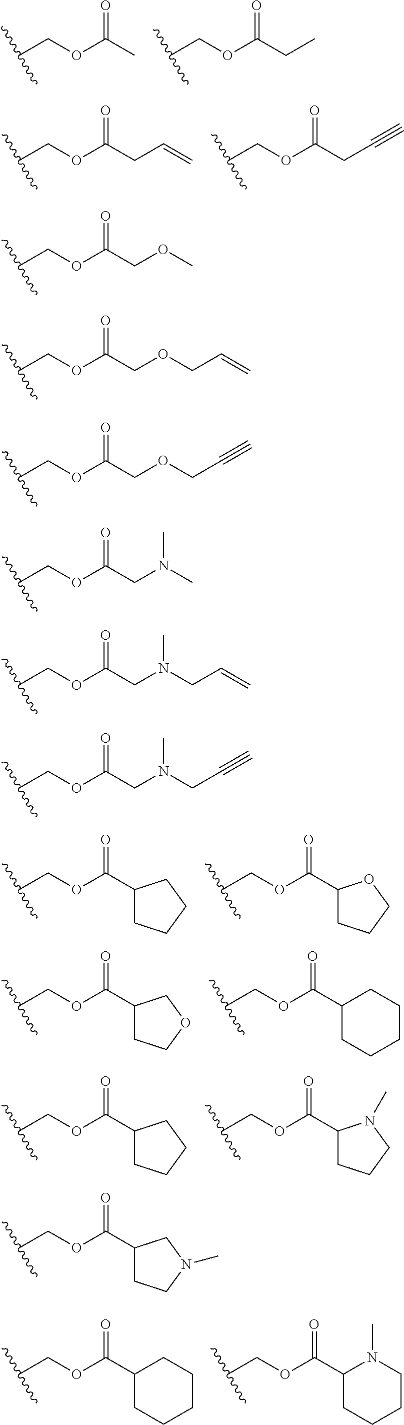

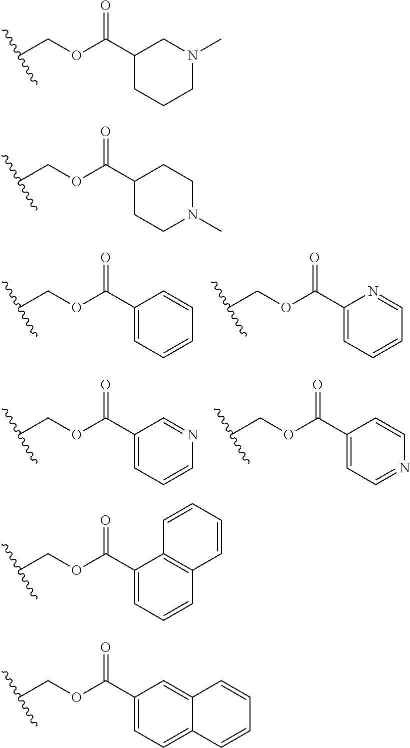

Each of the above groups may be substituted or unsubstituted.

When n in formula —[C(R₃)₂]ₙR₄ is 1, moiety [C(R₃)₂] is present. In some embodiments, each R₃ in moiety [C(R₃)₂] may be hydrogen. In other embodiments, one R₃ may be hydrogen and the other R₃ may be a C1-C10 alkyl group. In some other embodiments, each R₃ may be a C1-C10 alkyl group.

When n is 1, examples of R₁ groups may include:

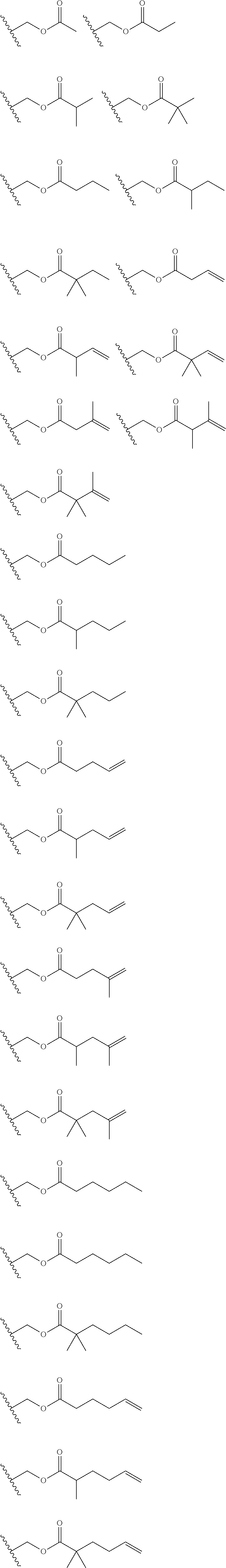

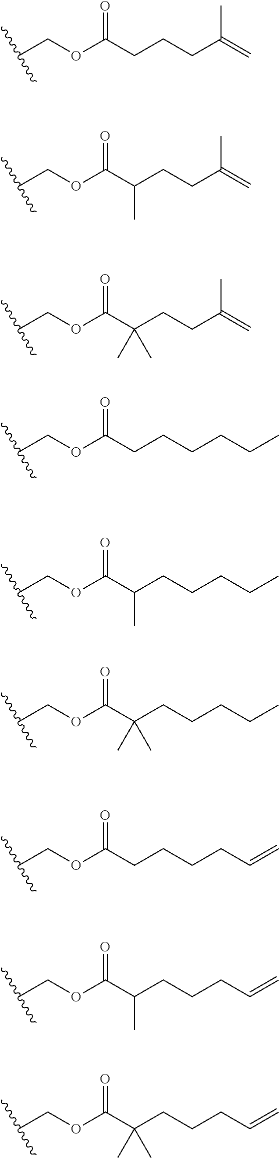

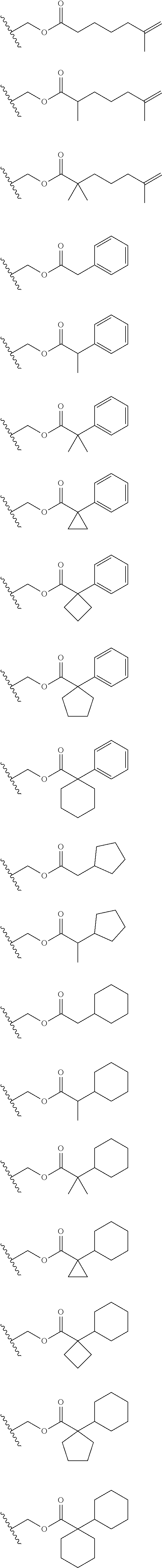

Each of the above groups may be substituted or unsubstituted.

In General Formula (1), R₂ may be —NR₃R₄, wherein R₃ and R₄ may be the same as described above. In an embodiment, R₃ and R₄ may be individual substituents. In another embodiment, R₃ and R₄ may be connected to form a ring. When R₂ is —NR₃R₄, examples of R₁ groups may include:

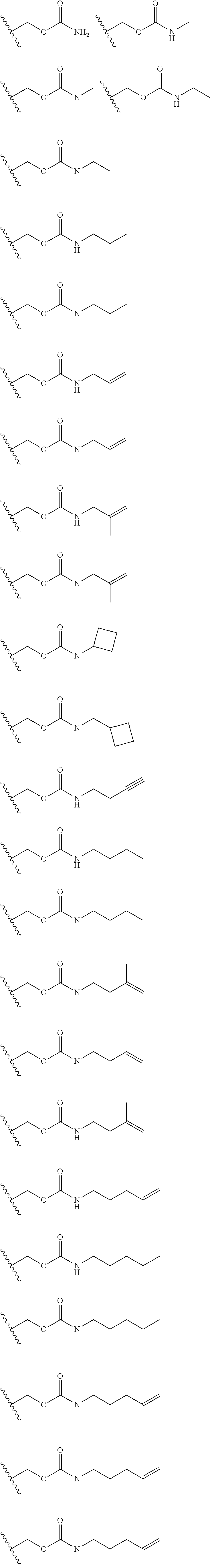

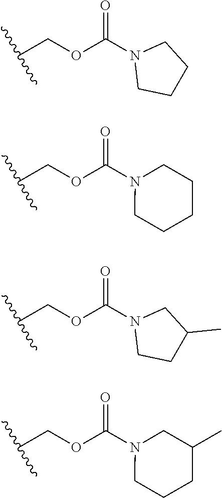

Each of the above groups may be substituted or unsubstituted.

In General Formula (1), R₂ may be —OR₄, wherein R₄ may be the same as described above. When R₂ is —OR₄, examples of R₁ groups may include:

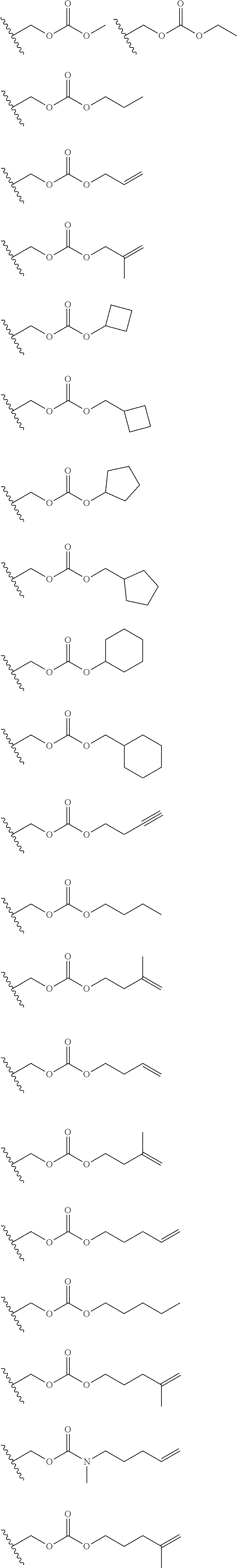

Each of the above groups may be substituted or unsubstituted.

In an embodiment, R₄ may be substituted with a natural amino acid that is linked to R₄ through an oxygen atom of a carboxylic acid group. The natural amino acid may be glycine, alanine, valine, leucine, isoleucine, serine, threonine, cysteine, cystine, methionine, aspartic acid, glutamic acid, asparagine, glutamine, lysine, hydroxylysine, arginine, histidine, phenylalanine, tyrosine, tryptophan, proline, or hydroxyproline. The natural amino acid may be attached at the terminal or internal carbon atom of the R₄ group.

In General Formula 1, R₁ may be —CH₂OP(═O)(OR^{b})₂, —CH₂OP(═O)(OR^{b})R^{a}, —CH₂OP(═O)R^{a}R^{b}, or —CH₂OP(═O)(OR^{b})—OP(═O)(OR^{b})₂. In these formulae, R^{a} and R^{b} may be independently selected from hydrogen, a substituted or unsubstituted C1-C20 alkyl group, a substituted or unsubstituted C3-C20 cycloalkyl group, a substituted or unsubstituted C6-C20 aryl group, a substituted or unsubstituted C1-C20 heteroaryl group. In an embodiment, R₁ may be —CH₂OP(═O)(OH)₂.

In some other embodiments, R₁ may independently be H or

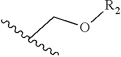

provided that at least one R₁ is

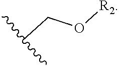

R₂ may be AA₁₋₃, C(═O)(CH₂)ₙX₁AA₁₋₃, or C(═O)AA. AA₁₋₃ may be a moiety consisting of 1 to 3 natural amino acids linked through a peptide bond. The natural amino acids may be the same as those described in connection with group R₄ above. n may be an integer of 1 to 5. X₁ may be S, O, or NH. R₃ and R₄ may each independently be H, a substituted or unsubstituted C1-C10 alkyl group, a substituted or unsubstituted C2-C10 alkenyl group, a substituted or unsubstituted C2-C10 alkynyl group, a substituted or unsubstituted C1-C10 heteroalkyl group, a substituted or unsubstituted C2-C10 heteroalkenyl group, or a substituted or unsubstituted C2-C10 heteroalkynyl group. AA may be a natural amino acid linked through a peptide bond.

General Formula (1) may include pharmaceutically acceptable salts of the compound, solvates of the compound, or solvates of salts of the compound.

Another embodiment of the present invention provides a compound having General Formula (2):

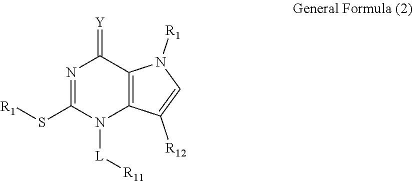

In General Formula (2), Y, L, R₁, R₁₁, and R₁₂ may be the same as that described above in connection with General Formula (1).

Another embodiment of the present invention provides a compound having General Formula (3):

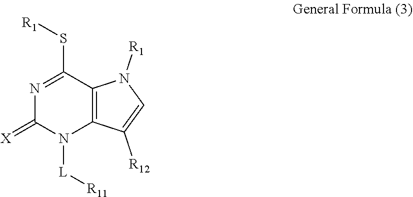

In General Formula (3), X, L, R₁, R₁₁, and R₁₂ may be the same as that described above in connection with General Formula (1).

The number of R₁ substituents in General Formulae (1), (2), and (3) can be determined by one skilled in the art, e.g., the desired degree of bioavailability. One or two R₁ groups may be present in the compound. If two R₁ groups are present, the R₁ groups may be the same or different.

In an embodiment, the compound maybe represented by Formula (I) or Formula (II):

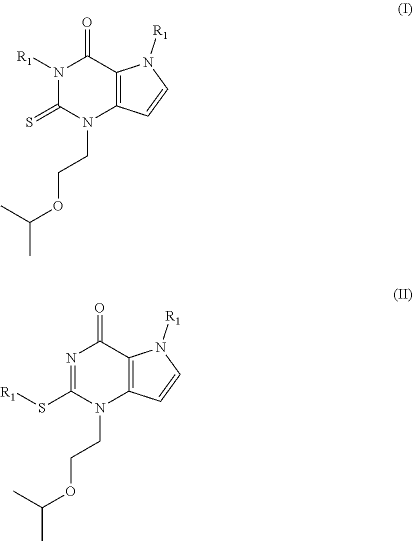

In Formulae (I) and (II),
each R₁ may independently be H or

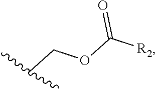

provided that at least one R₁ is

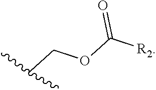

Thus, Formulae (I) and (II) require the presence of at least one group

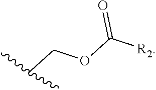

In Formulae (I) and (II), each R₁ may be

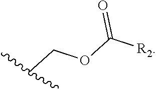

In Formulae (I) and (II), R₂ may be the same as that described in connection with General Formula (1) above.

In formula —[C(R₃)₂]ₙR₄, at least one R₃ may not be hydrogen, or each R₃ may not be hydrogen. For example, at least one R₃ may be a methyl group or each R₃ may be a methyl group. In these embodiments, R₂ may independently be —C(H)(CH₃)R₄ or —C(CH₃)₂R₄, wherein R₄ may be the same as that described above. In an embodiment, R₄ may each independently be a substituted or unsubstituted C1-C15 alkyl group, a substituted or unsubstituted C2-C15 alkenyl group, a substituted or unsubstituted C2-C15 alkynyl group, a substituted or unsubstituted C1-C15 heteroalkyl group, a substituted or unsubstituted C2-C15 heteroalkenyl group, a substituted or unsubstituted C2-C15 heteroalkynyl group, or a substituted or unsubstituted C6-C20 aryl group. In another embodiment, R₄ may each independently be a substituted or unsubstituted C1-C10 alkyl group, a substituted or unsubstituted C2-C10 alkenyl group, a substituted or unsubstituted C2-C10 alkynyl group, a substituted or unsubstituted C1-C10 heteroalkyl group, a substituted or unsubstituted C2-C10 heteroalkenyl group, a substituted or unsubstituted C2-C10 heteroalkynyl group, or a substituted or unsubstituted C6-C20 aryl group.

In Formulae (I) and (II), when each R₁ is

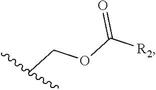

R₂ may be connected to form a ring.

When the compound has Formula (I), the compound may be represented by one of Formulae (Ia), (Ib), and (Ic):

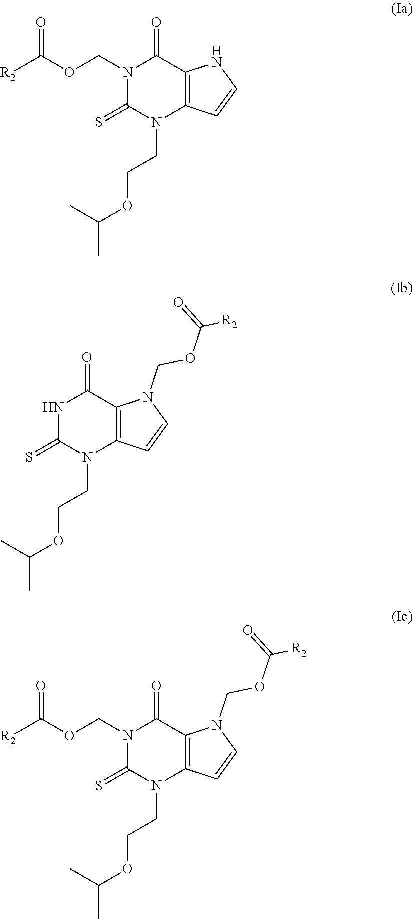

In Formulae (Ia), (Ib), and (Ic), groups R₂ may be the same as those described in connection with Formula (I).

When the compound has Formula (II), the compound may be represented by one of Formulae (IIa) and (IIb):

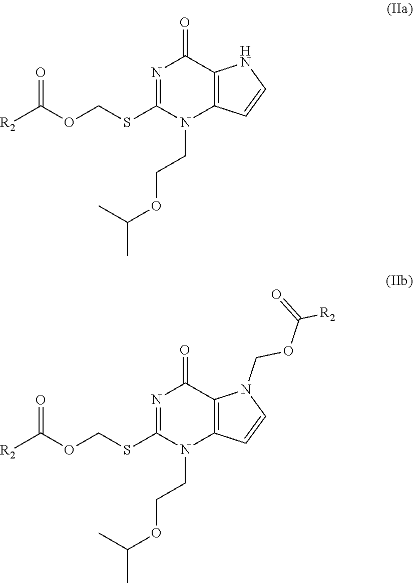

In Formulae (IIa) and (IIb), groups R₂ may be the same as those described in connection with Formula (II).

When the compound is represented by Formula (Ic), groups R₂ may be connected with a single bond to form a linking group L, as in Formula (Id):

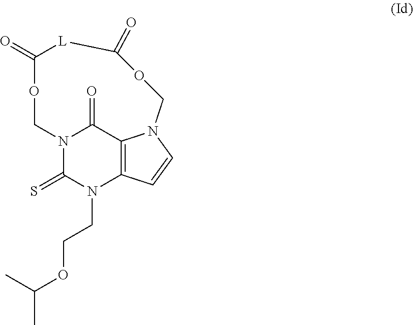

When the compound is represented by Formula (IIb), groups R₂ may be connected with a single bond to form a linking group L, as in Formula (IIc):

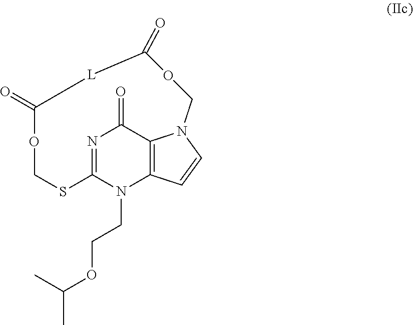

In the Formulae (Ic) and (IIb), the linking group L may be a substituted or unsubstituted C1-C10 alkylene group, a substituted or unsubstituted C2-C10 alkenylene group, a substituted or unsubstituted C2-C10 alkynylene group, a substituted or unsubstituted C1-C10 heteroalkylene group, a substituted or unsubstituted C2-C10 heteroalkenylene group, a substituted or unsubstituted C2-C10 heteroalkynylene group, a substituted or unsubstituted C3-C10 cycloalkylene group, a substituted or unsubstituted C3-C10 heterocycloalkylene group, a substituted or unsubstituted C6-C10 arylene group, or a substituted or unsubstituted C1-C10 heteroarylene group, or any combination thereof.

In the Formulae (Ic) and (IIb), the linking group L may be composed of two groups R₂ and may have the structure —R₂—R₂—. For example, the linking group may be represented by Formula (L-1):

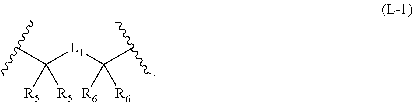

In Formula (L-1), L₁ may be a substituted or unsubstituted C1-C10 alkylene group, a substituted or unsubstituted C2-C10 alkenylene group, a substituted or unsubstituted C2-C10 alkynylene group, a substituted or unsubstituted C1-C10 heteroalkylene group, a substituted or unsubstituted C2-C10 heteroalkenylene group, a substituted or unsubstituted C2-C10 heteroalkynylene group, or a substituted or unsubstituted C6-C20 aryl group. For example, L₁ may be a substituted or unsubstituted C1-C10 alkylene group or a substituted or unsubstituted C2-C10 alkenylene group. R₅ and R₆ may each independently be hydrogen or a C1-C10 alkyl group, for example, a C1-C5 alkyl group, or a C1-C3 alkyl group.

In Formula (L-1), at least one R₅ may not hydrogen and at least one R₆ may not hydrogen. For example, at least one R₅ may be a methyl group and at least one R₆ may be a methyl group. Two geminal groups R₅ may be optionally connected with a single bond to form a ring. Also, two geminal groups R₆ may be optionally connected with a single bond to form a ring. In some embodiments, the linking group L may have one of Formulae (L-11), (L-12), (L-13), (L-14), and (L-15):

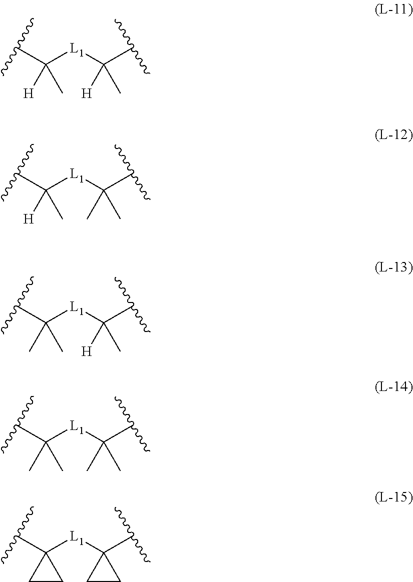

In Formulae (L-11) to (L-15) above, L₁ may be the same as that described in connection with Formula (L-1).

In an embodiment, the compound may be represented by Formula (III):

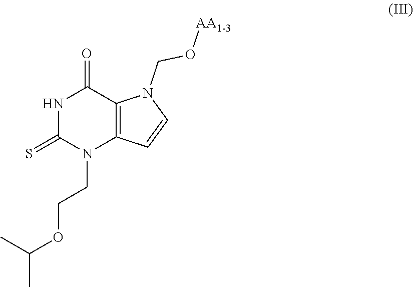

In Formula (III), AA₁₋₃ may be a moiety consisting of 1 to 3 natural amino acids linked through a peptide bond.

In another embodiment, the compound may be represented by Formula (IV):

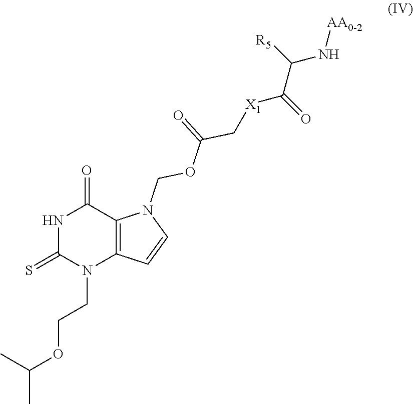

In Formula (IV), X₁ may be S, O, or NH, R₅ may be H or a side-chain group present in a natural amino acid, AA₀₋₂ may be H or a moiety consisting of 1 or 2 natural amino acids, which may be the same or different, that are linked through a peptide bond. n may be an integer of 1 to 5. The moiety AA₀₋₂ may include any combination of the natural amino acids.

In still another embodiment, the compound may be represented by Formula (V):

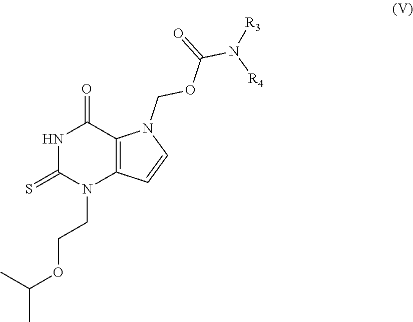

In Formula (V), R₃ and R₄ are each independently H, a substituted or unsubstituted C1-C10 alkyl group, a substituted or unsubstituted C2-C10 alkenyl group, a substituted or unsubstituted C2-C10 alkynyl group, a substituted or unsubstituted C1-C10 heteroalkyl group, a substituted or unsubstituted C2-C10 heteroalkenyl group, or a substituted or unsubstituted C2-C10 heteroalkynyl group.

In still another embodiment, the compound may be represented by Formula (VI):

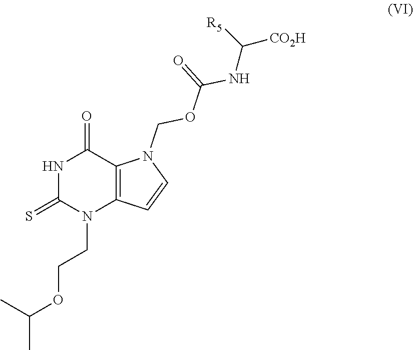

In Formula (VI), R₆ may be H or a side-chain group present in a natural amino acid. The compound may be an ester of the carboxylic acid represented by Formula (VI).

In still another embodiment, the compound may be represented by Formulae (VII), (VIII), (IX), or (X):

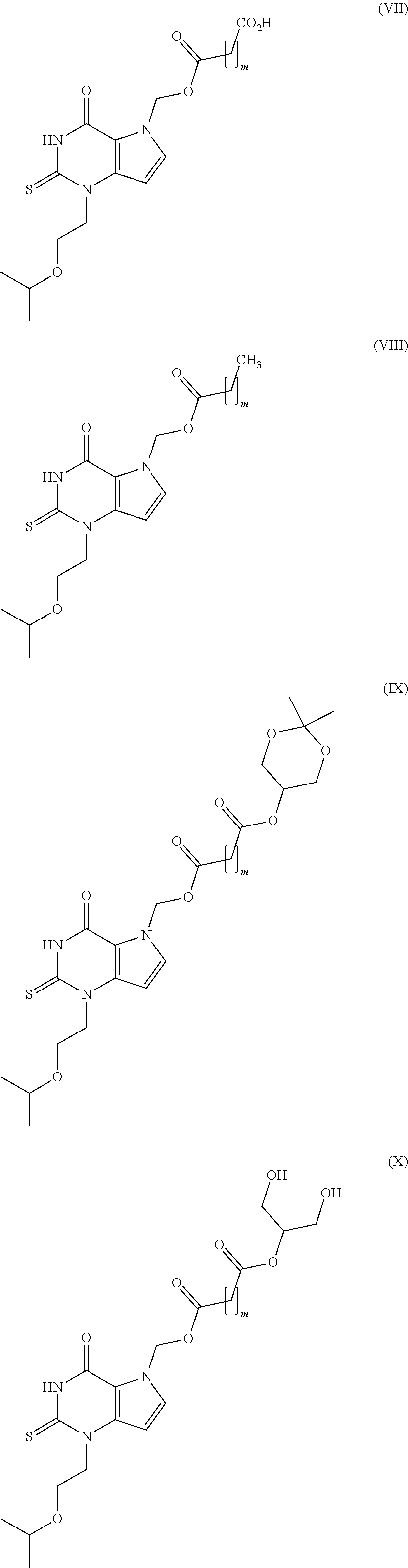

In Formulae (VII), (VIII), (IX), or (X), n may be an integer of 1 to 25. The compound may be an ester of the carboxylic acid represented by Formula (VII).

In still another embodiment, the compound may be represented by Formulae (XI) or (XII):

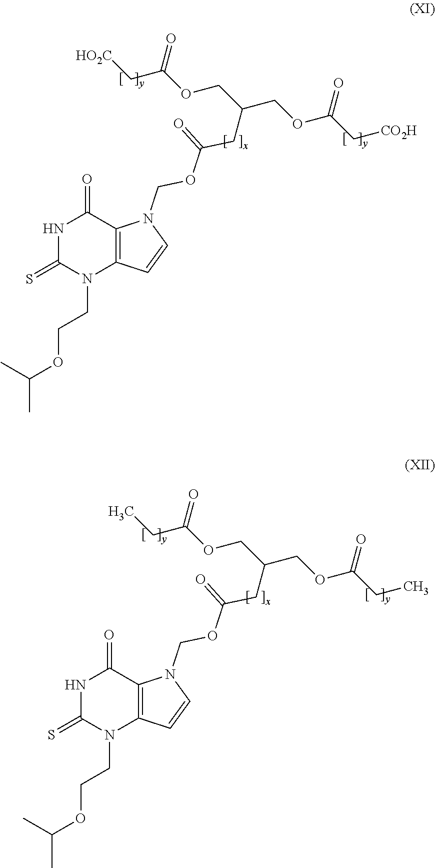

In Formulae (XI) and (XII), x and each y may independently be an integer of 1 to 25. The compound may be a mono- or di-ester of the carboxylic acid represented by Formula (XI).

In still another embodiment, the compound may be represented by Formula (XIII):

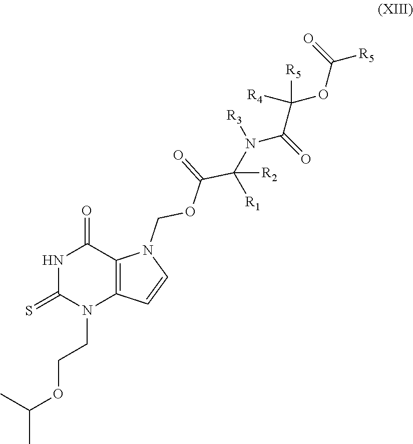

In Formula (XIII), R₁ to R₅ may each independently be hydrogen or C1-C10 alkyl, and R₆ may be hydrogen, C1-C10 alkyl, or C6-C10 aryl, wherein any of groups R₁ to R₅ may be optionally connected to form a ring.

In yet another embodiment, the compound may be represented by Formula (XIV):

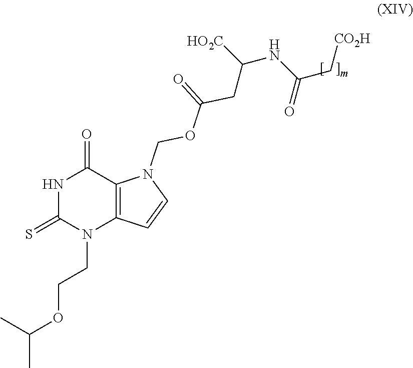

In Formula (XIV), m may be an integer of 1 to 25. The compound may be a mono- or di-ester of the carboxylic acid represented by Formula (XIV).

Non-limiting examples of the compounds according to the present invention may be:

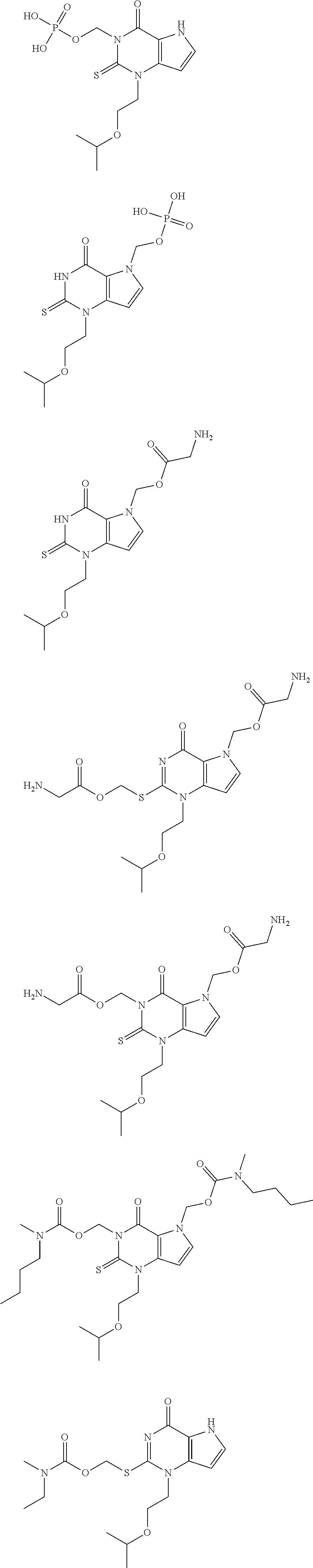

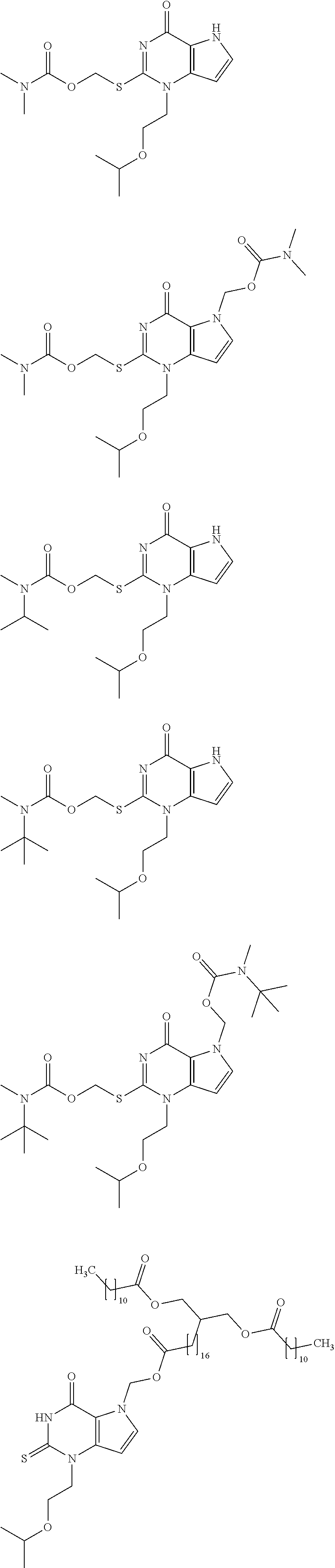

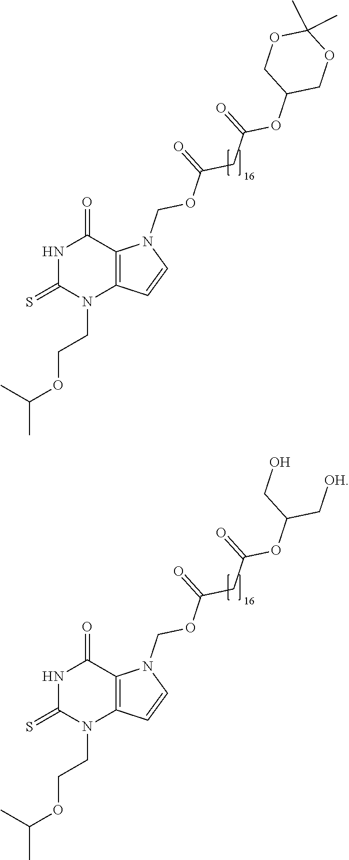

The compounds of the invention may be made in the form of pharmaceutically acceptable salts.

A person of ordinary skill in the art would readily understand how to make compounds of the present invention based on the available synthetic methods. For example, some of the compounds of the invention may be prepared according to Reaction Scheme 1:

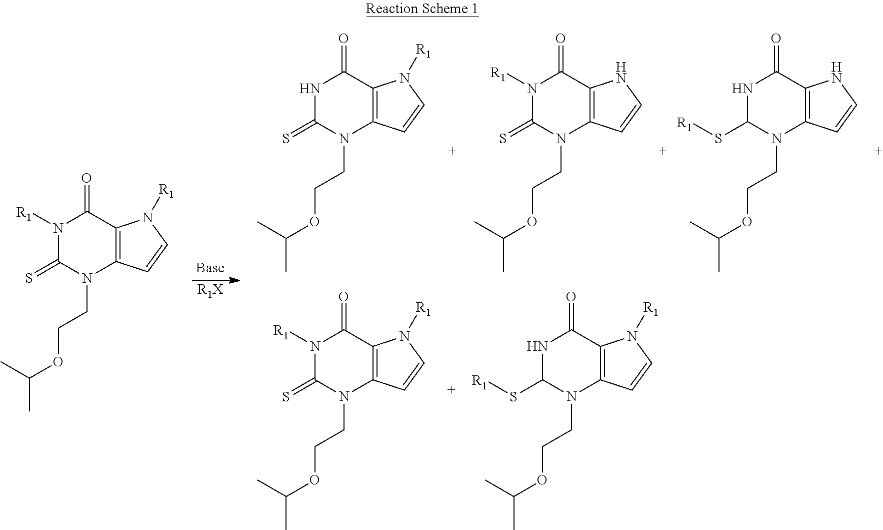

In Reaction Scheme 1, “Base” may be lithium hexamethyldisilyl amide or sodium hydride and “R₁X” may be alkyl halide or sulfonyl chloride, wherein R₁ is as described in the present application.

Other compounds of the invention may be prepared according to Reaction Scheme 2:

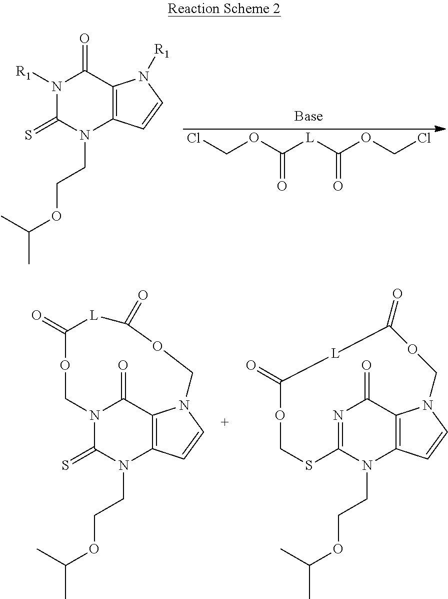

In Reaction Scheme 2, “Base” may be lithium hexamethyldisilyl amide or sodium hydride and L is as described in the present application.

The present invention is further directed to a pharmaceutical composition comprising a therapeutically effective amount of a compound. The pharmaceutical compositions of the present invention can be prepared in any suitable dosage form, but are typically prepared as tablets, capsules, powders, granules, or solutions.

The pharmaceutical compositions of the present invention comprising the compounds of the invention typically also include other pharmaceutically acceptable carriers and/or excipients such as, for example, binders, lubricants, diluents, coatings, disintegrants, barrier layer components, glidants, coloring agents, solubility enhancers, gelling agents, fillers, proteins, co-factors, emulsifiers, solubilizing agents, suspending agents, flavorants, preservatives and mixtures thereof. A skilled artisan in the art would know what other pharmaceutically acceptable carriers and/or excipients could be included in the formulations according to the invention. The choice of excipients would depend on the characteristics of the compositions and on the nature of other pharmacologically active compounds in the formulation. Appropriate excipients are known to those skilled in the art (see Handbook of Pharmaceutical Excipients, fifth edition, 2005 edited by Rowe et al., McGraw Hill) and have been utilized to yield a novel formulation with unexpected properties.

Examples of pharmaceutically acceptable carriers that may be used in preparing the pharmaceutical compositions of the present invention may include, but are not limited to, fillers such as sugars, including lactose, sucrose, mannitol, or sorbitol; cellulose preparations such as maize starch, wheat starch, rice starch, potato starch, gelatin, gum tragacanth, methyl cellulose, hydroxypropyl methyl-cellulose, sodium carboxymethylcellulose, polyvinyl-pyrrolidone (PVP), talc, calcium sulphate, vegetable oils, synthetic oils, polyols, alginic acid, phosphate buffered solutions, emulsifiers, isotonic saline, pyrogen-free water and combinations thereof. If desired, disintegrating agents may be combined as well, and exemplary disintegrating agents may be, but not limited to, cross-linked polyvinyl pyrrolidone, agar, or alginic acid or a salt thereof such as sodium alginate. In an embodiment, the flavoring agent may be selected from mint, peppermint, berries, cherries, menthol and sodium chloride flavoring agents, and combinations thereof. In an embodiment, the sweetener may be selected from sugar, sucralose, aspartame, acesulfame, neotame, and combinations thereof.

Typical routes of administering the pharmaceutical compositions of the invention include, without limitation, oral administration. The compositions may also be administered by parenteral (e.g., intramuscular, intraperitoneal, intravenous, intracerebroventricular, intracisternal injection or infusion, subcutaneous injection, or implant), by inhalation spray, nasal, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration. In addition to the treatment of warm-blooded animals the compounds of the invention are effective for use in humans.

Pharmaceutical compositions according to certain embodiments of the present invention are formulated so as to allow the active ingredients contained therein to be bioavailable upon administration of the composition to a patient. Compositions that will be administered to a subject or patient may take the form of one or more dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000).

The present invention is further directed to a method for the manufacture of a medicament for inhibition of MPO activity in humans and animals comprising combining a prodrug compound of the present invention with a pharmaceutical carrier or diluent. In general, the pharmaceutical compositions of the present invention may be manufactured in conventional methods known in the art, for example, by means of conventional mixing, dissolving, granulating, dragee-making, levigating, emulsifying, encapsulating, entrapping, lyophilizing processes and the like.

All methods include the step of bringing the active ingredient (or prodrug thereof) into association with the carrier which constitutes one or more accessory ingredients. In general, the pharmaceutical compositions are prepared by uniformly and intimately bringing the active ingredient (or prodrug thereof) into association with a liquid carrier or a finely divided solid carrier or both, and then, if necessary, shaping the product into the desired formulation. In the pharmaceutical composition, the active compound (or prodrug thereof) is included in an amount sufficient to produce the desired effect upon the process or condition of diseases. As used herein, the term “composition” is intended to encompass a product comprising the specified ingredients in the specified amounts, as well as any product which results, directly or indirectly, from combination of the specified ingredients in the specified amounts. Such term in relation to pharmaceutical composition, is intended to encompass a product comprising the prodrug of the invention, and the inert ingredient(s) that make up the carrier, as well as any product which results, directly or indirectly, from combination, complexation or aggregation of any two or more ingredients, or from dissociation of one or more ingredients, or from other types of reactions or interactions of one or more of the ingredients. Accordingly, the pharmaceutical compositions of the present invention encompass any composition made by admixing a compound of the present invention and a pharmaceutically acceptable carrier. By “pharmaceutically acceptable” it is meant the carrier, diluent or excipient must be compatible with the other ingredients of the formulation and not deleterious to the recipient thereof.

Preferably, the pharmaceutical compositions containing the active ingredient (or prodrug thereof) may be in a form suitable for oral use, for example, as tablets, troches, lozenges, aqueous or oily suspensions, dispersible powders or granules, emulsions, solutions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia; and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. No. 4,256,108 published Mar. 17, 1981; U.S. Pat. No. 4,160,452 published Jul. 10, 1979; and U.S. Pat. No. 4,265,874 published May 5, 1981; to form osmotic therapeutic tablets for control release. Oral tablets may also be formulated for immediate release, such as fast melt tablets or wafers, rapid dissolve tablets or fast dissolve films.

Formulations for oral use may also be presented as hard gelatin capsules wherein the active ingredient (or prodrug thereof) is mixed with an inert solid diluent, for example, calcium carbonate, calcium phosphate or kaolin, or as soft gelatin capsules wherein the active ingredient (or prodrug thereof) is mixed with water or an oil medium, for example peanut oil, liquid paraffin, or olive oil.

Aqueous suspensions contain the active materials (or prodrugs thereof) in admixture with excipients suitable for the manufacture of aqueous suspensions. Such excipients are suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending the active ingredient (or prodrug thereof) in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient (or prodrug thereof) in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents.

The compounds of the present invention may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical use, creams, ointments, jellies, solutions or suspensions and the like, containing the compounds of the present invention are used. Similarly, transdermal patches may also be used for topical administration.

The pharmaceutical composition and method of the present invention may further include other therapeutically active compounds (or prodrug thereof) as noted herein which are usually applied in the treatment of the above mentioned pathological conditions.

In another embodiment, the invention is directed to a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. In addition, fatty acids such as oleic acid find use in the preparation of injectables.

The invention is also directed to a therapeutically effective intravenous formulation of the compounds of the invention, which is solution stable and isotonic with human blood. The intravenous formulation preferably can be packaged in plastic or glass, and meets government and compendial (USP in the US) particulate standards, and can be used as effective therapeutic agents.

The intravenous formulation may contain a buffer which can maintain the pH of the intravenous formulation within a desirable range. The buffering agent may maintain the intravenous formulation in an acceptable particulate profile for storage and subsequent use.

Pharmaceutical injectable formulations (such as subcutaneous formulations) will generally include a therapeutically effective amount of a compound of the invention, in addition to one or more pharmaceutically acceptable excipients. The compositions are advantageously prepared together with liquid inert carriers, such as water. Suitable liquid excipients/carriers are Water for Injection (US Pharmacopeia) and saline solution. The solution should be pyrogen-free, and also should be absent of particulate matter. Limits for the amount of particulate matter (i.e., extraneous, mobile undissolved substances, other than gas bubbles) which may be found in IV fluids are defined in the US Pharmacopeia.

Other suitable excipients and other additives include solvents such as ethanol, glycerol, propylene glycol, and mixtures thereof; stabilizers such as EDTA (ethylene diamine tetraacetic acid), citric acid, and mixtures thereof; antimicrobial preservatives, such as benzyl alcohol, methyl paraben, propyl paraben, and mixtures thereof; buffering agents, such as citric acid/sodium citrate, potassium hydrogen tartrate, sodium hydrogen tartrate, acetic acid/sodium acetate, maleic acid/sodium maleate, sodium hydrogen phthalate, phosphoric acid/potassium dihydrogen phosphate, phosphoric acid/disodium hydrogen phosphate, and mixtures thereof; tonicity modifiers, such as sodium chloride, mannitol, dextrose, and mixtures thereof; fluid and nutrient replenishes such as synthetic amino acids, dextrose, sodium chloride, sodium lactate, Ringer's solution, and other electrolyte solutions.

The buffer system is generally a mixture of a weak acid and a soluble salt thereof, e.g., sodium citrate/citric acid; or the monocation or dication salt of a dibasic acid, e.g., potassium hydrogen tartrate; sodium hydrogen tartrate, phosphoric acid/potassium dihydrogen phosphate, and phosphoric acid/disodium hydrogen phosphate. The amount of buffer system used is dependent on the desired pH and the amount of the compound of the invention. The choice of appropriate buffer and pH of a formulation, depending on solubility of the drug to be administered, is readily made by a person having ordinary skill in the art.

In an embodiment, the injectable formulation may be suitable for use with a needle-free injection device. Solid compositions are normally formulated in dosage units providing from about 1 to about 1000 mg of the active ingredient per dose. Some examples of solid dosage units are 0.1 mg, 1 mg, 10 mg, 37.5 mg, 75 mg, 100 mg, 150 mg, 300 mg, 500 mg, 600 mg and 1000 mg. Typical dose ranges in accordance with the present invention include from about 10-600 mg, 25-300 mg, 25-150 mg, 50-100 mg, 60-90 mg, and 70-80 mg. Liquid compositions are generally in a unit dosage range of 1-100 mg/mL. Some examples of liquid dosage units are 0.1 mg/mL, 1 mg/mL, 10 mg/mL, 25 mg/mL, 50 mg/mL, and 100 mg/mL.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

In some embodiments, a method may comprise administering to a subject one or more additional agent(s) simultaneously or sequentially with the compounds of the invention. The above combinations include combinations of a compound of the present invention not only with one other active compound (or prodrug thereof), but also with two or more other active compounds (or prodrugs thereof). Likewise, compounds of the present invention may be used in combination with other drugs (or prodrugs thereof) that are used in the prevention, treatment, control, amelioration, or reduction of risk of the diseases or conditions for which compounds of the present invention are useful. Such other drugs may be administered, by a route and in an amount commonly used therefor, contemporaneously or sequentially with a compound of the present invention. When a compound of the present invention is used contemporaneously with one or more other drugs, a pharmaceutical composition containing such other drugs in addition to the compound of the present invention is preferred. Accordingly, the pharmaceutical compositions of the present invention include those that also contain one or more other active ingredients (or prodrug thereof), in addition to a compound of the present invention. The weight ratio of the compound of the compound of the present invention to the other active ingredient(s) (or prodrugs thereof) may be varied and will depend upon the effective dose of each ingredient. Generally, an effective dose of each will be used. Thus, for example, when a compound of the present invention is combined with another agent, the weight ratio of the compound of the present invention to the other agent will generally range from about 1000:1 to about 1:1000, or from about 200:1 to about 1:200. Combinations of a compound of the present invention and other active ingredients (or prodrugs thereof) will generally also be within the aforementioned range, but in each case, an effective dose of each active ingredient should be used.

In such combinations the compound of the present invention and other active agents may be administered separately or in conjunction. In addition, the administration of one element may be prior to, concurrent to, or subsequent to the administration of other agent(s), via the same or different routes of administration.

In some embodiments, a therapeutic effect may be greater as compared to use of a compound of the invention or one or more additional agent(s) alone. Accordingly, a synergistic effect between a compound of the invention and the one or more additional agents may be achieved. In some embodiments, the one or more additional agent(s) may be taken by a subject prophylactically.

In an aspect, the invention also provides kits for use in the instant methods. Kits can include one or more containers comprising a pharmaceutical composition described herein and instructions for use in accordance with any of the methods described herein. Generally, these instructions comprise a description of administration of the pharmaceutical composition to treat, ameliorate or prevent multiple system atrophy, Huntington's disease, or other MPO disorder, according to any of the methods described herein. The kit may, for example, comprise a description of selecting an individual suitable for treatment based on identifying whether that individual has the disease or whether the individual is at risk of having the disease. The instructions are typically provided in the form of a package insert, or label, in accordance with the requirements of the regulatory having authority over the jurisdiction where the pharmaceutical composition is to be provided to patients.

### EXAMPLES

The following examples illustrate the invention and are not intended to limit the scope of the invention. In some examples, abbreviations are used which are known to those skilled in the art or are readily accessible from the documents cited in the examples.

### General Experimentals

1. Analytical Methods

Method A: LC/MS data were determined with a Waters Alliance 2695 HPLC/MS (Waters Symmetry C18, 4.6×75 mm, 3.5 μm) with a 2996 diode array detector from 210-400 nm. The solvent system was 5-95% acetonitrile in water (with 0.1% TFA) over nine minutes using a linear gradient, and retention times are in minutes. Mass spectrometry was performed on a Waters ZQ using electrospray in positive mode.

Method A-2: LC/MS data were determined with a Waters Alliance 2695 HPLC/MS (Waters Symmetry C18, 4.6×75 mm, 3.5 μm) with a 2996 diode array detector from 210-400 nm; the solvent system is 40-95% acetonitrile in water (with 0.1% TFA) over nine minutes using a linear gradient, and retention times are in minutes. Mass spectrometry was performed on a Waters ZQ using electrospray in positive mode.

Method A-3: LC/MS data were determined with a Waters Alliance 2695 HPLC/MS (Waters Symmetry C18, 4.6×75 mm, 3.5 μm) with a 2996 diode array detector from 210-400 nm; the solvent system is 40-95% acetonitrile in water (with 0.1% TFA) over nine minutes using a linear gradient, then holding at 95% acetonitrile in water (with 0.1% TFA) for 10 minutes. Retention times are in minutes. Mass spectrometry was performed on a Waters ZQ using electrospray in positive mode.

Method B: Preparative reversed phase HPLC was performed on a Phenomenex LUNA column (19×100 mm, C18, 5 μm) with a 10 min mobile phase gradient of 10% acetonitrile/water to 90% acetonitrile/water with 0.1% TFA as buffer using 214 and 254 nm as detection wavelengths. Injection and fraction collection were performed with a Gilson 215 liquid handling apparatus using Trilution LC software.

Method C: Preparative reversed phase HPLC was performed on a Waters Sunfire column (19×50 mm, C18, 5 μm) with a 10 min mobile phase gradient of 10% acetonitrile/water to 90% acetonitrile/30 water with 0.1% TFA as buffer using 214 and 254 nm as detection wavelengths. Injection and fraction collection were performed with a Gilson 215 liquid handling apparatus using Trilution LC software.

Method D: Preparative reversed phase HPLC was performed on a Waters Sunfire column (30×150 mm, C18, 10 μm) with a 15 min mobile phase gradient of with 0.1% TFA as buffer using 214 and 254 nm as detection wavelengths. Injection and fraction collection were performed with a Gilson 215 liquid handling apparatus using Trilution LC software.

¹H-NMR spectra were taken on a Varian 300 MHz NMR using tetramethylsilane (TMS) as internal standard (d=0.00) with peaks reported downfield from TMS.

2. Experimental Procedures and Data

### Example 1

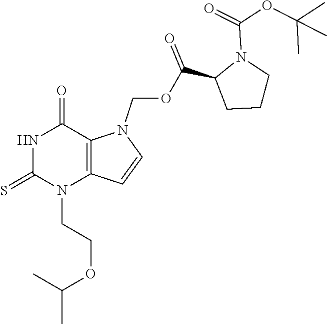 [SMILES: CC(C)OCCn1c(=S)[nH]c(=O)c2c1ccn2COC(=O)[C@@H]1CCCN1C(=O)OC(C)(C)C]

(S)-tert-Butyl (1,2,3,4-tetrahydro-1-(2-isopropoxyethyl)-4-oxo-2-thioxopyrrolo[3,2-d]pyrimidin-5-yl)methyl pyrrolidine-1,2-dicarboxylate. A solution of 2,3-dihydro-1-(2-isopropoxyethyl)-2-thioxo-1H-pyrrolo[3,2-d]pyrimidin-4(5H)-one (100 mg, 400 μmol) in THF (2.0 mL) was treated with lithium hexamethyldisilylamide (1.0 M in THF, 440 μL, 440 μmol) and stirred for 20 mins. (S)-tert-Butyl chloromethyl pyrrolidine-1,2-dicarboxylate (133 mg, 480 μmol) was added and the mixture was stirred overnight. The reaction was quenched by the addition of methanol and the solvent was removed in vacuo. Crude product was purified by normal phase chromatography (12 g column, 0-70% MeOH/DCM) where the product fractions were combined and concentrated in vacuo to provide the pure product as a solid (60 mg, 31%). ¹H NMR (300 MHz, DMSO) δ 7.41-7.65 (m, 1H), 6.32-6.48 (m, 1H), 5.99-6.32 (m, 2H), 4.47 (t, J=6.15 Hz, 2H), 4.06-4.24 (m, 1H), 3.63-3.81 (m, 2H), 3.47-3.63 (m, 1H), 3.17-3.37 (m, 3H), 2.06-2.34 (m, 1H), 1.66-1.93 (m, 3H), 1.18-1.45 (m, 9H), 0.92-1.10 (m, 6H). LC/MS method A: Rₜ=5.5 mins., (M+H)⁺=481, purity >95%. Caco-2 Pₐₚₚ=371 nm/s, SGF=99% remaining at 1 h, SIF=94% remaining at 1 h, hPlasma stability=92% remaining at 1 h.

Additional compounds prepared according to EXAMPLE 1:

| Ex- | | LCMS | | | | Caco-2 | | | human |
|---|---|---|---|---|---|---|---|---|---|
| am- | | retention | M + | LCMS | | Pₐₚₚ | | | Plasma |
| ple | Name | time | H⁺ | method | NMR | (nm/s) | SGF | SIF | stability |
| 2 | trifluoroacetic acid 1-tert-butyl 2- | 5.3 | 481 | A | 1H NMR (300 MHz, dmso) δ | | | | 73% |
| | {4-oxo-1-[2-(propan-2- | | | | 12.45 (s, 1H), 7.50-7.74 (m, | | | | remain- |
| | yloxy)ethyl]-2-sulfanylidene- | | | | 1H), 6.41-6.55 (m, 1H), 6.12- | | | | ing |
| | 1H,2H,3H,4H,5H-pyrrolo [3,2- | | | | 6.40 (m, 2H), 4.47-4.60 (m, | | | | at 1 h |
| | d]pyrimidin-5-yl}methyl(2S)- | | | | 2H), 4.10-4.33 (m, 1H), 3.78 (q, | | | | |
| | pyrrolidine-1,2-dicarboxylate | | | | J = 5.66 Hz, 2H), 3.53-3.70 (m, | | | | |
| | | | | | 1H), 3.18-3.51 (m, 2H), 2.08- | | | | |
| | | | | | 2.36 (m, 1H), 1.73-1.98 (m, | | | | |
| | | | | | 3H), 1.19-1.50 (m, 9H), 1.07 (d, | | | | |
| | | | | | J = 6.44 Hz, 6H) | | | | |
| 3 | 1-tert-butyl 2-{4-oxo-1-[2-(propan- | 5.3 | 481 | A | 1H NMR (300 MHz, dmso) δ | 419 | 84% | 89% | 99% |
| | 2-yloxy)ethyl]-2-sulfanylidene- | | | | 7.45-7.70 (m, 1H), 6.40 (d, | | remain- | remain- | remain- |
| | 1H,2H,3H,4H,5H-pyrrolo[3,2- | | | | J = 2.93 Hz, 1H), 5.95-6.34 (m, | | ing | ing | ing |
| | d]pyrimidin-5-yl} methyl (2R)- | | | | 2H), 4.36-4.59 (m, 2H), 4.04- | | at 1 h | at 1 h | at 1 h |
| | pyrrolidine-1,2-dicarboxylate | | | | 4.25 (m, 1H), 3.62-3.82 (m, | | | | |
| | | | | | 2H), 3.43-3.62 (m, 1H), 3.23- | | | | |
| | | | | | 3.39 (m, 4H), 2.03-2.31 (m, | | | | |
| | | | | | 1H), 1.67-1.93 (m, 3H), 1.14- | | | | |
| | | | | | 1.42 (m, 9H), 0.83-1.09 (m, 6H) | | | | |
| 4 | 1-benzyl 2-{4-oxo-1-[2-(propan-2- | 5.1 | 529 | A | 1H NMR (300 MHz, dmso) δ | 134 | 97% | 94% | 40% |
| | yloxy)ethyl]-2-sulfanylidene- | | | | 12.39 (s, 1H), 7.51 (d, J = 2.93 | | remain- | remain- | remain- |
| | 1H,2H,3H,4H,5H-pyrrolo[3,2- | | | | Hz, 1H), 7.22-7.41 (m, 5H), | | ing | ing | ing |
| | d]pyrimidin-5-yl}methyl (2S)-5- | | | | 6.33-6.45 (m, 1H), 6.14-6.32 | | at 1 h | at 1 h | at 1 h |
| | oxopyrrolidine-1,2-dicarboxylate | | | | (m, 2H), 4.98-5.27 (m, 2H), | | | | |
| | | | | | 4.72 (dd, J = 3.22, 9.08 Hz, 1H), | | | | |
| | | | | | 4.46 (t, J = 6.15 Hz, 2H), 3.69 (t, | | | | |
| | | | | | J = 5.86 Hz, 2H), 3.45-3.62 (m, | | | | |
| | | | | | 1H), 2.18-2.39 (m, 1H), 1.81- | | | | |
| | | | | | 2.02 (m, 1H), 0.84-1.09 (m, 6H) | | | | |
| 5 | 1-(9H-fluoren-9-ylmethyl) 2-{4- | 6 | 603 | A | 1H NMR (300 MHz, dmso) δ | 140 | 90% | 76% | 97% |
| | oxo-1-[2-(propan-2-yloxy)ethyl]- | | | | 12.04-12.39 (m, 1H), 7.76-8.02 | | remain- | remain- | remain- |
| | 2-sulfanylidene-1H, 2H,3H,4H,5H- | | | | (m, 2H), 7.13-7.70 (m, 6H), | | ing | ing | ing |
| | pyrrolo[3,2-d]pyrimidin-5- | | | | 5.96-6.40 (m, 3H), 5.81-5.83 | | at 1 h | at 1 h | at 1 h |
| | yl}methyl(2R)-pyrrolidine-1,2- | | | | (m, 1H), 3.76-4.56 (m, 6H), | | | | |
| | dicarboxylate | | | | 3.32-3.81 (m, 5H), 3.20-3.20 | | | | |
| | | | | | (m, 1H), 2.96-3.20 (m, 1H), | | | | |
| | | | | | 1.72-1.98 (m, 3H), 0.72-1.04 | | | | |
| | | | | | (m, 5H) | | | | |

### Example 6

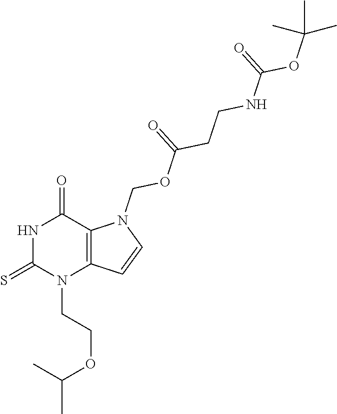 [SMILES: CC(C)OCCn1c(=S)[nH]c(=O)c2c1ccn2COC(=O)CCNC(=O)OC(C)(C)C]

{4-Oxo-1-[2-(propan-2-yloxy)ethyl]-2-sulfanylidene-1H,2H,3H,4H,5H-pyrrolo[3,2-d]pyrimidin-5-yl}methyl 3-{[(tert-butoxy)carbonyl]amino}propanoate. A solution of 2,3-dihydro-1-(2-isopropoxyethyl)-2-thioxo-1H-pyrrolo[3,2-d]pyrimidin-4(5H)-one (100 mg, 400 μmol) in THF (2.0 mL) was treated with lithium hexamethyldisilylamide (1.0 M in THF, 840 μL, 840 μmol) and stirred for 20 mins. tert-butyl 2-((chloromethoxy)carbonyl)ethylcarbamate (190 mg, 800 μmol) was added and the mixture was stirred overnight. The reaction was quenched by the addition of water and extracted with EtOAc. The organic layer was washed with brine and the solvent was removed in vacuo. Crude product was purified by normal phase chromatography (12 g column, 0-60% EtOAc/DCM) where the product fractions were combined and concentrated in vacuo to provide the pure product as a solid (120 mg, 66%). ¹H NMR (300 MHz, CHLOROFORM-d) δ 9.34-9.57 (m, 1H), 7.20-7.32 (m, 1H), 6.13-6.30 (m, 3H), 4.44-4.59 (m, 2H), 3.84 (s, 2H), 3.49-3.63 (m, 1H), 3.16-3.45 (m, 2H), 2.55 (t, J=6.15 Hz, 2H), 1.42 (s, 9H), 0.86-1.14 (m, 6H). LC/MS method A: Rₜ=5.0 mins (M+H)⁺=455, purity >95%. Caco-2 Pₐₚₚ=179 nm/s, SGF=82% remaining at 1 h, SIF=67% remaining at 1 h, hPlasma stability=59% remaining at 1 h.

Additional compounds prepared according to EXAMPLE 6:

| Ex- | | LCMS | | | | Caco-2 | | | human |
|---|---|---|---|---|---|---|---|---|---|
| am- | | retention | M + | LCMS | | Pₐₚₚ | | | Plasma |
| ple | Name | time | H⁺ | method | NMR | (nm/s) | SGF | SIF | stability |
| 7 | {4-oxo-1-[2-(propan-2-yloxy)ethyl]- | 5.2 | 483 | A | 1H NMR (300 MHz, cdcl3) δ | 147 | 66% | 40% | 4% |
| | 2-sulfanylidene-1H,2H,3H,4H,5H- | | | | 7.14-7.32 (m, 2H), 6.16-6.30 | | remain- | remain- | remain- |
| | pyrrolo[3,2-d]pyrimidin-5-yl}methyl | | | | (m, 3H), 4.42-4.67 (m, 3H), | | ing | ing | ing |
| | 5-{[(tert- | | | | 3.77-3.94 (m, 2H), 3.45-3.68 | | at 1 h | at 1 h | at 1 h |
| | butoxy)carbonyl]amino}pentanoate | | | | (m, 1H), 3.09 (q, J = 6.44 Hz, | | | | |
| | | | | | 2H), 2.26-2.46 (m, 2H), 1.46- | | | | |
| | | | | | 1.71 (m, 5H), 0.97-1.18 (m, | | | | |
| | | | | | 6H) | | | | |

### Example 8

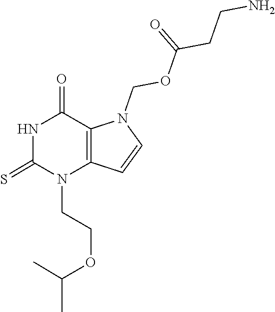 [SMILES: CC(C)OCCn1c(=S)[nH]c(=O)c2c1ccn2COC(=O)CCN]

Trifluoroacetic acid {4-oxo-1-[2-(propan-2-yloxy)ethyl]-2-sulfanylidene-1H,2H,3H,4H,5H-pyrrolo[3,2-d]pyrimidin-5-yl}methyl 3-aminopropanoate. To a solution of {4-oxo-1-[2-(propan-2-yloxy)ethyl]-2-sulfanylidene-1H,2H,3H,4H,5H-pyrrolo[3,2-d]pyrimidin-5-yl}methyl 3-{[(tert-butoxy)carbonyl]amino}propanoate (50 mg, 110 μmol) in 2.0 mL DCM was added 1.0 mL TFA. The reaction was stirred for 1 hour. The solvent was removed in vacuo and crude product was purified by RP-HPLC (method D) where the product fractions were combined and lyophilized to provide the pure product as a white solid (45 mg, 87%). ¹H NMR (300 MHz, CHLOROFORM-d) δ 8.15-8.40 (m, 2H), 7.19-7.30 (m, 2H), 6.10-6.38 (m, 3H), 4.45-4.61 (m, 2H), 3.84 (t, J=5.57 Hz, 2H), 3.48-3.65 (m, 1H), 3.31 (br. s., 2H), 2.73-2.95 (m, 2H), 0.98-1.18 (m, 6H). LC/MS method A: Rₜ=3.3 mins (M+H)⁺=355, purity >95%. Caco-2 Pₐₚₚ=4.6 nm/s, SGF=97% remaining at 1 h, SIF=68% remaining at 1 h, hPlasma stability=45% remaining at 1 h.

### Example 9

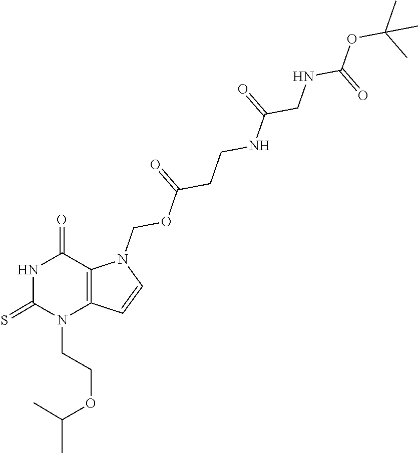 [SMILES: CC(C)OCCn1c(=S)[nH]c(=O)c2c1ccn2COC(=O)CCNC(=O)CNC(=O)OC(C)(C)C]

{4-Oxo-1-[2-(propan-2-yloxy)ethyl]-2-sulfanylidene-1H,2H,3H,4H,5H-pyrrolo[3,2-d]pyrimidin-5-yl}methyl 3-(2-{[(tert-butoxy)carbonyl]amino}acetamido)propanoate. To a solution of trifluoroacetic acid {4-oxo-1-[2-(propan-2-yloxy)ethyl]-2-sulfanylidene-1H,2H,3H,4H,5H-pyrrolo[3,2-d]pyrimidin-5-yl}methyl 3-aminopropanoate (36 mg, 80 μmol) in 300 μL DMF was added HATU (60 mg, 160 μmol), N-Boc-glycine (26 mg, 160 μmol), and DIPEA (56 μL, 320 μmol). The reaction stirred at room temperature overnight. 200 μL H₂O was added and the crude product was purified by RP-HPLC (method D) where the product fractions were combined and lyophilized to provide the pure product as a white solid (32 mg, 78%). ¹H NMR (300 MHz, METHANOL-d₄) δ 7.54-7.71 (m, 1H), 6.51-6.62 (m, 1H), 6.44 (s, 2H), 4.69-4.86 (m, 2H), 3.97-4.11 (m, 2H), 3.59-3.90 (m, 5H), 3.45-3.53 (m, 1H), 2.65-2.83 (m, 2H), 1.55-1.68 (m, 9H), 1.18-1.31 (m, 6H). LC/MS method A: Rₜ=4.5 mins (M+H)⁺=512, purity >95%. Caco-2 Pₐₚₚ=15.3 nm/s, SGF=100% remaining at 1 h, SIF=61% remaining at 1 h, hPlasma stability=52% remaining at 1 h.

Additional compounds prepared according to EXAMPLE 9:

| Ex- | | LCMS | | | | Caco-2 | | | human |
|---|---|---|---|---|---|---|---|---|---|
| am- | | retention | M + | LCMS | | Pₐₚₚ | | | Plasma |
| ple | Name | time | H⁺ | method | NMR | (nm/s) | SGF | SIF | stability |
| 10 | {4-oxo-1-[2-(propan-2-yloxy)- | 5.2 | 582 | A | 1H NMR (300 MHz, cdcl3) δ | 48.7 | 81% | 76% | 72% |
| | ethyl]-2-sulfanylidene- | | | | 7.25 (d, J = 3.51 Hz, 1H), 6.13- | | remain- | remain- | remain- |
| | 1H,2H,3H,4H,5H- | | | | 6.33 (m, 4H), 4.41-4.63 (m, | | ing | ing | ing |
| | pyrrolo[3,2-d]pyrimidin-5- | | | | 2H), 3.95 (dd, J = 6.74, 9.67 Hz, | | at 1 h | at 1 h | at 1 h |
| | yl}methyl5-[(2S)-2-{[(tert- | | | | 1H), 3.74-3.90 (m, 2H), 3.51- | | | | |
| | butoxy)carbonyl]amino}-3- | | | | 3.65 (m, 1H), 3.08-3.35 (m, | | | | |
| | methylbutanamido]pentanoate | | | | 2H), 2.28-2.46 (m, 2H), 1.49- | | | | |
| | | | | | 1.73 (m, 4H), 1.46 (s, 9H), 1.00- | | | | |
| | | | | | 1.14 (m, 6H), 0.86-1.00 (m, 6H) | | | | |
| 11 | {4-oxo-1-[2-(propan-2-yloxy)- | 5.1 | 554 | A | 1H NMR (300 MHz, dmso) δ | 14.4 | 74% | 69% | 99% |
| | ethyl]-2-sulfanylidene- | | | | 7.78 (d, J = 2.93 Hz, 1H), 6.67 | | remain- | remain- | remain- |
| | 1H,2H,3H,4H,5H- | | | | (d, J = 2.93 Hz, 1H), 6.45 (s, | | ing | ing | ing |
| | pyrrolo[3,2-d]pyrimidin-5- | | | | 2H), 4.75 (t, J = 6.15 Hz, 2H), | | at 1 h | at 1 h | at 1 h |
| | yl}methyl3-[(2S)-2-{[(tert- | | | | 3.74-4.09 (m, 4H), 3.38-3.69 | | | | |
| | butoxy)carbonyl]amino}-3- | | | | (m, 4H), 2.65-2.86 (m, 9H), | | | | |
| | methylbutanamido]propanoate | | | | 2.00-2.24 (m, 1H), 1.54-1.74 | | | | |
| | | | | | (m, 9H), 1.19-1.39 (m, 6H), | | | | |
| | | | | | 0.96-1.15 (m, 6H) | | | | |

### Example 12

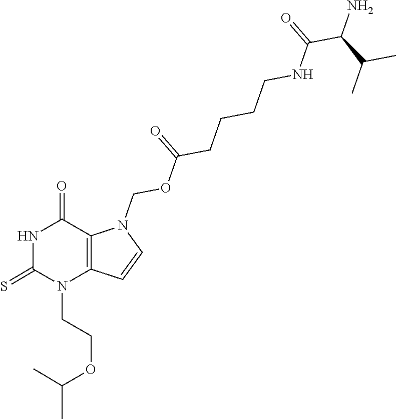 [SMILES: CC(C)OCCn1c(=S)[nH]c(=O)c2c1ccn2COC(=O)CCCCNC(=O)[C@@H](N)C(C)C]

Trifluoroacetic acid {4-oxo-1-[2-(propan-2-yloxy)ethyl]-2-sulfanylidene-1H,2H,3H,4H,5H-pyrrolo[3,2-d]pyrimidin-5-yl}methyl 5-[(2S)-2-amino-3-methylbutanamido]pentanoate. To a solution of {4-oxo-1-[2-(propan-2-yloxy)ethyl]-2-sulfanylidene-1H,2H,3H,4H,5H-pyrrolo[3,2-d]pyrimidin-5-yl}methyl 5-[(2S)-2-{[(tert-butoxy)carbonyl]amino}-3-methylbutanamido]pentanoate (17 mg, 29 μmol) in 1.0 mL DCM was added 300 μL TFA. The reaction was stirred for 1 hour. The solvent was removed in vacuo and crude product was purified by RP-HPLC (method D) where the product fractions were combined and lyophilized to provide the pure product as a white solid (15 mg, 86%). ¹H NMR (300 MHz, METHANOL-d₄) δ 7.53-7.73 (m, 1H), 6.51-6.60 (m, 1H), 6.36-6.47 (m, 2H), 4.64-4.85 (m, 2H), 3.98-4.12 (m, 2H), 3.66-3.85 (m, 2H), 3.25-3.47 (m, 2H), 2.45-2.65 (m, 2H), 2.27-2.42 (m, 1H), 1.63-1.93 (m, 4H), 1.11-1.29 (m, 12H). LC/MS method A: Rₜ=3.7 mins (M+H)⁺=482, purity >95%. Caco-2 Pₐₚₚ=4.0 nm/s, SGF=100% remaining at 1 h, SIF=94% remaining at 1 h, hPlasma stability=76% remaining at 1 h.

Additional compounds prepared according to EXAMPLE 12:

| | | LCMS | | | |
|---|---|---|---|---|---|
| | | retention | | LCMS | |
| Example | Name | time | M + H⁺ | method | NMR |
| 13 | {4-oxo-1-[2-(propan-2-yloxy)ethyl]- | 4.7 | 540 | A | 1H NMR (300 MHz, cd3od) δ |
| | 2-sulfanylidene-1H,2H,3H,4H,5H- | | | | 7.19-7.30 (m, 1H), 6.17 (d, |
| | pyrrolo[3,2-d]pyrimidin-5-yl}methyl | | | | J = 2.34 Hz, 1H), 6.05 (s, 2H), |
| | 5-(2-{[(tert- | | | | 4.38 (s, 2H), 3.55-3.75 (m, 2H), |
| | butoxy)carbonyl]amino}acetamido) | | | | 3.34-3.53 (m, 3H), 2.98 (t, |
| | pentanoate | | | | J = 6.74 Hz, 2H), 2.17 (t, J = 7.03 |
| | | | | | Hz, 2H), 1.28-1.53 (m, 4H), |
| | | | | | 1.22-1.28 (m, 9H), 0.78-0.94 |
| | | | | | (m, 6H) |
| 14 | trifluoroacetic acid {4-oxo-1-[2- | 3.5 | 454 | A | 1H NMR (300 MHz, cd3od) δ |
| | (propan-2-yloxy)ethyl]-2- | | | | 7.48-7.62 (m, 1H), 6.44-6.55 |
| | sulfanylidene-1H,2H,3H,4H,5H- | | | | (m, 1H), 6.37 (s, 2H), 4.69 (t, |
| | pyrrolo[3,2-d]pyrimidin-5-yl}methyl | | | | J = 5.57 Hz, 2H), 3.87-4.07 (m, |
| | 3-[(2S)-2-amino-3- | | | | 2H), 3.47-3.77 (m, 4H), 2.63- |
| | methylbutanamido]propanoate | | | | 2.82 (m, 2H), 2.11-2.35 (m, |
| | | | | | 1H), 0.98-1.23 (m, 12H) |
| 15 | trifluoroacetic acid {4-oxo-1-[2- | 3.3 | 440 | A | 1H NMR (300 MHz, cd3od) δ |
| | (propan-2-yloxy)ethyl]-2- | | | | 7.45-7.59 (m, 1H), 6.44 (s, 1H), |
| | sulfanylidene-1H,2H,3H,4H,5H- | | | | 6.33 (s, 2H), 4.60-4.70 (m, 2H), |
| | pyrrolo[3,2-d]pyrimidin-5-yl} methyl | | | | 3.88-4.01 (m, 2H), 3.74 (s, 3H), |
| | 5-(2-aminoacetamido)pentanoate | | | | 3.30 (t, J = 6.44 Hz, 2H), 2.34- |
| | | | | | 2.52 (m, 2H), 1.56-1.79 (m, |
| | | | | | 4H), 1.04-1.21 (m, 6H) |
| 16 | trifluoroacetic acid benzyl (5S)-2- | 4.8 | 628 | A | 1H NMR (300 MHz, cd3od) δ |
| | oxo-5-{[5-oxo-5-({4-oxo-1-[2- | | | | 7.48 (br. s., 1H), 7.30-7.43 (m, |
| | (propan-2-yloxy)ethyl]-2- | | | | 5H), 6.36-6.47 (m, 1H), 6.23- |
| | sulfanylidene-1H,2H,3H,4H,5H- | | | | 6.34 (m, 2H), 5.13-5.38 (m, |
| | pyrrolo[3,2-d]pyrimidin-5- | | | | 2H), 4.51-4.72 (m, 3H), 3.89 (t, |
| | yl}methoxy)pentyl]carbamoyl} | | | | J = 5.57 Hz, 2H), 3.48-3.76 (m, |
| | pyrrolidine-1-carboxylate | | | | 4H), 3.15 (t, J = 6.44 Hz, 2H), |
| | | | | | 2.20-2.81 (m, 5H), 1.84-2.07 |
| | | | | | (m, 1H), 1.37-1.69 (m, 4H), |
| | | | | | 1.04-1.18 (m, 6H) |
| 17 | trifluoroacetic acid benzyl (5S)-2- | 4.6 | 600 | A | 1H NMR (300 MHz, cd3od) δ |
| | oxo-5-{[3-oxo-3-({4-oxo-1-[2- | | | | 7.48-7.59 (m, 1H), 7.30-7.44 |
| | (propan-2-yloxy)ethyl]-2- | | | | (m, 5H), 6.44 (d, J = 2.93 Hz, |
| | sulfanylidene-1H,2H,3H,4H,5H- | | | | 1H), 6.31 (d, J = 1.17 Hz, 2H), |
| | pyrrolo[3,2-d]pyrimidin-5- | | | | 5.26-5.41 (m, 1H), 5.11-5.25 |
| | yl}methoxy)propyl]carbamoyl} | | | | (m, 1H), 4.58-4.78 (m, 3H), |
| | pyrrolidine-1-carboxylate | | | | 3.93 (t, J = 5.57 Hz, 2H), 3.67 (t, |
| | | | | | J = 5.86 Hz, 1H), 3.45 (dd, |
| | | | | | J = 1.76, 6.44 Hz, 2H), 2.29- |
| | | | | | 2.80 (m, 6H), 1.91-2.09 (m, |
| | | | | | 1H), 1.14 (d, J = 5.86 Hz, 6H) |
| 18 | trifluoroacetic acid {4-oxo-1-[2- | 5.7 | 644 | A | 1H NMR (300 MHz, cd3od) δ |
| | (propan-2-yloxy)ethyl]-2- | | | | 7.42 (d, J = 2.93 Hz, 1H), 7.08- |
| | sulfanylidene-1H,2H,3H,4H,5H- | | | | 7.33 (m, 6H), 6.29-6.41 (m, |
| | pyrrolo[3,2-d]pyrimidin-5-yl}methyl | | | | 1H), 6.20-6.29 (m, 2H), 4.73- |
| | 5-[(2S)-2-{[(tert- | | | | 4.80 (m, 1H), 4.57 (t, J = 5.57 |
| | butoxy)carbonyl]amino}-4- | | | | Hz, 2H), 3.91-4.08 (m, 1H), |
| | phenylbutanamido]pentanoate | | | | 3.79-3.91 (m, 2H), 3.52-3.67 |
| | | | | | (m, 1H), 3.08-3.29 (m, 3H), |
| | | | | | 2.49-2.82 (m, 2H), 2.29-2.43 |
| | | | | | (m, 2H), 1.74-2.15 (m, 2H), |
| | | | | | 1.49-1.73 (m, 4H), 1.46 (s, 9H), |
| | | | | | 1.00-1.12 (m, 6H) |
| 19 | trifluoroacetic acid {4-oxo-1-[2- | 5.6 | 616 | A | 1H NMR (300 MHz, cd3od) δ |
| | (propan-2-yloxy)ethyl]-2- | | | | 7.42-7.48 (m, 1H), 7.25-7.35 |
| | sulfanylidene-1H,2H,3H,4H,5H- | | | | (m, 2H), 7.17-7.25 (m, 3H), |
| | pyrrolo[3,2-d]pyrimidin-5-yl}methyl | | | | 6.31-6.38 (m, 1H), 6.26-6.31 |
| | 3-[(2S)-2-{[(tert- | | | | (m, 2H), 4.58 (t, J = 5.86 Hz, |
| | butoxy)carbonyl]amino}-4- | | | | 2H), 3.80-4.11 (m, 3H), 3.41- |
| | phenylbutanamido]propanoate | | | | 3.71 (m, 3H), 2.55-2.77 (m, |
| | | | | | 4H), 1.77-2.19 (m, 2H), 1.41- |
| | | | | | 1.58 (m, 9H), 1.10 (d, J = 5.86 |
| | | | | | Hz, 6H) |
| 20 | trifluoroacetic acid {4-oxo-1-[2- | 5.9 | 736 | A | 1H NMR (300 MHz, cd3od) δ |
| | (propan-2-yloxy)ethyl]-2- | | | | 7.87-7.98 (m, 1H), 7.75-7.87 |
| | sulfanylidene-1H,2H,3H,4H,5H- | | | | (m, 2H), 7.62-7.73 (m, 2H), |
| | pyrrolo[3,2-d]pyrimidin-5-yl}methyl | | | | 7.21-7.46 (m, 6H), 6.28-6.40 |
| | 5-[(2S)-2-{[(9H-fluoren-9- | | | | (m, 1H), 6.15-6.26 (m, 2H), |
| | ylmethoxy)carbonyl]amino}-4- | | | | 4.31-4.62 (m, 4H), 4.11-4.30 |
| | (methylsulfanyl)butanamido]pentanoate | | | | (m, 2H), 3.77-3.95 (m, 2H), |
| | | | | | 3.45-3.67 (m, 1H), 3.08-3.25 |
| | | | | | (m, 2H), 2.29-2.62 (m, 4H), |
| | | | | | 1.74-2.15 (m, 6H), 1.36-1.70 |
| | | | | | (m, 4H), 0.94-1.17 (m, 6H) |
| 21 | trifluoroacetic acid {4-oxo-1-[2- | 5.9 | 708 | A | 1H NMR (300 MHz, cd3od) δ |
| | (propan-2-yloxy)ethyl]-2- | | | | 7.94-8.13 (m, 1H), 7.81 (d, |
| | sulfanylidene-1H,2H,3H,4H,5H- | | | | J = 7.03 Hz, 2H), 7.61-7.73 (m, |
| | pyrrolo[3,2-d]pyrimidin-5-yl}methyl | | | | 2H), 7.21-7.48 (m, 5H), 6.33 (d, |
| | 3-[(2S)-2-{[(9H-fluoren-9- | | | | J = 2.93 Hz, 1H), 6.22 (s, 2H), |
| | ylmethoxy)carbonyl]amino}-4- | | | | 4.49-4.63 (m, 2H), 4.39-4.49 |
| | (methylsulfanyl) butanamido]propanoate | | | | (m, 2H), 4.14-4.30 (m, 2H), |
| | | | | | 3.79-3.91 (m, 2H), 3.38-3.66 |
| | | | | | (m, 3H), 2.36-2.67 (m, 4H), |
| | | | | | 2.09 (s, 3H), 1.78-2.03 (m, 2H), |
| | | | | | 1.07 (d, J = 5.86 Hz, 6H) |
| 22 | trifluoroacetic acid {4-oxo-1-[2- | 6.1 | 752 | A | 1H NMR (300 MHz, dmso) δ |
| | (propan-2-yloxy)ethyl]-2- | | | | 7.94 (d, J = 7.62 Hz, 3H), 7.07- |
| | sulfanylidene-1H,2H,3H,4H,5H- | | | | 7.80 (m, 14H), 6.38-6.50 (m, |
| | pyrrolo[3,2-d]pyrimidin-5-yl}methyl | | | | 1H), 6.23 (s, 2H), 4.47-4.63 (m, |
| | 5-[(2S)-2-{[(9H-fluoren-9- | | | | 2H), 4.14-4.36 (m, 4H), 3.69- |
| | ylmethoxy)carbonyl]amino}-3- | | | | 3.89 (m, 2H), 3.60 (t, J = 5.86 |
| | phenylpropanamido]pentanoate | | | | Hz, 1H), 2.80-3.21 (m, 4H), |
| | | | | | 2.28-2.44 (m, 2H), 1.36-1.62 |
| | | | | | (m, 4H), 1.06 (d, J = 6.44 Hz, |
| | | | | | 6H) |
| 23 | trifluoroacetic acid {4-oxo-1-[2- | 6 | 724 | A | 1H NMR (300 MHz, dmso) δ |
| | (propan-2-yloxy)ethyl]-2- | | | | 12.43 (s, 1H), 8.15 (s, 1H), 7.93 |
| | sulfanylidene-1H,2H,3H,4H,5H- | | | | (d, J = 7.03 Hz, 2H), 7.52-7.75 |
| | pyrrolo[3,2-d]pyrimidin-5-yl}methyl | | | | (m, 4H), 7.19-7.52 (m, 10H), |
| | 3-[(2S)-2-{[(9H-fluoren-9- | | | | 6.37-6.46 (m, 1H), 6.23-6.29 |
| | ylmethoxy)carbonyl]amino}-3- | | | | (m, 2H), 4.36-4.59 (m, 2H), |
| | phenylpropanamido]propanoate | | | | 4.12-4.33 (m, 4H), 3.71-3.80 |
| | | | | | (m, 3H), 3.49-3.69 (m, 1H), |
| | | | | | 3.23-3.47 (m, 2H), 2.73-3.02 |
| | | | | | (m, 2H), 2.49 (t, J = 6.74 Hz, |
| | | | | | 2H), 0.98-1.13 (m, 6H) |
| 24 | trifluoroacetic acid {4-oxo-1-[2- | 4 | 544 | A | 1H NMR (300 MHz, cd3od) δ |
| | (propan-2-yloxy)ethyl]-2- | | | | 7.19 (d, J = 2.93 Hz, 1H), 6.91- |
| | sulfanylidene-1H,2H,3H,4H,5H- | | | | 7.14 (m, 5H), 6.10-6.16 (m, |
| | pyrrolo [3,2-d]pyrimidin-5-yl} methyl | | | | 1H), 5.97-6.06 (m, 2H), 4.34 (t, |
| | 5-[(2S)-2-amino-4- | | | | J = 5.57 Hz, 2H), 3.57-3.71 (m, |
| | phenylbutanamido]pentanoate | | | | 3H), 3.37 (td, J = 6.30, 12.01 |
| | | | | | Hz, 1H), 2.92-3.06 (m, 2H), |
| | | | | | 2.41-2.54 (m, 2H), 2.09-2.24 |
| | | | | | (m, 2H), 1.82-2.03 (m, 2H), |
| | | | | | 1.30-1.56 (m, 4H), 0.85 (d, |
| | | | | | J = 5.86 Hz, 6H) |
| 25 | trifluoroacetic acid {4-oxo-1-[2- | 4 | 516 | A | 1H NMR (300 MHz, cd3od) δ |
| | (propan-2-yloxy)ethyl]-2- | | | | 7.48 (d, J = 2.93 Hz, 1H), 7.21- |
| | sulfanylidene-1H,2H,3H,4H,5H- | | | | 7.43 (m, 5H), 6.34-6.39 (m, |
| | pyrrolo[3,2-d]pyrimidin-5-yl}methyl | | | | 1H), 6.30-6.34 (m, 2H), 4.60 (t, |
| | 3-[(2S)-2-amino-4- | | | | J = 5.57 Hz, 2H), 3.84-4.03 (m, |
| | phenylbutanamido]propanoate | | | | 3H), 3.43-3.77 (m, 3H), 2.63- |
| | | | | | 2.84 (m, 4H), 2.05-2.29 (m, |
| | | | | | 2H), 1.03-1.22 (m, 6H) |
| 26 | {4-oxo-1-[2-(propan-2-yloxy)ethyl]- | 5.5 | 596 | A | 1H NMR (300 MHz, cd3od) δ |
| | 2-sulfanylidene-1H,2H,3H,4H,5H- | | | | 7.45-7.59 (m, 1H), 6.46 (d, |
| | pyrrolo[3,2-d]pyrimidin-5-yl}methyl | | | | J = 2.93 Hz, 1H), 6.34 (s, 2H), |
| | 5-[(2S)-2-{[(tert- | | | | 4.67 (s, 2H), 4.02-4.22 (m, 1H), |
| | butoxy)carbonyl]amino}-4- | | | | 3.96 (s, 2H), 3.56-3.80 (m, 2H), |
| | methylpentanamido]pentanoate | | | | 3.12-3.37 (m, 2H), 2.39-2.56 |
| | | | | | (m, 2H), 1.48-1.85 (m, 16H), |
| | | | | | 1.10-1.22 (m, 6H), 1.03 (t, |
| | | | | | J = 6.44 Hz, 6H) |
| 27 | {4-oxo-1-[2-(propan-2-yloxy)ethyl]- | 5.3 | 568 | A | 1H NMR (300 MHz, cd3od) δ |
| | 2-sulfanylidene-1H, 2H,3H,4H,5H- | | | | 7.41-7.52 (m, 1H), 6.39 (d, |
| | pyrrolo[3,2-d]pyrimidin-5-yl}methyl | | | | J = 2.93 Hz, 1H), 6.27 (d, |
| | 3-[(2S)-2-{[(tert- | | | | J = 2.93 Hz, 2H), 4.50-4.66 (m, |
| | butoxy)carbonyl]amino}-4- | | | | 2H), 3.94-4.13 (m, 1H), 3.79- |
| | methylpentanamido]propanoate | | | | 3.94 (m, 2H), 3.37-3.74 (m, |
| | | | | | 4H), 2.57 (t, J = 6.74 Hz, 2H), |
| | | | | | 1.61-1.80 (m, 1H), 1.46 (s, |
| | | | | | 11H), 1.09 (d, J = 5.86 Hz, 6H), |
| | | | | | 0.95 (t, J = 5.86 Hz, 6H) |
| 28 | {4-oxo-1-[2-(propan-2-yloxy)ethyl]- | 5.3 | 614 | A | 1H NMR (300 MHz, cd3od) δ |
| | 2-sulfanylidene-1H,2H,3H,4H,5H- | | | | 7.42-7.57 (m, 1H), 6.41 (d, |
| | pyrrolo[3,2-d]pyrimidin-5-yl}methyl | | | | J = 2.93 Hz, 1H), 6.30 (s, 2H), |
| | 5-[(2S)-2-{[(tert- | | | | 4.63 (s, 2H), 4.05-4.25 (m, 1H), |
| | butoxy)carbonyl]amino}-4- | | | | 3.92 (s, 2H), 3.55-3.80 (m, 1H), |
| | (methylsulfanyl) butanamido]pentanoate | | | | 3.14-3.30 (m, 2H), 2.42 (s, 4H), |
| | | | | | 2.13 (s, 3H), 1.79-2.08 (m, 2H), |
| | | | | | 1.51-1.73 (m, 4H), 1.49 (s, 9H), |
| | | | | | 1.12 (d, J = 6.44 Hz, 6H) |
| 29 | {4-oxo-1-[2-(propan-2-yloxy)ethyl]- | 5.1 | 586 | A | 1H NMR (300 MHz, cd3od) δ |
| | 2-sulfanylidene-1H, 2H,3H,4H,5H- | | | | 7.43-7.57 (m, 1H), 6.40 (d, |
| | pyrrolo [3,2-d]pyrimidin-5-yl}methyl | | | | J = 3.51 Hz, 1H), 6.28 (s, 2H), |
| | 3-[(2S)-2-{[(tert- | | | | 4.60 (s, 2H), 4.05-4.21 (m, 1H), |
| | butoxy)carbonyl]amino}-4- | | | | 3.89 (s, 2H), 3.39-3.70 (m, 3H), |
| | (methylsulfanyl) butanamido]propanoate | | | | 2.59 (s, 4H), 2.10 (s, 3H), 1.75- |
| | | | | | 2.04 (m, 2H), 1.75-2.04 (m, |
| | | | | | 2H), 1.47 (s, 9H), 1.10 (d, |
| | | | | | J = 5.86 Hz, 6H) |
| 30 | trifluoroacetic acid {4-oxo-1-[2- | 3.8 | 468 | A | 1H NMR (300 MHz, cd3od) δ |
| | (propan-2-yloxy)ethyl]-2- | | | | 7.38-7.52 (m, 1H), 6.39 (d, |
| | sulfanylidene-1H,2H,3H,4H,5H- | | | | J = 3.51 Hz, 1H), 6.27 (s, 2H), |
| | pyrrolo[3,2-d]pyrimidin-5-yl}methyl | | | | 4.59 (t, J = 5.86 Hz, 2H), 3.77- |
| | 3-[(2S)-2-amino-4- | | | | 3.92 (m, 3H), 3.39-3.72 (m, |
| | methylpentanamido]propanoate | | | | 4H), 2.61 (t, J = 6.44 Hz, 2H), |
| | | | | | 1.59-1.76 (m, 3H), 1.08 (d, |
| | | | | | J = 6.44 Hz, 6H), 0.99 (d, |
| | | | | | J = 4.69 Hz, 6H) |
| 31 | trifluoroacetic acid {4-oxo-1-[2- | 3.8 | 514 | A | 1H NMR (300 MHz, cd3od) δ |
| | (propan-2-yloxy)ethyl]-2- | | | | 7.42-7.56 (m, 1H), 6.42 (d, |
| | sulfanylidene-1H,2H,3H,4H,5H- | | | | J = 2.93 Hz, 1H), 6.30 (s, 2H), |
| | pyrrolo[3,2-d]pyrimidin-5-yl}methyl | | | | 4.63 (t, J = 5.86 Hz, 2H), 3.92 (t, |
| | 5-[(2S)-2-amino-4- | | | | J = 5.57 Hz, 3H), 3.53-3.77 (m, |
| | (methylsulfanyl)butanamido]pentanoate | | | | 1H), 3.21-3.35 (m, 2H), 2.62 (s, |
| | | | | | 2H), 2.44 (s, 2H), 2.17 (s, 5H), |
| | | | | | 1.50-1.80 (m, 5H), 1.12 (d, |
| | | | | | J = 5.86 Hz, 6H) |
| 32 | trifluoroacetic acid {4-oxo-1-[2- | 3.5 | 486 | A | 1H NMR (300 MHz, cd3od) δ |
| | (propan-2-yloxy)ethyl]-2- | | | | 7.35-7.55 (m, 1H), 6.35-6.47 |
| | sulfanylidene-1H,2H,3H,4H,5H- | | | | (m, 1H), 6.21-6.34 (m, 2H), |
| | pyrrolo [3,2-d]pyrimidin-5-yl} methyl | | | | 4.61 (t, J = 5.86 Hz, 2H), 3.80- |
| | 3-[(2S)-2-amino-4- | | | | 4.04 (m, 3H), 3.63 (s, 3H), 2.46- |
| | (methylsulfanyl) butanamido]propanoate | | | | 2.71 (m, 4H), 1.98-2.22 (m, |
| | | | | | 5H), 1.69 (d, J = 19.92 Hz, 1H), |
| | | | | | 1.10 (d, J = 6.44 Hz, 6H) |
| 33 | {4-oxo-1-[2-(propan-2-yloxy)ethyl]- | 5.4 | 596 | A | 1H NMR (300 MHz, cd3od) δ |
| | 2-sulfanylidene-1H,2H,3H,4H,5H- | | | | 7.97-8.11 (m, 1H), 7.42-7.63 |
| | pyrrolo [3,2-d]pyrimidin-5-yl}methyl | | | | (m, 1H), 6.41-6.51 (m, 1H), |
| | 5-[(2S,3S)-2-{[(tert- | | | | 6.23-6.39 (m, 2H), 4.59-4.76 |
| | butoxy)carbonyl]amino}-3- | | | | (m, 2H), 3.82-4.03 (m, 3H), |
| | methylpentanamido]pentanoate | | | | 3.55-3.76 (m, 1H), 3.13-3.35 |
| | | | | | (m, 2H), 2.36-2.54 (m, 2H), |
| | | | | | 1.45-1.90 (m, 16H), 1.07-1.24 |
| | | | | | (m, 7H), 0.92-1.06 (m, 6H) |
| 34 | {4-oxo-1-[2-(propan-2-yloxy)ethyl]- | 5.4 | 568 | A | 1H NMR (300 MHz, cd3od) δ |
| | 2-sulfanylidene-1H, 2H,3H,4H,5H- | | | | 7.38-7.59 (m, 1H), 6.38-6.50 |
| | pyrrolo[3,2-d]pyrimidin-5-yl}methyl | | | | (m, 1H), 6.24-6.35 (m, 2H), |
| | 3-[(2S,3S)-2-{[(tert- | | | | 4.55-4.66 (m, 2H), 3.73-3.99 |
| | butoxy)carbonyl]amino}-3- | | | | (m, 3H), 3.41-3.74 (m, 3H), |
| | methylpentanamido]propanoate | | | | 2.49-2.77 (m, 2H), 1.65-1.89 |
| | | | | | (m, 2H), 1.43-1.59 (m, 10H), |
| | | | | | 1.06-1.26 (m, 7H), 0.84-1.00 |
| | | | | | (m, 6H) |
| 35 | trifluoroacetic acid {4-oxo-1-[2- | 4.7 | 584 | A | 1H NMR (300 MHz, cd3od) δ |
| | (propan-2-yloxy)ethyl]-2- | | | | 7.38-7.61 (m, 1H), 6.42 (d, |
| | sulfanylidene-1H,2H,3H,4H,5H- | | | | J = 2.93 Hz, 1H), 6.30 (s, 2H), |
| | pyrrolo[3,2-d]pyrimidin-5-yl}methyl | | | | 4.63 (t, J = 5.57 Hz, 2H), 4.05- |
| | 5-[(2S,3R)-2-{[(tert- | | | | 4.29 (m, 1H), 3.88-4.03 (m, |
| | butoxy)carbonyl]amino}-3- | | | | 3H), 3.65 (t, J = 5.86 Hz, 1H), |
| | hydroxybutanamido]pentanoate | | | | 3.25 (dd, J = 6.74, 13.18 Hz, |
| | | | | | 2H), 2.42 (t, J = 7.03 Hz, 2H), |
| | | | | | 1.43-1.81 (m, 13H), 1.20-1.30 |
| | | | | | (m, 3H), 1.04-1.16 (m, 6H) |
| 36 | trifluoroacetic acid {4-oxo-1-[2- | 4.6 | 556 | A | 1H NMR (300 MHz, cd3od) δ |
| | (propan-2-yloxy)ethyl]-2- | | | | 7.45-7.57 (m, 1H), 6.43 (dd, |
| | sulfanylidene-1H,2H,3H,4H,5H- | | | | J = 1.17, 2.93 Hz, 1H), 6.32 (s, |
| | pyrrolo[3,2-d]pyrimidin-5-yl}methyl | | | | 2H), 4.64 (t, J = 5.57 Hz, 2H), |
| | 3-[(2S,3R)-2-{[(tert- | | | | 4.07-4.28 (m, 1H), 3.84-4.05 |
| | butoxy)carbonyl]amino}-3- | | | | (m, 3H), 3.42-3.77 (m, 3H), |
| | hydroxybutanamido]propanoate | | | | 2.64 (t, J = 6.15 Hz, 2H), 1.52 (s, |
| | | | | | 9H), 1.21 (d, J = 5.86 Hz, 3H), |
| | | | | | 1.07-1.17 (m, 6H) |
| 37 | trifluoroacetic acid {4-oxo-1-[2- | 3.9 | 496 | A | 1H NMR (300 MHz, cd3od) δ |
| | (propan-2-yloxy)ethyl]-2- | | | | 7.44-7.56 (m, 1H), 6.44 (d, |
| | sulfanylidene-1H,2H,3H,4H,5H- | | | | J = 2.93 Hz, 1H), 6.32 (s, 2H), |
| | pyrrolo [3,2-d]pyrimidin-5-yl}methyl | | | | 4.65 (t, J = 5.86 Hz, 2H), 3.94 (t, |
| | 5-[(2S,3S)-2-amino-3- | | | | J = 5.57 Hz, 2H), 3.56-3.77 (m, |
| | methylpentanamido]pentanoate | | | | 2H), 3.18-3.34 (m, 2H), 2.30- |
| | | | | | 2.55 (m, 2H), 1.87-2.10 (m, |
| | | | | | 1H), 1.45-1.84 (m, 6H), 1.24- |
| | | | | | 1.45 (m, 1H), 0.97-1.19 (m, |
| | | | | | 13H) |
| 38 | trifluoroacetic acid {4-oxo-1-[2- | 3.7 | 468 | A | 1H NMR (300 MHz, cd3od) δ |
| | (propan-2-yloxy)ethyl]-2- | | | | 7.53 (d, J = 2.93 Hz, 1H), 6.45 |
| | sulfanylidene-1H,2H,3H,4H,5H- | | | | (d, J = 2.93 Hz, 1H), 6.33 (s, |
| | pyrrolo [3,2-d]pyrimidin-5-yl}methyl | | | | 2H), 4.65 (t, J = 5.57 Hz, 2H), |
| | 3-[(2S,3S)-2-amino-3- | | | | 3.94 (t, J = 5.57 Hz, 2H), 3.43- |
| | methylpentanamido]propanoate | | | | 3.79 (m, 4H), 2.68 (t, J = 6.44 |
| | | | | | Hz, 2H), 1.81-2.07 (m, 1H), |
| | | | | | 1.72-1.78 (m, 1H), 1.48-1.71 |
| | | | | | (m, 1H), 0.94-1.43 (m, 14H) |
| 39 | trifluoroacetic acid {4-oxo-1-[2- | 3.4 | 484 | A | 1H NMR (300 MHz, cd3od) δ |
| | (propan-2-yloxy)ethyl]-2- | | | | 7.36-7.59 (m, 1H), 6.43 (d, |
| | sulfanylidene-1H,2H,3H,4H,5H- | | | | J = 2.93 Hz, 1H), 6.31 (s, 2H), |
| | pyrrolo[3,2-d]pyrimidin-5-yl}methyl | | | | 4.64 (t, J = 5.57 Hz, 2H), 4.00- |
| | 5-[(2S,3R)-2-amino-3- | | | | 4.15 (m, 1H), 3.85-3.98 (m, |
| | hydroxybutanamido]pentanoate | | | | 2H), 3.56-3.74 (m, 2H), 3.13- |
| | | | | | 3.34 (m, 2H), 2.32-2.53 (m, |
| | | | | | 2H), 1.50-1.81 (m, 5H), 1.24- |
| | | | | | 1.42 (m, 3H), 0.96-1.19 (m, 6H) |
| 40 | trifluoroacetic acid {4-oxo-1-[2- | 3.2 | 456 | A | 1H NMR (300 MHz, cd3od) δ |
| | (propan-2-yloxy)ethyl]-2- | | | | 7.29 (d, J = 3.51 Hz, 1H), 6.22 |
| | sulfanylidene-1H,2H,3H,4H,5H- | | | | (d, J = 3.51 Hz, 1H), 6.09 (s, |
| | pyrrolo[3,2-d]pyrimidin-5-yl}methyl | | | | 2H), 4.42 (t, J = 5.57 Hz, 2H), |
| | 3-[(2S,3R)-2-amino-3- | | | | 3.60-3.94 (m, 3H), 3.23-3.53 |
| | hydroxybutanamido]propanoate | | | | (m, 4H), 2.32-2.57 (m, 2H), |
| | | | | | 1.53 (s, 1H), 1.01-1.24 (m, 4H), |
| | | | | | 0.80-0.96 (m, 6H) |
| 41 | trifluoroacetic acid {4-oxo-1-[2- | 4.5 | 570 | A | 1H NMR (300 MHz, cd3od) δ |
| | (propan-2-yloxy)ethyl]-2- | | | | 7.52-7.68 (m, 1H), 6.52 (d, |
| | sulfanylidene-1H,2H,3H,4H,5H- | | | | J = 3.51 Hz, 1H), 6.40 (s, 2H), |
| | pyrrolo[3,2-d]pyrimidin-5-yl}methyl | | | | 4.73 (t, J = 5.57 Hz, 2H), 4.14- |
| | 5-[(2S)-2-{[(tert- | | | | 4.30 (m, 1H), 3.66-4.08 (m, |
| | butoxy)carbonyl]amino}-3- | | | | 6H), 3.22-3.44 (m, 2H), 2.39- |
| | hydroxypropanamido]pentanoate | | | | 2.60 (m, 2H), 1.50-1.92 (m, |
| | | | | | 13H), 1.11-1.30 (m, 6H) |
| 42 | trifluoroacetic acid {4-oxo-1-[2- | 4.4 | 542 | A | 1H NMR (300 MHz, cd3od) δ |
| | (propan-2-yloxy)ethyl]-2- | | | | 7.47-7.57 (m, 1H), 6.44 (d, |
| | sulfanylidene-1H,2H,3H,4H,5H- | | | | J = 3.51 Hz, 1H), 6.32 (s, 2H), |
| | pyrrolo[3,2-d]pyrimidin-5-yl}methyl | | | | 4.65 (t, J = 5.86 Hz, 2H), 4.06- |
| | 3-[(2S)-2-{[(tert- | | | | 4.20 (m, 1H), 3.86-4.03 (m, |
| | butoxy)carbonyl]amino}-3- | | | | 3H), 3.45-3.84 (m, 5H), 2.59- |
| | hydroxypropanamido]propanoate | | | | 2.74 (m, 2H), 1.52 (s, 9H), 1.14 |
| | | | | | (d, J = 6.44 Hz, 6H) |
| 43 | trifluoroacetic acid {4-oxo-1-[2- | 3.4 | 470 | A | 1H NMR (300 MHz, cd3od) δ |
| | (propan-2-yloxy)ethyl]-2- | | | | 7.39-7.58 (m, 1H), 6.43 (d, |
| | sulfanylidene-1H,2H,3H,4H,5H- | | | | J = 2.93 Hz, 1H), 6.30 (s, 2H), |
| | pyrrolo[3,2-d]pyrimidin-5-yl}methyl | | | | 4.64 (t, J = 5.57 Hz, 2H), 3.75- |
| | 5-[(2S)-2-amino-3- | | | | 4.02 (m, 5H), 3.65 (td, J = 5.93, |
| | hydroxypropanamido]pentanoate | | | | 12.16 Hz, 1H), 3.21-3.32 (m, |
| | | | | | 2H), 2.32-2.52 (m, 2H), 1.45- |
| | | | | | 1.87 (m, 5H), 1.05-1.21 (m, 6H) |
| 44 | trifluoroacetic acid {4-oxo-1-[2- | 3.2 | 442 | A | 1H NMR (300 MHz, cd3od) δ |
| | (propan-2-yloxy)ethyl]-2- | | | | 7.41-7.58 (m, 1H), 6.36-6.47 |
| | sulfanylidene-1H,2H,3H,4H,5H- | | | | (m, 1H), 6.25-6.36 (m, 2H), |
| | pyrrolo[3,2-d]pyrimidin-5-yl}methyl | | | | 4.52-4.69 (m, 2H), 3.44-4.02 |
| | 3-[(2S)-2-amino-3- | | | | (m, 8H), 2.57-2.76 (m, 2H), |
| | hydroxypropanamido]propanoate | | | | 0.97-1.17 (m, 6H) |
| 45 | trifluoroacetic acid {4-oxo-1-[2- | 4.9 | 646 | A | 1H NMR (300 MHz, cd3od) δ |
| | (propan-2-yloxy)ethyl]-2- | | | | 7.39-7.51 (m, 1H), 7.03 (d, |
| | sulfanylidene-1H, 2H,3H,4H,5H- | | | | J = 8.79 Hz, 2H), 6.70 (d, |
| | pyrrolo[3,2-d]pyrimidin-5-yl}methyl | | | | J = 8.20 Hz, 2H), 6.37 (d, |
| | 5-[(2S)-2-{[(tert- | | | | J = 2.93 Hz, 1H), 6.26 (s, 2H), |
| | butoxy)carbonyl]amino}-3-(4- | | | | 4.58 (t, J = 5.86 Hz, 2H), 4.03- |
| | hydroxyphenyl)propanamido]pentanoate | | | | 4.27 (m, 1H), 3.79-3.96 (m, |
| | | | | | 2H), 3.51-3.72 (m, 1H), 2.66- |
| | | | | | 3.28 (m, 5H), 2.25-2.45 (m, |
| | | | | | 2H), 1.31-1.62 (m, 14H), 0.98- |
| | | | | | 1.19 (m, 6H) |
| 46 | trifluoroacetic acid {4-oxo-1-[2- | 4.8 | 618 | A | 1H NMR (300 MHz, cd3od) δ |
| | (propan-2-yloxy)ethyl]-2- | | | | 7.41-7.59 (m, 1H), 6.98-7.17 |
| | sulfanylidene-1H,2H,3H,4H,5H- | | | | (m, 2H), 6.75 (d, J = 7.03 Hz, |
| | pyrrolo[3,2-d]pyrimidin-5-yl}methyl | | | | 2H), 6.36-6.44 (m, 1H), 6.30 (s, |
| | 3-[(2S)-2-{[(tert- | | | | 2H), 4.51-4.65 (m, 2H), 4.11- |
| | butoxy)carbonyl]amino}-3-(4- | | | | 4.29 (m, 1H), 3.78-3.95 (m, |
| | hydroxyphenyl)propanamido]propanoate | | | | 2H), 3.45-3.73 (m, 2H), 2.66- |
| | | | | | 3.00 (m, 2H), 2.53 (br. s., 2H), |
| | | | | | 1.29-1.53 (m, 9H), 1.12 (dd, |
| | | | | | J = 2.34, 5.86 Hz, 6H) |
| 47 | trifluoroacetic acid {4-oxo-1-[2- | 5.4 | 669 | A | 1H NMR (300 MHz, cd3od) δ |
| | (propan-2-yloxy)ethyl]-2- | | | | 7.65 (d, J = 7.62 Hz, 1H), 7.50 |
| | sulfanylidene-1H,2H,3H,4H,5H- | | | | (d, J = 3.51 Hz, 1H), 7.38 (d, |
| | pyrrolo[3,2-d]pyrimidin-5-yl}methyl | | | | J = 8.20 Hz, 1H), 6.99-7.22 (m, |
| | 5-[(2S)-2-{[(tert- | | | | 3H), 6.42 (d, J = 2.93 Hz, 1H), |
| | butoxy)carbonyl]amino}-3-(1H- | | | | 6.30 (s, 2H), 4.63 (t, J = 5.86 Hz, |
| | indol-3- | | | | 2H), 4.36 (s, 1H), 3.92 (t, |
| | yl)propanamido]pentanoate | | | | J = 5.57 Hz, 2H), 3.65 (t, J = 5.86 |
| | | | | | Hz, 1H), 2.83-3.33 (m, 4H), |
| | | | | | 2.20-2.46 (m, 2H), 1.20-1.56 |
| | | | | | (m, 13H), 1.01-1.19 (m, 6H) |
| 48 | trifluoroacetic acid {4-oxo-1-[2- | 5.3 | 641 | A | 1H NMR (300 MHz, cd3od) δ |
| | (propan-2-yloxy)ethyl]-2- | | | | 7.56-7.69 (m, 1H), 7.44-7.56 |
| | sulfanylidene-1H,2H,3H,4H,5H- | | | | (m, 1H), 7.36-7.44 (m, 1H), |
| | pyrrolo[3,2-d]pyrimidin-5-yl}methyl | | | | 7.00-7.21 (m, 3H), 6.20-6.42 |
| | 3-[(2S)-2-{[(tert- | | | | (m, 3H), 4.42-4.61 (m, 2H), |
| | butoxy)carbonyl]amino}-3-(1H- | | | | 4.26-4.42 (m, 1H), 3.80-3.95 |
| | indol-3- | | | | (m, 2H), 3.41-3.71 (m, 2H), |
| | yl)propanamido]propanoate | | | | 3.41-3.71 (m, 1H), 2.94-3.31 |
| | | | | | (m, 3H), 2.36-2.58 (m, 2H), |
| | | | | | 1.20-1.58 (m, 9H), 1.13 (dd, |
| | | | | | J = 3.22, 6.15 Hz, 6H) |
| 49 | trifluoroacetic acid {4-oxo-1-[2- | 3.7 | 546 | A | 1H NMR (300 MHz, cd3od) δ |
| | (propan-2-yloxy)ethyl]-2- | | | | 7.47 (d, J = 2.34 Hz, 1H), 7.09 |
| | sulfanylidene-1H,2H,3H,4H,5H- | | | | (d, J = 8.20 Hz, 2H), 6.79 (d, |
| | pyrrolo[3,2-d]pyrimidin-5-yl}methyl | | | | J = 7.62 Hz, 2H), 6.32-6.43 (m, |
| | 5-[(2S)-2-amino-3-(4- | | | | 1H), 6.28 (s, 2H), 4.60 (t, |
| | hydroxyphenyl)propanamido]pentanoate | | | | J = 5.57 Hz, 2H), 3.78-4.04 (m, |
| | | | | | 3H), 3.49-3.71 (m, 1H), 3.17- |
| | | | | | 3.29 (m, 1H), 2.91-3.15 (m, |
| | | | | | 3H), 2.27-2.41 (m, 2H), 1.34- |
| | | | | | 1.55 (m, 4H), 1.01-1.16 (m, 6H) |
| 50 | trifluoroacetic acid {4-oxo-1-[2- | 3.5 | 518 | A | 1H NMR (300 MHz, cd3od) δ |
| | (propan-2-yloxy)ethyl]-2- | | | | 7.41-7.57 (m, 1H), 6.98-7.14 |
| | sulfanylidene-1H,2H,3H,4H,5H- | | | | (m, 2H), 6.71-6.90 (m, 2H), |
| | pyrrolo[3,2-d]pyrimidin-5-yl}methyl | | | | 6.32-6.43 (m, 1H), 6.24-6.32 |
| | 3-[(2S)-2-amino-3-(4- | | | | (m, 2H), 4.44-4.65 (m, 2H), |
| | hydroxyphenyl)propanamido]propanoate | | | | 3.80-4.08 (m, 3H), 3.42-3.68 |
| | | | | | (m, 2H), 2.85-3.10 (m, 2H), |
| | | | | | 2.47-2.65 (m, 2H), 1.01-1.19 |
| | | | | | (m, 6H) |
| 51 | trifluoroacetic acid {4-oxo-1-[2- | 4 | 569 | A | 1H NMR (300 MHz, cd3od) δ |
| | (propan-2-yloxy)ethyl]-2- | | | | 7.62 (d, J = 8.20 Hz, 1H), 7.29- |
| | sulfanylidene-1H,2H,3H,4H,5H- | | | | 7.54 (m, 2H), 6.93-7.29 (m, |
| | pyrrolo[3,2-d]pyrimidin-5-yl}methyl | | | | 3H), 6.33-6.48 (m, 1H), 6.18- |
| | 5-[(2S)-2-amino-3-(1H-indol-3- | | | | 6.32 (m, 2H), 4.44-4.66 (m, |
| | yl)propanamido]pentanoate | | | | 2H), 3.96-4.18 (m, 1H), 3.75- |
| | | | | | 3.95 (m, 2H), 3.60 (s, 1H), 2.90- |
| | | | | | 3.30 (m, 4H), 2.10-2.37 (m, |
| | | | | | 2H), 1.14-1.48 (m, 5H), 0.97- |
| | | | | | 1.14 (m, 6H) |
| 52 | trifluoroacetic acid {4-oxo-1-[2- | 3.8 | 541 | A | 1H NMR (300 MHz, cd3od) δ |
| | (propan-2-yloxy)ethyl]-2- | | | | 7.55-7.68 (m, 1H), 7.34-7.51 |
| | sulfanylidene-1H,2H,3H,4H,5H- | | | | (m, 2H), 7.00-7.26 (m, 3H), |
| | pyrrolo[3,2-d]pyrimidin-5-yl}methyl | | | | 6.10-6.39 (m, 3H), 4.18-4.54 |
| | 3-[(2S)-2-amino-3-(1H-indol-3- | | | | (m, 2H), 3.95-4.12 (m, 1H), |
| | yl)propanamido]propanoate | | | | 3.70-3.88 (m, 2H), 3.44-3.65 |
| | | | | | (m, 2H), 3.02-3.30 (m, 2H), |
| | | | | | 2.36-2.61 (m, 2H), 1.07 (d, |
| | | | | | J = 5.86 Hz, 6H) |
| 53 | trifluoroacetic acid {4-oxo-1-[2- | 4.9 | 554 | A | 1H NMR (300 MHz, cd3od) δ |
| | (propan-2-yloxy)ethyl]-2- | | | | 7.37-7.60 (m, 1H), 6.36-6.48 |
| | sulfanylidene-1H,2H,3H,4H,5H- | | | | (m, 1H), 6.30 (d, J = 1.76 Hz, |
| | pyrrolo [3,2-d]pyrimidin-5-yl}methyl | | | | 2H), 4.56-4.70 (m, 2H), 3.82- |
| | 5-(2-{[(tert- | | | | 4.03 (m, 4H), 3.65 (s, 1H), 3.16- |
| | butoxy)carbonyl](methyl)amino}ace | | | | 3.31 (m, 2H), 2.97 (d, J = 1.76 |
| | tamido)pentanoate | | | | Hz, 3H), 2.43 (dt, J = 1.76, 7.03 |
| | | | | | Hz, 2H), 1.37-1.81 (m, 14H), |
| | | | | | 1.12 (dd, J = 2.05, 6.15 Hz, 6H) |
| 54 | trifluoroacetic acid {4-oxo-1-[2- | 4.7 | 526 | A | 1H NMR (300 MHz, cd3od) δ |
| | (propan-2-yloxy)ethyl]-2- | | | | 7.51 (d, J = 2.93 Hz, 1H), 6.43 |
| | sulfanylidene-1H,2H,3H,4H,5H- | | | | (d, J = 2.93 Hz, 1H), 6.31 (s, |
| | pyrrolo[3,2-d]pyrimidin-5-yl}methyl | | | | 2H), 6.23-6.35 (m, 1H), 4.55- |
| | 3-(2-{[(tert- | | | | 4.72 (m, 2H), 3.92 (s, 4H), 3.44- |
| | butoxy)carbonyl](methyl)amino}ace | | | | 3.72 (m, 3H), 2.94 (s, 3H), 2.63 |
| | tamido)propanoate | | | | (t, J = 6.44 Hz, 2H), 1.38-1.62 |
| | | | | | (m, 9H), 1.13 (d, J = 6.44 Hz, |
| | | | | | 6H) |
| 55 | trifluoroacetic acid {4-oxo-1-[2- | 3.3 | 454 | A | 1H NMR (300 MHz, cd3od) δ |
| | (propan-2-yloxy)ethyl]-2- | | | | 7.49-7.68 (m, 1H), 6.52 (d, |
| | sulfanylidene-1H,2H,3H,4H,5H- | | | | J = 2.93 Hz, 1H), 6.39 (s, 2H), |
| | pyrrolo[3,2-d]pyrimidin-5-yl}methyl | | | | 4.73 (t, J = 5.57 Hz, 2H), 4.02 (t, |
| | 5-[2- | | | | J = 5.86 Hz, 2H), 3.91 (s, 2H), |
| | (methylamino)acetamido]pentanoate | | | | 3.75 (td, J = 5.93, 12.16 Hz, |
| | | | | | 1H), 3.33-3.43 (m, 2H), 2.88 (s, |
| | | | | | 3H), 2.43-2.63 (m, 2H), 1.53- |
| | | | | | 1.90 (m, 5H), 1.09-1.29 (m, 6H) |
| 56 | trifluoroacetic acid {4-oxo-1-[2- | 3.2 | 426 | A | 1H NMR (300 MHz, cd3od) δ |
| | (propan-2-yloxy)ethyl]-2- | | | | 7.18-7.40 (m, 1H), 6.25 (d, |
| | sulfanylidene-1H,2H,3H,4H,5H- | | | | J = 1.17 Hz, 1H), 6.11 (br. s., |
| | pyrrolo [3,2-d]pyrimidin-5-yl}methyl | | | | 2H), 4.35-4.53 (m, 2H), 3.74 (d, |
| | 3-[2- | | | | J = 1.17 Hz, 2H), 3.54-3.67 (m, |
| | (methylamino)acetamido]propanoate | | | | 2H), 3.46-3.69 (m, 3H), 3.32- |
| | | | | | 3.54 (m, 3H), 3.23-3.23 (m, |
| | | | | | 1H), 2.55-2.65 (m, 3H), 2.39- |
| | | | | | 2.54 (m, 2H), 0.89-1.01 (m, 6H) |
| 57 | trifluoroacetic acid {4-oxo-1-[2- | 4.7 | 611 | A | 1H NMR (300 MHz, cd3od) δ |
| | (propan-2-yloxy)ethyl]-2- | | | | 7.43 (d, J = 2.93 Hz, 1H), 6.56- |
| | sulfanylidene-1H,2H,3H,4H,5H- | | | | 6.68 (m, 1H), 6.50-6.50 (m, |
| | pyrrolo[3,2-d]pyrimidin-5-yl}methyl | | | | 1H), 6.30-6.44 (m, 1H), 6.23 (s, |
| | 5-[2-(2-{[(tert- | | | | 2H), 4.56 (t, J = 5.57 Hz, 2H), |
| | butoxy)carbonyl]amino}-N- | | | | 3.91-4.11 (m, 2H), 3.78-3.91 |
| | methylacetamido)acetamido]pentanoate | | | | (m, 2H), 3.58 (quin, J = 6.15 Hz, |
| | | | | | 1H), 3.13-3.27 (m, 1H), 2.88- |
| | | | | | 3.11 (m, 2H), 2.27-2.47 (m, |
| | | | | | 2H), 2.08-2.08 (m, 1H), 1.32- |
| | | | | | 1.71 (m, 12H), 1.00-1.16 (m, |
| | | | | | 6H) |
| 58 | trifluoroacetic acid {4-oxo-1-[2- | 4.5 | 583 | A | 1H NMR (300 MHz, cd3od) δ |
| | (propan-2-yloxy)ethyl]-2- | | | | 7.82-7.95 (m, 2H), 7.28-7.45 |
| | sulfanylidene-1H,2H,3H,4H,5H- | | | | (m, 4H), 6.54 (d, J = 3.51 Hz, |
| | pyrrolo [3,2-d]pyrimidin-5-yl}methyl | | | | 2H), 6.09-6.25 (m, 2H), 3.21- |
| | 3-[2-(2-{[(tert- | | | | 3.28 (m, 4H), 2.87-3.03 (m, |
| | butoxy)carbonyl]amino}-N- | | | | 4H), 2.38 (d, J = 1.17 Hz, 6H), |
| | methylacetamido)acetamido]propanoate | | | | 2.18 (s, 6H) |
| 59 | trifluoroacetic acid {4-oxo-1-[2- | 3.4 | 511 | A | 1H NMR (300 MHz, cd3od) δ |
| | (propan-2-yloxy)ethyl]-2- | | | | 7.33-7.48 (m, 1H), 6.36 (d, |
| | sulfanylidene-1H,2H,3H,4H,5H- | | | | J = 2.93 Hz, 1H), 6.24 (s, 2H), |
| | pyrrolo[3,2-d]pyrimidin-5-yl}methyl | | | | 4.51-4.61 (m, 2H), 3.79-4.13 |
| | 5-[2-(2-amino-N- | | | | (m, 7H), 3.50-3.68 (m, 1H), |
| | methylacetamido)acetamido]pentanoate | | | | 3.12-3.26 (m, 3H), 3.03 (s, 2H), |
| | | | | | 2.28-2.43 (m, 2H), 1.41-1.70 |
| | | | | | (m, 4H), 1.00-1.09 (m, 6H) |
| 60 | trifluoroacetic acid methyl 2-{[3-oxo- | 4.1 | 510 | A | 1H NMR (300 MHz, cd3od) δ |
| | 3-({4-oxo-1-[2-(propan-2- | | | | 8.07 (br. s., 1H), 7.45 (d, |
| | yloxy)ethyl]-2-sulfanylidene- | | | | J = 2.93 Hz, 1H), 6.36 (d, |
| | 1H,2H,3H,4H,5H-pyrrolo[3,2- | | | | J = 2.93 Hz, 1H), 6.24 (s, 2H), |
| | d]pyrimidin-5- | | | | 4.49-4.62 (m, 2H), 4.16 (dd, |
| | yl}methoxy)propyl]carbamoyl}pyrrol | | | | J = 2.93, 8.20 Hz, 1H), 3.85 (t, |
| | idine-1-carboxylate | | | | J = 5.57 Hz, 2H), 3.35-3.72 (m, |
| | | | | | 9H), 2.43-2.65 (m, 2H), 1.74- |
| | | | | | 2.25 (m, 4H), 0.95-1.17 (m, 6H) |
| 61 | trifluoroacetic acid methyl 2-{[5-oxo- | 4.3 | 538 | A | 1H NMR (300 MHz, cd3od) δ |
| | 5-({4-oxo-1-[2-(propan-2- | | | | 7.36-7.55 (m, 1H), 6.35 (d, |
| | yloxy)ethyl]-2-sulfanylidene- | | | | J = 2.93 Hz, 1H), 6.24 (s, 2H), |
| | 1H,2H,3H,4H,5H-pyrrolo[3,2- | | | | 4.56 (t, J = 5.86 Hz, 2H), 4.10- |
| | d]pyrimidin-5- | | | | 4.23 (m, 1H), 3.85 (t, J = 5.57 |
| | yl}methoxy)pentyl]carbamoyl}pyrrol | | | | Hz, 2H), 3.34-3.72 (m, 6H), |
| | idine-1-carboxylate | | | | 3.16 (s, 2H), 2.26-2.43 (m, 2H), |
| | | | | | 1.80-2.00 (m, 3H), 1.40-1.71 |
| | | | | | (m, 5H), 0.99-1.11 (m, 6H) |
| 62 | trifluoroacetic acid {4-oxo-1-[2- | 3.6 | 495 | A | 1H NMR (300 MHz, cd3od) δ |
| | (propan-2-yloxy)ethyl]-2- | | | | 7.45 (d, J = 2.93 Hz, 1H), 6.36 |
| | sulfanylidene-1H,2H,3H,4H,5H- | | | | (d, J = 3.51 Hz, 1H), 6.24 (d, |
| | pyrrolo[3,2-d]pyrimidin-5-yl}methyl | | | | J = 2.93 Hz, 2H), 4.57 (t, J = 5.57 |
| | 3-{[(2S)-1-carbamoylpyrrolidin-2- | | | | Hz, 2H), 4.21-4.34 (m, 1H), |
| | yl]formamido}propanoate | | | | 3.85 (t, J = 5.86 Hz, 2H), 3.33- |
| | | | | | 3.71 (m, 5H), 2.43-2.61 (m, |
| | | | | | 2H), 2.03-2.22 (m, 1H), 1.86- |
| | | | | | 2.01 (m, 3H), 1.06 (d, J = 5.86 |
| | | | | | Hz, 6H) |
| 63 | trifluoroacetic acid {4-oxo-1-[2- | 4 | 508 | A | 1H NMR (300 MHz, cd3od) δ |
| | (propan-2-yloxy)ethyl]-2- | | | | 7.43 (d, J = 2.93 Hz, 1H), 6.35 |
| | sulfanylidene-1H,2H,3H,4H,5H- | | | | (d, J = 2.93 Hz, 1H), 6.24 (s, |
| | pyrrolo[3,2-d]pyrimidin-5-yl}methyl | | | | 2H), 4.57 (t, J = 5.86 Hz, 2H), |
| | 5-{[(2S)-1-methyl-5-oxopyrrolidin-2- | | | | 4.00-4.18 (m, 1H), 3.85 (t, |
| | yl]formamido}pentanoate | | | | J = 5.86 Hz, 2H), 3.58 (quin, |
| | | | | | J = 6.00 Hz, 1H), 3.11-3.25 (m, |
| | | | | | 3H), 2.67-2.81 (m, 3H), 2.17- |
| | | | | | 2.54 (m, 6H), 1.84-2.07 (m, |
| | | | | | 1H), 1.33-1.70 (m, 5H), 0.96- |
| | | | | | 1.13 (m, 6H) |
| 64 | trifluoroacetic acid {4-oxo-1-[2- | 3.7 | 480 | A | 1H NMR (300 MHz, cdcl3) δ |
| | (propan-2-yloxy)ethyl]-2- | | | | 8.67 (d, J = 4.69 Hz, 4H), 7.89 |
| | sulfanylidene-1H,2H,3H,4H,5H- | | | | (d, J = 8.20 Hz, 2H), 7.69 (s, |
| | pyrrolo[3,2-d]pyrimidin-5-yl}methyl | | | | 2H), 7.47-7.61 (m, 6H), 6.52 (d, |
| | 3-{[(2S)-1-methyl-5-oxopyrrolidin-2- | | | | J = 2.93 Hz, 2H), 6.14 (d, |
| | yl]formamido}propanoate | | | | J = 2.34 Hz, 2H), 3.15-3.47 (m, |
| | | | | | 4H), 2.98 (t, J = 7.62 Hz, 4H), |
| | | | | | 2.41 (s, 6H), 2.11-2.27 (m, 6H) |
| 65 | trifluoroacetic acid ethyl 2-{[5-oxo-5 | 4.5 | 552 | A | 1H NMR (300 MHz, cd3od) δ |
| | ({4-oxo-1-[2-(propan-2-yloxy)ethyl]- | | | | 7.38-7.51 (m, 1H), 6.32-6.39 |
| | 2-sulfanylidene-1H,2H,3H,4H,5H- | | | | (m, 1H), 6.19-6.29 (m, 2H), |
| | pyrrolo[3,2-d]pyrimidin-5- | | | | 4.49-4.59 (m, 2H), 3.96-4.25 |
| | yl}methoxy)pentyl]carbamoyl}pyrrol | | | | (m, 3H), 3.75-3.92 (m, 2H), |
| | idine-1-carboxylate | | | | 3.35-3.74 (m, 3H), 3.04-3.21 |
| | | | | | (m, 2H), 2.03-2.41 (m, 4H), |
| | | | | | 1.81-2.01 (m, 3H), 1.42-1.72 |
| | | | | | (m, 5H), 1.10-1.31 (m, 3H), |
| | | | | | 0.99-1.09 (m, 6H) |
| 66 | trifluoroacetic acid ethyl 2-{[3-oxo-3 | 4.3 | 524 | A | 1H NMR (300 MHz, cd3od) δ |
| | ({4-oxo-1-[2-(propan-2-yloxy)ethyl]- | | | | 7.45 (d, J = 2.93 Hz, 1H), 6.36 |
| | 2-sulfanylidene-1H,2H,3H,4H,5H- | | | | (d, J = 2.93 Hz, 1H), 6.24 (s, |
| | pyrrolo[3,2-d]pyrimidin-5- | | | | 2H), 4.44-4.63 (m, 2H), 3.92- |
| | yl}methoxy)propyl]carbamoyl}pyrrol | | | | 4.23 (m, 3H), 3.76-3.92 (m, |
| | idine-1-carboxylate | | | | 2H), 3.35-3.69 (m, 6H), 2.42- |
| | | | | | 2.61 (m, 2H), 2.03-2.28 (m, |
| | | | | | 1H), 1.71-1.97 (m, 3H), 1.09- |
| | | | | | 1.36 (m, 3H), 0.99-1.09 (m, 6H) |
| 67 | trifluoroacetic acid propan-2-yl 2- | 4.7 | 566 | A | 1H NMR (300 MHz, cd3od) δ |
| | {[5-oxo-5-({4-oxo-1-[2-(propan-2- | | | | 7.38-7.51 (m, 1H), 6.30-6.42 |
| | yloxy)ethyl]-2-sulfanylidene- | | | | (m, 1H), 6.17-6.30 (m, 2H), |
| | 1H,2H,3H,4H,5H-pyrrolo[3,2- | | | | 4.75-4.83 (m, 1H), 4.48-4.64 |
| | d]pyrimidin-5- | | | | (m, 2H), 4.08-4.26 (m, 1H), |
| | yl}methoxy)pentyl]carbamoyl}pyrrol | | | | 3.73-3.94 (m, 3H), 3.35-3.70 |
| | idine-1-carboxylate | | | | (m, 4H), 3.10-3.25 (m, 3H), |
| | | | | | 2.05-2.43 (m, 4H), 1.77-2.00 |
| | | | | | (m, 4H), 1.36-1.71 (m, 6H), |
| | | | | | 1.10-1.33 (m, 8H), 0.90-1.10 |
| | | | | | (m, 8H) |
| 68 | trifluoroacetic acid propan-2-yl 2- | 4.6 | 538 | A | 1H NMR (300 MHz, cd3od) δ |
| | {[3-oxo-3-({4-oxo-1-[2-(propan-2- | | | | 7.35-7.58 (m, 1H), 6.36 (d, |
| | yloxy)ethyl]-2-sulfanylidene- | | | | J = 2.93 Hz, 1H), 6.20-6.30 (m, |
| | 1H,2H,3H,4H,5H-pyrrolo[3,2- | | | | 2H), 4.78-4.84 (m, 1H), 4.40- |
| | d]pyrimidin-5- | | | | 4.65 (m, 2H), 4.07-4.25 (m, |
| | yl}methoxy)propyl]carbamoyl}pyrrol | | | | 1H), 3.75-3.93 (m, 2H), 3.35- |
| | idine-1-carboxylate | | | | 3.69 (m, 6H), 2.43-2.63 (m, |
| | | | | | 2H), 2.00-2.31 (m, 1H), 1.71- |
| | | | | | 1.96 (m, 4H), 1.10-1.31 (m, |
| | | | | | 7H), 1.01-1.08 (m, 6H) |
| 69 | trifluoroacetic acid {4-oxo-1-[2- | 3.8 | 523 | A | 1H NMR (300 MHz, cd3od) δ |
| | (propan-2-yloxy)ethyl]-2- | | | | 7.43 (d, J = 3.51 Hz, 1H), 6.35 |
| | sulfanylidene-1H,2H,3H,4H,5H- | | | | (d, J = 2.93 Hz, 1H), 6.23 (s, |
| | pyrrolo[3,2-d]pyrimidin-5-yl}methyl | | | | 2H), 6.22-6.32 (m, 2H), 4.49- |
| | 5-{[(2S)-1-carbamoylpyrrolidin-2- | | | | 4.67 (m, 2H), 4.15-4.38 (m, |
| | yl]formamido}pentanoate | | | | 1H), 3.79-3.92 (m, 2H), 3.33- |
| | | | | | 3.72 (m, 3H), 3.08-3.22 (m, |
| | | | | | 2H), 2.27-2.42 (m, 2H), 1.85- |
| | | | | | 2.27 (m, 4H), 1.35-1.70 (m, |
| | | | | | 4H), 0.91-1.15 (m, 6H) |

| | Caco-2 Pₐₚₚ | | | human Plasma |
|---|---|---|---|---|
| Example | (nm/s) | SGF | SIF | stability |
| 13 | 18.8 | 96% remaining | 94% remaining | 1% remaining |
| | | at 1 h | at 1 h | at 1 h |
| 14 | <2.03 | 95% remaining | 84% remaining | 55% remaining |
| | | at 1 h | at 1 h | at 1 h |
| 15 | >5.67 | 100% | 4% remaining | 9% remaining |
| | | remaining at 1 h | at 1 h | at 1 h |
| 16 | 9.03 | 96% remaining | 93% remaining | 21% remaining |
| | | at 1 h | at 1 h | at 1 h |
| 17 | 3.68 | 99% remaining | 3% remaining | 38% remaining |
| | | at 1 h | at 1 h | at 1 h |
| 18 | 75.9 | 100% | 65% remaining | 66% remaining |
| | | remaining at 1 h | at 1 h | at 1 h |
| 19 | 117 | 82% remaining | 57% remaining | 42% remaining |
| | | at 1 h | at 1 h | at 1 h |
| 20 | 9.85 | 80% remaining | 76% remaining | 64% remaining |
| | | at 1 h | at 1 h | at 1 h |
| 21 | 57.9 | 88% remaining | 75% remaining | 91% remaining |
| | | at 1 h | at 1 h | at 1 h |
| 22 | 6.47 | 87% remaining | 90% remaining | 74% remaining |
| | | at 1 h | at 1 h | at 1 h |
| 23 | 19.4 | 93% remaining | 84% remaining | 92% remaining |
| | | at 1 h | at 1 h | at 1 h |
| 24 | 3.02 | 99% remaining | 76% remaining | 1% remaining |
| | | at 1 h | at 1 h | at 1 h |
| 25 | 2.87 | 99% remaining | 43% remaining | 96% remaining |
| | | at 1 h | at 1 h | at 1 h |
| 26 | 127 | 99% remaining | 66% remaining | 32% remaining |
| | | at 1 h | at 1 h | at 1 h |
| 27 | 86.1 | 92% remaining | 61% remaining | 91% remaining |
| | | at 1 h | at 1 h | at 1 h |
| 28 | 67 | 99% remaining | 75% remaining | 62% remaining |
| | | at 1 h | at 1 h | at 1 h |
| 29 | 54.1 | 92% remaining | 62% remaining | 98% remaining |
| | | at 1 h | at 1 h | at 1 h |
| 30 | <2.69 | 93% remaining | 67% remaining | 40% remaining |
| | | at 1 h | at 1 h | at 1 h |
| 31 | 3.25 | 94% remaining | 85% remaining | 1% remaining |
| | | at 1 h | at 1 h | at 1 h |
| 32 | 1.47 | 97% remaining | 33% remaining | 17% remaining |
| | | at 1 h | at 1 h | at 1 h |
| 33 | 94.7 | 90% remaining | 78% remaining | 53% remaining |
| | | at 1 h | at 1 h | at 1 h |
| 34 | 107 | 85% remaining | 90% remaining | 93% remaining |
| | | at 1 h | at 1 h | at 1 h |
| 35 | 19.1 | 91% remaining | 99% remaining | 30% remaining |
| | | at 1 h | at 1 h | at 1 h |
| 36 | 8.35 | 95% remaining | 88% remaining | 79% remaining |
| | | at 1 h | at 1 h | at 1 h |
| 37 | 10.9 | 88% remaining | 89% remaining | 1% remaining |
| | | at 1 h | at 1 h | at 1 h |
| 38 | 3.1 | 92% remaining | 84% remaining | 65% remaining |
| | | at 1 h | at 1 h | at 1 h |
| 39 | <1.96 | 91% remaining | 91% remaining | 5% remaining |
| | | at 1 h | at 1 h | at 1 h |
| 40 | <2.35 | 100% | 86% remaining | 88% remaining |
| | | remaining at 1 h | at 1 h | at 1 h |
| 41 | 11.5 | 78% remaining | 87% remaining | 21% remaining |
| | | at 1 h | at 1 h | at 1 h |
| 42 | 5.07 | 86% remaining | 75% remaining | 51% remaining |
| | | at 1 h | at 1 h | at 1 h |
| 43 | 1.47 | 92% remaining | 87% remaining | 16% remaining |
| | | at 1 h | at 1 h | at 1 h |
| 44 | <2.22 | 96% remaining | 1% remaining | 5% remaining |
| | | at 1 h | at 1 h | at 1 h |
| 45 | 22.8 | 100% | 89% remaining | 66% remaining |
| | | remaining at 1 h | at 1 h | at 1 h |
| 46 | 11.2 | 98% remaining | 100% | 63% remaining |
| | | at 1 h | remaining at 1 h | at 1 h |
| 47 | 91.5 | 99% remaining | 46% remaining | 78% remaining |
| | | at 1 h | at 1 h | at 1 h |
| 48 | 59.3 | 90% remaining | 89% remaining | 74% remaining |
| | | at 1 h | at 1 h | at 1 h |
| 49 | 1.54 | 99% remaining | 92% remaining | 1% remaining |
| | | at 1 h | at 1 h | at 1 h |
| 50 | 2.15 | 99% remaining | 75% remaining | 25% remaining |
| | | at 1 h | at 1 h | at 1 h |
| 51 | 5.19 | 94% remaining | 83% remaining | 2% remaining |
| | | at 1 h | at 1 h | at 1 h |
| 52 | 2.3 | 95% remaining | 81% remaining | 61% remaining |
| | | at 1 h | at 1 h | at 1 h |
| 53 | 34.3 | 92% remaining | 98% remaining | 0% remaining |
| | | at 1 h | at 1 h | at 1 h |
| 54 | 31.4 | 93% remaining | 87% remaining | 74% remaining |
| | | at 1 h | at 1 h | at 1 h |
| 55 | 2.44 | 100% | 98% remaining | 1% remaining |
| | | remaining at 1 h | at 1 h | at 1 h |
| 56 | 1.44 | 64% remaining | 1% remaining | 0% remaining |
| | | at 1 h | at 1 h | at 1 h |
| 57 | 2.8 | 81% remaining | 96% remaining | 0% remaining |
| | | at 1 h | at 1 h | at 1 h |
| 58 | 2.87 | 77% remaining | 84% remaining | 49% remaining |
| | | at 1 h | at 1 h | at 1 h |
| 59 | <2.54 | 95% remaining | 98% remaining | 11% remaining |
| | | at 1 h | at 1 h | at 1 h |
| 60 | 6.11 | 99% remaining | 88% remaining | 37% remaining |
| | | at 1 h | at 1 h | at 1 h |
| 61 | 9.71 | 98% remaining | 92% remaining | 0% remaining |
| | | at 1 h | at 1 h | at 1 h |
| 62 | 1.62 | 99% remaining | 99% remaining | 91% remaining |
| | | at 1 h | at 1 h | at 1 h |
| 63 | 2 | 97% remaining | 98% remaining | 0% remaining |
| | | at 1 h | at 1 h | at 1 h |
| 64 | <1.90 | 98% remaining | 97% remaining | 76% remaining |
| | | at 1 h | at 1 h | at 1 h |
| 65 | 18.6 | 87% remaining | 89% remaining | 20% remaining |
| | | at 1 h | at 1 h | at 1 h |
| 66 | 7.36 | 96% remaining | 80% remaining | 75% remaining |
| | | at 1 h | at 1 h | at 1 h |
| 67 | 28.4 | 92% remaining | 93% remaining | 57% remaining |
| | | at 1 h | at 1 h | at 1 h |
| 68 | 22.6 | 76% remaining | 69% remaining | 100% |
| | | at 1 h | at 1 h | remaining at 1 h |
| 69 | 2.17 | 84% remaining | 94% remaining | 7% remaining |
| | | at 1 h | at 1 h | at 1 h |

### Example 70

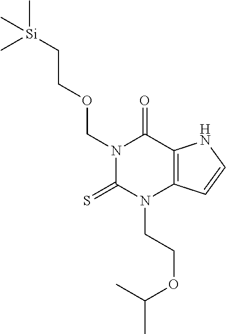 [SMILES: CC(C)OCCn1c(=S)n(COCC[Si](C)(C)C)c(=O)c2[nH]ccc21]

1-[2-(Propan-2-yloxy)ethyl]-2-sulfanylidene-3-{[2-(trimethylsilyl)ethoxy]methyl}-1H,2H,3H,4H,5H-pyrrolo[3,2-d]pyrimidin-4-one. To a solution of 2,3-dihydro-1-(2-isopropoxyethyl)-2-thioxo-1H-pyrrolo[3,2-d]pyrimidin-4(5H)-one (250 mg, 1.0 mmol) in 5.0 mL DMF was added DIPEA (365 μL, 2.1 mmol) and (2-(chloromethoxy)ethyl)trimethylsilane (195 μL, 1.1 mmol). The reaction was stirred overnight at room temperature. The reaction was quenched by the addition of water and extracted with EtOAc. The organic layer was washed with brine and the solvent was removed in vacuo. Crude product was purified by normal phase chromatography (12 g column, 0-70% MeOH/DCM) where the product fractions were combined and concentrated in vacuo to provide the pure product as a solid (58 mg, 15%). ¹H NMR (300 MHz, CHLOROFORM-d) δ 11.00-11.30 (m, 1H), 7.10-7.33 (m, 1H), 6.25 (t, J=2.34 Hz, 1H), 6.12 (s, 2H), 4.68 (t, J=5.86 Hz, 2H), 3.71-3.97 (m, 4H), 3.48-3.67 (m, 1H), 0.94-1.19 (m, 7H), −0.16-0.10 (m, 7H). LC/MS method A: Rₜ=6.2 mins (M+H)⁺=384, purity >95%. Caco-2 Pₐₚₚ=232, SGF=96% remaining at 1 h, SIF=99% remaining at 1 h, hPlasma 99% remaining at 1 h.

Additional compounds prepared according to EXAMPLE 70:

| Ex- | | LCMS | | | | Caco-2 | | | human |
|---|---|---|---|---|---|---|---|---|---|
| am- | | retention | M + | LCMS | | Pₐₚₚ | | | Plasma |
| ple | Name | time | H⁺ | method | NMR | (nm/s) | SGF | SIF | stability |
| 71 | 1-[2-(propan-2-yloxy)ethyl]-2- | 7.8 | 514 | A | 1H NMR (300 MHz, cdcl3) δ | 1.78 | 98% | 88% | 58% |
| | sulfanylidene-3,5-bis({[2- | | | | 7.09-7.23 (m, 1H), 6.25 (d, | | remain- | remain- | remain- |
| | (trimethylsilyl)ethoxy]methyl})- | | | | J = 2.93 Hz, 1H), 6.05 (s, 2H), | | ing | ing | ing |
| | 1H,2H,3H,4H,5H-pyrrolo[3,2- | | | | 5.74 (s, 2H), 4.58-4.74 (m, 2H), | | at 1 h | at 1 h | at 1 h |
| | d]pyrimidin-4-one | | | | 3.70-3.93 (m, 5H), 3.45-3.68 | | | | |
| | | | | | (m, 4H), 0.80-1.12 (m, 11H), - | | | | |
| | | | | | 0.12-0.12 (m, 18H) | | | | |
| 72 | 1-[2-(propan-2-yloxy)ethyl]-2- | 5.8 | 384 | A | 1H NMR (300 MHz, cdcl3) δ | 264 | 97% | 98% | 78% |
| | sulfanylidene-5-{[2- | | | | 9.19-9.38 (m, 1H), 7.20 (s, 1H), | | remain- | remain- | remain- |
| | (trimethylsilyl)ethoxy]methyl}- | | | | 6.27 (d, J = 3.52 Hz, 1H), 5.70 | | ing | ing | ing |
| | 1H,2H,3H,4H,5H-pyrrolo[3,2- | | | | (s, 2H), 4.55 (t, J = 5.57 Hz, 2H), | | at 1 h | at 1 h | at 1 h |
| | d]pyrimidin-4-one | | | | 3.86 (t, J = 5.86 Hz, 2H), 3.44- | | | | |
| | | | | | 3.68 (m, 3H), 1.07 (d, J = 5.86 | | | | |
| | | | | | Hz, 6H), 0.82-0.99 (m, 2H), | | | | |
| | | | | | -0.11-0.04 (m, 9H) | | | | |
| 73 | 1-[2-(propan-2-yloxy)ethyl]-5- | 5.8 | 514 | A | 1H NMR (300 MHz, cdcl3) δ | <7.17 | 35% | 55% | 99% |
| | {[2-(trimethylsilyl)ethoxy]- | | | | 7.12-7.21 (m, 1H), 6.21 (s, 1H), | | remain- | remain- | remain- |
| | methyl}-2-({[2- | | | | 5.89 (s, 2H), 5.61 (s, 2H), 4.21- | | ing | ing | ing |
| | (trimethylsilyl)ethoxy] | | | | 4.34 (m, 2H), 3.76 (s, 8H), 1.02- | | at 1 h | at 1 h | at 1 h |
| | methyl}sulfanyl)-1H,4H,5H- | | | | 1.17 (m, 6H), 0.92 (td, J = 8.20, | | | | |
| | pyrrolo[3,2-d]pyrimidin-4-one | | | | 17.57 Hz, 4H), -0.13-0.08 (m, | | | | |
| | | | | | 18H) | | | | |

### Example 74

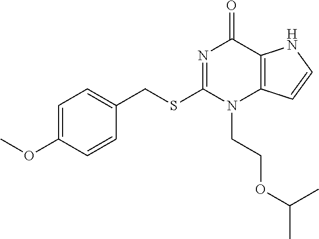 [SMILES: COc1ccc(CSc2nc(=O)c3[nH]ccc3n2CCOC(C)C)cc1]

2-{[(4-Methoxyphenyl)methyl]sulfanyl}-1-[2-(propan-2-yloxy)ethyl]-1H,4H,5H-pyrrolo[3,2-d]pyrimidin-4-one. A solution of 2,3-dihydro-1-(2-isopropoxyethyl)-2-thioxo-1H-pyrrolo[3,2-d]pyrimidin-4(5H)-one (100 mg, 400 μmol) in THF (2.0 mL) was treated with lithium hexamethyldisilylamide (1.0 M in THF, 840 μL, 840 μmol) and stirred for 20 mins. 1-(chloromethyl)-4-methoxybenzene (114 μL, 840 μmol) was added and the mixture was stirred overnight. The reaction was quenched by the addition of water and extracted with EtOAc. The organic layer was washed with brine and the solvent was removed in vacuo. Crude product was purified by normal phase chromatography (12 g column, 20-100% EtOAC/hexanes) but the material did not elute. The column was flushed with 10% MeOH/DCM where the product fractions were combined and concentrated in vacuo to provide the pure product as a solid (34 mg, 23%). ¹H NMR (300 MHz, CHLOROFORM-d) δ 7.30-7.48 (m, 3H), 6.76-6.96 (m, 2H), 6.09-6.27 (m, 1H), 4.59 (s, 2H), 4.27 (t, J=6.15 Hz, 2H), 3.67-3.83 (m, 5H), 3.51 (td, J=5.93, 12.16 Hz, 1H), 1.06 (d, J=6.44 Hz, 6H). LC/MS method A: Rₜ=4.4 mins (M+H)⁺=374, purity >95%. SGF=37% remaining at 1 h, SIF=97% remaining at 1 h, hPlasma stability=97% remaining at 1 h.

### Example 75

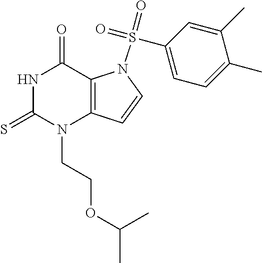 [SMILES: Cc1ccc(S(=O)(=O)n2ccc3c2c(=O)[nH]c(=S)n3CCOC(C)C)cc1C]

5-(3,4-Dimethylbenzenesulfonyl)-1-[2-(propan-2-yloxy)ethyl]-2-sulfanyl-1H,4H,5H-pyrrolo[3,2-d]pyrimidin-4-one. A solution of 2,3-dihydro-1-(2-isopropoxyethyl)-2-thioxo-1H-pyrrolo[3,2-d]pyrimidin-4(5H)-one (100 mg, 400 μmol) in THF (2.0 mL) was treated with lithium hexamethyldisilylamide (1.0 M in THF, 840 μL, 840 μmol) and stirred for 20 mins. 3,4-Dimethylbenzene-1-sulfonyl chloride (171 mg, 840 μmol) was added and the mixture was stirred overnight. The reaction was quenched by the addition of water and extracted with EtOAc. The organic layer was washed with brine and the solvent was removed in vacuo. Crude product was purified by normal phase chromatography (12 g column, 15-65% EtOAC/hexanes) where the product fractions were combined and concentrated in vacuo to provide the pure product as a solid (28 mg, 17%). ¹H NMR (300 MHz, CHLOROFORM-d) δ 9.12-9.29 (m, 1H), 7.67-7.95 (m, 3H), 7.30 (s, 1H), 6.44 (dd, J=1.17, 3.51 Hz, 1H), 4.35-4.58 (m, 2H), 3.70-3.88 (m, 2H), 3.40-3.62 (m, 1H), 2.18-2.41 (m, 7H), 1.47-1.64 (m, 2H), 0.96-1.07 (m, 6H). LC/MS method A: Rₜ=5.4 mins (M+H)⁺=422, purity >95%. SGF=99% remaining at 1 h, SIF=91% remaining at 1 h, hPlasma stability=100% remaining at 1 h.

Additional compounds prepared according to EXAMPLE 75:

| Ex- | | LCMS | | | | Caco-2 | | | human |
|---|---|---|---|---|---|---|---|---|---|
| am- | | retention | M + | LCMS | | Pₐₚₚ | | | Plasma |
| ple | Name | time | H⁺ | method | NMR | (nm/s) | SGF | SIF | stability |
| 76 | 5-methanesulfonyl-1-[2- | 4.1 | 332 | A | 1H NMR (300 MHz, cdcl3) δ | | 96% | 91% | 98% |
| | (propan-2-yloxy)ethyl]-2- | | | | 9.59-9.87 (m, 1H), 7.65 (d, | | remain- | remain- | remain- |
| | sulfanylidene- | | | | J = 3.51 Hz, 1H), 6.46 (d, | | ing | ing | ing |
| | 1H,2H,3H,4H,5H- | | | | J = 3.51 Hz, 1H), 4.55 (t, J = 5.27 | | at 1 h | at 1 h | at 1 h |
| | pyrrolo[3,2-d]pyrimidin- | | | | Hz, 2H), 3.66-3.96 (m, 5H), | | | | |
| | 4-one | | | | 3.55 (td, J = 5.93, 12.16 Hz, | | | | |
| | | | | | 1H), 1.25 (s, 2H), 0.97-1.16 (m, | | | | |
| | | | | | 6H) | | | | |
| 77 | 5-(ethanesulfonyl)-1-[2- | 4.4 | 346 | A | 1H NMR (300 MHz, cdcl3) δ | | 99% | 96% | 98% |
| | (propan-2-yloxy)ethyl]-2- | | | | 7.55-7.71 (m, 1H), 6.46 (d, | | remain- | remain- | remain- |
| | sulfanylidene- | | | | J = 3.52 Hz, 1H), 4.55 (t, J = 4.98 | | ing | ing | ing |
| | 1H,2H,3H,4H,5H- | | | | Hz, 2H), 4.01 (q, J = 7.62 Hz, | | at 1 h | at 1 h | at 1 h |
| | pyrrolo[3,2-d]pyrimidin- | | | | 2H), 3.85 (t, J = 5.27 Hz, 2H), | | | | |
| | 4-one | | | | 3.45-3.64 (m, 1H), 1.25-1.51 | | | | |
| | | | | | (m, 5H), 1.06 (d, J = 5.86 Hz, | | | | |
| | | | | | 6H) | | | | |
| 78 | 5-(benzenesulfonyl)-1-[2- | 4.9 | 394 | A | 1H NMR (300 MHz, cdcl3) δ | | 100% | 80% | 96% |
| | (propan-2-yloxy)ethyl]-2- | | | | 9.50-9.78 (m, 1H), 8.16 (d, | | remain- | remain- | remain- |
| | sulfanylidene- | | | | J = 7.62 Hz, 2H), 7.82 (d, | | ing | ing | ing |
| | 1H,2H,3H,4H,5H- | | | | J = 3.52 Hz, 1H), 7.43-7.71 (m, | | at 1 h | at 1 h | at 1 h |
| | pyrrolo[3,2-d]pyrimidin- | | | | 3H), 6.47 (d, J = 3.52 Hz, 1H), | | | | |
| | 4-one | | | | 4.41-4.60 (m, 2H), 3.73-3.88 | | | | |
| | | | | | (m, 2H), 3.37-3.62 (m, 1H), | | | | |
| | | | | | 1.04 (d, J = 5.86 Hz, 6H) | | | | |
| 79 | 5-(2-fluorobenzenesulfonyl)- | 5 | 412 | A | 1H NMR (300 MHz, cdcl3) δ | | 92% | 82% | 98% |
| | 1-[2-(propan-2-yloxy)ethyl]- | | | | 9.42 (br. s., 1H), 8.34-8.56 (m, | | remain- | remain- | remain- |
| | 2-sulfanylidene- | | | | 1H), 7.81-7.98 (m, 1H), 7.58- | | ing | ing | ing |
| | 1H,2H,3H,4H,5H- | | | | 7.76 (m, 1H), 7.32-7.49 (m, | | at 1 h | at 1 h | at 1 h |
| | pyrrolo[3,2-d]pyrimidin-4- | | | | 1H), 7.02-7.21 (m, 1H), 6.36- | | | | |
| | one | | | | 6.56 (m, 1H), 4.41-4.60 (m, | | | | |
| | | | | | 2H), 3.82 (t, J = 5.27 Hz, 2H), | | | | |
| | | | | | 3.34-3.66 (m, 1H), 0.95-1.12 | | | | |
| | | | | | (m, 6H) | | | | |
| 80 | 5-(2-methylbenzenesulfonyl)- | 5.2 | 408 | A | 1H NMR (300 MHz, cdcl3) δ | | 90% | 87% | 99% |
| | 1-[2-(propan-2-yloxy)ethyl]- | | | | 9.15-9.35 (m, 1H), 8.34-8.49 | | remain- | remain- | remain- |
| | 2-sulfanylidene- | | | | (m, 1H), 7.90 (dd, J = 2.05, 3.81 | | ing | ing | ing |
| | 1H,2H,3H,4H,5H- | | | | Hz, 1H), 7.28-7.65 (m, 3H), | | at 1 h | at 1 h | at 1 h |
| | pyrrolo[3,2-d]pyrimidin-4- | | | | 6.39-6.57 (m, 1H), 4.41-4.60 | | | | |
| | one | | | | (m, 2H), 3.82 (dt, J = 2.34, 5.27 | | | | |
| | | | | | Hz, 2H), 3.45-3.64 (m, 1H), | | | | |
| | | | | | 2.42 (s, 3H), 1.05 (dd, J = 2.05, | | | | |
| | | | | | 6.15 Hz, 6H) | | | | |

### Example 81

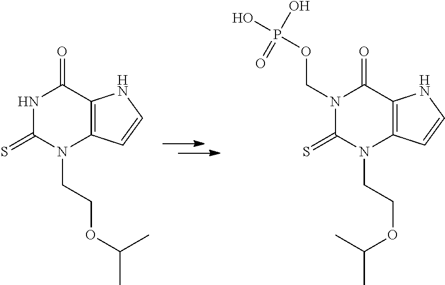 [SMILES: CC(C)OCCn1c(=S)[nH]c(=O)c2[nH]ccc21 ; CC(C)OCCn1c(=S)n(COP(=O)(O)O)c(=O)c2[nH]ccc21]

({4-Oxo-1-[2-(propan-2-yloxy)ethyl]-2-sulfanylidene-1H,2H,3H,4H,5H-pyrrolo[3,2-d]pyrimidin-3-yl}methoxy)phosphonic acid: A solution of 1-[2-(propan-2-yloxy)ethyl]-2-sulfanylidene-1H,2H,3H,4H,5H-pyrrolo[3,2-d]pyrimidin-4-one (506 mg, 2.0 mmol) in DMF (8 mL) under nitrogen was treated with lithium hexamethyldisilylamide (1.0 M in THF, 1.8 mL, 1.8 mmol) and stirred for 15 mins. Di-tert-butyl chloromethyl phosphate (520 mg, 2.0 mmol) was added via syringe with continued stirring of the reaction mixture overnight, followed by purification with RP-HPLC (Method D). The later fractions of similar retention time containing the desired product were combined and lyophilized to provide the di-tert-butyl protected intermediate (130 mg, 13.7%). Deprotection of tert-butyl protecting groups was carried out with glacial acetic acid and water (4:1 v/v) at 75° C. for 30 mins. followed by stirring at room temperature overnight. The reaction mixture was purified by RP-HPLC (Method D), and similar fractions were lyophilized to provide the title compound as a white solid (54 mg). ¹H NMR (300 MHz, DMSO-d₆) δ=12.50 (br s, 1H), 7.43 (t, J=2.9 Hz, 1H), 6.34 (t, J=2.6 Hz, 1H), 6.23 (d, J=4.7 Hz, 2H), 4.58 (t, J=6.4 Hz, 2H), 3.74 (t, J=6.2 Hz, 2H), 3.62-3.49 (m, 1H), 1.01 (d, J=6.1 Hz, 6H). LC/MS method A: Rₜ=3.02 mins., (M+H)⁺=364, purity=99%.

Additional compounds prepared according to EXAMPLE 81:

| Ex- | | LCMS | | | | Caco-2 | | | human |
|---|---|---|---|---|---|---|---|---|---|
| am- | | retention | M + | LCMS | | Pₐₚₚ | | | Plasma |
| ple | Name | time | H⁺ | method | NMR | (nm/s) | SGF | SIF | stability |
| 83 | ({4-oxo-1-[2-(propan-2- | 2.82 mins | 364 | | 1H NMR (300 MHz, dmso) δ | <10.7 | 91% | 100% | 95% @ |
| | yloxy)ethyl]-2-sulfanylidene- | | | | 12.26 (s, 1H), 7.50 (d, J = | | @ 1 h | @ 1 h | 1 h |
| | 1H,2H,3H,4H,5H- | | | | 3.1 Hz, 1H), 6.31 (d, J = 3.1 | | | | |
| | pyrrolo[3,2-d]pyrimidin-5- | | | | Hz, 1H), 5.84 (d, J = 10.5 Hz, | | | | |
| | yl}methoxy)phosphonic acid | | | | 2H), 4.48 (dd, J = 23.0, 16.8 | | | | |
| | | | | | Hz, 2H), 3.69 (dd, J = 12.2, | | | | |
| | | | | | 6.2 Hz, 2H), 3.54 (dt, J = | | | | |
| | | | | | 12.3, 6.2 Hz, 1H), 1.00 (dd, J | | | | |
| | | | | | = 6.1, 1.3 Hz, 6H). | | | | |

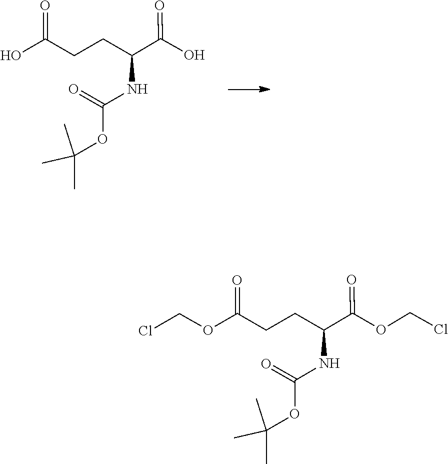 [SMILES: CC(C)(C)OC(=O)N[C@@H](CCC(=O)O)C(=O)O ; CC(C)(C)OC(=O)N[C@@H](CCC(=O)OCCl)C(=O)OCCl]

1,5-Dichloromethyl (2S)-2-{[(tert-butoxy)carbonyl]amino}pentanedioate: A solution of (2S)-2-{[(tert-butoxy)carbonyl]amino}pentanedioic acid (2.5 g, 10.11 mmol), tetrabutylammonium hydrogen sulfate (679 mg, 2.0 mmol), and sodium carbonate (4.54 g, 54 mmol) was dissolved in dichloromethane and water (50:50 v/v, 52 mL) and cooled at ° 0 C on an ice bath. Chloromethyl chlorosulfonate (2.42 mL, 24.3 mmol) was add dropwise and the reaction was warmed to room temperature with stirring overnight. The reaction was poured into a separatory funnel and separated. The aqueous layer was extracted one more time with dichloromethane and the combined organic fractions were washed with water and dried over magnesium sulfate and concentrated. The was purified by flash chromatography providing the titled compound (400 mg, 11.5%). ¹H NMR (300 MHz, CHLOROFORM-d) δ 5.84-5.59 (m, 4H), 5.07 (br d, J=7.6 Hz, 1H), 4.45-4.31 (m, 1H), 2.62-2.41 (m, 2H), 2.33-2.17 (m, 1H), 2.11-1.90 (m, 1H), 1.44 (s, 9H).

### Example 85

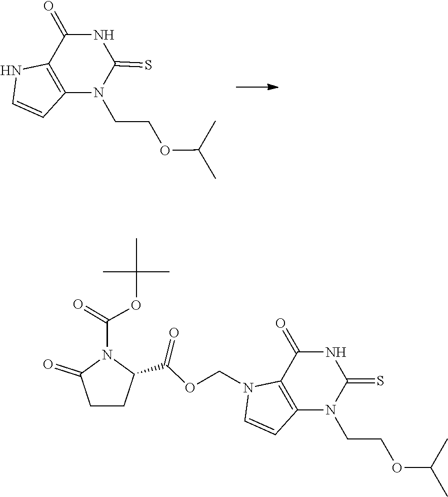 [SMILES: CC(C)OCCn1c(=S)[nH]c(=O)c2[nH]ccc21 ; CC(C)OCCn1c(=S)[nH]c(=O)c2c1ccn2COC(=O)[C@@H]1CCC(=O)N1C(=O)OC(C)(C)C]

1-tert-Butyl 2-{4-oxo-1-[2-(propan-2-yloxy)ethyl]-2-sulfanylidene-1H,2H,3H,4H,5H-pyrrolo[3,2-d]pyrimidin-5-yl}methyl (2S)-5-oxopyrrolidine-1,2-dicarboxylate: A solution of 1-[2-(propan-2-yloxy)ethyl]-2-sulfanylidene-1H,2H,3H,4H,5H-pyrrolo[3,2-d]pyrimidin-4-one (25 mg, 98.7 μmol) in DMF (1.5 mL) under nitrogen was treated with lithium hexamethyldisilylamide (1.0 M in THF, 197 μL, 197 μmol) and stirred for 15 mins. 1,5-Dichloromethyl (2S)-2-{[(tert-butoxy)carbonyl]amino}pentanedioate (34 mg, 197 μmol) in DMF (1.0 mL) was added via syringe with continued stirring of the reaction mixture overnight, followed by purification with RP-HPLC (Method D). Fractions containing the rearrangement product were freeze-dried an analysis determined the structure as the title compound, an orange solid (36.8 mg, 75.4%). ¹H NMR (300 MHz, DMSO-d₆) δ=12.40 (s, 1H), 7.56 (d, J=3.0 Hz, 1H), 6.41 (d, J=3.1 Hz, 1H), 6.26 (d, J=5.5 Hz, 2H), 4.64-4.54 (m, 1H), 4.46 (br t, J=5.9 Hz, 2H), 3.69 (br t, J=6.0 Hz, 2H), 3.63-3.32 (m, 1H), 2.44-2.32 (m, 2H), 2.25 (br s, 1H), 1.89 (br s, 1H), 1.40 (br d, J=5.6 Hz, 1H), 1.28 (s, 9H), 0.99 (d, J=6.1 Hz, 6H). LC/MS: Rₜ=4.34 mins., (M+H)⁺=395, minus t-Boc, purity=100%. Caco-2 Pₐₚₚ=65.4 nm/s, SGF=78% remaining at 1 h, SIF=90% remaining at 1 h, hPlasma stability=30% remaining at 1 h.

### Example 86

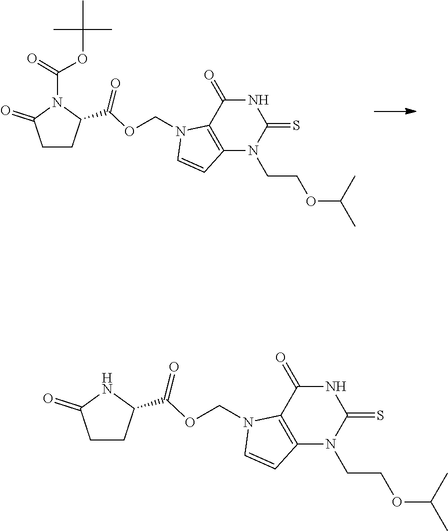 [SMILES: CC(C)OCCn1c(=S)[nH]c(=O)c2c1ccn2COC(=O)[C@@H]1CCC(=O)N1C(=O)OC(C)(C)C ; CC(C)OCCn1c(=S)[nH]c(=O)c2c1ccn2COC(=O)[C@@H]1CCC(=O)N1]

{4-Oxo-1-[2-(propan-2-yloxy)ethyl]-2-sulfanylidene-1H,2H,3H,4H,5H-pyrrolo[3,2-d]pyrimidin-5-yl}methyl(2S)-5-oxopyrrolidine-2-carboxylate: Trifluoroacetic acid in dichloromethane (30% v/v, 1 mL) was added to 1-tert-butyl 2-{4-oxo-1-[2-(propan-2-yloxy)ethyl]-2-sulfanylidene-1H,2H,3H,4H,5H-pyrrolo[3,2-d]pyrimidin-5-yl}methyl (2S)-5-oxopyrrolidine-1,2-dicarboxylate (15.5 mg, 31.4 μmol) and stirred at room temperature for 20 minutes. The reaction was concentrated and purified by RP-HPLC (Method B), with fractions containing product freeze-dried giving the titled compound as a white solid (5.3 mg, 42.9%). ¹H NMR (300 MHz, DMSO-d₆) δ 12.39 (s, 1H), 8.00 (s, 1H), 7.55 (d, J=3.1 Hz, 1H), 6.40 (d, J=3.2 Hz, 1H), 6.27-6.12 (m, 2H), 4.46 (t, J=6.0 Hz, 2H), 4.28-4.05 (m, 1H), 3.70 (t, J=6.0 Hz, 2H), 3.51 (tt, J=12.5, 6.3 Hz, 1H), 2.43-2.22 (m, 1H), 2.07 (dd, J=10.0, 6.2 Hz, 2H), 1.91 (ddd, J=11.7, 9.4, 5.8 Hz, 1H), 0.98 (d, J=6.0 Hz, 6H). LC/MS method A: Rₜ=3.45 mins., (M+H)⁺=395, purity >95%. Caco-2 Pₐₚₚ=2.4 nm/s, SGF=98% remaining at 1 h, SIF=17% remaining at 1 h, hPlasma stability=0% remaining at 1 h.

### Example 87

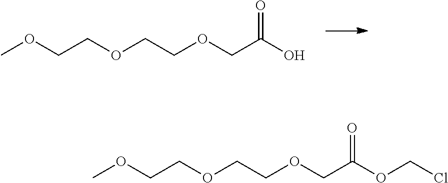

Chloromethyl 2-[2-(2-methoxyethoxy)ethoxy]acetate: A solution of 2-[2-(2-methoxyethoxy)ethoxy]acetic acid (2.08 mL, 13.5 mmol), tetrabutylammonium hydrogen sulfate (458 mg, 1.35 mmol), and sodium carbonate (4.54 g, 54 mmol) was dissolved in dichloromethane and water (50:50 v/v, 57 mL) and cooled at °0 C. on an ice bath. Chloromethyl chlorosulfonate (1.62 mL, 16.2 mmol) was add dropwise and the reaction was warmed to room temperature with stirring overnight. The reaction was poured into a separatory funnel and separated. The aqueous layer was extracted one more time with dichloromethane and the combined organic fractions were washed with water and dried over magnesium sulfate and concentrated to provide the crude product (1.67 g), which was purified by flash chromatography eluting with a gradient of hexane to 50% ethyl acetate in hexane. The product, chloromethyl 2-[2-(2-methoxyethoxy)ethoxy]acetate, was isolated as a clear oil in appearance (990 mg, 32.4%). ¹H NMR (300 MHz, CHLOROFORM-d) δ 5.77 (s, 2H), 4.25 (s, 2H), 3.77 (m, 2H), 3.73-3.68 (m, 2H), 3.68-3.61 (m, 2H), 3.60-3.50 (m, 2H), 3.39 (s, 3H). LC/MS method A: Rₜ=3.11 mins., (M+H)⁺=249 with sodium +23, purity=100%.

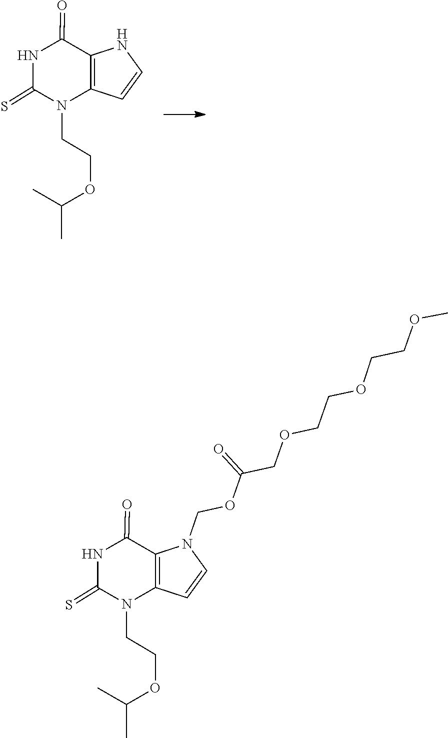 [SMILES: CC(C)OCCn1c(=S)[nH]c(=O)c2[nH]ccc21 ; COCCOCCOCC(=O)OCn1ccc2c1c(=O)[nH]c(=S)n2CCOC(C)C]

({4-Oxo-1-[2-(propan-2-yloxy)ethyl]-2-sulfanylidene-1H,2H,3H,4H,5H-pyrrolo[3,2-d]pyrimidin-3-yl}methoxy)phosphonic acid: A solution of 1-[2-(propan-2-yloxy)ethyl]-2-sulfanylidene-1H,2H,3H,4H,5H-pyrrolo[3,2-d]pyrimidin-4-one (150 mg, 592 μmol) in DMF (1 mL) under nitrogen was treated with lithium hexamethyldisilylamide (1.0 M in THF, 1.18 mL, 1.18 mmol) and stirred for 10 mins. Chloromethyl 2-[2-(2-methoxyethoxy)ethoxy]acetate (267 mg, 1.18 mmol) dissolved in DMF (1 mL) was added via syringe with continued stirring of the reaction mixture overnight, followed by purification with RP-HPLC (Method D). For further purification RP-HPLC (Method B) provided 130 mg, with pure fractions yielding the desired product for testing (10 mg). ¹H NMR (300 MHz, CHLOROFORM-d) δ 7.31-7.28 (m, 1H), 6.30 (s, 2H), 6.26 (d, J=3.2 Hz, 1H), 4.53 (t, J=5.6 Hz, 2H), 4.18 (s, 2H), 3.84 (t, J=5.6 Hz, 2H), 3.77-3.50 (m, 9H), 3.37 (s, 3H), 1.07 (d, J=6.1 Hz, 6H). LC/MS method A: Rₜ=3.92 mins., (M+H)⁺=444, purity=98%. Caco-2 Pₐₚₚ=70.5 nm/s, SGF=97% remaining at 1 h, SIF=28% remaining at 1 h, hPlasma stability=0% remaining at 1 h.

### Examples 88 and 89

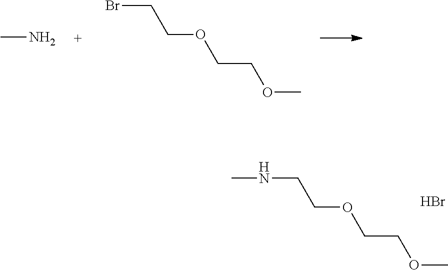

[2-(2-Methoxyethoxy)ethyl](methyl)amine hydrobromide: 1-Bromo-2-(2-methoxyethoxy)ethane (942 μL, 7 mmol) was dissolved in methyl ammonia in ethanol (10 mL, 33% by weight) and heated at 80° C. in a sealed tube overnight. The reaction was cooled to room temperature and concentrated to provide the desired product, in quantitative yield. ¹H NMR (300 MHz, CHLOROFORM-d) δ 6.02 (s, 1H), 3.90-3.83 (m, 2H), 3.73-3.65 (m, 2H), 3.63-3.54 (m, 2H), 3.40 (s, 3H), 3.17-3.10 (m, 2H), 2.71 (s, 3H).

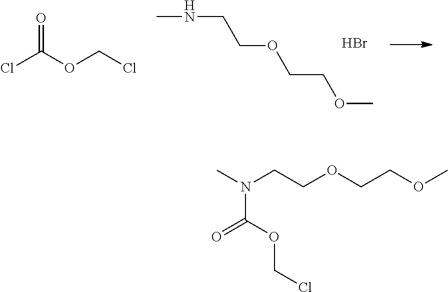

Chloromethyl N-[2-(2-methoxyethoxy)ethyl]-N-methylcarbamate: Di-isopropyl ethylamine (1.43 mL, 8.4 mmol) was added to [2-(2-Methoxyethoxy)ethyl](methyl)amine hydrobromide (642 mg, 3 mmol) in dichloromethane (10 mL) and cooled on an ice bath. Chloromethyl chloroformate was added dropwise and the reaction was warmed to room temperature overnight. The reaction was partitioned, separated, and the aqueous phase was extracted with dichloromethane (1×). The combined organic fractions were dried over magnesium sulfate and concentrated to provide 550 mg of crude product, which was purified using with flash chromatography eluting with a gradient of 10% ethyl acetate in hexane to 50% ethyl acetate in hexane. The titled compound was isolated as a clear oil (330 mg, 48.9%). ¹H NMR (300 MHz, CHLOROFORM-d) δ=5.79 (d, J=1.6 Hz, 2H), 3.82-3.42 (m, 8H), 3.38 (s, 3H), 3.02 (d, J=6.9 Hz, 3H).

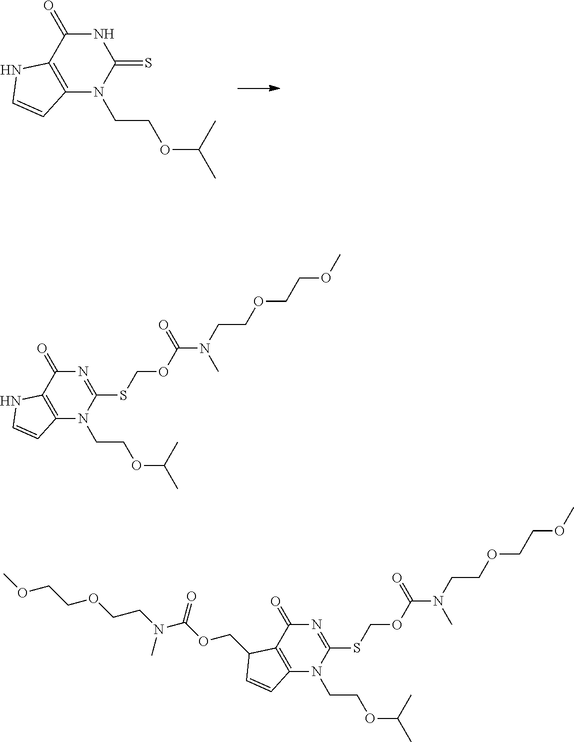

({4-Oxo-1-[2-(propan-2-yloxy)ethyl]-1H,4H,5H-pyrrolo[3,2-d]pyrimidin-2-yl}sulfanyl)methyl N-[2-(2-methoxyethoxy)ethyl]-N-methylcarbamate and (2-{[({[2-(2-methoxyethoxy)ethyl](methyl)carbamoyl}oxy)methyl]sulfanyl}-4-oxo-1-[2-(propan-2-yloxy)ethyl]-1H,4H,5H-pyrrolo[3,2-d]pyrimidin-5-yl)methyl N-[2-(2-methoxyethoxy)ethyl]-N-methylcarbamate: A solution of 1-[2-(propan-2-yloxy)ethyl]-2-sulfanylidene-1H,2H,3H,4H,5H-pyrrolo[3,2-d]pyrimidin-4-one (76 mg, 300 μmol) in DMF (0.5 mL) under nitrogen was treated with lithium hexamethyldisilylamide (1.0 M in THF, 300 μL, 300 μmol) and stirred for 10 mins. Chloromethyl N-[2-(2-methoxyethoxy)ethyl]-N-methylcarbamate (67.5 mg, 300 μmol) in DMF (0.5 mL) was added slowly and the reaction was stirred overnight, followed by purification with RP-HPLC (Method D).

Spectra for EXAMPLE 88: ({4-Oxo-1-[2-(propan-2-yloxy)ethyl]-1H,4H,5H-pyrrolo[3,2-d]pyrimidin-2-yl}sulfanyl)methyl N-[2-(2-methoxyethoxy)ethyl]-N-methylcarbamate. ¹H NMR (300 MHz, CHLOROFORM-d) δ 12.44 (s, 1H), 7.39 (t, J=2.9 Hz, 1H), 6.23-6.21 (m, 1H), 6.06 (d, J=2.1 Hz, 2H), 4.31 (t, J=6.0 Hz, 2H), 3.75 (t, J=6.0 Hz, 2H), 3.61 (m, 2H), 3.56-3.47 (m, 5H), 3.43 (m, 2H), 3.33 (d, J=18.5 Hz, 3H), 2.97 (d, J=17.8 Hz, 3H), 1.08-1.05 (m, 6H). LC/MS method A: Rₜ=3.49 mins., (M+H)⁺=443, purity=93%. Caco-2 Pₐₚₚ=7.51 nm/s, SGF=99% remaining at 1 h, SIF=64% remaining at 1 h, hPlasma stability=100% remaining at 1 h.

Spectra for EXAMPLE 89: (2-{[({[2-(2-methoxyethoxy)ethyl](methyl)carbamoyl}oxy)methyl]sulfanyl}-4-oxo-1-[2-(propan-2-yloxy)ethyl]-1H,4H,5H-pyrrolo[3,2-d]pyrimidin-5-yl)methyl N-[2-(2-methoxyethoxy)ethyl]-N-methylcarbamate. ¹H NMR (300 MHz, CHLOROFORM-d) δ 7.37 (t, J=3.2 Hz, 1H), 6.42 (d, J=5.0 Hz, 2H), 6.17 (d, J=3.2 Hz, 1H), 6.01 (s, 2H), 4.22 (t, J=6.0 Hz, 2H), 3.75-3.67 (m, 2H), 3.66-3.41 (m, 17H), 3.40-3.33 (m, 6H), 2.96 (dd, J=13.8, 9.1 Hz, 6H), 1.07 (d, J=6.1 Hz, 6H). LC/MS method A: Rₜ=3.87 mins., (M+H)⁺=632, purity=100%. Caco-2 Pₐₚₚ=4.88 nm/s, SGF=99% remaining at 1 h, SIF=65% remaining at 1 h, hPlasma stability=100% remaining at 1 h.

Additional compounds prepared according to EXAMPLES 87-89:

| | | LCMS | | | |
|---|---|---|---|---|---|
| | | retention | | LCMS | |
| Example | Name | time | M + H⁺ | method | NMR |
| 90 | {4-oxo-1-[2-(propan-2-yloxy)ethyl]- | 4.57 mins. | 370 | | 1H NMR (300 MHZ, CDCI3) δ |
| | 2-sulfanylidene-1H,2H,3H,4H,5H- | | | | 9.14 (s, 1H), 7.38-7.19 (m, |
| | pyrrolo[3,2-d]pyrimidin-5-yl}methyl | | | | 1H), 6.26 (m, 3H), 4.89 (dd, J = |
| | propan-2-yl carbonate | | | | 12.6, 6.3 Hz, 1H), 4.53 (t, J = |
| | | | | | 5.6 Hz, 2H), 3.84 (t, ] = |
| | | | | | 5.6 Hz, 2H), 3.55 (dd, ] = |
| | | | | | 12.3, 6.2 Hz, 1H), 1.28 (d, J = |
| | | | | | 6.2 Hz, 6H), 1.07 (d, ] = |
| | | | | | 6.1 Hz, 6H). |
| 91 | {4-oxo-2-[{{[(propan-2- | 4.82 mins. | 486 | | 1H NMR (300 MHZ, CDCI3) δ |
| | yloxy)carbonyl]oxy}methyl)sulfanyl]- | | | | 7.43-7.11 (m, 1H), 6.44 (s, |
| | 1-[2-(propan-2-yloxy)ethyl]- | | | | 2H), 6.29-6.14 (m, 1H), |
| | 1H,4H,5H-pyrrolo[3,2-d]pyrimidin-5- | | | | 6.00 (s, 2H), 5.09-4.73 (m, |
| | yl}methyl propan-2-yl carbonate | | | | 2H), 4.33-4.07 (m, 2H), |
| | | | | | 3.71 (t, J = 5.9 Hz, 2H), 3.51 |
| | | | | | (hept, J = 6.1 Hz, 1H), 1.42- |
| | | | | | 1.18 (m, 12H), 1.18-0.95 |
| | | | | | (m, 6H). |
| 92 | 1-{4-oxo-1-[2-(propan-2- | 4.77 mins | 384 | | 1H NMR (300 MHz, CDCI3) δ |
| | yloxy)ethyl]-2-sulfanylidene- | | | | 9.14 (s, 1H), 7.40 (q, J = 6.2 |
| | 1H,2H,3H,4H,5H-pyrrolo[3,2- | | | | Hz, 1H), 7.30 (d, J = 3.2 Hz, |
| | d]pyrimidin-5-yl}ethyl propan-2-yl | | | | 1H), 6.29 (d, J = 3.3 Hz, 1H), |
| | carbonate | | | | 4.95-4.79 (m, 1H), 4.52 (t, J = |
| | | | | | 5.8 Hz, 2H), 3.84 (t, J = 5.6 Hz, |
| | | | | | 2H), 3.55 (ddd, J = 17.4, 11.8, |
| | | | | | 5.7 Hz, 1H), 1.81 (d, J = 6.2 Hz, |
| | | | | | 3H), 1.28 (d, J = 6.2 Hz, 3H), |
| | | | | | 1.26-1.23 (m, 3H), 1.08 (d, J = |
| | | | | | 6.1 Hz, 6H). |
| 93 | {4-oxo-1-[2-(propan-2-yloxy)ethyl]- | 3.98 mins. | 414 | | 1H NMR (300 MHz, CDCI3) δ |
| | 2-sulfanylidene-1H,2H,3H,4H,5H- | | | | 9.16 (s, 1H), 7.29-7.27 (m, |
| | pyrrolo[3,2-d]pyrimidin-5-yl}methyl | | | | 1H), 6.26 (s, 2H), 6.25 (d, J = |
| | 3-(2-methoxyethoxy)propanoate | | | | 3.2 Hz, 1H), 4.53 (t, J = 5.7 Hz, |
| | | | | | 2H), 3.84 (t, J = 5.7 Hz, 2H), |
| | | | | | 3.73 (t, J = 6.3 Hz, 2H), 3.63- |
| | | | | | 3.52 (m, 4H), 3.51-3.46 (m, |
| | | | | | 3H), 3.35 (s, 3H), 2.64 (t, J = |
| | | | | | 6.3 Hz, 2H), 1.07 (d, J = 6.1 Hz, |
| | | | | | 6H). |
| 94 | ({4-oxo-1-[2-(propan-2-yloxy)ethyl]- | 4.05 mins. | 423 | | 1H NMR (300 MHz, CDCI3) δ |
| | 1H,4H,5H-pyrrolo[3,2-d]pyrimidin-2- | | | | 12.01 (s, 1H), 7.42 (t, J = 3.1 |
| | yl}sulfanyl)methyl N-methyl-N- | | | | Hz, 1H), 6.32 (m, 1H), 6.06 (d, |
| | (2,2,2-trifluoroethyl)carbamate | | | | J = 2.2 Hz, 2H), 4.37 (dd, J = |
| | | | | | 5.6, 3.7 Hz, 2H), 3.98-3.82 |
| | | | | | (m, 2H), 3.78 (dd, J = 9.5, 5.7 |
| | | | | | Hz, 2H), 3.57-3.45 (m, 1H), |
| | | | | | 3.04 (d, J = 14.2 Hz, 3H), 1.05 |
| | | | | | (d, J = 6.1 Hz, 6H). |
| 95 | {2-[{{[methyl(2,2,2- | 4.87 mins. | 592 | | 1H NMR (300 MHz, CDCI3) δ |
| | trifluoroethyl)carbamoyl]oxy}methyl | | | | 7.45 (dd, J = 13.0, 3.3 Hz, 1H), |
| | )sulfanyl]-4-oxo-1-[2-(propan-2- | | | | 6.42 (d, J = 5.3 Hz, 2H), 6.31- |
| | yloxy)ethyl]-1H, 4H,5H-pyrrolo[3,2- | | | | 6.27 (m, 1H), 5.98 (d, J = 7.0 |
| | d]pyrimidin-5-yl}methyl N-methyl-N- | | | | Hz, 2H), 4.32-4.24 (m, 2H), |
| | (2,2,2-trifluoroethyl)carbamate | | | | 3.96-3.81 (m, 4H), 3.74 (d, J = |
| | | | | | 3.7 Hz, 2H), 3.55-3.45 (m, |
| | | | | | 1H), 3.03 (d, J = 9.5 Hz, 6H), |
| | | | | | 1.04 (dt, J = 7.7, 2.3 Hz, 6H). |
| 96 | ({4-oxo-1-[2-(propan-2-yloxy)ethyl]- | 4.38 mins. | 491 | | 1H NMR (300 MHz, CDCI3) δ |
| | 1H,4H,5H-pyrrolo[3,2-d]pyrimidin-2- | | | | 7.45 (t, J = 3.8 Hz, 1H), 6.37 (t, |
| | yl}sulfanyl)methyl N,N-bis(2,2,2- | | | | J = 2.7 Hz, 1H), 6.09 (s, 2H), |
| | trifluoroethyl)carbamate | | | | 4.39 (t, J = 5.4 Hz, 2H), 4.03 |
| | | | | | (dq, J = 16.9, 8.5 Hz, 4H), 3.78 |
| | | | | | (t, J = 5.6 Hz, 2H), 3.49 (dt, J = |
| | | | | | 12.3, 6.1 Hz, 1H), 1.24-0.82 |
| | | | | | (m, 6H). |
| 97 | {2-[{{[bis(2,2,2- | 5.22 mins. | 728 | | 1H NMR (300 MHz, CDCI3) δ |
| | trifluoroethyl) carbamoyl]oxy}methyl | | | | 7.33 (d, J = 3.3 Hz, 1H), 6.49 |
| | )sulfanyl]-4-oxo-1-[2-(propan-2- | | | | (s, 2H), 6.26 (d, J = 3.3 Hz, 1H), |
| | yloxy)ethyl]-1H,4H,5H-pyrrolo[3,2- | | | | 6.06 (s, 2H), 4.24 (t, J = 5.6 Hz, |
| | d]pyrimidin-5-yl}methyl N,N- | | | | 2H), 4.11-3.93 (m, 8H), 3.72 |
| | bis(2,2,2-trifluoroethyl)carbamate | | | | (t, J = 5.6 Hz, 2H), 3.53-3.41 |
| | | | | | (m, 1H), 1.03-1.00 (m, 6H). |
| 98 | [3-({[bis(2,2,2- | 5.76 mins. | 728 | | 1H NMR (300 MHz, CDCI3) δ |
| | trifluoroethyl) carbamoyl]oxy}methyl)- | | | | 7.32 (d, J = 3.2 Hz, 1H), 6.69 |
| | 4-oxo-1-[2-(propan-2-yloxy)ethyl]- | | | | (s, 2H), 6.33 (s,2H), 6.26 (d, J = |
| | 2-sulfanylidene-1H, 2H,3H,4H,5H- | | | | 3.2 Hz, 1H), 4.62 (t, J = 5.5 Hz, |
| | pyrrolo[3,2-d]pyrimidin-5-yl]methyl | | | | 2H), 4.15-3.91 (m, 8H), 3.86 |
| | N,N-bis(2,2,2- | | | | (t, J = 5.5 Hz, 2H), 3.57-3.45 |
| | trifluoroethyl)carbamate | | | | (m, 1H), 1.02 (d, J = 6.1 Hz, |
| | | | | | 6H). |

| | Caco-2 Pₐₚₚ | | | human Plasma |
|---|---|---|---|---|
| Example | (nm/s) | SGF | SIF | stability |
| 90 | 180 | 77% remaining | 56% remaining | 0% remaining |
| | | At 1 h | At 1 h | @ 1 h |
| 91 | 142 | 98% remaining | 99% remaining | 1% remaining |
| | | At 1 h | At 1 h | @ 1 h |
| 92 | unstable | 1% remaining | 74% remaining | 0% remaining |
| | | At 1 h | At 1 h | @ 1 h |
| 93 | 168 | 99 | 38 | 0% remaining |
| | | | | at 1 h |
| 94 | 151 | 99 | 95 | 99% remaining |
| | | | | at 1 h |
| 95 | 188 | 100 | 95 | 99% remaining |
| | | | | at 1 h |
| 96 | 203 | 98 | 97 | 98% remaining |
| | | | | at 1 h |
| 97 | 308 | 98 | 96 | 99% remaining |
| | | | | at 1 h |
| 98 | 65.3 | 95 | 80 | 99% remaining |
| | | | | at 1 h |

### Example 99

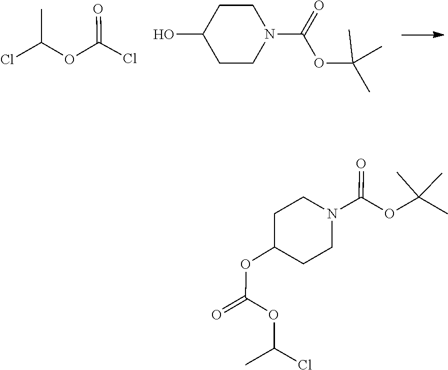

tert-Butyl 4-{[(1-chloroethoxy)carbonyl]oxy}piperidine-1-carboxylate: Pyridine (647 μL, 8 mmol) was added to tert-butyl 4-hydroxypiperidine-1-carboxylate (1.61 g, 8 mmol) in dichloromethane (10 mL) and cooled on an ice bath. Chloroethyl chloroformate (826 μL, 8 mmol) was added dropwise and the reaction was warmed to room temperature overnight. Additional dichloromethane was added (20 mL) and washed with water (4×5 mL) and brine. After drying over magnesium sulfate and concentration under vacuum, purification via flash chromatography using a gradient of hexane to 40% ethyl acetate in hexane yielded the titled compound isolated as a clear oil (2.44 g, 99.1%). ¹H NMR (300 MHz, CHLOROFORM-d) δ 6.42 (q, J=5.8 Hz, 1H), 4.85 (tt, J=7.9, 3.8 Hz, 1H), 3.75-3.65 (m, 2H), 3.31-3.17 (m, 2H), 2.0-1.85 (m, 2H), 1.83 (d, J=5.8 Hz, 3H), 1.80-1.60 (m, 2H), 1.45 (s, 9H).

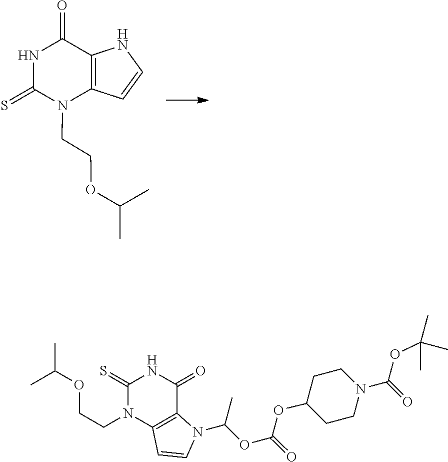 [SMILES: CC(C)OCCn1c(=S)[nH]c(=O)c2[nH]ccc21 ; CC(C)OCCn1c(=S)[nH]c(=O)c2c1ccn2C(C)OC(=O)OC1CCN(C(=O)OC(C)(C)C)CC1]

tert-Butyl 4-{[(1-{4-oxo-1-[2-(propan-2-yloxy)ethyl]-2-sulfanylidene-1H,2H,3H,4H,5H-pyrrolo[3,2-d]pyrimidin-5-yl}ethoxy)carbonyl]oxy}piperidine-1-carboxylate: A solution of 1-[2-(propan-2-yloxy)ethyl]-2-sulfanylidene-1H,2H,3H,4H,5H-pyrrolo[3,2-d]pyrimidin-4-one (101 mg, 400 μmol) in DMF (4.0 mL) under nitrogen was treated with lithium hexamethyldisilylamide (1.0 M in THF, 800 μL, 800 μmol) and stirred for 10 mins. tert-Butyl 4-{[(1-chloroethoxy)carbonyl]oxy}piperidine-1-carboxylate (246 mg, 800 μmol) in DMF (1.0 mL) was added and the reaction was stirred overnight, followed by purification with RP-HPLC (Method B). Freeze-drying provided the titled compound as a white solid (15 mg, 7.1%). ¹H NMR (300 MHz, CHLOROFORM-d) δ 9.13 (s, 1H), 7.40 (q, J=6.1 Hz, 1H), 7.30 (d, J=3.2 Hz, 1H), 6.29 (d, J=3.2 Hz, 1H), 4.75 (dt, J=12.2, 4.2 Hz, 1H), 4.52 (t, J=5.7 Hz, 2H), 3.83 (t, J=5.9 Hz, 2H), 3.76-3.63 (m, 2H), 3.56 (dt, J=12.2, 6.1 Hz, 1H), 3.26-3.06 (m, 2H), 1.95-1.85 (m, 2H), 1.82 (d, J=6.2 Hz, 3H), 1.70-1.53 (m, 2H), 1.44 (s, 9H), 1.08-1.06 (m, 6H). LC/MS method A: Rₜ=5.10 mins., (M+H)⁺=525, purity >95%. Caco-2 Pₐₚₚ=1.11 nm/s, SGF=0% remaining at 1 h, SIF=0% remaining at 1 h, hPlasma stability=5% remaining at 1 h.

Additional compounds prepared according to EXAMPLE 99:

| | | | | | | | | |
|---|---|---|---|---|---|---|---|---|
| 100 | {4-oxo-1-[2-(propan-2-yloxy)ethyl]- | 4.23 mins. | 409 | 1H NMR (300 MHz, CDCI3) δ | 277 | 99% | 97% | 97% |
| | 2-sulfanylidene-1H,2H,3H,4H,5H- | | | 9.18 (s, 1H), 7.33 (d, J = 3.2 | | remain- | remain- | remain- |
| | pyrrolo[3,2-d]pyrimidin-5-yl}methyl | | | Hz, 1H), 6.26-6.22 (m, 3H), | | ing | ing | ing |
| | N-(2,2,2-trifluoroethyl)carbamate | | | 5.21-5.17 (m, 1H), 4.53 (t, J = | | at 1 h | at 1 h | at 1 h |
| | | | | 5.6 Hz, 2H), 3.84 (t, J = 5.6 Hz, | | | | |
| | | | | 2H), 3.82-3.73 (m, 2H), 3.61- | | | | |
| | | | | 3.45 (m, 1H), 1.06 (d, J = 6.1 | | | | |
| | | | | Hz, 6H). | | | | |
| 101 | methyl (2S)-6-{[(tert- | 4.68 mins. | 570 | 1H NMR (300 MHz, DMSO-d6) | 14.6 | 75% | 96% | 97% |
| | butoxy)carbonyl]amino}-2-{[{{4- | | | δ 12.35 (s, 1H), 7.90 (d, J = 7.4 | | remain- | remain- | remain- |
| | oxo-1-[2-(propan-2-yloxy)ethyl]-2- | | | Hz, 1H), 7.46 (d, J = 3.2 Hz, | | ing | ing | ing |
| | sulfanylidene-1H,2H,3H,4H,5H- | | | 1H), 6.77-6.71 (m, 1H), 6.36 | | at 1 h | at 1 h | at 1 h |
| | pyrrolo[3,2-d]pyrimidin-5- | | | (d, J = 3.2 Hz, 1H), 6.10 (d, J = | | | | |
| | yl}methoxy)carbonyl]amino} | | | 1.8 Hz, 2H), 4.45 (t, J = 6.0 Hz, | | | | |
| | hexanoate | | | 2H), 3.93 (dd, J = 12.7, 8.8 Hz, | | | | |
| | | | | 1H), 3.68 (t, J = 6.0 Hz, 2H), | | | | |
| | | | | 3.58 (s, 3H), 3.56-3.47 (m, | | | | |
| | | | | 1H), 2.86-2.77 (m, 2H), 1.65- | | | | |
| | | | | 1.42 (m, 2H), 1.32 (s, 9H), | | | | |
| | | | | 1.31-1.15 (m, 4H), 0.97 (d, J = | | | | |
| | | | | 6.1 Hz, 6H). | | | | |

### Examples 102 and 103

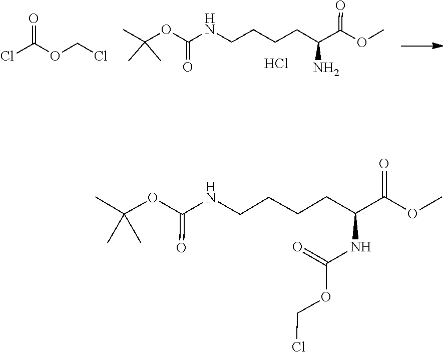

Methyl (2S)-6-{[(tert-butoxy)carbonyl]amino}-2-{[(chloromethoxy)carbonyl]amino}hexanoate: Pyridine (1.29 mL, 16 mmol) was added to methyl (2S)-2-amino-6-{[(tert-butoxy)carbonyl]amino}hexanoate hydrogen chloride (2.37 g, 8 mmol) in dichloromethane (10 mL) and cooled on an ice bath. Chloromethyl chloroformate (711 μL, 8 mmol) was added dropwise and the reaction was warmed to room temperature overnight. Additional dichloromethane was added (20 mL) and washed with water (4×5 mL) and brine. After drying over magnesium sulfate and concentration under vacuum, the titled compound was isolated (2.33 g, 82.6%), without need of further purification. ¹H NMR (300 MHz, DMSO-d₆) δ 8.16 (d, J=7.7 Hz, 1H), 6.75 (t, J=5.7 Hz, 1H), 5.82 (q, J=6.1 Hz, 2H), 4.04-3.94 (m, 1H), 3.61 (s, 3H), 2.84 (q, J=6.2 Hz, 2H), 1.71-1.45 (m, 2H), 1.33 (s, 10H), 1.28-1.20 (m, 3H).

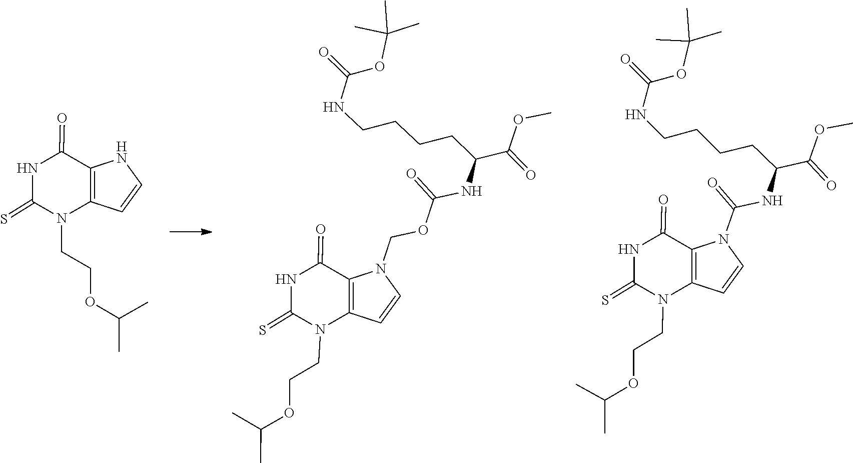 [SMILES: CC(C)OCCn1c(=S)[nH]c(=O)c2[nH]ccc21 ; COC(=O)[C@H](CCCCNC(=O)OC(C)(C)C)NC(=O)OCn1ccc2c1c(=O)[nH]c(=S)n2CCOC(C)C ; COC(=O)[C@H](CCCCNC(=O)OC(C)(C)C)NC(=O)n1ccc2c1c(=O)[nH]c(=S)n2CCOC(C)C]

Methyl (2S)-6-{[(tert-butoxy)carbonyl]amino}-2-{[({4-oxo-1-[2-(propan-2-yloxy)ethyl]-2-sulfanylidene-1H,2H,3H,4H,5H-pyrrolo[3,2-d]pyrimidin-5-yl}methoxy)carbonyl]amino}hexanoate and Methyl (2S)-6-{[(tert-butoxy)carbonyl]amino}-2-({4-oxo-1-[2-(propan-2-yloxy)ethyl]-2-sulfanylidene-1H,2H,3H,4H,5H-pyrrolo[3,2-d]pyrimidine-5-carbonyl}amino)hexanoate: A solution of 1-[2-(propan-2-yloxy)ethyl]-2-sulfanylidene-1H,2H,3H,4H,5H-pyrrolo[3,2-d]pyrimidin-4-one (101 mg, 400 μmol) in DMF (2.0 mL) under nitrogen was treated with lithium hexamethyldisilylamide (1.0 M in THF, 800 μL, 800 μmol) and stirred for 10 mins. Methyl (2S)-6-{[(tert-butoxy)carbonyl]amino}-2-{[(chloromethoxy)carbonyl]amino}hexanoate (141 mg, 400 μmol) in DMF (1.0 mL) was added and the reaction was stirred overnight, followed by purification with RP-HPLC (Method D).

Spectra for EXAMPLE 102: Methyl (2S)-6-{[(tert-butoxy)carbonyl]amino}-2-{[({4-oxo-1-[2-(propan-2-yloxy)ethyl]-2-sulfanylidene-1H,2H,3H,4H,5H-pyrrolo[3,2-d]pyrimidin-5-yl}methoxy)carbonyl]amino}hexanoate. ¹H NMR (300 MHz, DMSO-d₆) δ 12.35 (s, 1H), 7.90 (d, J=7.4 Hz, 1H), 7.46 (d, J=3.2 Hz, 1H), 6.77-6.71 (m, 1H), 6.36 (d, J=3.2 Hz, 1H), 6.10 (d, J=1.8 Hz, 2H), 4.45 (t, J=6.0 Hz, 2H), 3.93 (dd, J=12.7, 8.8 Hz, 1H), 3.68 (t, J=6.0 Hz, 2H), 3.58 (s, 3H), 3.56-3.47 (m, 1H), 2.86-2.77 (m, 2H), 1.65-1.42 (m, 2H), 1.32 (s, 9H), 1.31-1.15 (m, 4H), 0.97 (d, J=6.1 Hz, 6H). LC/MS method A: Rₜ=4.68 mins., (M+H)⁺=570, purity >95%. Caco-2 Pₐₚₚ=14.6 nm/s, SGF=75% remaining at 1 h, SIF=96% remaining at 1 h, hPlasma stability=97% remaining at 1 h.

Spectra for EXAMPLE 103: Methyl (2S)-6-{[(tert-butoxy)carbonyl]amino}-2-({4-oxo-1-[2-(propan-2-yloxy)ethyl]-2-sulfanylidene-1H,2H,3H,4H,5H-pyrrolo[3,2-cl]pyrimidine-5-carbonyl}amino)hexanoate. ¹H NMR (300 MHz, CHLOROFORM-d) δ 9.22 (s, 1H), 7.32 (s, 1H), 6.23 (d, J=3.1 Hz, 1H), 6.21-6.20 (m, 1H), 5.52 (s, 1H), 4.53 (dd, J=6.6, 5.1 Hz, 2H), 4.28 (s, 1H), 3.83 (t, J=5.7 Hz, 2H), 3.72 (s, 3H), 3.63-3.50 (m, 1H), 3.10-3.00 (m, 2H), 1.95-1.55 (m, 3H), 1.43 (s, 10H), 1.38-1.26 (m, 2H), 1.07 (d, J=6.1 Hz, 6H). LC/MS method A: Rₜ=5.03 mins., (M+H)⁺=540, purity >95%. Caco-2 Pₐₚₚ=135 nm/s, SGF=96% remaining at 1 h, SIF=96% remaining at 1 h, hPlasma stability=92% remaining at 1 h.

Additional compounds prepared according to EXAMPLES 102-103:

| | | | | | | | | |
|---|---|---|---|---|---|---|---|---|
| 104 | (2-{[{{[2-(2- | 4.00 mins. | 606 | 1H NMR (300 MHz, CDCI3) δ | 2.76 | 96% | 70% | 18% |
| | methoxyethoxy)ethoxy]carbonyl} | | | 7.37 (d, J = 3.3 Hz, 1H), 6.41 | | remain- | remain- | remain- |
| | oxy)methyl]sulfanyl}-4-oxo-1-[2- | | | (s, 2H), 6.29 (d, J = 3.3 Hz, 1H), | | ing | ing | ing |
| | (propan-2-yloxy)ethyl]-1H,4H,5H- | | | 5.98 (s, 2H), 4.35-4.23 (m, | | at 1 h | at 1 h | at 1 h |
| | pyrrolo[3,2-d]pyrimidin-5-yl)methyl | | | 6H), 3.77-3.67 (m, 6H), 3.66- | | | | |
| | 2-(2-methoxyethoxy)ethyl | | | 3.58 (m, 4H), 3.56-3.46 (m, | | | | |
| | carbonate | | | 5H), 3.36 (s, 3H), 3.35 (s, 3H), | | | | |
| | | | | 1.08-1.03 (m, 6H). | | | | |
| 105 | 2-(2-methoxyethoxy)ethyl {4-oxo-1- | 3.97 mins. | 430 | 1H NMR (300 MHz, CDCI3) δ | 243 | 95% | 84% | 14% |
| | [2-(propan-2-yloxy)ethyl]-2- | | | 9.18 (s, 1H), 7.31-7.27 (m, | | remain- | remain- | remain- |
| | sulfanylidene-1H,2H,3H,4H,5H- | | | 1H), 6.28-6.25 (m, 3H), 4.52 | | ing | ing | ing |
| | pyrrolo[3,2-d]pyrimidin-5-yl}methyl | | | (t, J = 5.7 Hz, 2H), 4.33-4.26 | | at 1 h | at 1 h | at 1 h |
| | carbonate | | | (m, 2H), 3.83 (t, J = 5.7 Hz, 2H), | | | | |
| | | | | 3.73-3.67 (m, 2H), 3.64- | | | | |
| | | | | 3.59 (m, 2H), 3.57 (dd, J = 9.8, | | | | |
| | | | | 3.7 Hz, 1H), 3.53-3.49 (m, | | | | |
| | | | | 2H), 3.36 (s, 3H), 1.09-1.04 | | | | |
| | | | | (m, 6H). | | | | |
| 106 | {3-[{{[2-(2- | 4.71 mins. | 606 | 1H NMR (300 MHz, CDCI3) δ | 139 | 100% | 1% | 2% |
| | methoxyethoxy)ethoxy]carbonyl} | | | 7.30 (d, J = 3.2 Hz, 1H), 6.63 | | remain- | remain- | remain- |
| | oxy)methyl]-4-oxo-1-[2-(propan-2- | | | (s, 2H), 6.27 (s, 2H), 6.25 (d, J | | ing | ing | ing |
| | yloxy)ethyl]-2-sulfanylidene- | | | = 3.2 Hz, 1H), 4.60 (t, J = 5.7 | | at 1 h | at 1 h | at 1 h |
| | 1H,2H,3H,4H,5H-pyrrolo[3,2- | | | Hz, 2H), 4.36-4.27 (m, 4H), | | | | |
| | d]pyrimidin-5-yl} methyl 2-(2- | | | 3.83 (t, J = 5.7 Hz, 2H), 3.75- | | | | |
| | methoxyethoxy)ethyl carbonate | | | 3.66 (m, 4H), 3.66-3.57 (m, | | | | |
| | | | | 4H), 3.56-3.48 (m, 5H), 3.36 | | | | |
| | | | | (s, 3H), 3.35 (s, 3H), 1.09- | | | | |
| | | | | 1.06 (m, 6H). | | | | |
| 107 | tert-butyl (2S)-3-methyl-2-{[{{4-oxo- | 5.03 mins. | 483 | 1H NMR (300 MHz, DMSO-d6) | 115 | 93% | 92% | 86% |
| | 1-[2-(propan-2-yloxy)ethyl]-2- | | | δ 12.35 (s, 1H), 7.73 (d, J = 8.2 | | remain- | remain- | remain- |
| | sulfanylidene-1H,2H,3H,4H,5H- | | | Hz, 1H), 7.47 (d, J = 3.2 Hz, | | ing | ing | ing |
| | pyrrolo[3,2-d]pyrimidin-5- | | | 1H), 6.36 (d, J = 3.1 Hz, 1H), | | at 1 h | at 1 h | at 1 h |
| | yl}methoxy)carbonyl]amino}butanoate | | | 6.11 (s, 2H), 4.45 (t, J = 5.8 Hz, | | | | |
| | | | | 2H), 3.72 (dd, J = 8.2, 6.1 Hz, | | | | |
| | | | | 1H), 3.68 (t, J = 5.9 Hz, 2H), | | | | |
| | | | | 3.57-3.46 (m, 1H), 1.34 (s, | | | | |
| | | | | 9H), 0.97 (d, J = 6.1 Hz, 6H), | | | | |
| | | | | 0.81 (dd, J = 6.8, 3.5 Hz, 6H). | | | | |

### Example 109

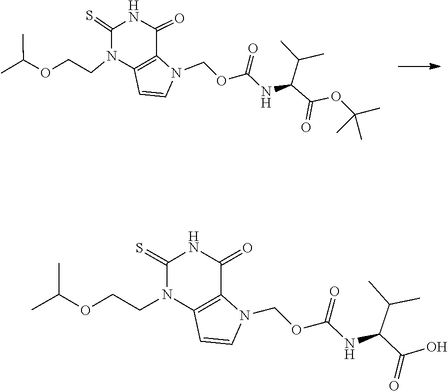

(2S)-3-methyl-2-{[({4-oxo-1-[2-(propan-2-yloxy)ethyl]-2-sulfanylidene-1H,2H,3H,4H,5H-pyrrolo[3,2-d]pyrimidin-5-yl}methoxy)carbonyl]amino}butanoic acid: tert-Butyl (2S)-3-methyl-2-{[({4-oxo-1-[2-(propan-2-yloxy)ethyl]-2-sulfanylidene-1H,2H,3H,4H,5H-pyrrolo[3,2-d]pyrimidin-5-yl}methoxy)carbonyl]amino}butanoate (33.2 mg, 69 μmol) was dissolved in trifluoroacetic acid in dichloromethane (30% v/v, 1.5 mL) and stirred for 1 hour. The reaction was concentrated and purified by RP-HPLC (Method B). Fractions containing the desired product were combined and freeze-dried to provide the titled compound (14.5 mg, 49.4%). ¹H NMR (300 MHz, DMSO-d₆) δ 12.58 (s, 1H), 12.36 (s, 1H), 7.71 (d, J=8.3 Hz, 1H), 7.47 (d, J=3.1 Hz, 1H), 6.36 (d, J=3.2 Hz, 1H), 6.16-6.07 (m, 2H), 4.45 (t, J=6.0 Hz, 2H), 3.80 (dd, J=8.4, 5.9 Hz, 1H), 3.68 (t, J=6.0 Hz, 2H), 3.56-3.47 (m, 1H), 1.98 (dd, J=13.0, 6.7 Hz, 1H), 0.97 (dd, J=6.1, 1.6 Hz, 6H), 0.81 (t, J=7.0 Hz, 6H). LC/MS method A: Rₜ=3.89 mins., (M+H)⁺=427, purity >95%. Caco-2 Pₐₚₚ=0.94 nm/s, SGF=94% remaining at 1 h, SIF=96% remaining at 1 h, hPlasma stability=100% remaining at 1 h.

Additional compounds prepared according to EXAMPLE 109:

| Ex- | | LCMS | | | | Caco- | | | human |
|---|---|---|---|---|---|---|---|---|---|
| am- | | retention | M + | LCMS | | 2 Pₐₚₚ | | | Plasma |
| ple | Name | time | H⁺ | method | NMR | (nm/s) | SGF | SIF | stability |
| 110 | trifluoroacetic acid methyl (2S)-6- | 3.20 mins. | 470 | | 1H NMR (300 MHz, DMSO-d6) | 1.42 | 81% | 45% | 62% |
| | amino-2-{[{{4-oxo-1-[2-(propan- | | | | δ 7.92 (d, J = 7.6 Hz, 1H), 7.55 | | remain- | remain- | remain- |
| | 2-yloxy)ethyl]-2-sulfanylidene- | | | | (br s, 2H), 7.46 (d, J = 3.1 Hz, | | ing | ing | ing |
| | 1H,2H,3H,4H,5H-pyrrolo[3,2- | | | | 1H), 6.37 (d, J = 3.2 Hz, 1H), | | at 1 h | at 1 h | at 1 h |
| | d]pyrimidin-5- | | | | 6.10 (q, J = 10.4 Hz, 2H), 4.45 | | | | |
| | yl}methoxy)carbonyl]amino} | | | | (t, J = 6.1 Hz, 2H), 4.00-3.92 | | | | |
| | hexanoate | | | | (m, 1H), 3.69 (t, J = 6.0 Hz, 2H), | | | | |
| | | | | | 3.59 (s, 3H), 3.52 (dt, J = 12.1, | | | | |
| | | | | | 6.0 Hz, 1H), 2.75-2.65 (m, | | | | |
| | | | | | 2H), 1.67-1.38 (m, 4H), 1.33- | | | | |
| | | | | | 1.23 (m, 2H), 0.98-0.96 (m, | | | | |
| | | | | | 6H) | | | | |
| 111 | methyl (2S)-6-[(2S)-2-{[(tert- | 4.69 mins. | 669 | | 1H NMR (300 MHz, CDCl3) δ | 5.75 | 87% | 74% | 85% |
| | butoxy)carbonyl]amino}-3- | | | | 7.32-7.30 (m, 1H), 6.28- | | remain- | remain- | remain- |
| | methylbutanamido]-2-{[{{4-oxo- | | | | 6.10 (m, 3H), 5.81-5.75 (m, | | ing | ing | ing |
| | 1-[2-(propan-2-yloxy)ethyl]-2- | | | | 1H), 5.28-5.22 (m, 1H), 4.57- | | at 1 h | at 1 h | at 1 h |
| | sulfanylidene-1H,2H,3H,4H,5H- | | | | 4.47 (m, 2H), 4.28-4.18 (m, | | | | |
| | pyrrolo[3,2-d]pyrimidin-5- | | | | 1H), 3.86-3.77 (m, 3H), 3.71 | | | | |
| | yl}methoxy)carbonyl]amino} | | | | (s, 3H), 3.57 (dt, J = 12.2, 6.1 | | | | |
| | hexanoate | | | | Hz, 1H), 3.33 (dd, J = 14.0, 6.9 | | | | |
| | | | | | Hz, 1H), 3.17 (dd, J = 13.4, 5.8 | | | | |
| | | | | | Hz, 1H), 2.15-2.00 (m, 1H), | | | | |
| | | | | | 1.57-1.43 (m, 3H), 1.41 (s, | | | | |
| | | | | | 9H), 1.35-1.28 (m, 3H), 1.08 | | | | |
| | | | | | (d, J = 6.1 Hz, 6H), 0.93 (d, J = | | | | |
| | | | | | 6.7 Hz, 6H). | | | | |
| 112 | 1-{4-oxo-1-[2-(propan-2- | 4.53 mins. | 505 | | 1H NMR (300 MHz, CDCl3) δ | 211 | 100% | 100% | 99% |
| | yloxy)ethyl]-2-sulfanylidene- | | | | 12.07-11.90 (m, 1H), 7.40- | | remain- | remain- | remain- |
| | 1H,2H,3H,4H,5H-pyrrolo[3,2- | | | | 7.35 (m, 1H), 7.08-6.98 (m, | | ing | ing | ing |
| | d]pyrimidin-5-yl}ethyl N,N- | | | | 1H), 6.30-6.25 (m, 1H), 4.47- | | at 1 h | at 1 h | at 1 h |
| | bis(2,2,2- | | | | 3.82 (m, 6H), 3.75 (t, J = 5.8 | | | | |
| | trifluoroethyl)carbamate | | | | Hz, 2H), 3.49 (dt, J = 8.3, 6.0 | | | | |
| | | | | | Hz, 1H), 1.85 (d, J = 6.6 Hz, | | | | |
| | | | | | 3H), 1.05 (t, J = 5.7 Hz, 6H). | | | | |
| 113 | tert-butyl 2-{methyl [(1-{4-oxo-1- | 4.35 mins. | 469 | | 1H NMR (300 MHz, CDCl3) δ | 78.6 | 44% | 96% | 99% |
| | [2-(propan-2-yloxy)ethyl]-2- | | | | 11.48 (s, 1H), 7.32-7.27 (m, | | remain- | remain- | remain- |
| | sulfanylidene-1H, 2H,3H,4H,5H- | | | | 1H), 6.95-6.81 (m, 1H), 6.21- | | ing | ing | ing |
| | pyrrolo[3,2-d]pyrimidin-5- | | | | 6.19 (m, 1H), 4.42-3.79 (m, | | at 1 h | at 1 h | at 1 h |
| | yl}ethoxy)carbonyl]amino}acetate | | | | 4H), 3.74 (t, J = 6.1 Hz, 2H), | | | | |
| | | | | | 3.57-3.45 (m, 1H), 2.93 (d, J = | | | | |
| | | | | | 7.1 Hz, 3H), 1.87 (dd, J = | | | | |
| | | | | | 11.2, 6.6 Hz, 3H), 1.44 (s, 9H), | | | | |
| | | | | | 1.07 (dd, J = 6.1, 2.0 Hz, 6H). | | | | |
| 114 | trifluoroacetic acid methyl (2S)-6- | 3.33 mins. | 569 | | 1H NMR (300 MHZ, DMSO-d6) | 1.02 | 98% | 89% | 93% |
| | [(2S)-2-amino-3- | | | | δ 12.36 (s, 1H), 8.35-8.28 | | remain- | remain- | remain- |
| | methylbutanamido]-2-{[{{4-oxo- | | | | (m,1H), 8.01 (br s, 3H), 7.92 (d, | | ing | ing | ing |
| | 1-[2-(propan-2-yloxy)ethyl]-2- | | | | J = 7.6 Hz, 1H), 7.46 (d, J = 3.1 | | at 1 h | at 1 h | at 1 h |
| | sulfanylidene-1H,2H,3H,4H,5H- | | | | Hz, 1H), 6.37 (d, J = 3.2 Hz, | | | | |
| | pyrrolo[3,2-d]pyrimidin-5- | | | | 1H), 6.10 (q, J = 10.4 Hz, 2H), | | | | |
| | yl}methoxy)carbonyl]amino} | | | | 4.45 (t, J = 6.0 Hz, 2H), 3.98- | | | | |
| | hexanoate | | | | 3.88 (m, 1H), 3.74-3.64 (m, | | | | |
| | | | | | 3H), 3.58 (s, 3H), 3.56-3.48 | | | | |
| | | | | | (m, 1H), 3.22-3.10 (m, 1H), | | | | |
| | | | | | 3.02-2.90 (m, 1H), 2.05- | | | | |
| | | | | | 1.92 (m, 1H), 1.61-1.25 (m, | | | | |
| | | | | | 6H), 0.99-0.96 (m, 6H), 0.90- | | | | |
| | | | | | 0.82 (m, 6H). | | | | |
| 115 | 1-{4-oxo-1-[2-(propan-2- | 4.88 mins. | 402 | | 1H NMR (300 MHz, CDCl3) δ | 80.5 | 63% | 67% | 59% |
| | yloxy)ethyl]-2-sulfanylidene- | | | | 9.21 (s, 1H), 8.04-7.99 (m, | | remain- | remain- | remain- |
| | 1H,2H,3H,4H,5H-pyrrolo[3,2- | | | | 2H), 7.68-7.53 (m, 2H), 7.47- | | ing | ing | ing |
| | d]pyrimidin-5-yl}ethyl benzoate | | | | 7.39 (m, 2H), 7.36 (d, J = 3.2 | | at 1 h | at 1 h | at 1 h |
| | | | | | Hz, 1H), 6.27 (d, J = 3.2 Hz, | | | | |
| | | | | | 1H), 4.52 (td, J = 5.8, 2.8 Hz, | | | | |
| | | | | | 2H), 3.83 (dt, J = 5.9, 3.1 Hz, | | | | |
| | | | | | 2H), 3.56 (dq, J = 12.2, 6.1 Hz, | | | | |
| | | | | | 1H), 1.93 (d, J = 6.2 Hz, 3H), | | | | |
| | | | | | 1.08-1.05 (m, 6H). | | | | |
| 116 | {4-oxo-1-[2-(propan-2-yloxy) | 4.78 mins. | 388 | | 1H NMR (300 MHZ, CDCl3) δ | 64.1 | 99% | 46% | 1% |
| | ethyl]-2-sulfanylidene- | | | | 9.21 (s, 1H), 8.04 (dt, J = 8.5, | | remain- | remain- | remain- |
| | 1H, 2H,3H,4H,5H- | | | | 1.6 Hz, 2H), 7.63-7.50 (m, | | ing | ing | ing |
| | pyrrolo[3,2-d]pyrimidin-5-yl} | | | | 1H), 7.47-7.36 (m, 3H), 6.49 | | at 1 h | at 1 h | at 1 h |
| | methylbenzoate | | | | (s, 2H), 6.25 (d, J = 3.2 Hz, 1H), | | | | |
| | | | | | 4.52 (t, J = 5.7 Hz, 2H), 3.83 (t, | | | | |
| | | | | | J = 5.7 Hz, 2H), 3.53 (tt, J = 7.6, | | | | |
| | | | | | 3.8 Hz, 1H), 1.06 (d, J = 6.1 Hz, | | | | |
| | | | | | 6H). | | | | |
| 117 | {3-[(benzoyloxy)methyl]-4-oxo- | 5.21 mins. | 522 | | 1H NMR (300 MHz, CDCI3) δ | 57.6 | 100% | 79% | 20% |
| | 1-[2-(propan-2-yloxy)ethyl]-2- | | | | 8.06 (ddd, J = 6.9, 3.2, 1.7 Hz, | | remain- | remain- | remain- |
| | sulfanylidene-1H,2H,3H,4H,5H- | | | | 4H), 7.61-7.52 (m, 2H), 7.47- | | ing | ing | ing |
| | pyrrolo[3,2-d]pyrimidin-5-yl} | | | | 7.37 (m,5H), 6.68 (s, 2H), | | at 1 h | at 1 h | at 1 h |
| | methylbenzoate | | | | 6.25 (d, J = 3.3 Hz, 1H), 6.21 | | | | |
| | | | | | (s, 2H), 4.24 (t, J = 5.8 Hz, 2H), | | | | |
| | | | | | 3.70 (t, J = 5.8 Hz, 2H), 3.54- | | | | |
| | | | | | 3.39 (m, 1H), 1.01 (d, J = 6.1 | | | | |
| | | | | | Hz, 6H). | | | | |

### Examples 118 and 119

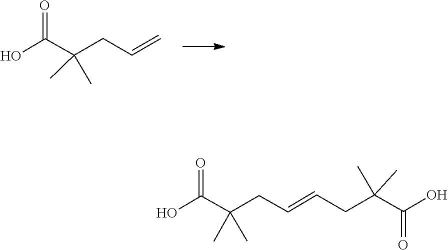

(4E)-2,2,7,7-Tetramethyloct-4-enedioic acid: 2,2-Dimethyl-4-pentenoic acid (7.7 g, 60 mmol) in dichloromethane (90 mL) with Grubbs 2n d generation catalyst (600 mg, 2.12 mmol) was purged with nitrogen for 10 minutes. The reaction was heated for 3 days at 41° C. then cooled to room temp. The dichloromethane was treated with saturated sodium bicarbonate. The dichloromethane layer was set aside and the aqueous layer was acidified to pH 1 with addition of 1 N HCl. This was extracted twice with ethyl acetate. The combined fractions were washed with brine and dried over magnesium sulfate, providing the title compound (4.28 g, 62.5%). ¹H NMR (300 MHz, CHLOROFORM-d) δ 5.53-5.47 (m, 2H), 2.21 (dd, J=4.2, 1.8 Hz, 4H), 1.18 (s, 12H).

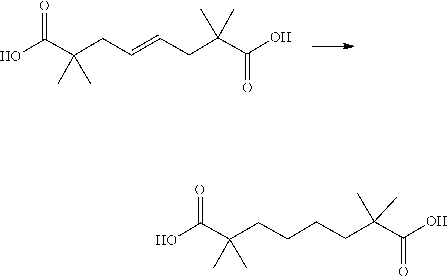 [SMILES: CC(C)(C/C=C/CC(C)(C)C(=O)O)C(=O)O ; CC(C)(CCCCC(C)(C)C(=O)O)C(=O)O]

2,2,7,7-Tetramethyloctanedioic acid: A round-bottom flask containing a solution of (4E)-2,2,7,7-tetramethyloct-4-enedioic acid (4.26 g, 18.66 mmol) in methanol (130 mL) with palladium on carbon (5%, wet, 50 mg), was equipped with a balloon filled with hydrogen. The flask was quickly purged under vacuum and filled with hydrogen (3×), and stirred overnight. The palladium was removed by filtration and solvent removed to provide the titled compound (4.24 g, 98.7%). ¹H NMR (300 MHz, DMSO-d₆) δ 12.00 (s, 2H), 1.49-1.24 (m, 4H), 1.15-1.07 (m, 4H), 1.02 (s, 12H).

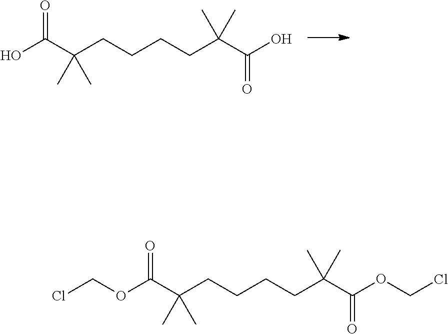 [SMILES: CC(C)(CCCCC(C)(C)C(=O)O)C(=O)O ; CC(C)(CCCCC(C)(C)C(=O)OCCl)C(=O)OCCl]

1,8-Dichloromethyl 2,2,7,7-tetramethyloctanedioate: A solution of 2,2,7,7-tetramethyloctanedioic acid (4.62 g, 20.06 mmol), tetrabutylammonium hydrogen sulfate (681 mg, 2.0 mmol), and sodium carbonate (11.8 g, 140.4 mmol) was dissolved in dichloromethane and water (50:50 v/v, 90 mL) and cooled at °0 C. on an ice bath. Chloromethyl chlorosulfonate (4.81 mL, 48.1 mmol) was add dropwise and the reaction was warmed to room temperature with stirring overnight. The reaction was poured into a separatory funnel and separated. The aqueous layer was extracted one more time with dichloromethane and the combined organic fractions were washed with brine and dried over magnesium sulfate and concentrated to provide the crude product (4.5 g), which was purified by flash chromatography eluting with a gradient of hexane to 10% ethyl acetate in hexane. The titled compound was isolated as a pale oil in appearance (3.1 g, 47.2%). ¹H NMR (300 MHz, CHLOROFORM-d) δ 5.70 (s, 4H), 1.54-1.49 (m, 4H), 1.24-1.18 (m, 4H), 1.18 (s, 12H).

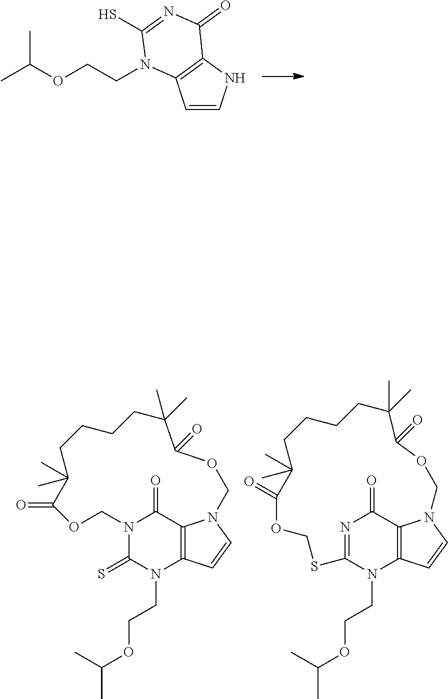

5,5,10,10-Tetramethyl-16-[2-(propan-2-yloxy)ethyl]-15-sulfanylidene-3,12-dioxa-1,14,16-triazatricyclo[12.5.2.0\hat{ }{17,20}]henicosa-17(20),18-diene-4,11,21-trione and 8,8,13,13-Tetramethyl-22-[2-(propan-2-yloxy)ethyl]-6,15-dioxa-17-thia-4,19,22-triazatricyclo[16.3.1.0\hat{ }{4,21}]docosa-1(21),2,18-triene-7,14,20-trione: A solution of 1-[2-(propan-2-yloxy)ethyl]-2-sulfanylidene-1H,2H,3H,4H,5H-pyrrolo[3,2-d]pyrimidin-4-one (50.6 mg, 200 μmol) in DMF (9.0 mL) under nitrogen was treated with lithium hexamethyldisilylamide (1.0 M in THF, 200 μL, 200 μmol) and stirred for 10 mins at 0° C. 1,8-Dichloromethyl 2,2,7,7-tetramethyloctanedioate (65.5 mg, 200 μmol) in DMF (1.0 mL) was added and the reaction was warmed to room temperature over 2 hours. Next, the reaction was cooled to 0° C. and lithium hexamethyldisilylamide (1.0 M in THF, 200 μL, 200 μmol) was added and warmed to room temperature overnight. The reaction was quenched by the addition of water and partitioned between ethyl acetate and water (25 mL each). After separation, the aqueous layer was extracted an additional time with ethyl acetate. The combined organic fractions were washed with brine and dried over magnesium sulfate. After concentration, the crude was purified by RP-HPLC (Method B).

Spectra for EXAMPLE 118: 5,5,10,10-Tetramethyl-16-[2-(propan-2-yloxy)ethyl]-15-sulfanylidene-3,12-dioxa-1,14,16-triazatricyclo[12.5.2.0\hat{ }{17,20}]henicosa-17(20),18-diene-4,11,21-trione. ¹H NMR (300 MHz, CHLOROFORM-d) δ 7.30-7.29 (m, 1H), 6.73 (s, 2H), 6.21 (d, J=3.1 Hz, 1H), 6.02 (s, 2H), 4.68-4.64 (m, 2H), 3.89 (t, J=5.7 Hz, 2H), 3.53 (td, J=11.8, 5.7 Hz, 1H), 1.40-1.20 (m, 6H), 1.19-1.10 (m, 2H), 1.13 (d, J=12.1 Hz, 12H), 1.06-1.02 (m, 6H). LC/MS method A: Rₜ=6.13 mins., (M+H)⁺=508, purity >95%. Caco-2 Pₐₚₚ=66 nm/s, SGF=94% remaining at 1 h, SIF=76% remaining at 1 h, hPlasma stability=73% remaining at 1 h.

Spectra for EXAMPLE 119: 8,8,13,13-Tetramethyl-22-[2-(propan-2-yloxy)ethyl]-6,15-dioxa-17-thia-4,19,22-triazatricyclo[16.3.1.0\hat{ }{4,21}]docosa-1(21),2,18-triene-7,14,20-trione. ¹H NMR (300 MHz, CHLOROFORM-d) δ 7.32 (d, J=3.2 Hz, 1H), 6.38 (d, J=9.6 Hz, 1H), 6.25 (d, J=11.0 Hz, 1H), 6.16 (d, J=3.2 Hz, 1H), 6.02 (d, J=10.0 Hz, 1H), 5.18 (d, J=10.9 Hz, 1H), 4.60-4.44 (m, 1H), 4.20-4.08 (m, 1H), 3.75-3.67 (m, 2H), 3.55-3.45 (m, 1H), 1.70-1.60 (m, 1H), 1.33-0.94 (m, 25H). LC/MS method A: Rₜ=5.04 mins., (M+H)⁺=508, purity >95%. Caco-2 Pₐₚₚ=317 nm/s, SGF=94% remaining at 1 h, SIF=98% remaining at 1 h, hPlasma stability=90% remaining at 1 h.

Additional compounds prepared according to EXAMPLES 118-119:

| | | LCMS | | | |
|---|---|---|---|---|---|
| | | retention | | LCMS | |
| Example | Name | time | M + H⁺ | method | NMR |
| 120 | 5-[{{[2-(2-{[(tert- | 4.58 mins. | 515 | | 1H NMR (300 MHz, DMSO-d6) |
| | butoxy)carbonyl]amino}ethoxy) | | | | δ 12.40 (s, 1H), 7.52 (d, J = 3.2 |
| | ethoxy]carbonyl}oxy)methyl]-1-[2- | | | | Hz, 1H), 6.78-6.72 (m, 1H), |
| | (propan-2-yloxy)ethyl]-2- | | | | 6.39 (d, J = 3.2 Hz, 1H), 6.17 |
| | sulfanylidene-1H,2H,3H,4H,5H- | | | | (s, 2H), 4.45 (t, J = 6.1 Hz, 2H), |
| | pyrrolo[3,2-d]pyrimidin-4-one | | | | 4.20-4.12 (m, 2H), 3.68 (t, J = |
| | | | | | 6.1 Hz, 2H), 3.57-3.47 (m, |
| | | | | | 3H), 3.35-3.25 (m, 2H), 3.00 |
| | | | | | (q, J = 5.9 Hz, 2H), 1.32 (s, 9H), |
| | | | | | 0.97 (d, J = 6.1 Hz, 6H). |
| 121 | 5-[{{[2-(2-{[(tert- | 4.82 mins. | 776 | | 1H NMR (300 MHz, DMSO-d6) |
| | butoxy)carbonyl]amino}ethoxy) | | | | δ 7.49 (d, J = 3.3 Hz, 1H), 6.75 |
| | ethoxy]carbonyl}oxy)methyl]-2- | | | | (br s, 2H), 6.50 (d, J = 3.3 Hz, |
| | {[{{[2-(2-{[(tert- | | | | 1H), 6.29 (s, 2H), 5.84 (s, 2H), |
| | butoxy)carbonyl]amino}ethoxy)etho | | | | 4.25 (t, J = 5.2 Hz, 2H), 4.21- |
| | xy]carbonyl}oxy)methyl]sulfanyl}-1- | | | | 4.14 (m, 4H), 3.66-3.61 (m, |
| | [2-(propan-2-yloxy)ethyl]-1H, 4H,5H- | | | | 2H), 3.58-3.51 (m, 4H), 3.5- |
| | pyrrolo[3,2-d]pyrimidin-4-one | | | | 3.35 (m, 5H), 3.01-2.99 (m, |
| | | | | | 4H), 1.31 (s, 18H), 0.95-0.92 |
| | | | | | (m, 6H). |
| 122 | 3,5-bis [{{[2-(2-{[(tert- | 5.34 mins. | 776 | | 1H NMR (300 MHz, CDCl3) δ |
| | butoxy)carbonyl]amino}ethoxy) | | | | 7.31 (d, J = 3.1 Hz, 1H), 6.65 |
| | ethoxy]carbonyl}oxy)methyl]-1-[2- | | | | (s, 2H), 6.28 (s, 2H), 6.27 (d, J = |
| | (propan-2-yloxy)ethyl]-2- | | | | 3.2 Hz, 1H), 4.88 (br s, 1H), |
| | sulfanylidene-1H,2H,3H,4H,5H- | | | | 4.60 (t, J = 5.6 Hz, 2H), 4.34- |
| | pyrrolo[3,2-d]pyrimidin-4-one | | | | 4.25 (m, 4H), 3.83 (t, J = 5.6 |
| | | | | | Hz, 2H), 3.66 (ddd, J = 6.3, 4.7, |
| | | | | | 3.1 Hz, 4H), 3.60-3.46 (m, |
| | | | | | 5H), 3.33-3.23 (m, 4H), 1.43 |
| | | | | | (s, 9H), 1.43 (s, 9H), 1.07 (d, J = |
| | | | | | 6.1 Hz, 6H). |
| 123 | trifluoroacetic acid 2-(2- | 3.12 mins. | 415 | | 1H NMR (300 MHz, DMSO-d6) |
| | aminoethoxy)ethyl {4-oxo-1-[2- | | | | δ 12.40 (s, 1H), 7.68 (br s, 2H), |
| | (propan-2-yloxy)ethyl]-2- | | | | 7.53 (d, J = 3.2 Hz, 1H), 6.40 |
| | sulfanylidene-1H,2H,3H,4H,5H- | | | | (d, J = 3.2 Hz, 1H), 6.17 (s, 2H), |
| | pyrrolo [3,2-d]pyrimidin-5-yl}methyl | | | | 4.46 (t, J = 5.9 Hz, 2H), 4.26- |
| | carbonate | | | | 4.19 (m, 2H), 3.69 (t, J = 6.0 |
| | | | | | Hz, 2H), 3.65-3.59 (m, 2H), |
| | | | | | 3.57-3.48 (m, 3H), 2.93 (dd, J = |
| | | | | | 10.4, 5.2 Hz, 2H), 0.97 (d, J = |
| | | | | | 6.1 Hz, 6H). |
| 124 | bis(trifluoroacetic acid) (2-{({[2-(2- | 2.70 mins. | 576 | | 1H NMR (300 MHz, DMSO-d6) |
| | aminoethoxy)ethoxy]carbonyl}oxy) | | | | δ 7.73 (br s, 4H), 7.52 (d, J = |
| | methyl]sulfanyl}-4-oxo-1-[2-(propan- | | | | 3.3 Hz, 1H), 6.52 (d, J = 3.3 Hz, |
| | 2-yloxy)ethyl]-1H,4H,5H-pyrrolo[3,2 | | | | 1H), 6.29 (s, 2H), 5.85 (s, 2H), |
| | d]pyrimidin-5-yl) methyl 2-(2- | | | | 4.30-4.19 (m, 6H), 3.68 |
| | aminoethoxy)ethyl carbonate | | | | 3.59 (m, 6H), 3.55 (td, J = 5.2, |
| | | | | | 2.3 Hz, 4H), 3.50-3.41 (m, |
| | | | | | 1H), 2.98-2.89 (m, 4H), 0.94 |
| | | | | | (d, J = 6.1 Hz, 1H). |
| 125 | tert-butyl 3-{[{{4-oxo-1-[2-(propan-2 | 4.71 mins | 483 | | 1H NMR (300 MHz, DMSO-d6) |
| | yloxy)ethyl]-2-sulfanylidene- | | | | δ 12.41 (s, 1H), 7.52 (d, J = 3.2 |
| | 1H,2H,3H,4H,5H-pyrrolo[3,2- | | | | Hz, 1H), 6.40 (d, J = 3.2 Hz, |
| | d]pyrimidin-5- | | | | 1H), 6.18 (s, 2H), 5.05 (dt, J = |
| | yl}methoxy)carbonyl]oxy}azetidine- | | | | 10.5, 3.4 Hz, 1H), 4.45 (t, J = |
| | 1-carboxylate | | | | 6.0 Hz, 2H), 4.17-4.07 (m, |
| | | | | | 2H), 3.78-3.72 (m, 2H), 3.69 |
| | | | | | (t, J = 6.0 Hz, 2H), 3.57-3.46 |
| | | | | | (m, 1H), 1.33 (s, 9H), 0.98- |
| | | | | | 0.95 (m, 6H). |
| 126 | 1-tert-butyl 4-{4-oxo-1-[2-(propan-2- | 4.82 mins. | 440 | | 1H NMR (300 MHz, CDCl3) δ |
| | yloxy)ethyl]-2-sulfanylidene- | | | | 9.27 (s, 1H), 7.27 (d, J = 3.2 |
| | 1H,2H,3H,4H,5H-pyrrolo[3,2- | | | | Hz, 1H), 6.26 (s, 2H), 6.24 (d, J = |
| | d]pyrimidin-5-yl}methyl | | | | 3.2 Hz, 1H), 4.52 (t, J = 5.7 |
| | butanedioate | | | | Hz, 2H), 3.83 (t, J = 5.7 Hz, |
| | | | | | 2H), 3.62-3.50 (m, 1H), 2.63- |
| | | | | | 2.55 (m, 2H), 2.55-2.48 (m, |
| | | | | | 2H), 1.40 (s, 9H), 1.09-1.05 |
| | | | | | (m, 6H). |
| 127 | 1-tert-butyl 3-{4-oxo-1-[2-(propan-2- | 4.65 mins. | 467 | | 1H NMR (300 MHZ, DMSO-d6) |
| | yloxy)ethyl]-2-sulfanylidene- | | | | δ 12.38 (s, 1H), 7.53 (d, J = 3.2 |
| | 1H,2H,3H,4H,5H-pyrrolo[3,2- | | | | Hz, 1H), 6.38 (d, J = 3.2 Hz, |
| | d]pyrimidin-5-yl}methyl azetidine- | | | | 1H), 6.19 (s, 2H), 4.45 (t, J = |
| | 1,3-dicarboxylate | | | | 6.0 Hz, 2H), 3.95 (t, J = 8.4 Hz, |
| | | | | | 2H), 3.80 (t, J = 7.0 Hz, 2H), |
| | | | | | 3.68 (t, J = 6.0 Hz, 2H), 3.56- |
| | | | | | 3.46 (m, 1H), 3.46-3.37 (m, |
| | | | | | 1H), 1.32 (s, 9H), 0.98-0.95 |
| | | | | | (m, 6H). |
| 128 | 1-tert-butyl 4-{4-oxo-1-[2-(propan-2- | 4.89 mins. | 495 | | 1H NMR (300 MHz, CDCl3) δ |
| | yloxy)ethyl]-2-sulfanylidene- | | | | 7.25-7.23 (m, 1H), 6.27- |
| | 1H,2H,3H,4H,5H-pyrrolo[3,2- | | | | 6.23 (m, 1H), 6.24 (s, 2H), 4.52 |
| | d]pyrimidin-5-yl} methyl piperidine- | | | | (t, J = 5.6 Hz, 2H), 4.03-3.93 |
| | 1,4-dicarboxylate | | | | (m, 2H), 3.84 (t, J = 5.6 Hz, 2H), |
| | | | | | 3.61-3.50 (m, 1H), 2.85- |
| | | | | | 2.71 (m, 2H), 2.52-2.42 (m, |
| | | | | | 1H), 1.87-1.77 (m, 2H), 1.65- |
| | | | | | 1.48 (m, 2H), 1.43 (s, 9H), |
| | | | | | 1.07-1.05 (m, 6H). |
| 129 | 1-tert-butyl 3-{4-oxo-1-[2-(propan-2- | 4.73 mins. | 481 | | 1H NMR (300 MHZ, DMSO-d6) |
| | yloxy)ethyl]-2-sulfanylidene- | | | | δ 12.37 (s, 1H), 7.52 (d, J = 3.1 |
| | 1H,2H,3H,4H,5H-pyrrolo[3,2- | | | | Hz, 1H), 6.38 (d, J = 3.2 Hz, |
| | d]pyrimidin-5-yl} methyl (3R)- | | | | 1H), 6.22-6.12 (m, 2H), 4.45 |
| | pyrrolidine-1,3-dicarboxylate | | | | (t, J = 6.0 Hz, 2H), 3.69 (t, J = |
| | | | | | 6.0 Hz, 2H), 3.57-3.46 (m, |
| | | | | | 1H), 3.33-3.25 (m, 2H), 3.24- |
| | | | | | 3.05 (m, 3H), 2.05-1.82 (m, |
| | | | | | 2H), 1.34 (s, 9H), 0.97 (d, J = |
| | | | | | 6.1 Hz, 6H). |
| 130 | 1-tert-butyl 3-{4-oxo-1-[2-(propan-2- | 4.77 mins. | 481 | | 1H NMR (300 MHz, DMSO-d6) |
| | yloxy)ethyl]-2-sulfanylidene- | | | | δ 12.37 (s, 1H), 7.52 (d, J = 3.2 |
| | 1H,2H,3H,4H,5H-pyrrolo[3,2- | | | | Hz, 1H), 6.38 (d, J = 3.2 Hz, |
| | d]pyrimidin-5-yl}methyl (3S)- | | | | 1H), 6.21-6.12 (m, 2H), 4.45 |
| | pyrrolidine-1,3-dicarboxylate | | | | (t, J = 6.0 Hz, 2H), 3.69 (t, J = |
| | | | | | 6.0 Hz, 2H), 3.58-3.45 (m, |
| | | | | | 1H), 3.32-3.27 (m, 2H), 3.23- |
| | | | | | 3.05 (m, 3H), 2.05-1.82 (m, |
| | | | | | 2H), 1.34 (s, 9H), 0.97 (d, J = |
| | | | | | 6.1 Hz, 6H). |
| 131 | 4-{[5-({[4-(tert-butoxy)-4- | 5.44 mins. | 681 | | 1H NMR (300 MHz, CDCl3) δ |
| | oxobutanoyl]oxy}methyl)-4-oxo-1- | | | | 7.36 (d, J = 3.3 Hz, 1H), 6.41 |
| | [2-(propan-2-yloxy)ethyl]-1H, 4H,5H- | | | | (s, 2H), 6.27 (d, J = 3.3 Hz, 1H), |
| | pyrrolo[3,2-d]pyrimidin-2- | | | | 5.92 (s, 2H), 4.25 (t, J = 5.7 Hz, |
| | yl]sulfanyl}methyl 1-tert-butyl | | | | 2H), 4.05-3.95 (m, 2H), 3.72 |
| | piperidine-1,4-dicarboxylate | | | | (t, J = 5.7 Hz, 2H), 3.56-3.44 |
| | | | | | (m, OH), 2.81 (t, J = 11.1 Hz, |
| | | | | | 2H), 2.60-2.45 (m, 5 H), 1.92- |
| | | | | | 1.83 (m, 2H), 1.67-1.53 (m, |
| | | | | | 2H), 1.43 (s, 9H), 1.40 (s, 9H), |
| | | | | | 1.07-1.04 (m, 6H). |
| 132 | 4-[5-({[4-(tert-butoxy)-4- | 6.47 mins. | 681 | | 1H NMR (300 MHz, CDCl3) |
| | oxobutanoyl]oxy}methyl)-4-oxo-1- | | | | 8.09-8.05 (m, 1H), 7.33 (s, |
| | [2-(propan-2-yloxy)ethyl]-2- | | | | 2H), 7.05 (s, 2H), 7.04-6.98 |
| | sulfanylidene-1H,2H,3H,4H,5H- | | | | (m, 1H), 5.40-5.35 (m, 2H), |
| | pyrrolo[3,2-d]pyrimidin-3-yl]methyl | | | | 4.73 (d, J = 12.6 Hz, 2H), 4.61 |
| | 1-tert-butyl piperidine-1,4- | | | | (t, J = 5.6 Hz, 2H), 4.41-4.28 |
| | dicarboxylate | | | | (m, 1H), 3.60 (t, J = 11.5 Hz, |
| | | | | | 2H), 3.40-3.20 (m, 5H), 2.54- |
| | | | | | 2.36 (m, 4H), 2.20 (s, 9H), |
| | | | | | 2.17 (s, 9H), 1.85 (d, J = 6.1 |
| | | | | | Hz, 6H). |
| 133 | 4-oxo-4-[(4-oxo-2-{[(piperidine-4- | 2.97 mins. | 525 | | 1H NMR (300 MHz, CDCl3) |
| | carbonyloxy)methyl]sulfanyl}-1-[2- | | | | 7.24-7.22 (m, 1H), 6.33 (s, |
| | (propan-2-yloxy)ethyl]-1H,4H,5H- | | | | 2H), 6.30 (d, J = 3.2 Hz, 1H), |
| | pyrrolo[3,2-d]pyrimidin-5- | | | | 5.74 (s, 2H), 4.27 (t, J = 5.6 Hz, |
| | yl)methoxy]butanoic acid; | | | | 2H), 3.77 (t, J = 5.7 Hz, 2H), |
| | trifluoroacetic acid | | | | 3.60-3.48 (m, 3H), 3.25- |
| | | | | | 3.15 (m, 2H), 2.83-2.75 (m, |
| | | | | | 1H), 2.63-2.57 (m, 2H), 2.54- |
| | | | | | 2.47 (m, 2H), 2.23-2.14 (m, |
| | | | | | 4H), 1.10-1.06 (m, 6H). |

| | Caco-2 | | | |
|---|---|---|---|---|
| | Pₐₚₚ | | | human Plasma |
| Example | (nm/s) | SGF | SIF | stability |
| 120 | 149 | 83% remaining | 64% remaining | 30% remaining |
| | | at 1 h | at 1 h | at 1 h |
| 121 | 11.9 | 69% remaining | 56% remaining | 34% remaining |
| | | at 1 h | at 1 h | at 1 h |
| 122 | 193 | 85% remaining | 22% remaining | 50% remaining |
| | | at 1 h | at 1 h | at 1 h |
| 123 | 24.4 | 97% remaining | 1% remaining at | 65% remaining |
| | | at 1 h | at 1 h | at 1 h |
| 124 | 11.8 | 67% remaining | 57% remaining | 28% remaining |
| | | at 1 h | at 1 h | at 1 h |
| 125 | 249 | 85% remaining | 17% remaining | 3% remaining |
| | | at 1 h | at 1 h | at 1 h |
| 126 | 135 | 100% | 67% remaining | 2% remaining |
| | | remaining at 1 h | at 1 h | at 1 h |
| 127 | 77.9 | 80% remaining | 6% remaining | 1% remaining |
| | | at 1 h | at 1 h | at 1 h |
| 128 | 119 | 78% remaining | 83% remaining | 31% remaining |
| | | at 1 h | at 1 h | at 1 h |
| 129 | 121 | 84% remaining | 21% remaining | 1% remaining |
| | | at 1 h | at 1 h | at 1 h |
| 130 | 176 | 61% remaining | 64% remaining | 4% remaining |
| | | at 1 h | at 1 h | at 1 h |
| 131 | 65.8 | 99% remaining | 75% remaining | 1% remaining |
| | | at 1 h | at 1 h | at 1 h |
| 132 | 42.9 | 9% remaining | 71% remaining | 11% remaining |
| | | at 1 h | at 1 h | at 1 h |
| 133 | <1.98 | 82% remaining | 97% remaining | 7% remaining |
| | | at 1 h | at 1 h | at 1 h |

### Examples 134-137

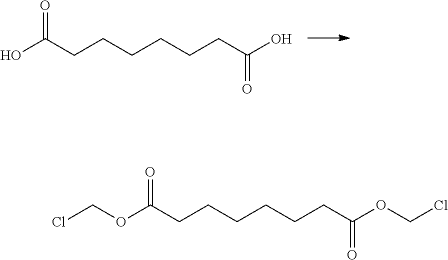 [SMILES: O=C(O)CCCCCCC(=O)O ; O=C(CCCCCCC(=O)OCCl)OCCl]

1,8-Dichloromethyl octanedioate: A solution of suberic acid (3.49 g, 20.06 mmol), tetrabutylammonium hydrogen sulfate (681 mg, 2.0 mmol), and sodium carbonate (11.8 g, 140.4 mmol) was dissolved in dichloromethane and water (50:50 v/v, 90 mL) and cooled at °0 C. on an ice bath. Chloromethyl chlorosulfonate (4.81 mL, 48.1 mmol) was add dropwise and the reaction was warmed to room temperature with stirring overnight. The reaction was poured into a separatory funnel and separated. The aqueous layer was extracted one more time with dichloromethane and the combined organic fractions were washed with brine and dried over magnesium sulfate and concentrated to provide the crude product (4.5 g), which was purified by flash chromatography eluting with a gradient of hexane to 10% ethyl acetate in hexane. The titled compound was isolated as a pale oil in appearance (170 mg). ¹H NMR (300 MHz, DMSO-d₆) δ 5.81 (s, 4H), 2.37 (t, J=7.3 Hz, 4H), 1.56-1.43 (m, 4H), 1.29-1.21 (m, 4H).

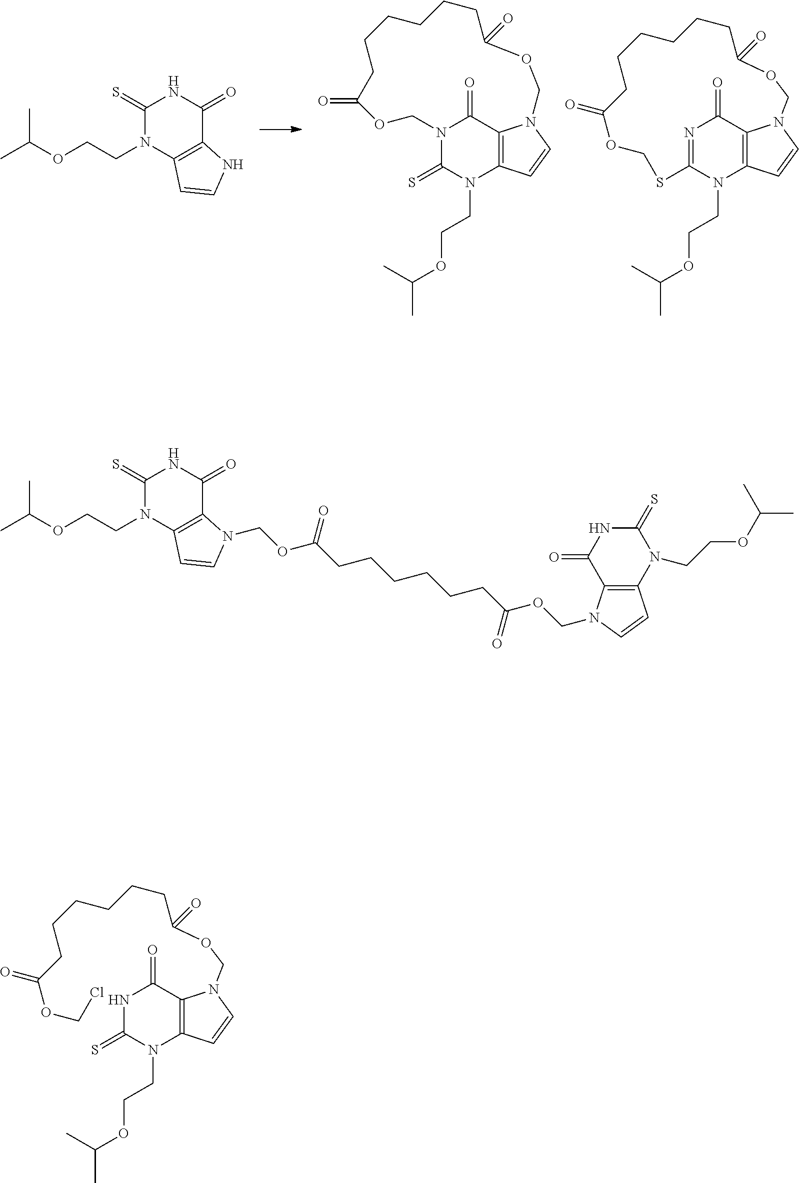

16-[2-(Propan-2-yloxy)ethyl]-15-sulfanylidene-3,12-dioxa-1,14,16-triazatricyclo[12.5.2.0\hat{ }{17,20}]henicosa-17(20),18-diene-4,11,21-trione and 22-[2-(propan-2-yloxy)ethyl]-6,15-dioxa-17-thia-4,19,22-triazatricyclo[16.3.1.0\hat{ }{4,21}]docosa-1(21),2,18-triene-7,14,20-trione and 1,8-bis({4-oxo-1-[2-(propan-2-yloxy)ethyl]-2-sulfanylidene-1H,2H,3H,4H,5H-pyrrolo[3,2-d]pyrimidin-5-yl}methyl) octanedioate and 1-chloromethyl 8-{4-oxo-1-[2-(propan-2-yloxy)ethyl]-2-sulfanylidene-1H,2H,3H,4H,5H-pyrrolo[3,2-d]pyrimidin-5-yl}methyl octanedioate: A solution of 1-[2-(propan-2-yloxy)ethyl]-2-sulfanylidene-1H,2H,3H,4H,5H-pyrrolo[3,2-d]pyrimidin-4-one (149 mg, 590 μmol) in DMF (28.0 mL) under nitrogen was treated with lithium hexamethyldisilylamide (1.0 M in THF, 590 μL, 590 μmol) and stirred for 10 mins at 0° C. 1,8-Dichloromethyl octanedioate (160 mg, 590 μmol) in DMF (2.0 mL) was added and the reaction was warmed to room temperature overnight. The reaction was quenched by the addition of water and partitioned between ethyl acetate and water (100 mL each). After separation, the aqueous layer was extracted an additional time with ethyl acetate. The combined organic fractions were washed with water and brine and dried over magnesium sulfate. Purification with RP-HPLC (Method B) in 5 separate runs provided cyclic compounds JTL-2-121-1, 26 mg; JTL-2-121-2, 12 mg; JTL-2-121-3, 29 mg, JTL-2-121-5, 8 mg. Repurification provided the following batches for testing: FC-12108 (6.7 mg), FC-12109 (5 mg), FC-12110 (23 mg), and FC-12111 (4 mg).

Spectra for EXAMPLE 134: 16-[2-(Propan-2-yloxy)ethyl]-15-sulfanylidene-3,12-dioxa-1,14,16-triazatricyclo[12.5.2.0\hat{ }{17,20}]henicosa-17(20),18-diene-4,11,21-trione. ¹H NMR (300 MHz, CHLOROFORM-d) δ 7.22 (d, J=3.2 Hz, 1H), 6.77 (s, 2H), 6.24 (d, J=3.1 Hz, 1H), 6.10 (s, 2H), 4.64 (t, J=5.7 Hz, 2H), 3.88 (t, J=5.7 Hz, 2H), 3.61-3.51 (m, 1H), 2.32 (dd, J=12.4, 5.2 Hz, 4H), 1.39-1.10 (m, 8H), 1.06 (d, J=6.1 Hz, 6H). LC/MS method A: Rₜ=5.08 mins., (M+H)⁺=452, purity >95%. SGF=99% remaining at 1 h, SIF=1% remaining at 1 h, hPlasma stability=1% remaining at 1 h.

Spectra for EXAMPLE 135: 22-[2-(Propan-2-yloxy)ethyl]-6,15-dioxa-17-thia-4,19,22-triazatricyclo[16.3.1.0\hat{ }{4,21}]docosa-1(21),2,18-triene-7,14,20-trione. ¹H NMR (300 MHz, CHLOROFORM-d) δ 7.30-7.27 (m, 1H), 6.35-6.20 (m, 2H), 6.18 (d, J=3.2 Hz, 1H), 5.96-5.53 (m, 2H), 4.35-4.20 (m, 2H), 3.71 (t, J=5.5 Hz, 2H), 3.47 (hept, J=6.1 Hz, 1H), 1.70-1.51 (m, 1H), 1.50-1.35 (m 2H), 1.34-1.20 (m, 3H), 1.19-1.07 (m, 2H), 1.05-1.01 (m, 6H). LC/MS method A: Rₜ=4.06 mins., (M+H)⁺=452, purity >95%. SGF=86% remaining at 1 h, SIF=67% remaining at 1 h, hPlasma stability=67% remaining at 1 h.

Spectra for EXAMPLE 136: 1,8-Bis({4-oxo-1-[2-(propan-2-yloxy)ethyl]-2-sulfanylidene-1H,2H,3H,4H,5H-pyrrolo[3,2-d]pyrimidin-5-yl}methyl) octanedioate. ¹H NMR (300 MHz, DMSO-d₆) δ 12.35 (s, 2H), 7.49 (d, J=3.1 Hz, 2H), 6.36 (d, J=3.2 Hz, 2H), 6.13 (s, 4H), 4.44 (t, J=6.0 Hz, 4H), 3.68 (t, J=6.1 Hz, 4H), 3.60-3.44 (m, 2H), 2.22 (t, J=7.2 Hz, 4H), 1.42-1.32 (m, 4H), 1.24 (s, 2H), 1.10 (s, 2H), 0.98-0.95 (m, 12H). LC/MS method A: Rₜ=5.11 mins., (M+H)⁺=705, purity >95%. SGF=77% remaining at 1 h, SIF=2% remaining at 1 h, hPlasma stability=2% remaining at 1 h.

Spectra for EXAMPLE 137: 1-Chloromethyl 8-{4-oxo-1-[2-(propan-2-yloxy)ethyl]-2-sulfanylidene-1H,2H,3H,4H,5H-pyrrolo[3,2-d]pyrimidin-5-yl}methyl octanedioate. ¹H NMR (300 MHz, CHLOROFORM-d) δ 9.23 (s, 1H), 6.24 (d, J=3.2 Hz, 1H), 6.22 (s, 2H), 5.69 (s, 2H), 4.52 (t, J=5.6 Hz, 2H), 3.83 (t, J=5.6 Hz, 2H), 3.54 (tt, J=9.3, 4.7 Hz, 1H), 2.33 (dt, J=9.8, 7.4 Hz, 4H), 1.67-1.52 (m, 4H), 1.35-1.20 (m, 4H), 1.09-1.04 (m, 6H). LC/MS method A: Rₜ=4.96 mins., (M+H)⁺=488 and 490, purity >95%, SGF=74% remaining at 1 h, SIF=56% remaining at 1 h, hPlasma stability=1% remaining at 1 h.

Additional compounds prepared according to EXAMPLES 134-137:

| | | LCMS | | | | Caco-2 | | | human |
|---|---|---|---|---|---|---|---|---|---|
| | | retention | | LCMS | | Pₐₚₚ | | | Plasma |
| Example | Name | time | M + H⁺ | method | NMR | (nm/s) | SGF | SIF | stability |
| 138 | 25-[2-(propan-2-yloxy)ethyl]-3,15- | 3.42 mins. | 507 | | 1H NMR (300 MHz, CDCl3) δ | | 100% remaining | 98% remaining | 66% remaining |
| | dioxa-5-thia-7,13,20,25- | | | | 7.34 (d, J = 3.4 Hz, 1H), 6.44 | | at 1 h | at 1 h | at 1 h |
| | tetraazatetra- | | | | (br s, 1H), 6.29 (s, 2H), 6.17 (d, | | | | |
| | cyclo[18.2.2.1\hat{ }{6,10}.0\hat{ }{9,13}]penta- | | | | J = 3.2 Hz, 1H), 5.22 (br s, 1H), | | | | |
| | cosa-6,9,11-triene- | | | | 4.48-4.37 (m, 1H), 4.14 (dt, J = | | | | |
| | 2,8,16,19-tetrone | | | | 15.1, 5.0 Hz, 1H), 3.76-3.64 | | | | |
| | | | | | (m, 2H), 3.60 3.43 (m, 2H), | | | | |
| | | | | | 3.31-2.92 (m, 3H), 2.80- | | | | |
| | | | | | 2.58 (m, 3H), 2.49-2.39 (m, | | | | |
| | | | | | 1H), 2.33-2.27 (m, 1H), 1.80- | | | | |
| | | | | | 1.49 (m, 3H), 1.05 (dd, J = | | | | |
| | | | | | 6.1, 4.1 Hz, 6H). | | | | |
| 139 | 4-oxo-4-({4-oxo-3-[(piperidine-4- | 3.23 mins. | 525 | | 1H NMR (300 MHz, DMSO-d6) | | 99% remaining | 98% remaining | 8% remaining |
| | carbonyloxy)methyl]-1-[2-(propan-2- | | | | δ 7.63 (d, J = 3.2 Hz, 1H), 6.47 | | at 1 h | at 1 h | at 1 h |
| | yloxy)ethyl]-2-sulfanylidene- | | | | (d, J = 3.2 Hz, 1H), 6.37 (s, 2H), | | | | |
| | 1H,2H,3H,4H,5H-pyrrolo[3,2- | | | | 6.18 (s, 2H), 4.56 (t, J = 5.7 Hz, | | | | |
| | d]pyrimidin-5-yl}methoxy)butanoic | | | | 2H), 3.72 (t, J = 5.9 Hz, 2H), | | | | |
| | acid; trifluoroacetic acid | | | | 3.59-3.48 (m, 1H), 3.25- | | | | |
| | | | | | 3.15 (m, 2H), 2.95-2.80 (m, | | | | |
| | | | | | 2H), 2.75-2.65 (m, 1H), 2.52- | | | | |
| | | | | | 2.37 (m, 4H), 1.97-1.87 (m, | | | | |
| | | | | | 2H), 1.77-1.65 (m, 2H), 0.98 | | | | |
| | | | | | (d, J = 6.1 Hz, 6H). | | | | |
| 140 | 19-[2-(propan-2-yloxy)ethyl]-20- | 4.38 mins. | 507 | | 1H NMR (300 MHz, CDCl3) δ | | 99% remaining | 79% remaining | 10% remaining |
| | sulfanylidene-3,13-dioxa-1,8,15,19- | | | | 7.31-7.26 (m, 1H), 6.90 (br s, | | at 1 h | at 1 h | at 1 h |
| | tetraazatetra- | | | | 2H), 6.28 (d, J = 10.4 Hz, 1H), | | | | |
| | cyclo[13.5.2.2\hat{ }{5,8}.0\hat{ }{18,22}]tetra- | | | | 6.24 (d, J = 3.2 Hz, 1H), 6.18 | | | | |
| | cosa-16,18(22)-diene- | | | | (br s, 1H), 4.64 (br s, 2H), 3.87 | | | | |
| | 4,9,12,21-tetrone | | | | (td, J = 5.7, 2.3 Hz, 2H), 3.56 | | | | |
| | | | | | (dt, J = 12.2, 6.1 Hz, 1H), 3.50- | | | | |
| | | | | | 3.05 (m, 4H), 2.85-2.75 (m, | | | | |
| | | | | | 2H), 2.48 (d, J = 14.9 Hz, 1H), | | | | |
| | | | | | 2.25-2.13 (m, 1H), 2.10- | | | | |
| | | | | | 2.00 (m, 2H), 1.80-1.57 (m, | | | | |
| | | | | | 3H), 1.07 (dd, J = 6.1, 2.7 Hz, | | | | |
| | | | | | 6H). | | | | |
| 141 | 1-tert-butyl 4-{4-oxo-1-[2-(propan-2- | 6.31 mins. | 723 | | 1H NMR (300 MHz, DMSO-d6) | | | | |
| | yloxy)ethyl]-2-sulfanylidene- | | | | δ 12.40 (s, 1H), 8.11-8.08 (m, | | | | |
| | 1H,2H,3H,4H,5H-pyrrolo[3,2- | | | | 1H), 7.47 (d, J = 1.7 Hz, 1H), | | | | |
| | d]pyrimidin-5-yl}methyl (2S)-2-[12- | | | | 6.37 (d, J = 3.2 Hz, 1H), 6.18- | | | | |
| | (tert-butoxy)-12- | | | | 6.14 (m, 2H), 4.48-4.39 (m, | | | | |
| | oxododecanamido]butanedioate | | | | 3H), 3.70-3.64 (m, 2H), 3.57- | | | | |
| | | | | | 3.48 (m, 1H), 2.76-2.55 (m, | | | | |
| | | | | | 2H), 2.15-2.09 (m, 2H), 2.04- | | | | |
| | | | | | 1.98 (m, 2H), 1.46-1.37 (m, | | | | |
| | | | | | 4H), 1.35 (s, 9H), 1.28 (s, 9H), | | | | |
| | | | | | 1.19 (s, 12H), 1.00-0.97 (m, | | | | |
| | | | | | 6H). | | | | |
| 142 | 11-{[(1S)-1-carboxy-3-oxo-3-({4- | 4.28 mins. | 611 | A | 1H NMR (300 MHz, DMSO-d6) | <1.9 | 100% remaining | 97% remaining | 97% remaining |
| | oxo-1-[2-(propan-2-yloxy)ethyl]-2- | | | | δ 12.38 (s, 1H), 8.06 (d, J = 8.2 | | after 1 h | after 1 h | after 1 h |
| | sulfanylidene-1H,2H,3H,4H,5H- | | | | Hz, 1H), 7.46 (d, J = 3.2 Hz, | | | | |
| | pyrrolo[3,2-d]pyrimidin-5- | | | | 1H), 6.37 (d, J = 3.2 Hz, 1H), | | | | |
| | yl}methoxy)propyl]carbamoyl}undecanoic | | | | 6.15 (s, 2H), 4.51-4.40 (m, | | | | |
| | acid | | | | 3H), 3.67 (t, J = 6.0 Hz, 2H), | | | | |
| | | | | | 3.60-3.46 (m, 1H), 2.66 (ddd, | | | | |
| | | | | | J = 23.7, 16.4, 6.7 Hz, 2H), | | | | |
| | | | | | 2.14 (t, J = 7.3 Hz, 2H), 2.00 (t, | | | | |
| | | | | | J = 7.3 Hz, 2H), 1.51-1.31 (m, | | | | |
| | | | | | 4H), 1.18 (s, 12H), 0.98 (d, J = | | | | |
| | | | | | 6.1 Hz, 6H). | | | | |

### Example 143

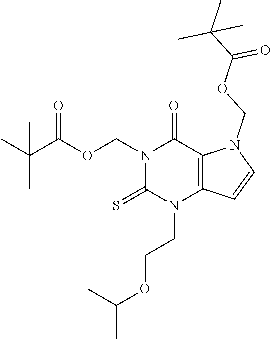 [SMILES: CC(C)OCCn1c(=S)n(COC(=O)C(C)(C)C)c(=O)c2c1ccn2COC(=O)C(C)(C)C]

2,2-Dimethyl-propionic acid 5-(2,2-dimethyl-propionyloxymethyl)-1-(2-isopropoxy-ethyl)-4-oxo-2-thioxo-1,2,4,5-tetrahydro-pyrrolo[3,2-d]pyrimidin-3-ylmethyl ester: Added potassium carbonate (0.120 g, 0.868 mmole) to a solution of 1-[2-(propan-2-yloxy)ethyl]-2-sulfanylidene-1H,2H,3H,4H,5H-pyrrolo[3,2-d]pyrimidin-4-one (0.1 g, 0.394 mmole) and chloromethyl pivalate (0.125 mL, 0.868 mmole) at RT under N₂ and magnetically stirred in 5 mL of acetone. After 2 days, filtered off solids, and concentrated the filtrate in vacuo. Purified using a 20 g Agela silica gel column, eluting with 0-45% ethyl acetate in hexanes to yield 2,2-Dimethyl-propionic acid 5-(2,2-dimethyl-propionyloxymethyl)-1-(2-isopropoxy-ethyl)-4-oxo-2-thioxo-1,2,4,5-tetrahydro-pyrrolo[3,2-d]pyrimidin-3-ylmethyl ester: (13 mg, 6.8%), ¹H NMR (CHLOROFORM-d) δ: 7.28 (d, J=3.1 Hz, 1H), 6.56 (s, 2H), 6.26 (s, 2H), 6.24 (d, J=3.2 Hz, 1H), 4.62 (t, J=5.7 Hz, 2H), 3.83-3.91 (m, 2H), 3.55 (quin, J=6.1 Hz, 1H), 1.20 (s, 9H), 1.15 (s, 9H), 1.05 (d, J=6.0 Hz, 6H). LC/MS method A: Rₜ=6.47 mins., (M+H)⁺=482, purity=95. Caco-2 Pₐₚₚ=30 nm/s, SGF=62% remaining at 1 h, SIF=82% remaining at 1 h, hPlasma stability=21% remaining at 1 h.

Additional compounds prepared similarly to EXAMPLE 143:

| | | LCMS | | | | Caco-2 | | | human |
|---|---|---|---|---|---|---|---|---|---|
| | | retention | | LCMS | | Pₐₚₚ | | | Plasma |
| Example | Name | time | M + H⁺ | method | NMR | (nm/s) | SGF | SIF | stability |
| 144 | [(5-{[(2,2- | 5.38 | 482 | | ¹H NMR (CHLOROFORM-d) δ: | 51.1 | | | |
| | dimethylpropanoyl)oxy]methyl}-4- | | | | 7.21 (d, J = 3.3 Hz, 1H), 6.42 | | | | |
| | oxo-1-[2-(propan-2-yloxy)ethyl]- | | | | (s, 2H), 6.17 (d, J = 3.3 Hz, | | | | |
| | 1H,4H,5H-pyrrolo[3,2-d]pyrimidin-2- | | | | 1H), 5.97 (s, 2H), 4.19 (t, | | | | |
| | yl)sulfanyl]methyl 2,2- | | | | J = 5.9 Hz, 2H), 3.65-3.74 (m, | | | | |
| | dimethylpropanoate | | | | 2H), 3.47 (quin, J = 6.1 Hz, | | | | |
| | | | | | 1H), 1.14-1.20 (m, 9H), 1.12 | | | | |
| | | | | | (s, 9H), 1.01 (d, J = 6.1 Hz, | | | | |
| | | | | | 6H) | | | | |
| 145 | {[5-(hydroxymethyl)-4-oxo-1-[2- | 4.37 | 398 | | ¹H NMR (CHLOROFORM-d) δ: | | | | |
| | (propan-2-yloxy)ethyl]-1H,4H,5H- | | | | 7.26 (s, 2H), 7.09 (d, J = 3.0 | | | | |
| | pyrrolo[3,2-d]pyrimidin-2- | | | | Hz, 1H), 6.70-6.85 (m, 1H), | | | | |
| | yl]sulfanyl}methyl 2,2- | | | | 6.14 (d, J = 3.0 Hz, 1H), 5.99 | | | | |
| | dimethylpropanoate | | | | (s, 2H), 5.56 (s, 2H), 4.26 (t, | | | | |
| | | | | | J = 5.9 Hz, 2H), 3.73 (t, J = 5.8 | | | | |
| | | | | | Hz, 2H), 3.51 (quin, J = 5.9 Hz, | | | | |
| | | | | | 1H), 1.17-1.21 (m, 9H), 1.02- | | | | |
| | | | | | 1.09 (m, 6H) | | | | |
| 146 | {4-oxo-1-[2-(propan-2-yloxy)ethyl]- | 5.1 min | 368 | | ¹H NMR (CHLOROFORM-d) δ: | | | | |
| | 2-sulfanylidene-1H,2H,3H,4H,5H- | | | | 9.39 (s, 1H), 7.25 (s, 1H), | | | | |
| | pyrrolo[3,2-d]pyrimidin-5-yl}methyl | | | | 6.24 (s, 3H), 4.53 (t, J = 5.6 | | | | |
| | 2,2-dimethylpropanoate | | | | Hz, 2H), 3.85 (t, J = 5.6 Hz, | | | | |
| | | | | | 2H), 3.54 (spt, J = 6.1 Hz, 1H), | | | | |
| | | | | | 1.15 (s, 9H), 1.05 (d, J = 6.0 | | | | |
| | | | | | Hz, 6H) | | | | |
| 147 | ({4-oxo-1-[2-(propan-2-yloxy)ethyl]- | 4.37 | 368 | | ¹H NMR (CHLOROFORM-d) δ: | | | | |
| | 1H,4H,5H-pyrrolo[3,2-d]pyrimidin-2- | | | | 12.69 (s, 1H), 7.42 (t, J = 3.0 | | | | |
| | yl}sulfanyl)methyl 2,2- | | | | Hz, 1H), 6.22 (dd, J = 3.0, 1.9 | | | | |
| | dimethylpropanoate | | | | Hz, 1H), 6.03 (s, 2H), 4.30 (t, | | | | |
| | | | | | J = 6.0 Hz, 2H), 3.70-3.79 (m, | | | | |
| | | | | | 2H), 3.51 (quin, J = 6.1 Hz, | | | | |
| | | | | | 1H), 1.18 (s, 9H), 1.06 (d, | | | | |
| | | | | | J = 5.9 Hz, 6H) | | | | |
| 148 | {4-oxo-1-[2-(propan-2-yloxy)ethyl]- | 8.35 Method: | 522 | | 1H NMR (CHLOROFORM-d) δ: | | | | |
| | 2-sulfanylidene-1H,2H,3H,4H,5H- | 40-95% | | | 9.69 (s, 1H), 7.30 (d, J = 3.2 | | | | |
| | pyrrolo[3,2-d]pyrimidin-5-yl}methyl | ACN with a | | | Hz, 1H), 6.20-6.29 (m, 3H), | | | | |
| | hexadecanoate | hold for 10 | | | 4.53 (t, J = 5.7 Hz, 2H), 3.84 | | | | |
| | | min at | | | (t, J = 5.7 Hz, 2H), 3.46-3.66 | | | | |
| | | 95% ACN | | | (m, 1H), 2.31 (t, J = 7.5 Hz, | | | | |
| | | | | | 2H), 1.57-1.73 (br t, J = 7.2 | | | | |
| | | | | | Hz, 2 H), 1.19-1.31 (m, 24H), | | | | |
| | | | | | 1.04-1.10 (d, J = 6.1 Hz, 6H), | | | | |
| | | | | | 0.83-0.93 (m, 3H) | | | | |
| 149 | {4-oxo-1-[2-(propan-2-yloxy)ethyl]- | 9.9 Method: | 550 | | 1H NMR (CHLOROFORM-d) δ: | | | | |
| | 2-sulfanylidene-1H,2H,3H,4H,5H- | 40-95% | | | 9.31 (s, 1H), 7.28 (d, J = 3.2 | | | | |
| | pyrrolo[3,2-d]pyrimidin-5-yl}methyl | ACN with a | | | Hz, 1H), 6.23-6.26 (m, 2H), | | | | |
| | octadecanoate | hold for 10 | | | 4.53 (t, J = 5.7 Hz, 2H), 3.84 | | | | |
| | | min at | | | (t, J = 5.7 Hz, 2H), 3.56 (quin, | | | | |
| | | 95% ACN | | | J = 6.1 Hz, 1H), 2.32 (t, J = 7.5 | | | | |
| | | | | | Hz, 2H), 1.58 (s, 2H), 1.21- | | | | |
| | | | | | 1.30 (m, 28H), 1.07 (d, J = 6.0 | | | | |
| | | | | | Hz, 6H), 0.84-0.92 (m, 3H) | | | | |
| 150 | {4-oxo-1-[2-(propan-2-yloxy)ethyl]- | 10.88 Method: | 565 | | ¹H NMR (CHLOROFORM-d) δ: | | | | |
| | 2-sulfanylidene-1H,2H,3H,4H,5H- | 40-95% | | | 9.31 (s, 1H), 7.28 (d, J = 3.2 | | | | |
| | pyrrolo[3,2-d]pyrimidin-5-yl}methyl | ACN with a | | | Hz, 2H), 6.21-6.29 (m, 3H), | | | | |
| | nonadecanoate | hold for 10 | | | 4.53 (t, J = 5.7 Hz, 2H), 3.84 | | | | |
| | | min at | | | (t, J = 5.7 Hz, 2H), 3.56 (quin, | | | | |
| | | 95% ACN | | | J = 6.1 Hz, 1H), 2.32 (t, J = 7.5 | | | | |
| | | | | | Hz, 2H), 1.58 (m, 4H), 1.19- | | | | |
| | | | | | 1.31 (m, 28H), 1.07 (d, J = 5.9 | | | | |
| | | | | | Hz, 6H), 0.88 (s, 3H) | | | | |
| 151 | {4-oxo-1-[2-(propan-2-yloxy)ethyl]- | 12.4 Method: | 578 | | ¹H NMR (CHLOROFORM-d) δ: | | | | |
| | 2-sulfanylidene-1H,2H,3H,4H,5H- | 40-95% | | | 9.40 (s, 1H), 7.28 (d, J = 3.2 | | | | |
| | pyrrolo[3,2-d]pyrimidin-5-yl}methyl | ACN with a | | | Hz, 1H), 6.18-6.42 (m, 3H), | | | | |
| | icosanoate | hold for 10 | | | 4.53 (t, J = 5.7 Hz, 2H), 3.84 | | | | |
| | | min at | | | (t, J = 5.7 Hz, 2H), 3.56 (spt, | | | | |
| | | 95% ACN | | | J = 6.1 Hz, 1H), 2.32 (t, J = 7.5 | | | | |
| | | | | | Hz, 2H), 1.51-1.70 (m, 4H), | | | | |
| | | | | | 1.25 (m, 30H), 1.07 (d, J = 6.2 | | | | |
| | | | | | Hz, 6H), 0.84-0.92 (m, 3H) | | | | |
| 152 | {4-oxo-1-[2-(propan-2-yloxy)ethyl]- | Compound | Compound | | 1H NMR (CHLOROFORM-d) δ: | | | | |
| | 2-sulfanylidene-1H,2H,3H,4H,5H- | did not | did not | | 9.32 (s, 1H), 7.27-7.30 (m, | | | | |
| | pyrrolo[3,2-d]pyrimidin-5-yl}methyl | elute on | elute on | | 1H), 6.24 (s, 3H), 4.49-4.58 | | | | |
| | henicosanoate | the LCMS | the LCMS | | (m, 2H), 3.84 (t, J = 5.7 Hz, | | | | |
| | | | | | 2H), 3.56 (quin, J = 6.1 Hz, | | | | |
| | | | | | 1H), 2.32 (t, J = 7.9 Hz, 2H), | | | | |
| | | | | | 1.47-1.74 (m, 4H), 1.19-1.35 | | | | |
| | | | | | (m, 32H), 1.07 (d, J = 6.1 Hz, | | | | |
| | | | | | 6H), 0.82-0.93 (m, 3H) | | | | |
| 153 | {4-oxo-1-[2-(propan-2-yloxy)ethyl]- | Compound | Compound | | ¹H NMR (CHLOROFORM-d) δ: | | | | |
| | 2-sulfanylidene-1H,2H,3H,4H,5H- | did not | did not | | 9.27 (s, 1H), 7.27-7.29 (m, | | | | |
| | pyrrolo[3,2-d]pyrimidin-5-yl}methyl | elute on | elute on | | 1H), 6.24 (s, 3H), 4.53 (t, | | | | |
| | docosanoate | the LCMS | the LCMS | | J = 5.6 Hz, 2H), 3.84 (t, J = 5.6 | | | | |
| | | | | | Hz, 2H), 3.49-3.63 (m, 1H), | | | | |
| | | | | | 2.32 (t, J = 7.5 Hz, 2H), 1.57 | | | | |
| | | | | | (s, 4H), 1.24 (br d, J = 4.9 Hz, | | | | |
| | | | | | 36H), 1.03-1.11 (m, 6H), 0.83 | | | | |
| | | | | | 0.92 (m, 3H) | | | | |
| 154 | {4-oxo-1-[2-(propan-2-yloxy)ethyl]- | 4.58 | 354 | | 1H NMR (CHLOROFORM-d) δ: | 40.3 | 82% @ 1 h | 0% @ 1 h | 9% @ 1 h |
| | 2-sulfanylidene-1H,2H,3H,4H,5H- | | | | 9.33 (s, 1H), 7.28 (d, J = 3.2 | | | | |
| | pyrrolo[3,2-d]pyrimidin-5-yl}methyl | | | | Hz, 1H), 6.22-6.27 (m, 3H), | | | | |
| | butanoate | | | | 4.53 (t, J = 5.6 Hz, 2H), 3.84 | | | | |
| | | | | | (t, J = 5.6 Hz, 2H), 3.56 (dt, | | | | |
| | | | | | J = 12.2, 6.1 Hz, 1H), 2.31 (t, | | | | |
| | | | | | J = 7.4 Hz, 2H), 1.55-1.71 (m, | | | | |
| | | | | | 2H), 1.06 (d, J = 6.2 Hz, 6H), | | | | |
| | | | | | 0.89 (t, J = 7.4 Hz, 3H). | | | | |
| 155 | ({5-[(butanoyloxy)methyl]-4-oxo-1- | 4.88 | 454 | | ¹H NMR (CHLOROFORM-d) δ: | 27.9 | 73% @ 1 h | 0% @ 1 h | 1% @ 1 h |
| | [2-(propan-2-yloxy)ethyl]-1H,4H,5H- | | | | 7.25 (s, 1H), 6.44 (s, 2H), | | | | |
| | pyrrolo[3,2-d]pyrimidin-2- | | | | 6.19 (d, J = 3.2 Hz, 1H), 5.98 | | | | |
| | yl}sulfanyl)methyl butanoate | | | | (s, 2H), 4.21 (t, J = 5.9 Hz, | | | | |
| | | | | | 2H), 3.69 (s, 2H), 3.51 (quin, | | | | |
| | | | | | J = 6.1 Hz, 1H), 2.31 (q, J = 7.6 | | | | |
| | | | | | Hz, 4H), 1.61-1.71 (m, 4H), | | | | |
| | | | | | 1.06 (d, J = 6.1 Hz, 6H), 0.84- | | | | |
| | | | | | 0.96 (m, 6H). | | | | |
| 156 | {4-oxo-1-[2-(propan-2-yloxy)ethyl]- | 4.25 | 340 | | 1H NMR (CHLOROFORM-d) δ: | 65.5 | 99% @ 1 h | 1% @ 1 h | 0% @ 1 h |
| | 2-sulfanylidene-1H,2H,3H,4H,5H- | | | | 9.40 (s, 1H), 7.28 (d, J = 3.2 | | | | |
| | pyrrolo[3,2-d]pyrimidin-5-yl}methyl | | | | Hz, 1H), 6.20-6.32 (m, 3H), | | | | |
| | propanoate | | | | 4.53 (t, J = 5.7 Hz, 2H), 3.84 | | | | |
| | | | | | (t, J = 5.7 Hz, 2H), 3.56 (quin, | | | | |
| | | | | | J = 6.1 Hz, 1H), 2.36 (q, J = 7.5 | | | | |
| | | | | | Hz, 2H), 1.11 (t, J = 7.6 Hz, | | | | |
| | | | | | 3H), 1.07 (d, J = 6.2 Hz, 6H) | | | | |
| 157 | trifluoroacetic acid {4-oxo-1-[2- | 5.67 | 388 | | ¹H NMR (CHLOROFORM-d) δ: | 73 | 96% @ 1 h | 20% @ 1 h | 0% @ 1 h |
| | (propan-2-yloxy)ethyl]-2- | | | | 9.25 (s, 1H), 8.00-8.07 (m, | | | | |
| | sulfanylidene-1H,2H,3H,4H,5H- | | | | 2H), 7.53-7.61 (m, 1H), 7.39- | | | | |
| | pyrrolo[3,2-d]pyrimidin-5-yl}methyl | | | | 7.48 (m, 3H), 6.49 (s, 2H), | | | | |
| | benzoate | | | | 6.26 (d, J = 3.1 Hz, 1H), 4.53 | | | | |
| | | | | | (t, J = 5.7 Hz, 2H), 3.83 (t, | | | | |
| | | | | | J = 5.7 Hz, 2H), 3.56 (quin, | | | | |
| | | | | | J = 6.1 Hz, 1H), 1.06 (d, J = 5.9 | | | | |
| | | | | | Hz, 6H) | | | | |
| 158 | trifluoroacetic acid (2- | 6.08 | 522 | | ¹H NMR (CHLOROFORM-d) δ: | 53.7 | | | |
| | {[(benzoyloxy)methyl]sulfanyl}-4- | | | | 8.01-8.15 (m, 4H), 7.52-7.64 | | | | |
| | oxo-1-[2-(propan-2-yloxy)ethyl]- | | | | (m, 2H), 7.34-7.50 (m, 4H), | | | | |
| | 1H,4H,5H-pyrrolo[3,2-d]pyrimidin-5- | | | | 6.69 (s, 2H), 6.25 (d, J = 3.4 | | | | |
| | yl)methyl benzoate | | | | Hz, 1H), 6.22 (s, 2H), 4.24 (t, | | | | |
| | | | | | J = 5.8 Hz, 2H), 3.71 (t, J = 5.8 | | | | |
| | | | | | Hz, 2H), 3.48 (dt, J = 12.1, 6.1 | | | | |
| | | | | | Hz, 1H), 1.02 (d, J = 6.2 Hz, | | | | |
| | | | | | 6H) | | | | |
| 159 | {4-oxo-1-[2-(propan-2-yloxy)ethyl]- | 4.77 | 326 | | ¹H NMR (CHLOROFORM-d) δ: | 84.2 | 79% @ 1 h | 83% @ 1 h | 0% @ 1 h |
| | 2-sulfanylidene-1H,2H,3H,4H,5H- | | | | 9.23 (br s, 1H), 7.28 (d, J = 3.2 | | | | |
| | pyrrolo [3,2-d]pyrimidin-5-yl}methyl | | | | Hz, 1H), 6.25 (d, J = 3.4 Hz, | | | | |
| | acetate | | | | 1H), 6.22-6.24 (m, 2H), 4.53 | | | | |
| | | | | | (t, J = 5.7 Hz, 2H), 3.84 (t, | | | | |
| | | | | | J = 5.7 Hz, 2H), 3.57 (dt, | | | | |
| | | | | | J = 12.2, 6.1 Hz, 1H), 2.09 (s, | | | | |
| | | | | | 3H), 1.08 (d, J = 6.4 Hz, 6H) | | | | |
| 160 | trifluoroacetic acid ({4-oxo-1-[2- | 5.02 | 397 | | ¹H NMR (CHLOROFORM-d) δ: | 158 | 92% @ 1 h | 95% @ 1 h | 99% @ 1 h |
| | (propan-2-yloxy)ethyl]-1H,4H,5H- | | | | 12.52 (br s, 1H), 7.48 (t, | | | | |
| | pyrrolo[3,2-d]pyrimidin-2- | | | | J = 3.0 Hz, 1H), 6.32-6.37 (m, | | | | |
| | yl}sulfanyl)methyl N-butyl-N- | | | | 1H), 6.02 (d, J = 1.2 Hz, 2H), | | | | |
| | methylcarbamate | | | | 4.39 (t, J = 5.7 Hz, 2H), 4.04- | | | | |
| | | | | | 4.26 (m, 4H), 3.78 (t, J = 5.6 | | | | |
| | | | | | Hz, 2H), 3.50 (quin, J = 6.1 Hz, | | | | |
| | | | | | 1H), 3.24 (dt, J = 19.2, 7.3 Hz, | | | | |
| | | | | | 2H), 2.88 (d, J = 17.3 Hz, 3H), | | | | |
| | | | | | 1.36-1.57 (m, 2H), 1.12-1.35 | | | | |
| | | | | | (m, 2H), 1.04 (d, J = 6.1 Hz, | | | | |
| | | | | | 6H), 0.74-0.96 (m, 3H) | | | | |
| 161 | {2-[{{[methyl(2- | 6.07 | 540 | | ¹H NMR (CHLOROFORM-d) δ: | 313 | 80% @ 1 h | 80% @ 1 h | 99% @ 1 h |
| | methylpropyl)carbamoyl]oxy}methyl)sulfanyl]- | | | | 7.37 (d, J = 3.2 Hz, 1H), 6.44 | | | | |
| | 4-oxo-1-[2-(propan-2- | | | | (s, 2H), 6.16 (d, J = 3.2 Hz, | | | | |
| | yloxy)ethyl]-1H,4H,5H-pyrrolo[3,2- | | | | 1H), 5.99-6.08 (m, 2H), 4.15- | | | | |
| | d]pyrimidin-5-yl}methyl N-methyl-N- | | | | 4.28 (m, 2H), 3.61-3.81 (m, | | | | |
| | (2-methylpropyl)carbamate | | | | 2H), 3.43-3.58 (m, 1H), 2.99- | | | | |
| | | | | | 3.14 (m, 4H), 2.80-2.94 (m, | | | | |
| | | | | | 6H), 1.72-2.01 (m, 2H), 1.00- | | | | |
| | | | | | 1.09 (m, 6H), 0.83-0.92 (m, | | | | |
| | | | | | 6H), 0.77 (dd, J = 19.2, 6.7 Hz, | | | | |
| | | | | | 6H) | | | | |
| 162 | {2- | 6.93 | 540 | | ¹H NMR (CHLOROFORM-d) δ: | 287 | 98% @ 1 h | 96% @ 1 h | 96% @ 1 h |
| | [{{[butyl(methyl)carba- | | | | 7.36 (m, 1H), 6.42 (d, 2H), | | | | |
| | moyl]oxy}methyl)sulfanyl]- | | | | 6.15 (m, 1H), 5.94-6.06 (m, | | | | |
| | 4-oxo-1-[2-(propan-2- | | | | 2H), 4.21 (m, 2H), 3.70 (m, | | | | |
| | yloxy)ethyl]-1H,4H,5H-pyrrolo[3,2- | | | | 2H), 3.42-3.58 (m, 1H), 3.11- | | | | |
| | d]pyrimidin-5-yl}methyl N-butyl-N- | | | | 3.34 (m, 4H), 2.86 (m, 6H), | | | | |
| | methylcarbamate | | | | 1.09-1.58 (m, 8H), 1.02-1.08 | | | | |
| | | | | | (m, 6H), 0.85-0.96 (m, 3H), | | | | |
| | | | | | 0.74-0.84 (m, 3H). | | | | |
| 163 | ({4-oxo-1-[2-(propan-2-yloxy)ethyl]- | 5.2 | 369 | | ¹H NMR (CHLOROFORM-d) δ: | 42 | 99% @ 1 h | 96% @ 1 h | 90% @ 1 h |
| | 1H,4H,5H-pyrrolo[3,2-d]pyrimidin-2- | | | | 12.46 (br s, 1H), 7.40 (m, | | | | |
| | yl}sulfanyl)methyl N-ethyl-N- | | | | 1H), 6.22 (br s, 1H), 6.06 (s, | | | | |
| | methylcarbamate | | | | 2H), 4.20-4.38 (m, 2H), 3.75 | | | | |
| | | | | | (m, 2H), 3.43-3.63 (m, 1H), | | | | |
| | | | | | 3.14-3.40 (m, 2H), 2.88 (d, | | | | |
| | | | | | 3H), 1.06 (d, 6H). | | | | |
| 164 | trifluoroacetic acid ({4-oxo-1-[2- | 5.22 | 355 | | 1H NMR (300 MHz, | 42 | 100% @ 1 h | 99% @ 1 h | 98% @ 1 h |
| | (propan-2-yloxy)ethyl]-1H,4H,5H- | | | | CHLOROFORM-d) δ = 12.64 (s, | | | | |
| | pyrrolo[3,2-d]pyrimidin-2- | | | | 1H), 7.52 (t, J = 3.0 Hz, 1H), | | | | |
| | yl}sulfanyl)methyl N,N- | | | | 6.48-6.25 (m, 1H), 6.00 (s, | | | | |
| | dimethylcarbamate | | | | 2H), 4.39 (t, J = 5.6 Hz, 2H), | | | | |
| | | | | | 3.78 (t, J = 5.6 Hz, 2H), 3.60- | | | | |
| | | | | | 3.39 (m, 1H), 2.90 (d, J = 13.6 | | | | |
| | | | | | Hz, 6H), 1.03 (d, J = 6.2 Hz, | | | | |
| | | | | | 6H). | | | | |
| 165 | trifluoroacetic acid [2- | 5.28 | 455 | | ¹H NMR (300 MHz, | 25.6 | 84% @ 1 h | 99% @ 1 h | 97% @ 1 h |
| | ({[(dimethylcarbamoyl)oxy]methyl}sulfanyl)- | | | | CHLOROFORM-d) δ = 7.51 (d, | | | | |
| | 4-oxo-1-[2-(propan-2- | | | | J = 3.3 Hz, 1H), 6.37 (s, 2H), | | | | |
| | yloxy)ethyl]-1H,4H,5H-pyrrolo[3,2- | | | | 6.29-6.22 (m, 1H), 5.95 (s, | | | | |
| | d]pyrimidin-5-yl]methyl N,N- | | | | 2H), 4.28 (t, J = 5.8 Hz, 2H), | | | | |
| | dimethylcarbamate | | | | 3.74 (t, J = 5.9 Hz, 2H), 3.60- | | | | |
| | | | | | 3.44 (m, 1H), 2.90 (t, J = 6.4 | | | | |
| | | | | | Hz, 11H), 1.06 (d, J = 6.1 Hz, | | | | |
| | | | | | 6H). | | | | |
| 166 | trifluoroacetic acid ({4-oxo-1-[2- | 5.45 | 383 | | ¹H NMR (300 MHz, | 70.8 | 99% @ 1 h | 99% @ 1 h | 100% @ 1 h |
| | (propan-2-yloxy)ethyl]-1H,4H,5H- | | | | CHLOROFORM-d) δ = 12.48 (s, | | | | |
| | pyrrolo[3,2-d]pyrimidin-2- | | | | 1H), 7.48-7.44 (m, 1H), 7.49- | | | | |
| | yl}sulfanyl)methyl N-methyl-N- | | | | 7.43 (m, 1H), 7.48 (t, J = 3.0 | | | | |
| | (propan-2-yl)carbamate | | | | Hz, 1H), 6.38 (t, J = 2.5 Hz, | | | | |
| | | | | | 1H), 6.15-5.89 (m, 2H), 4.41 | | | | |
| | | | | | (t, J = 5.5 Hz, 2H), 4.34-4.18 | | | | |
| | | | | | (m, 1H), 3.80 (t, J = 5.6 Hz, | | | | |
| | | | | | 2H), 3.57-3.44 (m, 1H), 1.09 | | | | |
| | | | | | (dd, J = 6.2, 11.7 Hz, 6H), | | | | |
| | | | | | 1.04 (d, J = 6.1 Hz, 12H) | | | | |
| 167 | trifluoroacetic acid ({4-oxo-1-[2- | 5.92 | 397 | | 1H NMR (300 MHz, | 178 | 99% @ 1 h | 99% @ 1 h | 98% @ 1 h |
| | (propan-2-yloxy)ethyl]-1H,4H,5H- | | | | CHLOROFORM-d) δ = 12.76- | | | | |
| | pyrrolo[3,2-d]pyrimidin-2- | | | | 12.39 (m, 1H), 7.60-7.41 | | | | |
| | yl}sulfanyl)methyl N-tert-butyl-N- | | | | (m, 1H), 6.35 (dd, J = 1.9, 3.0 | | | | |
| | methylcarbamate | | | | Hz, 1H), 5.99 (s, 2H), 4.39 (t, | | | | |
| | | | | | J = 5.7 Hz, 2H), 3.89-3.67 | | | | |
| | | | | | (m, 2H), 3.50 (t, J = 6.1 Hz, | | | | |
| | | | | | 1H), 2.89 (s, 3H), 1.37 (s, | | | | |
| | | | | | 9H), 1.04 (d, J = 6.0 Hz, 6H) | | | | |
| 168 | trifluoroacetic acid {2-[{{[tert- | 7 | 540 | | 1H NMR (300 MHz, | 281 | 100% @ 1 h | 94% @ 1 h | 88% @ 1 h |
| | butyl(methyl)carbamoyl]oxy}methyl)sulfanyl]- | | | | CHLOROFORM-d) δ = 7.48 (d, | | | | |
| | 4-oxo-1-[2-(propan-2- | | | | J = 3.3 Hz, 1H), 6.36 (s, 2H), | | | | |
| | yloxy)ethyl]-1H,4H,5H-pyrrolo[3,2- | | | | 6.24 (d, J = 3.3 Hz, 1H), 5.94 | | | | |
| | d]pyrimidin-5-yl}methyl N-tert-butyl- | | | | (s, 2H), 4.27 (t, J = 5.8 Hz, | | | | |
| | N-methylcarbamate | | | | 2H), 3.74 (br t, J = 5.6 Hz, | | | | |
| | | | | | 2H), 3.56-3.44 (m, 1H), | | | | |
| | | | | | 2.89 (s, 3H), 2.87 (s, 3H), | | | | |
| | | | | | 1.37 (s, 9H), 1.35-1.32 (m, | | | | |
| | | | | | 9H), 1.04 (d, J = 6.0 Hz, 6H) | | | | |
| 169 | trifluoroacetic acid {2- | 4.25 | 484 | | 1H NMR (300 MHz, | 109 | 99% @ 1 h | 81% @ 1 h | 89% @ 1 h |
| | [{{[ethyl(methyl)carba- | | | | CHLOROFORM-d) δ = 7.53- | | | | |
| | moyl]oxy}methyl)sulfanyl]- | | | | 7.43 (m, 1H), 6.38 (s, 2H), | | | | |
| | 4-oxo-1-[2-(propan-2- | | | | 6.24 (d, J = 3.2 Hz, 1H), 5.96 | | | | |
| | yloxy)ethyl]-1H,4H,5H-pyrrolo[3,2- | | | | (d, J = 2.3 Hz, 2H), 4.26 (t, | | | | |
| | d]pyrimidin-5-yl}methyl N-ethyl-N- | | | | J = 5.8 Hz, 2H), 3.73 (t, J = 5.8 | | | | |
| | methylcarbamate | | | | Hz, 2H), 3.57-3.42 (m, 1H), | | | | |
| | | | | | 3.39-3.17 (m, 4H), 2.86 (dd, | | | | |
| | | | | | J = 7.4, 11.5 Hz, 3H), 2.63 (t, | | | | |
| | | | | | J = 5.6 Hz, 3H), 1.38 (d, J = 6.6 | | | | |
| | | | | | Hz, 6H), 1.05 (d, J = 6.1 Hz, | | | | |
| | | | | | 6H) | | | | |
| 170 | trifluoroacetic acid {2- | 4.45 | 498 | | 1H NMR (300 MHz, | 141 | 99% @ 1 h | 92% @ 1 h | 99% @ 1 h |
| | [{{[methyl(propan-2- | | | | CHLOROFORM-d) δ = 7.51 (d, | | | | |
| | yl)carbamoyl]oxy}methyl)sulfanyl]-4- | | | | J = 3.2 Hz, 1H), 6.46-6.34 | | | | |
| | oxo-1-[2-(propan-2-yloxy)ethyl]- | | | | (m, 2H), 6.26 (d, J = 3.2 Hz, | | | | |
| | 1H,4H,5H-pyrrolo[3,2-d]pyrimidin-5- | | | | 1H), 6.06-5.94 (m, 2H), | | | | |
| | yl}methyl N-ethyl-N- | | | | 4.28 (t, J = 5.7 Hz, 2H), 3.79- | | | | |
| | methylcarbamate | | | | 3.69 (m, 2H), 3.59-3.43 (m, | | | | |
| | | | | | 1H), 3.38-3.20 (m, 3H), | | | | |
| | | | | | 2.97-2.66 (m, 6H), 1.18- | | | | |
| | | | | | 0.98 (m, 15H) | | | | |
| 171 | trifluoroacetic acid {2- | 4.65 | 512 | | 1H NMR (300 MHz, | 217 | 100% @ 1 h | 89% @ 1 h | 97% @ 1 h |
| | [{{[methyl(propan-2- | | | | CHLOROFORM-d) δ = 7.49 (d, | | | | |
| | yl)carbamoyl]oxy}methyl)sulfanyl]-4- | | | | J = 3.2 Hz, 1H), 6.40 (d, J = 6.3 | | | | |
| | oxo-1-[2-(propan-2-yloxy)ethyl]- | | | | Hz, 2H), 6.24 (d, J = 3.2 Hz, | | | | |
| | 1H,4H,5H-pyrrolo[3,2-d]pyrimidin-5- | | | | 1H), 6.03-5.93 (m, 2H), | | | | |
| | yl}methyl N-methyl-N-(propan-2- | | | | 4.40 (ddd, J = 6.9, 7.0, 13.6 | | | | |
| | yl)carbamate | | | | Hz, 2H), 4.27 (t, J = 5.8 Hz, | | | | |
| | | | | | 2H), 3.73 (t, J = 5.8 Hz, 2H), | | | | |
| | | | | | 3.57-3.44 (m, 1H), 2.82- | | | | |
| | | | | | 2.66 (m, 6H), 1.17-0.98 (m, | | | | |
| | | | | | 18H) | | | | |
| 172 | ({4-oxo-1-[2-(propan-2-yloxy)ethyl]- | 4.17 | 397 | | 1H NMR (300 MHz, | 237 | 100% @ 1 h | 95% @ 1 h | 98% @ 1 h |
| | 1H,4H,5H-pyrrolo[3,2-d]pyrimidin-2- | | | | CHLOROFORM-d) δ = 12.92 (s, | | | | |
| | yl}sulfanyl)methyl N-methyl-N-(2- | | | | 1H), 7.42 (t, J = 3.0 Hz, 1H), | | | | |
| | methylpropyl)carbamate | | | | 6.20 (dd, J = 1.9, 3.0 Hz, 1H), | | | | |
| | | | | | 6.05 (s, 2H), 4.34-4.24 (m, | | | | |
| | | | | | 2H), 3.79-3.65 (m, 2H), | | | | |
| | | | | | 3.56-3.41 (m, 1H), 3.05 (dd, | | | | |
| | | | | | J = 7.5, 17.5 Hz, 2H), 2.87 (d, | | | | |
| | | | | | J = 19.9 Hz, 3H), 1.96-1.72 | | | | |
| | | | | | (m, 1H), 1.04 (d, J = 6.1 Hz, | | | | |
| | | | | | 6H), 0.87 (d, J = 6.8 Hz, 3H), | | | | |
| | | | | | 0.77 (d, J = 6.7 Hz, 3H) | | | | |
| 173 | trifluoroacetic acid ({4-oxo-1-[2- | 4.23 | 431 | | 1H NMR (300 MHz, | 205 | 95% @ 1 h | 99% @ 1 h | 99% @ 1 h |
| | (propan-2-yloxy)ethyl]-1H,4H,5H- | | | | CHLOROFORM-d) δ = 12.68 | | | | |
| | pyrrolo[3,2-d]pyrimidin-2- | | | | (br d, J = 14.7 Hz, 1H), 8.21 | | | | |
| | yl}sulfanyl)methyl N-benzyl-N- | | | | (s, 1H), 7.50 (q, J = 3.4 Hz, | | | | |
| | methylcarbamate | | | | 1H), 7.38-7.19 (m, 2H), | | | | |
| | | | | | 7.17-6.99 (m, 2H), 6.33- | | | | |
| | | | | | 6.32 (m, 1H), 6.32 (q, J = 2.6 | | | | |
| | | | | | Hz, 1H), 6.10-6.00 (m, 2H), | | | | |
| | | | | | 4.53-4.27 (m, 4H), 3.89- | | | | |
| | | | | | 3.65 (m, 2H), 3.58-3.40 (m, | | | | |
| | | | | | 1H), 2.99-2.75 (m, 3H), | | | | |
| | | | | | 1.04 (dd, J = 1.8, 5.9 Hz, 6H) | | | | |
| 174 | trifluoroacetic acid {2- | 5.12 | 608 | | 1H NMR (300 MHz, | 304 | 99% @ 1 h | 99% @ 1 h | 99% @ 1 h |
| | [{{[benzyl(methyl)carba- | | | | CHLOROFORM-d) δ = 7.58- | | | | |
| | moyl]oxy}methyl)sulfanyl]- | | | | 7.41 (m, 1H), 7.37-7.00 (m, | | | | |
| | 4-oxo-1-[2-(propan- | | | | 10H), 6.48-6.38 (m, 2H), | | | | |
| | 2-yloxy)ethyl]-1H,4H,5H-pyrrolo[3,2- | | | | 6.33-6.18 (m, 1H), 6.07- | | | | |
| | d]pyrimidin-5-yl}methyl N-benzyl-N- | | | | 5.95 (m, 2H), 4.56-4.34 (m, | | | | |
| | methylcarbamate | | | | 4H), 4.33-4.15 (m, 2H), | | | | |
| | | | | | 3.82-3.62 (m, 2H), 3.58- | | | | |
| | | | | | 3.43 (m, 1H), 2.90 (dd, | | | | |
| | | | | | J = 1.9, 12.3 Hz, 3H), 2.85- | | | | |
| | | | | | 2.77 (m, 3H), 1.30-0.87 (m, | | | | |
| | | | | | 6H) | | | | |
| 175 | trifluoroacetic acid ({4-oxo-1-[2- | 4.45 | 445 | | 1H NMR (300 MHz, | 309 | 99% @ 1 h | 92% @ 1 h | 95% @ 1 h |
| | (propan-2-yloxy)ethyl]-1H,4H,5H- | | | | CHLOROFORM-d) δ = 12.78 | | | | |
| | pyrrolo[3,2-d]pyrimidin-2- | | | | (br d, J = 12.6 Hz, 1H), 7.49 (t, | | | | |
| | yl}sulfanyl)methyl N-methyl-N-(2- | | | | J = 3.0 Hz, 1H), 7.37-6.94 | | | | |
| | phenylethyl)carbamate | | | | (m, 5H), 6.29 (dd, J = 1.8, 3.0 | | | | |
| | | | | | Hz, 1H), 6.06-5.95 (m, 2H), | | | | |
| | | | | | 4.35 (q, J = 6.0 Hz, 2H), 3.86- | | | | |
| | | | | | 3.68 (m, 2H), 3.57-3.37 (m, | | | | |
| | | | | | 3H), 2.88-2.64 (m, J = 16.2 | | | | |
| | | | | | Hz, 5H), 1.08-1.00 (m, 6H) | | | | |
| 176 | trifluoroacetic acid {2-[{{[methyl(2- | 5.37 | 637 | | 1H NMR (300 MHz, | 404 | 86% @ 1 h | 100% @ 1 h | 99% @ 1 h |
| | phenylethyl)carbamoyl]oxy}methyl)sulfanyl]- | | | | CHLOROFORM-d) δ = 7.44 | | | | |
| | 4-oxo-1-[2-(propan-2- | | | | (dd, J = 3.2, 7.1 Hz, 1H), 7.34- | | | | |
| | yloxy)ethyl]-1H,4H,5H-pyrrolo[3,2- | | | | 6.98 (m, 10H), 6.38 (d, J = 4.8 | | | | |
| | d]pyrimidin-5-yl}methyl N-methyl-N- | | | | Hz, 1H), 6.32-6.21 (m, 2H), | | | | |
| | (2-phenylethyl)carbamate | | | | 6.00-5.87 (m, 2H), 4.34- | | | | |
| | | | | | 4.16 (m, 2H), 3.73 (q, J = 5.9 | | | | |
| | | | | | Hz, 2H), 3.60-3.32 (m, 5H), | | | | |
| | | | | | 2.92-2.59 (m, 10H), 1.08- | | | | |
| | | | | | 1.01 (m, 6H) | | | | |
| 177 | trifluoroacetic acid ({4-oxo-1-[2- | 3.85 | 381 | | 1H NMR (300 MHz, | 152 | 99% @ 1 h | 89% @ 1 h | 99% @ 1 h |
| | (propan-2-yloxy)ethyl]-1H,4H,5H- | | | | CHLOROFORM-d) δ = 12.68 (s, | | | | |
| | pyrrolo[3,2-d]pyrimidin-2- | | | | 1H), 7.47 (t, J = 3.0 Hz, 1H), | | | | |
| | yl}sulfanyl)methyl N-methyl-N- | | | | 6.28 (t, J = 2.4 Hz, 1H), 6.03 | | | | |
| | (prop-2-en-1-yl)carbamate | | | | (d, J = 2.7 Hz, 2H), 5.84-5.57 | | | | |
| | | | | | (m, 1H), 5.20-4.95 (m, 2H), | | | | |
| | | | | | 4.43-4.27 (m, 2H), 3.94- | | | | |
| | | | | | 3.69 (m, 4H), 3.56-3.42 (m, | | | | |
| | | | | | 1H), 2.86 (d, J = 19.6 Hz, 3H), | | | | |
| | | | | | 1.09-1.00 (m, 6H) | | | | |
| 178 | trifluoroacetic acid {2- | 4.5 | 508 | | 1H NMR (300 MHz, | 194 | 99% @ 1 h | 75% @ 1 h | 86% @ 1 h |
| | [{{[methyl(prop-2-en-1- | | | | CHLOROFORM-d) δ = 7.47 (t, | | | | |
| | yl)carbamoyl]oxy}methyl)sulfanyl]-4- | | | | J = 3.5 Hz, 1H), 6.39 (d, J = 3.6 | | | | |
| | oxo-1-[2-(propan-2-yloxy)ethyl]- | | | | Hz, 2H), 6.25 (br d, J = 3.2 Hz, | | | | |
| | 1H,4H,5H-pyrrolo[3,2-d]pyrimidin-5- | | | | 1H), 5.98 (s, 2H), 5.85-5.54 | | | | |
| | yl}methyl N-methyl-N-(prop-2-en-1- | | | | (m, 2H), 5.25-4.96 (m, 4H), | | | | |
| | yl)carbamate | | | | 4.26 (t, J = 5.8 Hz, 2H), 3.94- | | | | |
| | | | | | 3.77 (m, 4H), 3.73 (t, J = 5.5 | | | | |
| | | | | | Hz, 2H), 3.59-3.42 (m, 1H), | | | | |
| | | | | | 2.85 (dd, J = 6.9, 12.0 Hz, | | | | |
| | | | | | 6H), 1.05 (d, J = 6.1 Hz, 6H). | | | | |
| 179 | trifluoroacetic acid ({4-oxo-1-[2- | 4.03 | 395 | | 1H NMR (300 MHz, | 145 | 95% @ 1 h | 91% @ 1 h | 99% @ 1 h |
| | (propan-2-yloxy)ethyl]-1H,4H,5H- | | | | CHLOROFORM-d) δ = 12.61 | | | | |
| | pyrrolo[3,2-d]pyrimidin-2- | | | | (br s, 1H), 7.49 (t, J = 2.9 Hz, | | | | |
| | yl}sulfanyl)methyl N-(but-3-en-1-yl)- | | | | 1H), 6.33 (dd, J = 1.5, 2.9 Hz, | | | | |
| | N-methylcarbamate | | | | 1H), 6.02 (s, 2H), 5.86-5.57 | | | | |
| | | | | | (m, 1H), 5.20-4.88 (m, 2H), | | | | |
| | | | | | 4.45-4.31 (m, 2H), 3.78 (t, | | | | |
| | | | | | J = 5.7 Hz, 2H), 3.56-3.43 | | | | |
| | | | | | (m, 1H), 3.40-3.20 (m, 2H), | | | | |
| | | | | | 2.89 (d, J = 17.3 Hz, 3H), 2.38- | | | | |
| | | | | | 2.10 (m, 2H), 1.04 (d, J = 6.1 | | | | |
| | | | | | Hz, 6H) | | | | |
| 180 | trifluoroacetic acid {2-[{{[(but-3-en-1- | 4.77 | 536 | | 1H NMR (300 MHz, | 259 | 99% @ 1 h | 82% @ 1 h | 99% @ 1 h |
| | yl)(methyl)carbamoyl]oxy}methyl)sulfanyl]- | | | | CHLOROFORM-d) δ = 7.49 (br | | | | |
| | 4-oxo-1-[2-(propan-2- | | | | t, J = 4.0 Hz, 1H), 6.38 (s, 2H), | | | | |
| | yloxy)ethyl]-1H,4H,5H-pyrrolo[3,2- | | | | 6.26 (d, J = 3.2 Hz, 1H), 5.95 | | | | |
| | d]pyrimidin-5-yl}methyl N-(but-3-en- | | | | (s, 2H), 5.84-5.46 (m, 2H), | | | | |
| | 1-yl)-N-methylcarbamate | | | | 5.17-4.72 (m, 4H), 4.35- | | | | |
| | | | | | 4.18 (m, 2H), 3.73 (br t, | | | | |
| | | | | | J = 5.6 Hz, 2H), 3.58-3.41 | | | | |
| | | | | | (m, 1H), 3.30 (br t, J = 8.2 Hz, | | | | |
| | | | | | 4H), 2.93-2.79 (m, 6H), | | | | |
| | | | | | 2.37-2.07 (m, 4H), 1.12- | | | | |
| | | | | | 0.98 (m, 6H) | | | | |

### Example 181

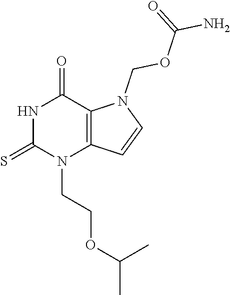 [SMILES: CC(C)OCCn1c(=S)[nH]c(=O)c2c1ccn2COC(N)=O]

5-Hydroxymethyl-1-[2-(propan-2-yloxy)ethyl]-2-sulfanylidene-1H,2H,3H,4H,5H-pyrrolo[3,2-d]pyrimidin-4-one: Dissolved 1-[2-(propan-2-yloxy)ethyl]-2-sulfanylidene-1H,2H,3H,4H,5H-pyrrolo[3,2-d]pyrimidin-4-one (2.0 g, 7.9 mmole) in DMF (10 mL) and warmed to 130° C. After heating for 10 minutes, added the formaldehyde 37% solution (1.9 mL, 24 mmole). Continued heating for 2 hours, then cooled to RT and concentrated in vacuo. Purified on a 120 g Silacel siica gel column, eluting with 0-4% MeOH in CH₂Cl₂ to yield 5-hydroxymethyl-1-[2-(propan-2-yloxy)ethyl]-2-sulfanylidene-1H,2H,3H,4H,5H-pyrrolo[3,2-d]pyrimidin-4-one: (2.1 g, 91%), ¹H NMR (300 MHz, CHLOROFORM-d) δ=9.75 (br s, 1H), 7.13 (d, J=3.0 Hz, 1H), 6.18 (d, J=2.9 Hz, 1H), 5.61-5.48 (m, 2H), 5.43-5.30 (m, 1H), 4.55 (t, J=5.7 Hz, 2H), 3.85 (t, J=5.6 Hz, 2H), 3.62-3.45 (m, 1H), 1.07 (d, J=6.0 Hz, 6H). LC/MS Method A: Rₜ=3.47 mins., (M+H)⁺=284, purity >99%.

Carbamic acid 1-(2-isopropoxy-ethyl)-4-oxo-2-thioxo-1,2,3,4-tetrahydro-pyrrolo[3,2-d]pyrimidin-5-ylmethyl ester: At 23° C., added trichloroacetylisocyanate (0.073 mL, 0.12 g, 0.62 mmole) dropwise to a solution of 5-hydroxymethyl-1-[2-(propan-2-yloxy)ethyl]-2-sulfanylidene-1H,2H,3H,4H,5H-pyrrolo[3,2-d]pyrimidin-4-one (0.07 g. 0.25 mmol) in CHCl₃ (3 mL). Let stir for 2 hours, then concentrated the reaction in vacuo and repeated by redissolving in CH₂Cl₂ and concentrating the reaction mixture down. Suspended the solid in 3 mL of the 10% H₂O/90% MeOH. Added Na₂CO₃, and let stir at 23° C. for 18 hours, then filtered, washing first with H₂O, then with diethyl ether to yield carbamic acid 1-(2-isopropoxy-ethyl)-4-oxo-2-thioxo-1,2,3,4-tetrahydro-pyrrolo[3,2-d]pyrimidin-5-ylmethyl ester: (56 mg 69%). ¹H NMR (300 MHz, DMSO-d₆) δ=12.33 (br s, 1H), 7.47 (d, J=3.1 Hz, 1H), 7.08-6.55 (m, 2H), 6.35 (d, J=3.1 Hz, 1H), 6.06 (s, 2H), 4.46 (br t, J=5.9 Hz, 2H), 3.69 (t, J=6.0 Hz, 2H), 3.59-3.48 (m, 1H), 0.99 (d, J=7.0 Hz, 6H). LC/MS Method A: Rₜ=3.57 mins., (M+H)⁺=327, purity >98%. Caco-2 Pₐₚₚ=218 nm/s, SGF=82% remaining at 1 h, SIF=97% remaining at 1 h, hPlasma stability=99% remaining at 1 h.

Additional compounds prepared similarly to EXAMPLE 181:

| | | LCMS | | | | Caco-2 | | | human |
|---|---|---|---|---|---|---|---|---|---|
| | | retention | | LCMS | | Pₐₚₚ | | | Plasma |
| Example | Name | time | M + H⁺ | method | NMR | (nm/s) | SGF | SIF | stability |
| 182 | ({4-oxo-1-[2-(propan-2-yloxy)ethyl]- | 4.28 | 369 | | 1H NMR (300 MHz, DMSO-d6) | | 81% @ 1 h | 69% @ 1 h | 94% @ 1 h |
| | 1H,4H,5H-pyrrolo[3,2-d]pyrimidin-2- | | | | δ ppm 12.34 (s, 1 H), 7.48 | | | | |
| | yl}sulfanyl)methyl N- | | | | (d, J = 2.93 Hz, 1 H), 7.44 (t, | | | | |
| | propylcarbamate | | | | J = 5.57 Hz, 1 H), 6.36 (d, | | | | |
| | | | | | J = 2.93 Hz, 1 H), 6.09 (s, 2 | | | | |
| | | | | | H), 4.46 (t, J = 5.86 Hz, 2 H), | | | | |
| | | | | | 3.70 (t, J = 5.86 Hz, 2 H), 3.49- | | | | |
| | | | | | 3.58 (m, 1 H), 2.84-2.97 | | | | |
| | | | | | (m, 2 H), 1.28-1.42 (m, 2 | | | | |
| | | | | | H), 0.98 (d, J = 6.45 Hz, 6 H), | | | | |
| | | | | | 0.68-0.84 (m, 3 H) | | | | |
| 183 | {4-oxo-1-[2-(propan-2-yloxy)ethyl]-2- | 4.32 | 484 | | 1H NMR (300 MHz, | 45.9 | 94% @ 1 h | 99% @ 1 h | 92% @ 1 h |
| | ({[(propylcarbamoyl)oxy]meth- | | | | CHLOROFORM-d) δ = 7.34 (d, | | | | |
| | yl}sulfanyl)- | | | | J = 3.2 Hz, 1H), 6.37 (s, 2H), | | | | |
| | 1H,4H,5H-pyrrolo[3,2- | | | | 6.16 (d, J = 3.2 Hz, 1H), 5.96 | | | | |
| | d]pyrimidin-5-yl}methyl N- | | | | (s, 2H), 4.92 (q, J = 5.9 Hz, | | | | |
| | propylcarbamate | | | | 2H), 4.21 (t, J = 5.9 Hz, 2H), | | | | |
| | | | | | 3.78-3.65 (m, 2H), 3.58- | | | | |
| | | | | | 3.43 (m, 1H), 3.23-3.04 (m, | | | | |
| | | | | | 4H), 1.60-1.38 (m, 4H), | | | | |
| | | | | | 1.06 (d, J = 6.1 Hz, 6H), 0.96- | | | | |
| | | | | | 0.78 (m, 6H) | | | | |
| 184 | ({4-oxo-1-[2-(propan-2-yloxy)ethyl]- | 4.77 | 454 | | 1H NMR (300 MHz, DMSO-d6) | | 99% @ 1 h | 86% @ 1 h | 95% @ 1 h |
| | 5-(propylcarbamoyl)-1H,4H,5H- | | | | δ ppm 12.18 (t, J = 4.98 Hz, 1 | | | | |
| | pyrrolo[3,2-d]pyrimidin-2- | | | | H), 8.07 (d, J = 3.52 Hz, 1 H), | | | | |
| | yl}sulfanyl)methyl N- | | | | 7.55 (t, J = 5.86 Hz, 1 H), 6.72 | | | | |
| | propylcarbamate | | | | (d, J = 3.52 Hz, 1 H), 5.78 (s, | | | | |
| | | | | | 2 H), 4.34 (t, J = 4.98 Hz, 2 | | | | |
| | | | | | H), 3.67 (t, J = 5.27 Hz, 2 H), | | | | |
| | | | | | 3.46 (t, J = 5.86 Hz, 1 H), 3.21- | | | | |
| | | | | | 3.30 (m, 2 H), 2.87-2.99 | | | | |
| | | | | | (m, 2 H), 1.50-1.65 (m, 2 | | | | |
| | | | | | H), 1.30-1.45 (m, 2 H), 0.89- | | | | |
| | | | | | 1.00 (m, 9 H), 0.80 (t, | | | | |
| | | | | | J = 7.33 Hz, 3 H) | | | | |
| 185 | ({4-oxo-1-[2-(propan-2-yloxy)ethyl]- | 5.28 | 482 | | 1H NMR (300 MHz, | | 78% @ 1 h | 67% @ 1 h | 96% @ 1 h |
| | 1H,4H,5H-pyrrolo[3,2-d]pyrimidin-2 | | | | CHLOROFORM-d) δ = 12.23- | | | | |
| | yl}sulfanyl)methyl N- | | | | 12.03 (m, 1H), 8.13 (d, J = 3.6 | | | | |
| | butylcarbamate | | | | Hz, 1H), 6.34 (d, J = 3.6 Hz, | | | | |
| | | | | | 1H), 5.95 (s, 2H), 4.93-4.70 | | | | |
| | | | | | (m, 1H), 4.27 (t, J = 5.6 Hz, | | | | |
| | | | | | 2H), 3.74 (t, J = 5.6 Hz, 2H), | | | | |
| | | | | | 3.57-3.34 (m, 3H), 3.27- | | | | |
| | | | | | 2.98 (m, 2H), 1.74-1.56 (m, | | | | |
| | | | | | 3H), 1.55-1.40 (m, 3H), | | | | |
| | | | | | 1.39-1.23 (m, 2H), 1.05 (d, | | | | |
| | | | | | J = 6.0 Hz, 6H), 0.93 (td, | | | | |
| | | | | | J = 7.6, 12.9 Hz, 6H) | | | | |
| 186 | ({4-oxo-1-[2-(propan-2-yloxy)ethyl]- | 4.52 | 383 | | %). 1H NMR (300 MHz, DMSO- | 72.3 | 86% @ 1 h | 100% @ 1 h | 98% @ 1 h |
| | 1H,4H,5H-pyrrolo[3,2-d]pyrimidin-2- | | | | d6) δ = 12.54-12.19 (m, | | | | |
| | yl}sulfanyl)methyl N-(2- | | | | 1H), 7.68-7.42 (m, 2H), | | | | |
| | methylpropyl)carbamate | | | | 6.36 (d, J = 3.3 Hz, 1H), 6.09 | | | | |
| | | | | | (s, 2H), 4.62-4.34 (m, 2H), | | | | |
| | | | | | 3.84-3.65 (m, 2H), 3.59- | | | | |
| | | | | | 3.47 (m, 1H), 2.76 (t, J = 6.4 | | | | |
| | | | | | Hz, 2H), 1.60 (ddd, J = 6.7, | | | | |
| | | | | | 6.8, 13.5 Hz, 1H), 0.98 (d, | | | | |
| | | | | | J = 6.1 Hz, 6H), 0.77 (d, J = 6.7 | | | | |
| | | | | | Hz, 6H) | | | | |
| 187 | ({5-[(2-methylpropyl)carbamoyl]-4- | 5.27 | 482 | | 1H NMR (300 MHz, DMSO-d6) δ = | 331 | 97% @ 1 h | 97% @ 1 h | 96% @ 1 h |
| | oxo-1-[2-(propan-2-yloxy)ethyl]- | | | | 12.20 (t, J = 5.2 Hz, 1H), | | | | |
| | 1H,4H,5H-pyrrolo[3,2-d]pyrimidin-2- | | | | 8.08 (d, J = 3.6 Hz, 1H), 7.59 | | | | |
| | yl}sulfanyl) methyl N-(2- | | | | (t, J = 5.9 Hz, 1H), 6.72 (d, | | | | |
| | methylpropyl)carbamate | | | | J = 3.7 Hz, 1H), 5.79 (s, 2H), | | | | |
| | | | | | 4.33 (t, J = 5.1 Hz, 2H), 3.67 | | | | |
| | | | | | (t, J = 5.2 Hz, 2H), 3.52-3.40 | | | | |
| | | | | | (m, 1H), 3.15 (t, J = 5.8 Hz, | | | | |
| | | | | | 2H), 2.79 (t, J = 6.4 Hz, 2H), | | | | |
| | | | | | 1.83 (ddd, J = 6.6, 6.7, 13.3 | | | | |
| | | | | | Hz, 1H), 1.63 (td, J = 6.7, 13.4 | | | | |
| | | | | | Hz, 1H), 1.00-0.89 (m, 12H), | | | | |
| | | | | | 0.79 (d, J = 6.7 Hz, 6H) | | | | |
| 188 | {2-[{{[(2- | 4.68 | 512 | | 1H NMR (300 MHz, | 117 | 98% @ 1 h | 97% @ 1 h | 99% @ 1 h |
| | methylpropyl)carbamoyl]oxy}meth- | | | | CHLOROFORM-d) δ = 7.33 (d, | | | | |
| | yl)sulfanyl]-4-oxo-1-[2-(propan-2- | | | | J = 3.2 Hz, 1H), 6.37 (s, 2H), | | | | |
| | yloxy)ethyl]-1H,4H,5H-pyrrolo[3,2- | | | | 6.15 (d, J = 3.3 Hz, 1H), 5.96 | | | | |
| | d]pyrimidin-5-yl}methyl N-(2- | | | | (s, 2H), 5.10-4.89 (m, 2H), | | | | |
| | methylpropyl)carbamate | | | | 4.20 (t, J = 5.9 Hz, 2H), 3.77- | | | | |
| | | | | | 3.65 (m, 2H), 3.59-3.41 (m, | | | | |
| | | | | | 1H), 3.12-2.88 (m, 4H), | | | | |
| | | | | | 1.87-1.59 (m, 2H), 1.05 (d, | | | | |
| | | | | | J = 6.2 Hz, 6H), 0.88 (dd, | | | | |
| | | | | | J = 6.7, 9.4 Hz, 12H) | | | | |
| 189 | {2-[{{[ethyl(propan-2- | 4.98 | 540 | | 1H NMR (300 MHz, | 306 | 99% @ 1 h | 97% @ 1 h | 98% @ 1 h |
| | yl)carbamoyl]oxy}methyl)sulfanyl]-4- | | | | CHLOROFORM-d) δ = 7.52 (br | | | | |
| | oxo-1-[2-(propan-2-yloxy)ethyl]- | | | | s, 2H), 6.39 (s, 2H), 6.27 (d, | | | | |
| | 1H,4H,5H-pyrrolo[3,2-d]pyrimidin-5- | | | | J = 3.2 Hz, 1H), 5.97 (s, 2H), | | | | |
| | yl}methyl N-ethyl-N-(propan-2- | | | | 4.29 (t, J = 5.6 Hz, 3H), 3.83- | | | | |
| | yl)carbamate | | | | 3.64 (m, 2H), 3.55-3.44 (m, | | | | |
| | | | | | 1H), 3.28-3.03 (m, 4H), | | | | |
| | | | | | 1.18-1.05 (m, 18H), 1.03 (d, | | | | |
| | | | | | J = 6.1 Hz, 6H) | | | | |
| 190 | ({4-oxo-1-[2-(propan-2-yloxy)ethyl]- | 4.1 | 397 | | 1H NMR (300 MHz, | 138 | 99% @ 1 h | 98% @ 1 h | 99% @ 1 h |
| | 1H,4H,5H-pyrrolo[3,2-d]pyrimidin-2- | | | | CHLOROFORM-d) δ = 12.69 | | | | |
| | yl}sulfanyl)methyl N-ethyl-N- | | | | (br s, 1H), 7.52 (t, J = 3.1 Hz, | | | | |
| | (propan-2-yl)carbamate | | | | 1H), 6.42-6.20 (m, 1H), | | | | |
| | | | | | 6.03 (s, 2H), 4.38 (t, J = 5.6 | | | | |
| | | | | | Hz, 2H), 4.33-4.00 (m, 1H), | | | | |
| | | | | | 3.77 (t, J = 5.6 Hz, 2H), 3.53- | | | | |
| | | | | | 3.39 (m, 1H), 3.28-2.98 (m, | | | | |
| | | | | | 2H), 1.16-1.04 (m, 9H), | | | | |
| | | | | | 1.00 (d, J = 6.2 Hz, 6H). | | | | |
| 191 | tert-butyl 4-oxo-1-[2-(propan-2- | 4.83 | 354 | | 1H NMR (300 MHz, | 10.8 | 15% @ 1 h | 82% @ 1 h | 16% @ 1 h |
| | yloxy)ethyl]-2-sulfanylidene- | | | | CHLOROFORM-d) δ = 9.56 (s, | | | | |
| | 1H,2H,3H,4H,5H-pyrrolo[3,2- | | | | 1H), 7.62 (d, J = 3.6 Hz, 1H), | | | | |
| | d]pyrimidine-5-carboxylate | | | | 6.38 (d, J = 3.6 Hz, 1H), 4.53 | | | | |
| | | | | | (t, J = 5.4 Hz, 2H), 3.83 (t, | | | | |
| | | | | | J = 5.4 Hz, 2H), 3.63-3.46 | | | | |
| | | | | | (m, 1H), 1.64 (s, 9H), 1.05 | | | | |
| | | | | | (d, J = 6.0 Hz, 6H) | | | | |
| 192 | ({4-oxo-1-[2-(propan-2-yloxy)ethyl]- | 4.58 | 417 | | 1H NMR (300 MHz, DMSO-d6) δ = | 38.4 | 100% remaining | 50% remaining | 97% remaining |
| | 1H,4H,5H-pyrrolo[3,2-d]pyrimidin-2- | | | | 12.36 (s, 1H), 8.02 (t, | | At 1 h | At 1 h | @ 1 h |
| | yl}sulfanyl)methyl N- | | | | J = 6.2 Hz, 1H), 7.49 (d, J = 3.1 | | | | |
| | benzylcarbamate | | | | Hz, 1H), 7.27 (br d, J = 7.2 Hz, | | | | |
| | | | | | 2H), 7.24-7.15 (m, 3H), | | | | |
| | | | | | 6.37 (d, J = 3.1 Hz, 1H), 6.12 | | | | |
| | | | | | (s, 2H), 4.56-4.39 (m, 2H), | | | | |
| | | | | | 4.15 (d, J = 6.1 Hz, 2H), 3.70 | | | | |
| | | | | | (t, J = 6.0 Hz, 2H), 3.59-3.44 | | | | |
| | | | | | (m, 1H), 0.99 (d, J = 6.1 Hz, | | | | |
| | | | | | 6H) | | | | |
| 193 | {[5-(benzylcarbamoyl)-4-oxo-1-[2- | 5.28 | 550 | | . 1H NMR (300 MHz, DMSO-d6) | 316 | 98% remaining | 57% remaining | 95% remaining |
| | (propan-2-yloxy)ethyl]-1H,4H,5H- | | | | δ = 12.69 (t, J = 5.4 Hz, 1H), | | At 1 h | At 1 h | @ 1 h |
| | pyrrolo[3,2-d]pyrimidin-2- | | | | 8.25-7.93 (m, 2H), 7.40- | | | | |
| | yl]sulfanyl}methyl N- | | | | 7.16 (m, 10H), 6.76 (d, J = 3.7 | | | | |
| | benzylcarbamate | | | | Hz, 1H), 5.80 (s, 2H), 4.53 (d, | | | | |
| | | | | | J = 5.3 Hz, 2H), 4.35 (t, J = 5.3 | | | | |
| | | | | | Hz, 2H), 4.18 (d, J = 6.1 Hz, | | | | |
| | | | | | 2H), 3.67 (t, J = 5.2 Hz, 2H), | | | | |
| | | | | | 3.53-3.39 (m, 1H), 0.93 (d, | | | | |
| | | | | | J = 6.0 Hz, 6H). LC/MS: Rt = | | | | |
| | | | | | 5.28 mins., (M + H)+ = 550, | | | | |
| | | | | | purity >95%. | | | | |
| 194 | {[5-(butylcarbamoyl)-4-oxo-1-[2- | 5.28 | 482 | | 1H NMR (300 MHz, | 429 | 99% remaining | 59% remaining | 97% remaining |
| | (propan-2-yloxy)ethyl]-1H,4H,5H- | | | | CHLOROFORM-d) δ = 12.23- | | At 1 h | At 1 h | @ 1 h |
| | pyrrolo[3,2-d]pyrimidin-2- | | | | 12.03 (m, 1H), 8.13 (d, J = 3.6 | | | | |
| | yl]sulfanyl}methyl N- | | | | Hz, 1H), 6.34 (d, J = 3.6 Hz, | | | | |
| | butylcarbamate | | | | 1H), 5.95 (s, 2H), 4.93-4.70 | | | | |
| | | | | | (m, 1H), 4.27 (t, J = 5.6 Hz, | | | | |
| | | | | | 2H), 3.74 (t, J = 5.6 Hz, 2H), | | | | |
| | | | | | 3.57-3.34 (m, 3H), 3.27- | | | | |
| | | | | | 2.98 (m, 2H), 1.74-1.56 (m, | | | | |
| | | | | | 3H), 1.55-1.40 (m, 3H), | | | | |
| | | | | | 1.39-1.23 (m, 2H), 1.05 (d, | | | | |
| | | | | | J = 6.0 Hz, 6H), 0.93 (td, | | | | |
| | | | | | J = 7.6, 12.9 Hz, 6H) | | | | |
| 195 | ({4-oxo-5-[(propan-2-yl)carbamoyl]- | 4.7 | 454 | | 1H NMR (300 MHz, | 300 | 100% remaining | 99% remaining | 97% remaining |
| | 1-[2-(propan-2-yloxy)ethyl]- | | | | CHLOROFORM-d) δ = 12.13 | | At 1 h | At 1 h | @ 1 h |
| | 1H,4H,5H-pyrrolo[3,2-d]pyrimidin-2- | | | | (br d, J = 6.6 Hz, 1H), 8.13 (d, | | | | |
| | yl}sulfanyl)methyl N-(propan-2- | | | | J = 3.7 Hz, 1H), 6.34 (d, J = 3.6 | | | | |
| | yl)carbamate | | | | Hz, 1H), 5.92 (s, 2H), 4.72 (br | | | | |
| | | | | | d, J = 8.0 Hz, 1H), 4.28 (t, | | | | |
| | | | | | J = 5.6 Hz, 2H), 4.13-3.94 | | | | |
| | | | | | (m, 1H), 3.89-3.78 (m, 1H), | | | | |
| | | | | | 3.74 (t, J = 5.5 Hz, 2H), 3.55- | | | | |
| | | | | | 3.42 (m, 1H), 1.33 (d, J = 6.6 | | | | |
| | | | | | Hz, 6H), 1.15 (d, J = 6.5 Hz, | | | | |
| | | | | | 6H), 1.05 (d, J = 6.1 Hz, 6H) | | | | |
| 196 | ({4-oxo-1-[2-(propan-2-yloxy)ethyl]- | 4.28 | 369 | | 1H NMR (300 MHz, DMSO-d6) δ = | 195 | 99% remaining | 98% remaining | 99% remaining |
| | 1H,4H,5H-pyrrolo[3,2-d]pyrimidin-2- | | | | 12.34 (s, 1H), 7.48 (d, | | At 1 h | At 1 h | @ 1 h |
| | yl}sulfanyl)methyl N-(propan-2- | | | | J = 3.1 Hz, 1H), 7.37 (d, J = 7.7 | | | | |
| | yl)carbamate | | | | Hz, 1H), 6.36 (d, J = 3.1 Hz, | | | | |
| | | | | | 1H), 6.08 (s, 2H), 4.46 (t, | | | | |
| | | | | | J = 6.0 Hz, 2H), 3.70 (t, J = 6.0 | | | | |
| | | | | | Hz, 2H), 3.62-3.42 (m, 2H), | | | | |
| | | | | | 0.99 (dd, J = 4.2, 6.3 Hz, 12H) | | | | |
| 197 | 5-(hydroxymethyl)-1-[2-(propan-2- | 3.47 | 284 | | 1H NMR (300 MHz, | unstable | 98% remaining | 55% remaining | 86% remaining |
| | yloxy)ethyl]-2-sulfanylidene- | | | | CHLOROFORM-d) δ = 10.63- | | At 1 h | At 1 h | @ 1 h |
| | 1H,2H,3H,4H,5H-pyrrolo[3,2- | | | | 9.52 (bs, 1H), 7.13 (d, J = 3.0 | | | | |
| | d]pyrimidin-4-one | | | | Hz, 1H), 6.18 (d, J = 2.9 Hz, | | | | |
| | | | | | 1H), 5.54 (s, 2H), 4.55 (t, | | | | |
| | | | | | J = 5.7 Hz, 2H), 3.85 (t, J = 5.6 | | | | |
| | | | | | Hz, 2H), 3.64-3.49 (m, 1H), | | | | |
| | | | | | 1.07 (d, J = 6.5 Hz, 6H) | | | | |
| 198 | [2-({[(methoxycarbonyl)oxy]meth- | 4.08 | 430 | | 1H NMR (300 MHz, | 114 | 79% remaining | 74% remaining | 0% remaining |
| | yl}sulfanyl)- | | | | CHLOROFORM-d) δ = 7.39 (d, | | At 1 h | At 1 h | @ 1 h |
| | 4-oxo-1-[2-(propan-2- | | | | J = 3.3 Hz, 1H), 6.41 (s, 2H), | | | | |
| | yloxy)ethyl]-1H,4H, 5H-pyrrolo[3,2- | | | | 6.30 (d, J = 3.3 Hz, 1H), 5.98 | | | | |
| | d]pyrimidin-5-yl]methyl methyl | | | | (s, 2H), 4.36-4.20 (m, 2H), | | | | |
| | carbonate | | | | 3.80 (d, J = 6.1 Hz, 6H), 3.74 | | | | |
| | | | | | (t, J = 5.6 Hz, 2H), 3.63-3.39 | | | | |
| | | | | | (m, 1H), 1.17-0.94 (m, 6H) | | | | |
| 199 | methyl ({4-oxo-5-[{{4-oxo-1-[2- | 4.58 | 607 | | 1H NMR (300 MHz, | 55.4 | 47% remaining | 79% remaining | 46% remaining |
| | (propan-2-yloxy)ethyl]-1H,4H,5H- | | | | CHLOROFORM-d) δ = 9.18 (s, | | At 1 h | At 1 h | @ 1 h |
| | pyrrolo[3,2-d]pyrimidin-2- | | | | 1H), 7.91-7.75 (m, 1H), | | | | |
| | yl}sulfanyl)methyl]-1-[2-(propan-2- | | | | 7.47-7.32 (m, 1H), 6.44 (s, | | | | |
| | yloxy)ethyl]-1H,4H,5H-pyrrolo[3,2- | | | | 2H), 6.28 (d, J = 3.3 Hz, 1H), | | | | |
| | d]pyrimidin-2-yl}sulfanyl)methyl | | | | 6.22 (s, 2H), 6.13-6.08 (m, | | | | |
| | carbonate | | | | 1H), 4.50 (t, J = 5.9 Hz, 2H), | | | | |
| | | | | | 4.20 (t, J = 5.5 Hz, 2H), 3.86- | | | | |
| | | | | | 3.76 (m, 5H), 3.69 (t, J = 5.5 | | | | |
| | | | | | Hz, 2H), 3.63-3.52 (m, 1H), | | | | |
| | | | | | 3.52-3.42 (m, 1H), 1.10- | | | | |
| | | | | | 1.05 (m, 6H), 1.05-0.99 (m, | | | | |
| | | | | | 6H) | | | | |
| 200 | methyl ({4-oxo-1-[2-(propan-2- | 4.25 | 342 | | 1H NMR (300 MHz, | 97.6 | 100% remaining | 75% remaining | 2% remaining |
| | yloxy)ethyl]-1H,4H,5H-pyrrolo[3,2- | | | | CHLOROFORM-d) δ = 9.32 (s, | | At 1 h | At 1 h | At 1 h |
| | d]pyrimidin-2-yl}sulfanyl)methyl | | | | 1H), 7.30 (d, J = 3.1 Hz, 1H), | | | | |
| | carbonate | | | | 6.26 (s, 3H), 4.53 (t, J = 5.6 | | | | |
| | | | | | Hz, 2H), 3.84 (t, J = 5.6 Hz, | | | | |
| | | | | | 2H), 3.80 (s, 3H), 3.63-3.48 | | | | |
| | | | | | (m, 1H), 1.07 (d, J = 6.1 Hz, | | | | |
| | | | | | 6H) | | | | |
| 201 | ethyl ({4-oxo-1-[2-(propan-2- | 4.55 | 356 | | 1H NMR (300 MHz, | | 100% remaining | 43% remaining | 0% remaining |
| | yloxy)ethyl]-1H,4H,5H-pyrrolo[3,2- | | | | CHLOROFORM-d) δ ppm 9.19 | | At 1 h | At 1 h | At 1 h |
| | d]pyrimidin-2-yl}sulfanyl)methyl | | | | (br d, J = 1.17 Hz, 1 H) 7.30 | | | | |
| | carbonate | | | | (d, J = 2.93 Hz, 1 H) 6.20- | | | | |
| | | | | | 6.34 (m, 3 H) 4.53 (t, J = 5.57 | | | | |
| | | | | | Hz, 2 H) 4.22 (q, J = 7.03 Hz, 2 | | | | |
| | | | | | H) 3.84 (t, J = 5.57 Hz, 2 H) | | | | |
| | | | | | 3.45-3.62 (m, 1 H) 1.19- | | | | |
| | | | | | 1.38 (t, J = 5.9 Hz, 3 H) 1.07 | | | | |
| | | | | | (d, J = 5.86 Hz, 6 H) | | | | |
| 202 | ethyl ({4-oxo-5-[{{4-oxo-1-[2- | 4.82 | 621 | | 1H NMR (300 MHz, | | 45% remaining | 52% remaining | 3% remaining |
| | (propan-2-yloxy)ethyl]-1H,4H,5H- | | | | CHLOROFORM-d) δ ppm 9.16 | | At 1 h | At 1 h | At 1 h |
| | pyrrolo[3,2-d]pyrimidin-2- | | | | (s, 1 H) 7.82 (d, J = 2.93 Hz, 1 | | | | |
| | yl}sulfanyl)methyl]-1-[2-(propan-2- | | | | H) 7.34 (d, J = 2.93 Hz, 1 H) | | | | |
| | yloxy)ethyl]-1H,4H,5H-pyrrolo[3,2- | | | | 6.44 (s, 2 H) 6.27 (d, J = 2.93 | | | | |
| | d]pyrimidin-2-yl}sulfanyl)methyl | | | | Hz, 1 H) 6.22 (s, 2 H) 6.10 (d, | | | | |
| | carbonate | | | | J = 3.52 Hz, 1 H) 4.50 (t, | | | | |
| | | | | | J = 5.86 Hz, 2 H) 4.12-4.31 | | | | |
| | | | | | (m, 4 H) 3.81 (t, J = 5.86 Hz, 2 | | | | |
| | | | | | H) 3.69 (t, J = 5.57 Hz, 2 H) | | | | |
| | | | | | 3.40-3.61 (m, 2 H) 1.29 (t, | | | | |
| | | | | | J = 7.03 Hz, 3 H) 1.07 (d, | | | | |
| | | | | | J = 5.86 Hz, 6 H) 1.02 (d, | | | | |
| | | | | | J = 5.86 Hz, 6 H) | | | | |
| 203 | ({4-oxo-1-[2-(propan-2-yloxy)ethyl]- | 4.82 | 370 | | 1H NMR (300 MHz, | 134 | 99% remaining | 63% remaining | 1% remaining |
| | 1H,4H,5H-pyrrolo[3,2-d]pyrimidin-2- | | | | CHLOROFORM-d) δ = 9.15 (s, | | At 1 h | At 1 h | At 1 h |
| | yl}sulfanyl)methyl propan-2-yl | | | | 1H), 7.30 (d, J = 3.1 Hz, 1H), | | | | |
| | carbonate | | | | 6.27 (d, J = 3.2 Hz, 1H), 6.25 | | | | |
| | | | | | (s, 2H), 4.95-4.82 (m, 1H), | | | | |
| | | | | | 4.53 (t, J = 5.6 Hz, 2H), 3.84 | | | | |
| | | | | | (t, J = 5.6 Hz, 2H), 3.63-3.48 | | | | |
| | | | | | (m, 1H), 1.28 (d, J = 6.3 Hz, | | | | |
| | | | | | 6H), 1.07 (d, J = 6.1 Hz, 6H) | | | | |
| 204 | butyl ({4-oxo-1-[2-(propan-2- | 5.15 | 384 | | 1H NMR (300 MHz, | 63.5 | 85 | 2 | 1% remaining |
| | yloxy)ethyl]-1H,4H,5H-pyrrolo[3,2- | | | | CHLOROFORM-d) δ = 9.44 (s, | | | | at 1 h |
| | d]pyrimidin-2-yl}sulfanyl)methyl | | | | 1H), 7.30 (d, J = 3.2 Hz, 1H), | | | | |
| | carbonate | | | | 6.26 (s, 3H), 4.53 (t, J = 5.6 | | | | |
| | | | | | Hz, 2H), 4.15 (t, J = 6.6 Hz, | | | | |
| | | | | | 2H), 3.84 (t, J = 5.6 Hz, 2H), | | | | |
| | | | | | 3.65-3.43 (m, 1H), 1.65- | | | | |
| | | | | | 1.56 (m, 2H), 1.43-1.28 (m, | | | | |
| | | | | | 2H), 1.07 (d, J = 5.9 Hz, 6H), | | | | |
| | | | | | 0.95-0.86 (m, 3H) | | | | |
| 205 | ({4-oxo-1-[2-(propan-2-yloxy)ethyl]- | 5.07 | 404 | | 1H NMR (300 MHz, | | 61 | 10 | 2% remaining |
| | 1H,4H,5H-pyrrolo[3,2-d]pyrimidin-2- | | | | CHLOROFORM-d) δ = 9.23 (s, | | | | at 1 h |
| | yl}sulfanyl)methyl phenyl | | | | 1H), 7.43-7.34 (m, 2H), | | | | |
| | carbonate | | | | 7.35-7.34 (m, 1H), 7.34- | | | | |
| | | | | | 7.27 (m, 1H), 7.24-7.12 (m, | | | | |
| | | | | | 2H), 6.37 (s, 2H), 6.30 (br d, | | | | |
| | | | | | J = 3.2 Hz, 1H), 4.54 (s, 2H), | | | | |
| | | | | | 3.86 (t, J = 5.5 Hz, 2H), 3.66- | | | | |
| | | | | | 3.51 (m, 1H), 1.08 (d, J = 6.2 | | | | |
| | | | | | Hz, 6H). | | | | |
| 206 | octyl ({4-oxo-1-[2-(propan-2- | 6.37 | 440 | | 1H NMR (300 MHz, | 1.38 | 91 | 1 | 99% remaining |
| | yloxy)ethyl]-1H,4H,5H-pyrrolo[3,2- | | | | CHLOROFORM-d) δ = 9.39 (s, | | | | at 1 h |
| | d]pyrimidin-2-yl}sulfanyl)methyl | | | | 1H), 7.30 (d, J = 3.2 Hz, 1H), | | | | |
| | carbonate | | | | 6.26 (s, 3H), 4.53 (s, 2H), | | | | |
| | | | | | 4.14 (t, J = 6.7 Hz, 2H), 3.84 | | | | |
| | | | | | (t, J = 5.6 Hz, 2H), 3.67-3.45 | | | | |
| | | | | | (m, 1H), 1.70-1.57 (m, 2H), | | | | |
| | | | | | 1.40-1.18 (m, 10H), 1.07 (d, | | | | |
| | | | | | J = 6.0 Hz, 6H), 0.86 (t, J = 7.0 | | | | |
| | | | | | Hz, 3H) | | | | |
| 207 | octyl {2- | 7.47 | 626 | | 1H NMR (300 MHz, | 2.67 | 90 | 29 | 92% remaining |
| | [{{[(octyloxy)carbon- | | | | CHLOROFORM-d) δ = 7.30 (d, | | | | at 1 h |
| | yl]oxy}methyl)sulfanyl]- | | | | J = 3.2 Hz, 1H), 6.43 (s, 2H), | | | | |
| | 4-oxo-1-[2-(propan-2- | | | | 6.23 (d, J = 3.3 Hz, 1H), 6.03 | | | | |
| | yloxy)ethyl]-1H,4H,5H-pyrrolo[3,2- | | | | (s, 2H), 4.25 (t, J = 5.8 Hz, | | | | |
| | d]pyrimidin-5-yl} methyl carbonate | | | | 2H), 4.18-4.08 (m, 4H), | | | | |
| | | | | | 3.76-3.68 (m, 2H), 3.57- | | | | |
| | | | | | 3.44 (m, 1H), 1.72-1.56 (m, | | | | |
| | | | | | 4H), 1.42-1.19 (m, 20H), | | | | |
| | | | | | 1.06 (d, J = 6.0 Hz, 6H), 0.86 | | | | |
| | | | | | (t, J = 6.8 Hz, 6H) | | | | |

### Example 208

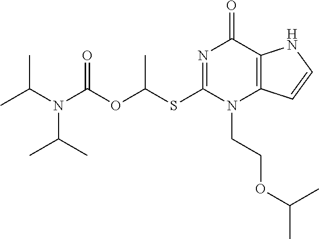 [SMILES: CC(C)OCCn1c(SC(C)OC(=O)N(C(C)C)C(C)C)nc(=O)c2[nH]ccc21]

1-Chloroethyl N,N-bis(propan-2-yl)carbamate: Dissolved diisopropyl amine (0.12 g, 0.18 mL, 2.1 mmole) in 2 mL CH₂Cl₂, then added diisopropyl ethyl amine (0.73 mL, 2.5 mmole). Cooled in an ice bath, then added dropwise, over 1 min, 1-chloro-ethyl chloroformate (0.23 mL, 2.1 mmole). After 2 hours, partitioned between CH₂Cl₂ and H₂O. Separated layers and washed the org. layer with brine. Dried over H₂O filtered and concentrated in vacuo to yield 1-chloroethyl N,N-bis(propan-2-yl)carbamate: ¹H NMR (300 MHz, CHLOROFORM-d) δ=6.78-6.49 (m, 1H), 4.10 (s, 1H), 3.74 (s, 1H), 1.82 (d, J=5.7 Hz, 3H), 1.22 (d, J=6.9 Hz, 12H). Took on as is. (0.35 g, 100%).

1-({4-Oxo-1-[2-(propan-2-yloxy)ethyl]-1H,4H,5H-pyrrolo[3,2-d]pyrimidin-2-yl}sulfanyl)ethyl N,N-bis(propan-2-yl)carbamate: Added sodium hydride (60% dispersion in mineral oil (20 mg, 0.829 mmole) to a solution of 1-[2-(propan-2-yloxy)ethyl]-2-sulfanylidene-1H,2H,3H,4H,5H-pyrrolo[3,2-d]pyrimidin-4-one (0.10 g, 0.39 mmole) in THF (3 mL). After gas evolution had ceased, added 1-chloroethyl N,N-bis(propan-2-yl)carbamate (0.35 g, 2.1 mmole) in 2 mL THF. After 18 hours, concentrated the reaction in vacuo, then purified on a 40 g Agela silica gel column, eluting with 0-5% MeOH in CH₂Cl₂. Concentrated desired fractions in vacuo. Repeated purification using same conditions. To yield 1-({4-oxo-1-[2-(propan-2-yloxy)ethyl]-1H,4H,5H-pyrrolo[3,2-cl]pyrimidin-2-yl}sulfanyl)ethyl N,N-bis(propan-2-yl)carbamate: (55.4 mg, 33%). ¹H NMR (300 MHz, CHLOROFORM-d) δ=12.46 (s, 1H), 7.49-7.43 (m, 1H), 6.89 (d, J=6.5 Hz, 1H), 6.29-6.23 (m, 1H), 4.50-4.20 (m, 2H), 4.06 (s, 1H), 3.77 (t, J=5.9 Hz, 2H), 3.72-3.59 (m, 1H), 3.58-3.41 (m, 1H), 1.90 (d, J=6.5 Hz, 3H), 1.18 (dd, J=3.6, 6.8 Hz, 12H), 1.05 (dd, J=2.0, 6.1 Hz, 6H). LC/MS Method A: Rₜ=4.52 mins., (M+H)⁺=425, purity 95%. Caco-2 Pₐₚₚ=183 nm/s, SGF=1% remaining at 1 h, SIF=93% remaining at 1 h, hPlasma stability=98% remaining at 1 h.

Additional compounds prepared similarly to EXAMPLE 208:

| | | LCMS | | | | Caco-2 | | | human |
|---|---|---|---|---|---|---|---|---|---|
| | | retention | | LCMS | | Pₐₚₚ | | | Plasma |
| Example | Name | time | M + H⁺ | method | NMR | (nm/s) | SGF | SIF | stability |
| 209 | ({4-oxo-1-[2-(propan-2-yloxy)ethyl]- | 4.18 | 417 | | 1H NMR (300 MHz, DMSO-d6) δ = | 161 | 99% remaining | 99% remaining | 97% remaining |
| | 1H,4H,5H-pyrrolo[3,2-d]pyrimidin-2- | | | | 12.03 (s, 1H), 7.40-7.00 | | at 1 h | at 1 h | at 1 h |
| | yl}sulfanyl)methyl N-methyl-N- | | | | (m, 6H), 6.35 (s, 1H), 5.81 | | | | |
| | phenylcarbamate | | | | (d, J = 2.2 Hz, 2H), 4.23 (q, | | | | |
| | | | | | J = 5.7 Hz, 2H), 3.76-3.56 | | | | |
| | | | | | (m, 2H), 3.51-3.36 (m, 1H), | | | | |
| | | | | | 3.30 (s, 3H), 0.93 (dd, J = 2.1, | | | | |
| | | | | | 6.0 Hz, 6H) | | | | |
| 210 | ({4-oxo-1-[2-(propan-2-yloxy)ethyl]- | 4.3 | 435 | | 1H NMR (300 MHz, DMSO-d6) δ = | 183 | 100% remaining | 97% remaining | 97% remaining |
| | 1H,4H,5H-pyrrolo[3,2-d]pyrimidin-2- | | | | 12.03 (br s, 1H), 7.34- | | at 1 h | at 1 h | at 1 h |
| | yl}sulfanyl)methyl N-(3- | | | | 7.24 (m, 2H), 7.20-7.07 (m, | | | | |
| | fluorophenyl)-N-methylcarbamate | | | | 2H), 7.05-6.93 (m, 1H), | | | | |
| | | | | | 6.35 (d, J = 2.9 Hz, 1H), 5.84 | | | | |
| | | | | | (s, 2H), 4.24 (t, J = 5.5 Hz, | | | | |
| | | | | | 2H), 3.65 (t, J = 5.4 Hz, 2H), | | | | |
| | | | | | 3.52-3.38 (m, 1H), 3.20 (s, | | | | |
| | | | | | 3H), 0.98-0.88 (m, 6H) | | | | |
| 211 | ({4-oxo-1-[2-(propan-2-yloxy)ethyl]- | 4.22 | 435 | | 1H NMR (300 MHz, DMSO-d6) δ = | 200 | 100% remaining | 96% remaining | 99% remaining |
| | 1H,4H,5H-pyrrolo[3,2-d]pyrimidin-2- | | | | 12.08 (s, 1H), 7.30 (t, | | at 1 h | at 1 h | at 1 h |
| | yl}sulfanyl)methyl N-(4- | | | | J = 3.0 Hz, 2H), 7.26 (br d, | | | | |
| | fluorophenyl)-N-methylcarbamate | | | | J = 6.3 Hz, 1H), 7.14-7.00 | | | | |
| | | | | | (m, 2H), 6.39-6.34 (m, 1H), | | | | |
| | | | | | 5.81 (s, 2H), 4.25 (t, J = 5.4 | | | | |
| | | | | | Hz, 2H), 3.65 (t, J = 5.4 Hz, | | | | |
| | | | | | 2H), 3.52-3.39 (m, 1H), | | | | |
| | | | | | 3.17 (s, 3H), 0.93 (d, J = 6.1 | | | | |
| | | | | | Hz, 6H) | | | | |

### Example 212

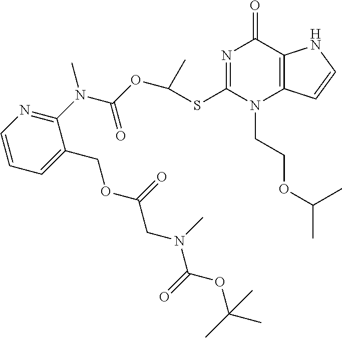 [SMILES: CC(C)OCCn1c(SC(C)OC(=O)N(C)c2ncccc2COC(=O)CN(C)C(=O)OC(C)(C)C)nc(=O)c2[nH]ccc21]

Step 1-4 (2-{[(1-chloroethoxy)carbonyl](methyl)amino}pyridin-3-yl)methyl 2-{[(tert-butoxy) carbonyl]-(methyl)amino}acetate was synthesized by following the procedure in J. Ohwada et al. Bioorg. Med. Chem. Lett. 13 (2003) 191-196.

(tert-Butoxycarbonyl-methyl-amino)-acetic acid 2-({1-[1-(2-isopropoxy-ethyl)-4-oxo-4,5-dihydro-1H-pyrrolo[3,2-d]pyrimidin-2-ylsulfanyl]-ethoxycarbonyl}-methyl-amino)-pyridin-3-ylmethyl ester: Added sodium hydride, 60% dispersion in mineral oil (20 mg, 0.83 mmole) to a solution of 1-[2-(propan-2-yloxy)ethyl]-2-sulfanylidene-1H,2H,3H,4H,5H-pyrrolo[3,2-d]pyrimidin-4-one (0.10 g, 0.39 mmole) in THF (3 mL). After gas evolution had ceased, added (2{[(1chloroethoxy)carbonyl](methyl)-amino}pyridin-3-yl)methyl 2-{[(tert-butoxy)carbonyl](methyl)amino}acetate (0.18 g, 0.434 mmole) in 2 mL THF. Monitored by LCMS and TLC. Let go for 18 hours while warming to 40° C. Added sodium hydride (60% dispersion in mineral oil (5 mg, 0.21 mmole) and warmed to 50° C. for 4 hours. Cooled to RT and Concentrated in vacuo. Took up in CH₂Cl₂ and purified on the ISCO using a 20 g column eluting with 0-10% MeOH in CH₂Cl₂. Concentrated desired fractions in vacuo to yield (tert-Butoxycarbonyl-methyl-amino)-acetic acid 2-({1-[1-(2-isopropoxy-ethyl)-4-oxo-4,5-dihydro-1H-pyrrolo[3,2-d]pyrimidin-2-ylsulfanyl]-ethoxycarbonyl}-methyl-amino)-pyridin-3-ylmethyl ester: (43 mg, 17%). ¹H NMR (300 MHz, CHLOROFORM-d) δ=12.80 (br d, J=13.3 Hz, 1H), 8.37 (br s, 1H), 7.73 (br d, J=7.7 Hz, 1H), 7.37 (s, 1H), 7.24-7.13 (m, 1H), 7.10-6.77 (m, 1H), 6.60-6.60 (m, 1H), 6.16 (t, J=2.4 Hz, 1H), 5.11 (s, 2H), 4.46-4.10 (m, 2H), 3.97-3.79 (m, 2H), 3.72 (br t, J=5.7 Hz, 2H), 3.56-3.45 (m, 1H), 3.36-3.23 (m, 3H), 2.83 (s, 3H), 1.97-1.66 (m, 3H), 1.44-1.27 (m, 9H), 1.04 (dd, J=3.5, 6.1 Hz, 6H). LC/MS Method A: Rₜ=4.47 mins., (M+H)⁺=633.78. Caco-2 Pₐₚₚ=8.7 nm/s, SGF=89% remaining at 1 h, SIF=97% remaining at 1 h, hPlasma stability=97% remaining at 1 h.

Additional compounds prepared similarly to EXAMPLE 212:

| | | LCMS | | | | Caco-2 | | | human |
|---|---|---|---|---|---|---|---|---|---|
| | | retention | | LCMS | | Pₐₚₚ | | | Plasma |
| Example | Name | time | M + H⁺ | method | NMR | (nm/s) | SGF | SIF | stability |
| 213 | hexyl {4-oxo-1-[2-(propan-2- | 5.82 | 412 | | 1H NMR (300 MHz, DMSO-d6) | 30.3 | 76% remaining | 1% remaining | 44% remaining |
| | yloxy)ethyl]-2-sulfanyl-1H,4H,5H- | | | | δ ppm 12.26-12.61 (m, 1 | | at 1 h | at 1 h | at 1 h |
| | pyrrolo[3,2-d]pyrimidin-5-yl}methyl | | | | H), 7.52 (d, J = 3.52 Hz, 1 H), | | | | |
| | carbonate | | | | 6.39 (d, J = 3.52 Hz, 1 H), | | | | |
| | | | | | 6.16 (s, 2 H), 4.45 (t, J = 5.86 | | | | |
| | | | | | Hz, 2 H), 4.06 (t, J = 6.74 Hz, | | | | |
| | | | | | 2 H), 3.69 (t, J = 5.86 Hz, 2 | | | | |
| | | | | | H), 3.51 (dt, J = 12.16, 5.93 | | | | |
| | | | | | Hz, 1 H), 1.45-1.62 (m, 2 | | | | |
| | | | | | H), 1.15-1.33 (m, 6 H), 0.97 | | | | |
| | | | | | (d, J = 6.45 Hz, 6 H), 0.76- | | | | |
| | | | | | 0.86 (m, 3 H) | | | | |
| 214 | hexyl {2- | 6.55 | 570 | | 1H NMR (300 MHz, DMSO-d6) | 16.3 | 80% remaining | 2% remaining | 77% remaining |
| | [{{[(hexyloxy)carbon- | | | | δ = 7.49 (d, J = 3.3 Hz, 1H), | | at 1 h | at 1 h | at 1 h |
| | yl]oxy}methyl)sulfanyl]- | | | | 6.50 (d, J = 3.2 Hz, 1H), 6.28 | | | | |
| | 4-oxo-1-[2-(propan-2- | | | | (s, 2H), 5.83 (s, 2H), 4.25 (t, | | | | |
| | yloxy)ethyl]-1H,4H,5H-pyrrolo[3,2- | | | | J = 5.3 Hz, 2H), 4.13-4.01 | | | | |
| | d]pyrimidin-5-yl}methyl carbonate | | | | (m, 4H), 3.67-3.61 (m, 2H), | | | | |
| | | | | | 3.50-3.38 (m, 1H), 1.62- | | | | |
| | | | | | 1.44 (m, 4H), 1.30-1.15 (m, | | | | |
| | | | | | 12H), 0.93 (d, J = 6.2 Hz, 6H), | | | | |
| | | | | | 0.81 (s, 6H) | | | | |
| 215 | hexyl [3- | 7.22 | 570 | | 1H NMR (300 MHz, DMSO-d6) δ = | 2.14 | 94% remaining | 1% remaining | 88% remaining |
| | ({[(hexyloxy)carbonyl]oxy}methyl)-4- | | | | 7.65 (d, J = 3.2 Hz, 1H), | | at 1 h | at 1 h | at 1 h |
| | oxo-1-[2-(propan-2-yloxy)ethyl]-2- | | | | 6.47 (d, J = 3.2 Hz, 1H), 6.38 | | | | |
| | sulfanylidene-1H,2H,3H,4H,5H- | | | | (s, 2H), 6.18 (s, 2H), 4.56 (t, | | | | |
| | pyrrolo [3,2-d]pyrimidin-5-yl]methyl | | | | J = 5.8 Hz, 2H), 4.07 (q, J = 6.4 | | | | |
| | carbonate | | | | Hz, 4H), 3.73 (t, J = 5.8 Hz, | | | | |
| | | | | | 2H), 3.59-3.46 (m, 1H), | | | | |
| | | | | | 1.64-1.44 (m, 4H), 1.34- | | | | |
| | | | | | 1.14 (m, 12H), 0.97 (d, J = 6.1 | | | | |
| | | | | | Hz, 6H), 0.87-0.76 (m, 6H) | | | | |
| 216 | {4-oxo-1-[2-(propan-2-yloxy)ethyl]- | 5.48 | 398 | | 1H NMR (300 MHz, DMSO-d6) δ = | 45.9 | 91% remaining | 1% remaining | 1% remaining |
| | 2-sulfanyl-1H,4H,5H-pyrrolo[3,2- | | | | 12.39 (s, 1H), 7.52 (d, | | at 1 h | at 1 h | at 1 h |
| | d]pyrimidin-5-yl}methyl pentyl | | | | J = 3.1 Hz, 1H), 6.39 (d, J = 3.2 | | | | |
| | carbonate | | | | Hz, 1H), 6.15 (s, 2H), 4.45 (t, | | | | |
| | | | | | J = 6.0 Hz, 2H), 4.05 (t, J = 6.6 | | | | |
| | | | | | Hz, 2H), 3.68 (t, J = 6.0 Hz, | | | | |
| | | | | | 2H), 3.58-3.44 (m, 1H), | | | | |
| | | | | | 1.60-1.44 (m, 2H), 1.29- | | | | |
| | | | | | 1.14 (m, 4H), 0.96 (d, J = 6.0 | | | | |
| | | | | | Hz, 6H), 0.85-0.76 (m, 3H) | | | | |
| 217 | {4-oxo-2- | 6.13 | 542 | | 1H NMR (300 MHz, DMSO-d6) δ = | 53.2 | 88% remaining | 1% remaining | 5% remaining |
| | [{{[(pentyloxy)carbonyl]oxy}methyl) | | | | 7.65 (d, J = 3.1 Hz, 1H), | | at 1 h | at 1 h | at 1 h |
| | sulfanyl]-1-[2-(propan-2- | | | | 6.47 (d, J = 3.2 Hz, 1H), 6.37 | | | | |
| | yloxy)ethyl]-1H,4H,5H-pyrrolo[3,2- | | | | (s, 2H), 6.18 (s, 1H), 4.56 (br | | | | |
| | d]pyrimidin-5-yl}methyl pentyl | | | | t, J = 5.2 Hz, 2H), 4.07 (q, | | | | |
| | carbonate | | | | J = 6.0 Hz, 3H), 3.72 (br t, | | | | |
| | | | | | J = 5.5 Hz, 2H), 3.62-3.38 | | | | |
| | | | | | (m, 3H), 1.68-1.43 (m, 4H), | | | | |
| | | | | | 1.33-1.12 (m, 8H), 1.02- | | | | |
| | | | | | 0.90 (m, 6H), 0.82 (q, J = 5.9 | | | | |
| | | | | | Hz, 6H) | | | | |
| 218 | [4-oxo-3- | 6.8 | 542 | | 1H NMR (300 MHz, DMSO-d6) δ = | 6.5 | 100% remaining | 2% remaining | 74% remaining |
| | ({[(pentyloxy)carbonyl]oxy}methyl)- | | | | 7.49 (d, J = 3.2 Hz, 1H), | | at 1 h | at 1 h | at 1 h |
| | 1-[2-(propan-2-yloxy)ethyl]-2- | | | | 6.50 (d, J = 3.3 Hz, 1H), 6.28 | | | | |
| | sulfanylidene-1H,2H,3H,4H,5H- | | | | (s, 2H), 5.83 (s, 2H), 4.25 (t, | | | | |
| | pyrrolo [3,2-d]pyrimidin-5-yl]methyl | | | | J = 5.3 Hz, 2H), 4.13-3.98 | | | | |
| | pentyl carbonate | | | | (m, 4H), 3.64 (t, J = 5.3 Hz, | | | | |
| | | | | | 2H), 3.52-3.35 (m, 1H), | | | | |
| | | | | | 1.62-1.42 (m, 4H), 1.32- | | | | |
| | | | | | 1.10 (m, 8H), 0.93 (d, J = 6.1 | | | | |
| | | | | | Hz, 6H), 0.86-0.73 (m, 6H) | | | | |
| 219 | heptyl {4-oxo-1-[2-(propan-2- | 6.07 | 426 | | 1H NMR (300 MHz, DMSO-d6) δ = | 8.47 | 98% remaining | 23% remaining | 21% remaining |
| | yloxy)ethyl]-2-sulfanyl-1H,4H,5H- | | | | 12.40 (s, 1H), 7.52 (d, | | at 1 h | at 1 h | at 1 h |
| | pyrrolo[3,2-d]pyrimidin-5-yl}methyl | | | | J = 3.1 Hz, 1H), 6.39 (d, J = 3.1 | | | | |
| | carbonate | | | | Hz, 1H), 6.15 (s, 2H), 4.58- | | | | |
| | | | | | 4.29 (m, 2H), 4.06 (t, J = 6.5 | | | | |
| | | | | | Hz, 2H), 3.69 (t, J = 5.9 Hz, | | | | |
| | | | | | 2H), 3.58-3.44 (m, 1H), | | | | |
| | | | | | 1.53 (br s, 2H), 1.20 (br d, | | | | |
| | | | | | J = 4.7 Hz, 8H), 0.97 (d, J = 6.2 | | | | |
| | | | | | Hz, 6H), 0.83 (br dd, J = 9.4, | | | | |
| | | | | | 12.2 Hz, 3H) | | | | |
| 220 | heptyl {2- | 6.92 | 598 | | 1H NMR (300 MHz, DMSO-d6) δ = | | 77% remaining | 5% remaining | 55% remaining |
| | [{{[(heptyloxy)carbonyl]oxy}methyl) | | | | 7.49 (d, J = 3.1 Hz, 1H), | | at 1 h | at 1 h | at 1 h |
| | sulfanyl]-4-oxo-1-[2-(propan-2- | | | | 6.50 (d, J = 3.2 Hz, 1H), 6.28 | | | | |
| | yloxy)ethyl]-1H,4H,5H-pyrrolo[3,2- | | | | (s, 2H), 5.83 (s, 2H), 4.25 (t, | | | | |
| | d]pyrimidin-5-yl}methyl carbonate | | | | J = 5.3 Hz, 2H), 4.13-4.00 | | | | |
| | | | | | (m, 4H), 3.64 (t, J = 5.3 Hz, | | | | |
| | | | | | 2H), 3.50-3.41 (m, 1H), | | | | |
| | | | | | 1.54 (q, J = 6.3 Hz, 4H), 1.30- | | | | |
| | | | | | 1.13 (m, 16H), 0.93 (d, J = 6.0 | | | | |
| | | | | | Hz, 6H), 0.80 (q, J = 5.7 Hz, | | | | |
| | | | | | 6H) | | | | |
| 221 | (2-{methyl[(1-{4-oxo-1-[2-(propan-2 | 4.33 | 503 | | 1H NMR (300 MHz, DMSO-d6) | 25.4 | 67% remaining | 68% remaining | 94% remaining |
| | yloxy)ethyl]-2-sulfanyl-1H,4H,5H- | | | | δ ppm 12.03 (br s, 1 H) 7.19- | | at 1 h | at 1 h | at 1 h |
| | pyrrolo[3,2-d]pyrimidin-5- | | | | 7.49 (m, 4 H) 6.96-7.17 (m, | | | | |
| | yl}ethoxy)carbonyl]amino}phenyl)methyl | | | | 1 H) 6.49-6.67 (m, 1 H) | | | | |
| | acetate | | | | 6.33 (d, J = 2.34 Hz, 1 H) 4.84- | | | | |
| | | | | | 5.06 (m, 2 H) 3.99-4.35 | | | | |
| | | | | | (m, 2 H) 3.36-3.75 (m, 3 H) | | | | |
| | | | | | 3.08 (d, J = 8.21 Hz, 3 H) 2.01 | | | | |
| | | | | | (s, 2 H) 1.92-1.96 (m, 1 H) | | | | |
| | | | | | 1.68-1.82 (m, 1 H) 1.49- | | | | |
| | | | | | 1.63 (m, 2 H) 0.86-1.02 (m, | | | | |
| | | | | | 6 H) | | | | |
| 222 | (2-{methyl[{{4-oxo-1-[2-(propan-2- | 4.07 | 489 | | 1H NMR (300 MHz, DMSO-d6) | 20.5 | 98% remaining | 88% remaining | 22% remaining |
| | yloxy)ethyl]-2-sulfanyl-1H,4H,5H- | | | | δ ppm 11.77-12.39 (m, 1 H) | | at 1 h | at 1 h | at 1 h |
| | pyrrolo[3,2-d]pyrimidin-5- | | | | 6.80-7.79 (m, 5 H) 6.34 (br | | | | |
| | yl}methoxy)carbonyl]amino}phenyl) | | | | d, J = 2.93 Hz, 1 H) 5.71 (d, | | | | |
| | methyl acetate | | | | J = 5.86 Hz, 2 H) 4.56-5.13 | | | | |
| | | | | | (m, 2 H) 4.03-4.42 (m, 2 H) | | | | |
| | | | | | 3.54-3.75 (m, 2 H) 3.46 (dt, | | | | |
| | | | | | J = 11.87, 6.08 Hz, 1 H) 3.24- | | | | |
| | | | | | 3.40 (m, 32 H) 3.01-3.17 | | | | |
| | | | | | (m, 3 H) 1.83-2.00 (m, 3 H) | | | | |
| | | | | | 0.86-1.02 (m, 6 H) | | | | |
| 223 | 4-oxo-N-(propan-2-yl)-1-[2-(propan- | 5 | 339 | | 1H NMR (300 MHz, DMSO-d6) | 370 | 87% remaining | 70% remaining | 99% remaining |
| | 2-yloxy)ethyl]-2-sulfanylidene- | | | | δ ppm 12.93 (s, 1 H) 10.67- | | at 1 h | at 1 h | at 1 h |
| | 1H,2H,3H,4H,5H-pyrrolo[3,2- | | | | 11.03 (m, 1 H) 8.05 (d, | | | | |
| | d]pyrimidine-5-carboxamide | | | | J = 3.52 Hz, 1 H) 6.59 (d, | | | | |
| | | | | | J = 3.52 Hz, 1 H) 4.51 (t, | | | | |
| | | | | | J = 5.86 Hz, 2 H) 3.78-3.98 | | | | |
| | | | | | (m, 1 H) 3.70 (t, J = 5.57 Hz, 2 | | | | |
| | | | | | H) 3.51 (quin, J = 6.01 Hz, 1 | | | | |
| | | | | | H) 1.19 (d, J = 6.45 Hz, 6 H) | | | | |
| | | | | | 0.96 (d, J = 5.86 Hz, 6 H) | | | | |
| 224 | N-butyl-4-oxo-1-[2-(propan-2- | 5.37 | 353 | | 1H NMR (300 MHz, DMSO-d6) | 126 | 83% remaining | 100% remaining | 89% remaining |
| | yloxy)ethyl]-2-sulfanylidene- | | | | δ ppm 12.94 (s, 1 H) 10.87 | | at 1 h | at 1 h | at 1 h |
| | 1H,2H,3H,4H,5H-pyrrolo[3,2- | | | | (t, J = 5.28 Hz, 1 H) 8.05 (d, | | | | |
| | d]pyrimidine-5-carboxamide | | | | J = 3.52 Hz, 1 H) 6.59 (d, | | | | |
| | | | | | J = 3.52 Hz, 1 H) 4.51 (t, | | | | |
| | | | | | J = 5.86 Hz, 2 H) 3.70 (t, | | | | |
| | | | | | J = 5.86 Hz, 2 H) 3.51 (quin, | | | | |
| | | | | | J = 6.01 Hz, 1 H) 3.24-3.31 | | | | |
| | | | | | (m, 2 H) 1.43-1.57 (m, 2 H) | | | | |
| | | | | | 1.27-1.42 (m, 2 H) 0.96 (d, | | | | |
| | | | | | J = 5.86 Hz, 6 H) 0.82-0.92 | | | | |
| | | | | | (m, 3 H) | | | | |
| 225 | 4-oxo-1-[2-(propan-2-yloxy)ethyl]-N- | 5 | 339 | | 1H NMR (300 MHz, DMSO-d6) | 243 | 98% remaining | 78% remaining | 99% remaining |
| | propyl-2-sulfanylidene- | | | | δ ppm 12.94 (s, 1 H) 10.88 | | at 1 h | at 1 h | at 1 h |
| | 1H,2H,3H,4H,5H-pyrrolo[3,2- | | | | (t, J = 4.98 Hz, 1 H) 8.06 (d, | | | | |
| | d]pyrimidine-5-carboxamide | | | | J = 3.52 Hz, 1 H) 6.59 (d, | | | | |
| | | | | | J = 4.10 Hz, 1 H) 4.51 (t, | | | | |
| | | | | | J = 5.86 Hz, 2 H) 3.65-3.76 | | | | |
| | | | | | (m, 2 H) 3.51 (quin, J = 6.15 | | | | |
| | | | | | Hz, 1 H) 3.24 (td, J = 6.74, | | | | |
| | | | | | 5.27 Hz, 2 H) 1.54 (q, J = 7.03 | | | | |
| | | | | | Hz, 2 H) 0.96 (d, J = 5.86 Hz, 6 | | | | |
| | | | | | H) 0.88-0.94 (m, 3 H) | | | | |
| 226 | N-(butan-2-yl)-4-oxo-1-[2-(propan- | 5.25 | 353 | | 1H NMR (300 MHz, DMSO-d6) | 351 | 91% remaining | 83% remaining | 97% remaining |
| | 2-yloxy)ethyl]-2-sulfanylidene- | | | | δ ppm 12.93 (s, 1 H) 10.86 | | at 1 h | at 1 h | at 1 h |
| | 1H,2H,3H,4H,5H-pyrrolo[3,2- | | | | (br d, J = 7.03 Hz, 1 H) 8.05 | | | | |
| | d]pyrimidine-5-carboxamide | | | | (d, J = 3.52 Hz, 1 H) 6.59 (d, | | | | |
| | | | | | J = 3.52 Hz, 1 H) 4.51 (t, | | | | |
| | | | | | J = 5.86 Hz, 2 H) 3.70 (t, | | | | |
| | | | | | J = 5.86 Hz, 3 H) 3.51 (quin, | | | | |
| | | | | | J = 6.01 Hz, 1 H) 1.52 (t, | | | | |
| | | | | | J = 7.33 Hz, 2 H) 1.15 (d, | | | | |
| | | | | | J = 6.45 Hz, 3 H) 0.94-1.02 | | | | |
| | | | | | (m, 6 H) 0.88 (t, J = 7.33 Hz, 3 | | | | |
| | | | | | H) | | | | |
| 227 | 4-oxo-N-phenyl-1-[2-(propan-2- | 5.62 | 373 | | 1H NMR (300 MHz, DMSO-d6) | 16.7 | 100% remaining | 95% remaining | 99% remaining |
| | yloxy)ethyl]-2-sulfanylidene- | | | | δ ppm 13.08-13.32 (m, 1 H) | | at 1 h | at 1 h | at 1 h |
| | 1H,2H,3H,4H,5H-pyrrolo[3,2- | | | | 8.18 (d, J = 3.52 Hz, 1 H) 7.53- | | | | |
| | d]pyrimidine-5-carboxamide | | | | 7.62 (m, 2 H) 7.33-7.43 | | | | |
| | | | | | (m, 2 H) 7.22-7.29 (m, 1 H) | | | | |
| | | | | | 7.09-7.20 (m, 1 H) 6.69 (d, | | | | |
| | | | | | J = 3.52 Hz, 1 H) 4.55 (t, | | | | |
| | | | | | J = 5.86 Hz, 2 H) 3.73 (t, | | | | |
| | | | | | J = 5.86 Hz, 2 H) 3.53 (quin, | | | | |
| | | | | | J = 6.15 Hz, 1 H) 0.98 (d, | | | | |
| | | | | | J = 6.45 Hz, 6 H) | | | | |
| 228 | N-benzyl-4-oxo-1-[2-(propan-2- | 5.38 | 387 | | 1H NMR (300 MHz, DMSO-d6) | 103 | 95% remaining | 100% remaining | 99% remaining |
| | yloxy)ethyl]-2-sulfanylidene- | | | | δ ppm 12.95 (s, 1 H) 11.36 | | at 1 h | at 1 h | at 1 h |
| | 1H,2H,3H,4H,5H-pyrrolo[3,2- | | | | (br s, 1 H) 8.09 (d, J = 3.52 | | | | |
| | d]pyrimidine-5-carboxamide | | | | Hz, 1 H) 7.07-7.47 (m, 5 H) | | | | |
| | | | | | 6.62 (d, J = 3.52 Hz, 1 H) 4.51 | | | | |
| | | | | | (d, J = 5.86 Hz, 4 H) 3.65- | | | | |
| | | | | | 3.75 (m, 2 H) 3.44-3.57 (m, | | | | |
| | | | | | 1 H) 0.96 (d, J = 5.86 Hz, 6 H) | | | | |

### Example 229

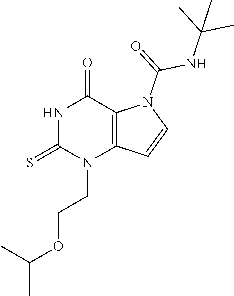 [SMILES: CC(C)OCCn1c(=S)[nH]c(=O)c2c1ccn2C(=O)NC(C)(C)C]

N-tert-butyl-4-oxo-1-[2-(propan-2-yloxy)ethyl]-2-sulfanylidene-1H,2H,3H,4H,5H-pyrrolo[3,2-d]pyrimidine-5-carboxamide: Added sodium hydride 60% dispersion in mineral oil (12 mg, 0.49 mmole) to a solution of 1-[2-(propan-2-yloxy)ethyl]-2-sulfanylidene-1H,2H,3H,4H,5H-pyrrolo[3,2-d]pyrimidin-4-one (0.050 g, 0.39 mmole) in DMF (3 mL). After gas evolution had ceased, added t-butyl isocyanate (0.024 mL, 0.22 mmole). After 2 hours, partitioned reactions between ethyl acetate and H₂O. Washed the organic layer with H₂O, then washed the combined organic layers with brine. Dried over Na₂SO₄, filtered and concentrated in vacuo. Purified on the ISCO, eluting with 0-70%, ethyl acetate in hexanes. Repurified on the RP HPLC Gilson, method B, eluting with 30-95% instead of 5 to 95%. Lyophilized desired fractions to N-tert-butyl-4-oxo-1-[2-(propan-2-yloxy)ethyl]-2-sulfanylidene-1H,2H,3H,4H,5H-pyrrolo[3,2-d]pyrimidine-5-carboxamide: (13.5 mg, 19.4%). ¹H NMR (300 MHz, CHLOROFORM-d) δ ppm 9.01-9.49 (m, 1H) 7.68 (br s, 1H) 6.44 (br s, 1H) 4.25-4.65 (m, 3H) 3.86 (br d, J=4.69 Hz, 2H) 3.53 (quin, J=6.15 Hz, 1H) 1.36-1.51 (m, 6H) 1.05 (d, J=5.86 Hz, 9H). LC/MS Method A: Rₜ=5.43 mins., (M+H)⁺=353. Caco-2 Pₐₚₚ=264 nm/s, SGF=98% remaining at 1 h, SIF=76% remaining at 1 h, hPlasma stability=96% remaining at 1 h.

Additional compounds prepared similarly to EXAMPLE 229:

| | | LCMS | | | | Caco-2 | | | human |
|---|---|---|---|---|---|---|---|---|---|
| | | retention | | LCMS | | Pₐₚₚ | | | Plasma |
| Example | Name | time | M + H⁺ | method | NMR | (nm/s) | SGF | SIF | stability |
| 230 | ethyl 4-oxo-1-[2-(propan-2- | 4.32 | 326 | | 1H NMR (300 MHz, | 4.56 | 99% remaining | 33% remaining | 2% remaining |
| | yloxy)ethyl]-2-sulfanylidene- | | | | CHLOROFORM-d) δ ppm 9.01- | | at 1 h | at 1 h | at 1 h |
| | 1H,2H,3H,4H,5H-pyrrolo[3,2- | | | | 9.49 (m, 1 H) 7.68 (br s, 1 H) | | | | |
| | d]pyrimidine-5-carboxylate | | | | 6.44 (br s, 1 H) 4.25-4.65 | | | | |
| | | | | | (m, 4 H) 3.86 (br d, J = 4.69 | | | | |
| | | | | | Hz, 2 H) 3.53 (quin, J = 6.15 | | | | |
| | | | | | Hz, 1 H) 1.36-1.51 (m, 3 H) | | | | |
| | | | | | 1.05 (d, J = 5.86 Hz, 6 H) | | | | |
| 231 | propan-2-yl 4-oxo-1-[2-(propan-2- | 4.65 | 340 | | 1H NMR (300 MHz, | 36.3 | 100% remaining | 40% remaining | 0% remaining |
| | yloxy)ethyl]-2-sulfanylidene- | | | | CHLOROFORM-d) δ ppm 9.25 | | at 1 h | at 1 h | at 1 h |
| | 1H,2H,3H,4H,5H-pyrrolo[3,2- | | | | (br d, J = 2.93 Hz, 1 H) 7.66 | | | | |
| | d]pyrimidine-5-carboxylate | | | | (br s, 1 H) 6.42 (br s, 1 H) | | | | |
| | | | | | 5.24 (quin, J = 6.30 Hz, 1 H) | | | | |
| | | | | | 4.54 (br s, 2 H) 3.85 (br s, 2 | | | | |
| | | | | | H) 3.54 (quin, J = 6.15 Hz, 1 | | | | |
| | | | | | H) 1.44 (d, J = 6.45 Hz, 6 H) | | | | |
| | | | | | 0.88-1.20 (m, 6 H). | | | | |
| 232 | 2-methylpropyl 4-oxo-1-[2-(propan- | 5.05 | 354 | | 1H NMR (300 MHz, | 2.56 | 98% remaining | 3% remaining | 2% remaining |
| | 2-yloxy)ethyl]-2-sulfanylidene- | | | | CHLOROFORM-d) δ ppm 9.27 | | at 1 h | at 1 h | at 1 h |
| | 1H,2H,3H,4H,5H-pyrrolo[3,2- | | | | (br s, 1 H) 7.68 (br d, J = 2.93 | | | | |
| | d]pyrimidine-5-carboxylate | | | | Hz, 1 H) 6.44 (br d, J = 1.76 | | | | |
| | | | | | Hz, 1 H) 4.54 (br s, 2 H) 4.22 | | | | |
| | | | | | (d, J = 6.45 Hz, 2 H) 3.85 (t, | | | | |
| | | | | | J = 5.27 Hz, 2 H) 3.54 (quin, | | | | |
| | | | | | J = 6.01 Hz, 1 H) 2.14 (dt, | | | | |
| | | | | | J = 13.33, 6.52 Hz, 1 H) 1.03- | | | | |
| | | | | | 1.07 (m, 6 H) 1.02 (d, J = 6.45 | | | | |
| | | | | | Hz, 6 H | | | | |
| 233 | methyl 4-oxo-1-[2-(propan-2- | 3.95 | 312 | | 1H NMR (300 MHz, | 3.14 | 98% remaining | 70% remaining | 2% remaining |
| | yloxy)ethyl]-2-sulfanylidene- | | | | CHLOROFORM-d) δ ppm 9.02- | | at 1 h | at 1 h | at 1 h |
| | 1H,2H,3H,4H,5H-pyrrolo[3,2- | | | | 9.54 (m, 1 H) 7.68 (br s, 1 H) | | | | |
| | d]pyrimidine-5-carboxylate | | | | 6.45 (br s, 1 H) 4.55 (br s, 2 | | | | |
| | | | | | H) 4.06 (s, 3 H) 3.85 (br s, 2 | | | | |
| | | | | | H) 3.53 (dt, J = 12.16, 5.93 | | | | |
| | | | | | Hz, 1 H) 1.04 (d, J = 6.45 Hz, 6 | | | | |
| | | | | | H) | | | | |
| 234 | 3-fluorophenyl {4-oxo-1-[2-(propan- | 5.12 | 422 | | 1H NMR (300 MHz, | <3.30 | 34% remaining | 8% remaining | 2% remaining |
| | 2-yloxy)ethyl]-2-sulfanylidene- | | | | CHLOROFORM-d) δ ppm 9.27 | | at 1 h | at 1 h | at 1 h |
| | 1H,2H,3H,4H,5H-pyrrolo[3,2- | | | | (br s, 1 H) 7.28-7.41 (m, 3 | | | | |
| | d]pyrimidin-5-yl}methyl carbonate | | | | H) 6.87-7.03 (m, 2 H) 6.36 | | | | |
| | | | | | (s, 2 H) 6.31 (br s, 1 H) 4.54 | | | | |
| | | | | | (br s, 2 H) 3.85 (br t, J = 5.27 | | | | |
| | | | | | Hz, 2 H) 3.56 (dt, J = 12.02, | | | | |
| | | | | | 6.30 Hz, 1 H) 0.94-1.19 (m, | | | | |
| | | | | | 6 H) | | | | |
| 235 | 4-fluorophenyl {4-oxo-1-[2-(propan- | 5.1 | 422 | | 1H NMR (300 MHz, | <3.69 | 60% remaining | 1% remaining at | 2% remaining |
| | 2-yloxy)ethyl]-2-sulfanylidene- | | | | CHLOROFORM-d) δ ppm 9.25 | | at 1 h | 1 h | at 1 h |
| | 1H,2H,3H,4H,5H-pyrrolo[3,2- | | | | (br s, 1 H) 7.30 (br s, 1 H) | | | | |
| | d]pyrimidin-5-yl}methyl carbonate | | | | 6.99-7.18 (m, 4 H) 6.35 (s, | | | | |
| | | | | | 2 H) 6.30 (br s, 1 H) 4.54 (br | | | | |
| | | | | | s, 2 H) 3.86 (br d, J = 4.69 Hz, | | | | |
| | | | | | 2 H) 3.56 (dt, J = 12.16, 5.93 | | | | |
| | | | | | Hz, 1 H) 1.07 (d, J = 5.86 Hz, 6 | | | | |
| | | | | | H) | | | | |
| 236 | 4-methylphenyl {4-oxo-1-[2- | 5.3 | 418 | | 1H NMR (300 MHz, | 5.9 | 53% remaining | 1% remaining at | 1% remaining |
| | (propan-2-yloxy)ethyl]-2- | | | | CHLOROFORM-d) δ ppm 9.23 | | at 1 h | 1 h | at 1 h |
| | sulfanylidene-1H,2H,3H,4H,5H- | | | | (br s, 1 H) 7.31 (br d, J = 2.34 | | | | |
| | pyrrolo[3,2-d]pyrimidin-5-yl}methyl | | | | Hz, 1 H) 7.16 (d, J = 8.21 Hz, 2 | | | | |
| | carbonate | | | | H) 7.01 (d, J = 8.79 Hz, 2 H) | | | | |
| | | | | | 6.35 (s, 2 H) 6.29 (br s, 1 H) | | | | |
| | | | | | 4.45-4.61 (m, 2 H) 3.85 (br | | | | |
| | | | | | t, J = 5.27 Hz, 2 H) 3.56 (quin, | | | | |
| | | | | | J = 6.15 Hz, 1 H) 2.33 (s, 3 H) | | | | |
| | | | | | 1.02-1.16 (m, 6 H) | | | | |
| 237 | 3-methylphenyl {4-oxo-1-[2- | 5.27 | 418 | | 1H NMR (300 MHz, | 7.23 | 74% remaining | 2% remaining | 1% remaining |
| | (propan-2-yloxy)ethyl]-2- | | | | CHLOROFORM-d) δ ppm 9.25 | | at 1 h | at 1 h | at 1 h |
| | sulfanylidene-1H,2H,3H,4H,5H- | | | | (br s, 1 H) 7.32 (br d, J = 1.76 | | | | |
| | pyrrolo[3,2-d]pyrimidin-5-yl}methyl | | | | Hz, 1 H) 7.27 (m, J = 1.17 Hz, | | | | |
| | carbonate | | | | 1 H) 7.06 (d, J = 7.62 Hz, 1 H) | | | | |
| | | | | | 6.87-6.98 (m, 2 H) 6.35 (s, | | | | |
| | | | | | 2 H) 6.29 (br d, J = 2.34 Hz, 1 | | | | |
| | | | | | H) 4.54 (br s, 2 H) 3.85 (br t, | | | | |
| | | | | | J = 5.27 Hz, 2 H) 3.56 (quin, | | | | |
| | | | | | J = 6.01 Hz, 1 H) 2.34 (s, 3 H) | | | | |
| | | | | | 1.07 (d, J = 5.86 Hz, 6 H) | | | | |
| 238 | 2-fluorophenyl {4-oxo-1-[2-(propan- | 5.05 | 422 | | 1H NMR (300 MHz, | <3.39 | 13% remaining | 1% remaining | 1% remaining |
| | 2-yloxy)ethyl]-2-sulfanylidene- | | | | CHLOROFORM-d) δ ppm 9.24 | | at 1 h | at 1 h | at 1 h |
| | 1H,2H,3H,4H,5H-pyrrolo[3,2- | | | | (br s, 1 H) 7.32 (br s, 1 H) | | | | |
| | d]pyrimidin-5-yl}methyl carbonate | | | | 7.07-7.24 (m, 4 H) 6.39 (s, | | | | |
| | | | | | 2 H) 6.31 (br s, 1 H) 4.54 (br | | | | |
| | | | | | s, 2 H) 3.86 (br t, J = 4.98 Hz, | | | | |
| | | | | | 2 H) 3.36-3.69 (m, 1 H) | | | | |
| | | | | | 1.07 (d, J = 5.86 Hz, 6 H) | | | | |
| 239 | ({4-oxo-1-[2-(propan-2-yloxy)ethyl]- | 4.22 | 435 | | 1H NMR (300 MHz, DMSO-d6) | 117 | 99% remaining | 96% remaining | 85% remaining |
| | 1H,4H,5H-pyrrolo[3,2-d]pyrimidin-2 | | | | δ ppm 11.86-12.29 (m, 1 H) | | at 1 h | at 1 h | at 1 h |
| | yl}sulfanyl)methyl N-(2- | | | | 7.19-7.45 (m, 3 H) 7.09 (br | | | | |
| | fluorophenyl)-N-methylcarbamate | | | | s, 2 H) 6.35 (d, J = 2.93 Hz, 1 | | | | |
| | | | | | H) 5.58-6.04 (m, 2 H) 4.24 | | | | |
| | | | | | (br t, J = 5.27 Hz, 2 H) 3.65 (br | | | | |
| | | | | | t, J = 4.98 Hz, 2 H) 3.45 (dt, | | | | |
| | | | | | J = 12.16, 5.93 Hz, 1 H) 3.13 | | | | |
| | | | | | (s, 3 H) 0.94 (d, J = 5.86 Hz, 6 | | | | |
| | | | | | H) | | | | |
| 240 | ({4-oxo-1-[2-(propan-2-yloxy)ethyl]- | 4.33 | 451 | | 1H NMR (300 MHz, DMSO-d6) | 154 | 99% remaining | 97% remaining | 83% remaining |
| | 1H,4H,5H-pyrrolo[3,2-d]pyrimidin-2- | | | | δ ppm 12.06 (br s, 1 H) 7.15- | | at 1 h | at 1 h | at 1 h |
| | yl}sulfanyl)methyl N-(2- | | | | 7.61 (m, 5 H) 6.30-6.42 (m, | | | | |
| | chlorophenyl)-N-methylcarbamate | | | | 1 H) 5.65-6.04 (m, 2 H) | | | | |
| | | | | | 4.18-4.34 (m, 2 H) 3.57- | | | | |
| | | | | | 3.77 (m, 2 H) 3.36-3.53 (m, | | | | |
| | | | | | 1 H) 3.01-3.14 (m, 3 H) | | | | |
| | | | | | 0.84-1.07 (m, 6 H) | | | | |
| 241 | ({4-oxo-1-[2-(propan-2-yloxy)ethyl]- | 4.15 | 447 | | 1H NMR (300 MHz, DMSO-d6) | 105 | 57% remaining | 97% remaining | 100% remaining |
| | 1H,4H,5H-pyrrolo[3,2-d]pyrimidin-2- | | | | δ ppm 12.05 (br s, 1 H) 7.28 | | at 1 h | at 1 h | at 1 h |
| | yl}sulfanyl)methyl N-(4- | | | | (d, J = 2.34 Hz, 1 H) 7.10 (br | | | | |
| | methoxyphenyl)-N- | | | | s, 2 H) 6.78 (br s, 2 H) 6.35 | | | | |
| | methylcarbamate | | | | (d, J = 2.34 Hz, 1 H) 5.68- | | | | |
| | | | | | 5.87 (m, 2 H) 4.24 (t, J = 5.27 | | | | |
| | | | | | Hz, 2 H) 3.60-3.70 (m, 5 H) | | | | |
| | | | | | 3.45 (quin, J = 6.15 Hz, 1 H) | | | | |
| | | | | | 3.13 (s, 3 H) 0.94 (d, J = 5.86 | | | | |
| | | | | | Hz, 6 H) | | | | |
| 242 | ({4-oxo-1-[2-(propan-2-yloxy)ethyl]- | 4.28 | 431 | | 1H NMR (300 MHz, DMSO-d6) | 156 | 52% remaining | 96% remaining | 81% remaining |
| | 1H,4H,5H-pyrrolo[3,2-d]pyrimidin-2- | | | | δ ppm 11.89-12.14 (m, 1 H) | | at 1 h | at 1 h | at 1 h |
| | yl}sulfanyl)methyl N-methyl-N-(2- | | | | 7.26 (br d, J = 2.93 Hz, 1 H) | | | | |
| | methylphenyl)carbamate | | | | 7.18-7.23 (m, 1 H) 7.08- | | | | |
| | | | | | 7.17 (m, 3 H) 6.21-6.51 (m, | | | | |
| | | | | | 1 H) 5.73 (d, J = 1.17 Hz, 2 H) | | | | |
| | | | | | 4.13-4.35 (m, 2 H) 3.55- | | | | |
| | | | | | 3.79 (m, 2 H) 3.39-3.52 (m, | | | | |
| | | | | | 1 H) 3.04-3.13 (m, 3 H) | | | | |
| | | | | | 2.01 (s, 3 H) 0.83-1.04 (m, | | | | |
| | | | | | 6 H) | | | | |
| 243 | {2-[{{[methyl(2- | 6.15 | 608 | | 1H NMR (300 MHz, DMSO-d6) | 314 | 88% remaining | 99% remaining | 99% remaining |
| | methylphenyl)carbamoyl]oxy}methyl) | | | | δ ppm 7.34-7.52 (m, 2 H) | | at 1 h | at 1 h | at 1 h |
| | sulfanyl]-4-oxo-1-[2-(propan-2- | | | | 7.01-7.32 (m, 8 H) 6.03- | | | | |
| | yloxy)ethyl]-1H,4H,5H-pyrrolo[3,2- | | | | 6.61 (m, 2 H) 5.71 (br d, | | | | |
| | d]pyrimidin-5-yl}methyl N-methyl-N- | | | | J = 11.14 Hz, 2 H) 4.13-4.31 | | | | |
| | (2-methylphenyl)carbamate | | | | (m, 2H) 3.55-3.73 (m, 2H) | | | | |
| | | | | | 3.36-3.53 (m, 1 H) 2.96- | | | | |
| | | | | | 3.14 (m, 6 H) 1.78-2.19 (m, | | | | |
| | | | | | 6H) 0.84-0.99 (m, 6 H) | | | | |
| 244 | [3-({[methyl(2- | 6.15 | 608 | | 1H NMR (300 MHz, DMSO-d6) | 74.7 | 98% remaining | 99% remaining | 52% remaining |
| | methylphenyl)carbamoyl]oxy}methyl)- | | | | δ ppm 7.43-7.68 (m, 2 H) | | at 1 h | at 1 h | at 1 h |
| | 4-oxo-1-[2-(propan-2- | | | | 6.99-7.35 (m, 8H) 5.91- | | | | |
| | yloxy)ethyl]-2-sulfanylidene- | | | | 6.55 (m, 6 H) 5.73 (s, 1 H) | | | | |
| | 1H,2H,3H,4H,5H-pyrrolo[3,2- | | | | 4.34-4.71 (m, 2 H) 3.60- | | | | |
| | d]pyrimidin-5-yl]methyl N-methyl-N- | | | | 3.84 (m, 2 H) 3.36-3.57 (m, | | | | |
| | (2-methylphenyl)carbamate | | | | 1 H) 2.95-3.14 (m, 6 H) | | | | |
| | | | | | 2.13 (br s, 6 H)-1.03 (m, 6 | | | | |
| | | | | | H) | | | | |
| 245 | ({4-oxo-1-[2-(propan-2-yloxy)ethyl]- | 4.37 | 431 | | 1H NMR (300 MHz, DMSO-d6) | 216 | 99% remaining | 99% remaining | 63% remaining |
| | 1H,4H,5H-pyrrolo[3,2-d]pyrimidin-2- | | | | δ ppm 12.04 (br s, 1 H) 7.28 | | at 1 h | at 1 h | at 1 h |
| | yl}sulfanyl)methyl N-methyl-N-(3- | | | | (d, J = 2.93 Hz, 1 H) 7.06- | | | | |
| | methylphenyl)carbamate | | | | 7.18 (m, 1 H) 6.90-7.04 (m, | | | | |
| | | | | | 3 H) 6.35 (d, J = 2.93 Hz, 1 H) | | | | |
| | | | | | 5.80 (s, 2 H) 4.25 (t, J = 5.57 | | | | |
| | | | | | Hz, 2 H) 3.66 (t, J = 5.57 Hz, 2 | | | | |
| | | | | | H) 3.45 (dt, J = 12.16, 5.93 | | | | |
| | | | | | Hz, 1 H) 3.16 (s, 3 H) 2.12 (s, | | | | |
| | | | | | 3 H) 0.94 (d, J = 6.45 Hz, 6 H) | | | | |
| 246 | {2-[{{[methyl(3- | 6.15 | 609 | | 1H NMR (300 MHz, DMSO-d6) | 217 | 98% remaining | 99% remaining | 99% remaining |
| | methylphenyl)carbamoyl]oxy}meth- | | | | δ ppm 7.46 (d, J = 2.93 Hz, 1 | | at 1 h | at 1 h | at 1 h |
| | yl)sulfanyl]-4-oxo-1-[2-(propan-2- | | | | H) 7.07-7.22 (m, 3 H) 6.88- | | | | |
| | yloxy)ethyl]-1H,4H,5H-pyrrolo[3,2- | | | | 7.03 (m, 5 H) 6.45 (d, J = 3.52 | | | | |
| | d]pyrimidin-5-yl}methyl N-methyl-N- | | | | Hz, 1 H) 6.25 (s, 2 H) 5.79 (s, | | | | |
| | (3-methylphenyl)carbamate | | | | 2 H) 4.25 (t, J = 5.27 Hz, 2 H) | | | | |
| | | | | | 3.64 (t, J = 5.27 Hz, 2 H) 3.44 | | | | |
| | | | | | (dt, J = 12.16, 5.93 Hz, 1 H) | | | | |
| | | | | | 3.15 (d, J = 9.38 Hz, 6 H) 2.01- | | | | |
| | | | | | 2.22 (m, 6 H) 0.92 (d, | | | | |
| | | | | | J = 6.45 Hz, 6 H) | | | | |
| 247 | [3-({[methyl(3- | 6.15 | 609 | | 1H NMR (300 MHz, DMSO-d6) | 39.9 | 64% remaining | 81% remaining | 31% remaining |
| | methylphenyl)carbamoyl]oxy}methyl)- | | | | δ ppm 7.59 (d, J = 3.52 Hz, 1 | | at 1 h | at 1 h | at 1 h |
| | 4-oxo-1-[2-(propan-2- | | | | H) 7.02-7.22 (m, 4 H) 6.92 | | | | |
| | yloxy)ethyl]-2-sulfanylidene- | | | | (br d, J = 4.10 Hz, 4 H) 6.42 | | | | |
| | 1H,2H,3H,4H,5H-pyrrolo[3,2- | | | | (d, J = 2.93 Hz, 1 H) 6.34 (s, 2 | | | | |
| | d]pyrimidin-5-yl]methyl N-methyl-N- | | | | H) 6.13 (s, 2 H) 4.56 (t, | | | | |
| | (3-methylphenyl)carbamate | | | | J = 5.86 Hz, 2 H) 3.73 (t, | | | | |
| | | | | | J = 5.86 Hz, 2 H) 3.49 (dt, | | | | |
| | | | | | J = 12.16, 5.93 Hz, 1 H) 3.14 | | | | |
| | | | | | (d, J = 11.72 Hz, 6 H) 2.10- | | | | |
| | | | | | 2.25 (m, 6 H) 0.93 (d, J = 5.86 | | | | |
| | | | | | Hz, 6 H). | | | | |
| 248 | ({4-oxo-1-[2-(propan-2-yloxy)ethyl]- | 4.22 | 431 | | 1H NMR (300 MHz, DMSO-d6) | 235 | 99% remaining | 98% remaining | 96% remaining |
| | 1H,4H,5H-pyrrolo[3,2-d]pyrimidin-2- | | | | δ ppm 12.03 (br s, 1 H), 7.28 | | at 1 h | at 1 h | at 1 h |
| | yl}sulfanyl)methyl N-methyl-N-(4- | | | | (d, J = 2.93 Hz, 1 H), 6.97- | | | | |
| | methylphenyl)carbamate | | | | 7.17 (m, 4 H), 6.29-6.42 (m, | | | | |
| | | | | | 1 H), 5.80 (s, 2 H), 4.24 (t, | | | | |
| | | | | | J = 5.57 Hz, 2 H), 3.65 (t, | | | | |
| | | | | | J = 5.57 Hz, 2 H), 3.38-3.51 | | | | |
| | | | | | (m, 1 H), 3.15 (s, 3 H), 2.21 | | | | |
| | | | | | (s, 3 H), 0.94 (d, J = 5.86 Hz, | | | | |
| | | | | | 6 H) | | | | |
| 249 | {2-[{{[(2- | 5.38 | 648 | | 1H NMR (300 MHz, DMSO-d6) | 284 | 89% remaining | 95% remaining | 98% remaining |
| | chlorophenyl)(methyl)carbamoyl]oxy}meth- | | | | δ ppm 7.05-7.74 (m, 9 H) | | at 1 h | at 1 h | at 1 h |
| | yl)sulfanyl]-4-oxo-1-[2- | | | | 6.09-6.55 (m, 3 H) 5.61- | | | | |
| | (propan-2-yloxy)ethyl]-1H,4H,5H- | | | | 5.96 (m, 2 H) 4.07-4.37 (m, | | | | |
| | pyrrolo[3,2-d]pyrimidin-5-yl}methyl | | | | 2 H) 3.52-3.78 (m, 2 H) | | | | |
| | N-(2-chlorophenyl)-N- | | | | 3.37-3.52 (m, 1 H) 3.00- | | | | |
| | methylcarbamate | | | | 3.11 (m, 6 H) 0.83-0.98 (m, | | | | |
| | | | | | 6 H) | | | | |
| 250 | {4-oxo-1-[2-(propan-2-yloxy)ethyl]- | 5.07 | M + Na⁺ = 473 | | 1H NMR (300 MHz, DMSO-d6) | | 99% remaining | 93% remaining | 100% remaining |
| | 2-sulfanylidene-1H,2H,3H,4H,5H- | | | | δ ppm 12.27 (br s, 1 H) 7.39- | | at 1 h | at 1 h | at 1 h |
| | pyrrolo[3,2-d]pyrimidin-5-yl}methyl | | | | 7.58 (m, 3H) 7.26-7.38 (m, | | | | |
| | N-(2-chlorophenyl)-N- | | | | 2 H) 6.29-6.42 (m, 1 H) | | | | |
| | methylcarbamate | | | | 5.98-6.25 (m, 2 H) 4.35- | | | | |
| | | | | | 4.55 (m, 2 H) 3.64-3.77 (m, | | | | |
| | | | | | 2 H) 3.49 (dt, J = 12.16, 5.93 | | | | |
| | | | | | Hz, 1 H) 2.99-3.09 (m, 3 H) | | | | |
| | | | | | 0.86-1.02 (m, 6 H) | | | | |
| 251 | {4-oxo-1-[2-(propan-2-yloxy)ethyl]- | 5.05 | 431 | | 1H NMR (300 MHz, DMSO-d6) | | 99% remaining | 100% remaining | 96% remaining |
| | 2-sulfanylidene-1H,2H,3H,4H,5H- | | | | δ ppm 12.29 (br s, 1 H) 7.43 | | at 1 h | at 1 h | at 1 h |
| | pyrrolo[3,2-d]pyrimidin-5-yl}methyl | | | | (d, J = 2.93 Hz, 1 H) 7.04- | | | | |
| | N-methyl-N-(2- | | | | 7.28 (m, 4 H) 6.29-6.44 (m, | | | | |
| | methylphenyl)carbamate | | | | 1 H) 6.19 (s, 1 H) 5.83-6.26 | | | | |
| | | | | | (m, 2 H) 4.35-4.54 (m, 2 H) | | | | |
| | | | | | 3.62-3.77 (m, 2 H) 3.49 (dq, | | | | |
| | | | | | J = 12.02, 5.96 Hz, 1 H) 2.97- | | | | |
| | | | | | 3.09 (m, 3 H) 1.85-2.15 (m, | | | | |
| | | | | | 3 H) 0.94 (d, J = 5.86 Hz, 6 H) | | | | |
| 252 | {4-oxo-1-[2-(propan-2-yloxy)ethyl]- | 5.12 | 431 | | 1H NMR (300 MHz, DMSO-d6) | | 100% remaining | 80% remaining | 100% remaining |
| | 2-sulfanylidene-1H,2H,3H,4H,5H- | | | | δ ppm 12.38 (s, 1 H) 7.49 (d, | | at 1 h | at 1 h | at 1 h |
| | pyrrolo[3,2-d]pyrimidin-5-yl}methyl | | | | J = 2.93 Hz, 1 H) 7.15 (t, | | | | |
| | N-methyl-N-(3- | | | | J = 7.62 Hz, 1 H) 6.97 (br d, | | | | |
| | methylphenyl)carbamate | | | | J = 8.21 Hz, 3 H) 6.29-6.42 | | | | |
| | | | | | (m, 1 H) 6.12 (s, 2 H) 4.45 (t, | | | | |
| | | | | | J = 6.15 Hz, 2 H) 3.63-3.75 | | | | |
| | | | | | (m, 2 H) 3.50 (quin, J = 6.15 | | | | |
| | | | | | Hz, 1 H) 3.13 (s, 3 H) 2.19 (s, | | | | |
| | | | | | 3 H) 0.95 (d, J = 5.86 Hz, 6 H) | | | | |
| 253 | {2-[{{[(4- | 5.03 | 640 | | 1H NMR (300 MHz, DMSO-d6) | | | | |
| | methoxyphenyl)(methyl)carbamoyl] | | | | δ ppm 7.45 (d, J = 2.93 Hz, 1 | | | | |
| | oxy}methyl)sulfanyl]-4-oxo-1-[2- | | | | H) 7.10 (br s, 4 H) 6.80 (br s, | | | | |
| | (propan-2-yloxy)ethyl]-1H,4H,5H- | | | | 4 H) 6.45 (d, J = 2.93 Hz, 1 H) | | | | |
| | pyrrolo [3,2-d]pyrimidin-5-yl}methyl | | | | 6.24 (br s, 2 H) 5.79 (br d, | | | | |
| | N-(4-methoxyphenyl)-N- | | | | J = 7.03 Hz, 2 H) 4.23 (br t, | | | | |
| | methylcarbamate | | | | J = 5.28 Hz, 2 H) 3.58-3.75 | | | | |
| | | | | | (m, 8 H) 3.39-3.52 (m, 1 H) | | | | |
| | | | | | 3.14 (s, 3 H) 3.10 (s, 3 H) | | | | |
| | | | | | 0.93 (d, J = 5.86 Hz, 6 H) | | | | |
| 254 | ({4-oxo-1-[2-(propan-2-yloxy)ethyl]- | 4.13 | 447 | | 1H NMR (300 MHz, DMSO-d6) | 85.3 | 95% remaining | 94% remaining | 88% remaining |
| | 1H,4H,5H-pyrrolo[3,2-d]pyrimidin-2- | | | | δ ppm 12.05 (br s, 1 H) 7.28 | | after 1 h | after 1 h | after 1 h |
| | yl}sulfanyl)methyl N-(4- | | | | (t, J = 2.93 Hz, 1 H) 7.10 (br s, | | | | |
| | methoxyphenyl)-N- | | | | 2 H) 6.77 (br d, J = 1.76 Hz, 2 | | | | |
| | methylcarbamate | | | | H) 6.29-6.41 (m, 1 H) 5.80 | | | | |
| | | | | | (br d, J = 8.21 Hz, 2 H) 4.12- | | | | |
| | | | | | 4.37 (m, 2 H) 3.57-3.74 (m, | | | | |
| | | | | | 5 H) 3.45 (quin, J = 6.15 Hz, 1 | | | | |
| | | | | | H) 3.13 (s, 3 H) 0.94 (d, | | | | |
| | | | | | J = 6.45 Hz, 6 H) | | | | |

### Example 255

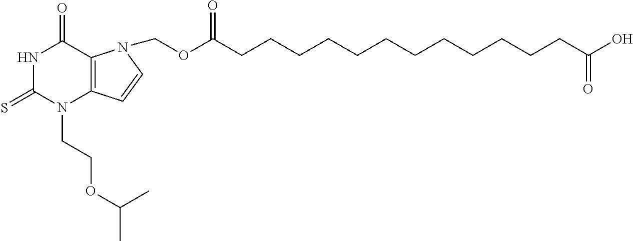 [SMILES: CC(C)OCCn1c(=S)[nH]c(=O)c2c1ccn2COC(=O)CCCCCCCCCCCCC(=O)O]

Charged a 250 mL round bottom flask with hexanedioic acid (5 g, 17.4 mmole), t-butanol (43 mL, 272 mmole), EDC (2.71 g, 17.46 mmole) and DMAP (2.13 g, 17.46 mmole). Dissolved in 30 mL of CH₂Cl₂, at ambient temperature. After 18 hours, partitioned between ethyl acetate 50 mL and 20 mL of 0.01 N HCl. Separated layers and washed the organic layer with brine. Dried over Na₂SO₄, filtered and concentrated in vacuo. Purified on a 40 g silica gel column, eluting with 0-100% ethyl acetate in hexanes. Combined desired fractions in vacuo to yield 045b: (2.18 g, 40%). ¹H NMR (300 MHz, DMSO-d₆) δ ppm 2.08-2.20 (m, 4H) 1.37-1.51 (m, 4H) 1.35 (s, 5H) 1.20 (s, 21H) 1.11-1.16 (m, 2H) 1.07 (s, 9H).

To a solution of MAM-6-386-045 (0.50 g, 1.6 mmol) in DCM:H₂O (12:8 mL) at RT was added NaHCO₃ (0.53 g, 6.3 mmole), then tetrabutylammonium hydrogen sulfate (0.054 g, 0.16 mmole). Cooled to 0° C. and let stir for 15 minutes. then added chloromethylchlorosulfate (5.07 mmole, 0.513 mL). Let warm slowly to RT over the 3 days. Partitioned reaction between CH₂Cl₂ and H₂O. Separated layers and washed the aqueous layer with 2×20 mL of CH₂Cl₂. Product has some solubility issues. Added MeOH to help solubilize, and EtOAc and Hexanes. Washed the combined organic layers with brine. Dried over Na₂SO₄, filtered and concentrated in vacuo. Purified on the ISCO using an 40 g SiliaSep column, eluting with 0-100% EtOAc/hexanes. Concentrated the first spot in vacuo to yield 047 (212 mg, 36%), ¹H NMR (300 MHz, CHLOROFORM-d) δ ppm 5.69 (s, 2H) 2.11-2.43 (m, 4H) 1.51-1.75 (m, 4H) 1.43 (s, 9H) 1.13-1.35 (m, 20H).

Added sodium hydride 60% dispersion in mineral oil (33 mg, 1.4 mmole) to a solution of 1-[2-(propan-2-yloxy)ethyl]-2-sulfanylidene-1H,2H,3H,4H,5H-pyrrolo[3,2-d]pyrimidin-4-one (0.14 g, 0.55 mmole) in DMF (3 mL). After gas evolution had ceased, added mam-6-386-047 (0.135 g, 0.592 mmole) in 1 mL DMF. After 24 hours, partitioned between ethyl acetate (40 mL) and H₂O (20 mL). Separated layers and washed the aqueous layer with 20 mL of ethyl acetate. Washed the combined organic layers with 2×20 mL H₂O, then brine. Dried over Na₂SO₄, filtered and concentrated in vacuo. Purified on a 40 g silica gel column, eluting with 0-100% EtOAc in hexanes to yield 067 (0.10 g, 30%). ¹H NMR (300 MHz, CHLOROFORM-d) δ ppm 9.52 (br s, 1H) 7.27 (d, J=3.52 Hz, 1H) 6.22 (s, 3H) 4.52 (t, J=5.57 Hz, 2H) 3.83 (t, J=5.57 Hz, 2H) 3.54 (dq, J=12.09, 6.13 Hz, 3H) 2.10-2.43 (m, 6H) 1.49-1.74 (m, 6H) 1.43 (s, 9H) 1.14-1.29 (m, 35H) 1.06 (d, J=5.86 Hz, 7H). LC/MS Method A-2: Rₜ=7.95 mins., (M+H)⁺=609, purity=90%.

Added TFA (0.94 mmole, 0.63 mL) to a solution of MAM-6-386-067 (0.100 g, 0.395 mmole) in CH₂Cl₂, 4 mL. After 2 hours, partitioned between CH₂Cl₂ (40 mL) and H₂O 20 mL. Separated layers and washed the aqueous layer with 20 mL of CH₂Cl₂. Washed the combined organic layers with 1×20 mL H₂O, then brine. Dried over Na₂SO₄, filtered and concentrated in vacuo. Purified on a 40 g silica gel column, eluting with 0-100% EtOAc in hexanes, to give 16-oxo-16-({4-oxo-1-[2-(propan-2-yloxy)ethyl]-2-sulfanylidene-1H,2H,3H,4H,5H-pyrrolo[3,2-d]pyrimidin-5-yl}methoxy)hexadecanoic acid: (29 mg, 32%), ¹H NMR (300 MHz, DMSO-d₆) δ ppm 11.63-12.77 (m, 2H), 7.49 (d, J=3.52 Hz, 1H), 6.36 (d, J=2.93 Hz, 1H), 6.14 (s, 2H), 4.45 (t, J=6.15 Hz, 2H), 3.69 (t, J=6.15 Hz, 2H), 3.52 (dt, J=12.16, 5.93 Hz, 1H), 2.25 (t, J=7.33 Hz, 2H), 2.14 (t, J=7.33 Hz, 2H), 1.43 (br d, J=3.52 Hz, 4H), 1.08-1.27 (m, 20H), 0.97 (d, J=5.86 Hz, 6H). LC/MS Method A: Rₜ=6.33 mins., (M+H)⁺=552, purity >95%. Caco-2 Pₐₚₚ=27.2 nm/s, SGF=95% remaining at 1 h, SIF=2% remaining at 1 h, hPlasma stability=76% remaining at 1 h.

Additional compounds prepared similarly to EXAMPLE 255:

| | | LCMS | | | | Caco-2 | | | human |
|---|---|---|---|---|---|---|---|---|---|
| | | retention | | LCMS | | Pₐₚₚ | | | Plasma |
| Example | Name | time | M + H⁺ | method | NMR | (nm/s) | SGF | SIF | stability |
| 256 | 1-{4-oxo-1-[2-(propan-2- | 7.52 | 595 | | 1H NMR (300 MHz, DMSO-d6) | 2.5 | 99% remaining | 9% remaining | 88% remaining |
| | yloxy)ethyl]-2-sulfanylidene- | | | | δ ppm 12.34 (br s, 1 H) 7.49 | | after 1 h | after 1 h | after 1 h |
| | 1H,2H,3H,4H,5H-pyrrolo[3,2- | | | | (d, J = 3.52 Hz, 1 H) 6.36 (d, | | | | |
| | d]pyrimidin-5-yl}methyl 16-propan- | | | | J = 3.52 Hz, 1 H) 6.14 (s, 2 H) | | | | |
| | 2-yl hexadecanedioate | | | | 4.68-4.99 (m, 1 H) 4.45 (t, | | | | |
| | | | | | J = 5.86 Hz, 2 H) 3.68 (t, | | | | |
| | | | | | J = 5.86 Hz, 2 H) 3.52 (quin, | | | | |
| | | | | | J = 6.15 Hz, 1 H) 2.15-2.29 | | | | |
| | | | | | (m, 4H) 1.44 (br dd, J = 7.33, | | | | |
| | | | | | 2.05 Hz, 4 H) 1.06-1.26 (m, | | | | |
| | | | | | 26 H) 0.90-1.02 (m, 6 H) | | | | |
| 257 | {4-oxo-1-[2-(propan-2-yloxy)ethyl]- | 4.85 | 447 | | 1H NMR (300 MHz, DMSO-d6) | | | | |
| | 2-sulfanylidene-1H,2H,3H,4H,5H- | | | | δ ppm 12.34 (s, 1 H) 7.48 (br | | | | |
| | pyrrolo[3,2-d]pyrimidin-5-yl}methyl | | | | d, J = 2.93 Hz, 1 H) 7.09 (br s, | | | | |
| | N-(4-methoxyphenyl)-N- | | | | 2 H) 6.61-6.93 (m, 2 H) | | | | |
| | methylcarbamate | | | | 6.35 (d, J = 2.93 Hz, 1 H) 6.10 | | | | |
| | | | | | (br s, 2 H) 4.45 (t, J = 6.15 Hz, | | | | |
| | | | | | 2 H) 3.65-3.74 (m, 5 H) | | | | |
| | | | | | 3.45-3.57 (m, 1 H) 3.10 (s, | | | | |
| | | | | | 3 H) 0.96 (d, J = 6.45 Hz, 6 H) | | | | |
| 258 | {4-oxo-1-[2-(propan-2-yloxy)ethyl]- | 4.87 | 435 | | 1H NMR (300 MHz, DMSO-d6) | | | | |
| | 2-sulfanylidene-1H,2H,3H,4H,5H- | | | | δ ppm 12.29 (br s, 1 H) 7.44 | | | | |
| | pyrrolo[3,2-d]pyrimidin-5-yl}methyl | | | | (br s, 1 H) 7.06-7.37 (m, 4 | | | | |
| | N-(2-fluorophenyl)-N- | | | | H) 6.35 (br s, 1 H) 6.03-6.22 | | | | |
| | methylcarbamate | | | | (m, 2 H) 4.44 (br t, J = 5.86 | | | | |
| | | | | | Hz, 2 H) 3.69 (t, J = 6.15 Hz, 2 | | | | |
| | | | | | H) 3.43-3.58 (m, 1 H) 3.10 | | | | |
| | | | | | (s, 3 H) 0.96 (d, J = 6.45 Hz, 6 | | | | |
| | | | | | H) | | | | |
| 259 | {4-oxo-1-[2-(propan-2-yloxy)ethyl]- | 4.95 | 435 | | 1H NMR (300 MHz, DMSO-d6) | | | | |
| | 2-sulfanylidene-1H,2H,3H,4H,5H- | | | | δ ppm 12.36 (s, 1 H) 7.51 (d, | | | | |
| | pyrrolo[3,2-d]pyrimidin-5-yl}methyl | | | | J = 3.52 Hz, 1 H) 7.26-7.39 | | | | |
| | N-(3-fluorophenyl)-N- | | | | (m, 1 H) 6.94-7.18 (m, 3 H) | | | | |
| | methylcarbamate | | | | 6.36 (d, J = 3.52 Hz, 1 H) 6.15 | | | | |
| | | | | | (s, 2 H) 4.45 (t, J = 5.86 Hz, 2 | | | | |
| | | | | | H) 3.69 (t, J = 5.86 Hz, 2 H) | | | | |
| | | | | | 3.45-3.56 (m, 1 H) 3.17 (s, | | | | |
| | | | | | 3 H) 0.92-1.00 (m, 6 H) | | | | |
| 260 | {4-oxo-1-[2-(propan-2-yloxy)ethyl]- | 4.95 | 435 | | 1H NMR (300 MHz, DMSO-d6) | | | | |
| | 2-sulfanylidene-1H,2H,3H,4H,5H- | | | | δ ppm 12.34 (s, 1 H) 7.48 (d, | | | | |
| | pyrrolo[3,2-d]pyrimidin-5-yl}methyl | | | | J = 2.93 Hz, 1 H) 7.23 (br s, 2 | | | | |
| | N-(4-fluorophenyl)-N- | | | | H) 7.06-7.17 (m, 2 H) 6.35 | | | | |
| | methylcarbamate | | | | (d, J = 3.52 Hz, 1 H) 6.07- | | | | |
| | | | | | 6.16 (m, 2 H) 4.44 (t, J = 6.15 | | | | |
| | | | | | Hz, 2 H) 3.69 (t, J = 6.15 Hz, 2 | | | | |
| | | | | | H) 3.45-3.56 (m, 1 H) 3.13 | | | | |
| | | | | | (s, 3 H) 0.90-1.04 (m, 6 H) | | | | |
| 261 | 12-oxo-12-({4-oxo-1-[2-(propan-2- | 5.3 | 496 | | 1H NMR (300 MHz, DMSO-d6) | 117 | 99% remaining | 2% remaining | 94% remaining |
| | yloxy)ethyl]-2-sulfanylidene- | | | | δ ppm 11.75-12.65 (m, 2 H) | | after 1 h | after 1 h | after 1 h |
| | 1H,2H,3H,4H,5H-pyrrolo[3,2- | | | | 7.49 (d, J = 2.93 Hz, 1 H) 6.37 | | | | |
| | d]pyrimidin-5- | | | | (d, J = 2.93 Hz, 1 H) 6.14 (s, 2 | | | | |
| | yl}methoxy)dodecanoic acid | | | | H) 4.45 (t, J = 6.15 Hz, 2 H) | | | | |
| | | | | | 3.68 (t, J = 5.86 Hz, 2 H) 3.52 | | | | |
| | | | | | (quin, J = 6.15 Hz, 1 H) 2.25 | | | | |
| | | | | | (t, J = 7.33 Hz, 2 H) 2.14 (t, | | | | |
| | | | | | J = 7.33 Hz, 2 H) 1.36-1.51 | | | | |
| | | | | | (m, 4 H) 1.06-1.22 (m, 12 | | | | |
| | | | | | H) 0.97 (d, J = 6.45 Hz, 6 H) | | | | |
| 262 | 1-{4-oxo-1-[2-(propan-2- | 6.53 | 538 | | 1H NMR (300 MHz, DMSO-d6) | <2.14 | 92% remaining | 2% remaining | 41% remaining |
| | yloxy)ethyl]-2-sulfanylidene- | | | | δ ppm 12.34 (br s, 1 H) 7.49 | | after 1 h | after 1 h | after 1 h |
| | 1H,2H,3H,4H,5H-pyrrolo[3,2- | | | | (d, J = 3.52 Hz, 1 H) 6.36 (d, | | | | |
| | d]pyrimidin-5-yl}methyl 12-propan- | | | | J = 3.52 Hz, 1 H) 6.14 (s, 2 H) | | | | |
| | 2-yl dodecanedioate | | | | 4.68-4.99 (m, 1 H) 4.45 (t, | | | | |
| | | | | | J = 5.86 Hz, 2 H) 3.68 (t, | | | | |
| | | | | | J = 5.86 Hz, 2 H) 3.52 (quin, | | | | |
| | | | | | J = 6.15 Hz, 1 H) 2.15-2.29 | | | | |
| | | | | | (m, 4 H) 1.44 (br dd, J = 7.33, | | | | |
| | | | | | 2.05 Hz, 4 H) 1.06-1.26 (m, | | | | |
| | | | | | 18 H) 0.90-1.02 (m, 6 H) | | | | |
| 263 | 13-oxo-13-({4-oxo-1-[2-(propan-2- | 5.5 | 510 | | 1H NMR (300 MHz, DMSO-d6) | | | | |
| | yloxy)ethyl]-2-sulfanylidene- | | | | δ ppm 12.36 (br d, J = 1.17 | | | | |
| | 1H,2H,3H,4H,5H-pyrrolo[3,2- | | | | Hz, 2 H) 7.50 (d, J = 2.93 Hz, | | | | |
| | d]pyrimidin-5- | | | | 1 H) 6.38 (d, J = 2.93 Hz, 1 H) | | | | |
| | yl}methoxy)tridecanoic acid | | | | 6.15 (s, 2 H) 4.45 (t, J = 6.15 | | | | |
| | | | | | Hz, 2 H) 3.61-3.80 (m, 2 H) | | | | |
| | | | | | 3.52 (quin, J = 6.15 Hz, 1 H) | | | | |
| | | | | | 2.26 (t, J = 7.33 Hz, 2 H) 2.15 | | | | |
| | | | | | (t, J = 7.33 Hz, 2 H) 1.37- | | | | |
| | | | | | 1.57 (m, 4 H) 1.09-1.27 (m, | | | | |
| | | | | | 14 H) 0.98 (d, J = 6.45 Hz, 6 | | | | |
| | | | | | H) | | | | |
| 264 | 15-oxo-15-({4-oxo-1-[2-(propan-2- | 5.98 | 539 | | 1H NMR (300 MHz, DMSO-d6) | | | | |
| | yloxy)ethyl]-2-sulfanylidene- | | | | δ ppm 12.36 (br s, 2 H) 7.50 | | | | |
| | 1H,2H,3H,4H,5H-pyrrolo[3,2- | | | | (d, J = 3.52 Hz, 1 H) 6.38 (d, | | | | |
| | d]pyrimidin-5- | | | | J = 3.52 Hz, 1 H) 6.15 (s, 2 H) | | | | |
| | yl}methoxy)pentadecanoic acid | | | | 4.45 (t, J = 6.15 Hz, 2 H) 3.69 | | | | |
| | | | | | (t, J = 6.15 Hz, 2 H) 3.52 (dt, | | | | |
| | | | | | J = 12.02, 6.30 Hz, 1 H) 2.26 | | | | |
| | | | | | (t, J = 7.03 Hz, 2 H) 2.15 (t, | | | | |
| | | | | | J = 7.33 Hz, 2 H) 1.44 (br d, | | | | |
| | | | | | J = 4.10 Hz, 4 H) 1.17 (br d, | | | | |
| | | | | | J = 18.76 Hz, 18 H) 0.98 (d, | | | | |
| | | | | | J = 6.45 Hz, 6 H) | | | | |
| 265 | 1-{4-oxo-1-[2-(propan-2- | 7.1 | 581 | | 1H NMR (300 MHz, DMSO-d6) | | | | |
| | yloxy)ethyl]-2-sulfanylidene- | | | | δ ppm 12.37 (s, 1 H) 7.50 (d, | | | | |
| | 1H,2H,3H,4H,5H-pyrrolo[3,2- | | | | J = 2.93 Hz, 1 H) 6.38 (d, | | | | |
| | d]pyrimidin-5-yl}methyl 15-propan- | | | | J = 2.93 Hz, 1 H) 6.15 (s, 2 H) | | | | |
| | 2-yl pentadecanedioate | | | | 4.77-4.90 (m, 1 H) 4.45 (t, | | | | |
| | | | | | J = 5.86 Hz, 2 H) 3.59-3.74 | | | | |
| | | | | | (m, 2 H) 3.52 (quin, J = 6.01 | | | | |
| | | | | | Hz, 1 H) 2.26 (t, J = 7.33 Hz, 2 | | | | |
| | | | | | H) 2.20 (t, J = 7.33 Hz, 2 H) | | | | |
| | | | | | 1.45 (br dd, J = 7.03, 2.34 Hz, | | | | |
| | | | | | 4 H) 1.10-1.24 (m, 24 H) | | | | |
| | | | | | 0.94-1.00 (m, 6 H) | | | | |
| 266 | 18-oxo-18-({4-oxo-1-[2-(propan-2- | 5.79 Method: | 581 | | 1H NMR (300 MHz, DMSO-d6) | | | | |
| | yloxy)ethyl]-2-sulfanylidene- | 40-95% | | | δ ppm 12.01-12.68 (m, 1 H) | | | | |
| | 1H,2H,3H,4H,5H-pyrrolo[3,2- | ACN | | | 7.50 (d, J = 3.52 Hz, 1 H) 6.37 | | | | |
| | d]pyrimidin-5- | | | | (d, J = 2.93 Hz, 1 H) 6.27 (s, 1 | | | | |
| | yl}methoxy)octadecanoic acid | | | | H) 6.15 (s, 2 H) 4.45 (t, | | | | |
| | | | | | J = 5.86 Hz, 2 H) 3.69 (t, | | | | |
| | | | | | J = 6.15 Hz, 2 H) 3.46-3.58 | | | | |
| | | | | | (m, 1 H) 2.22-2.30 (m, 2 H) | | | | |
| | | | | | 2.15 (t, J = 7.33 Hz, 2 H) 1.44 | | | | |
| | | | | | (br d, J = 4.10 Hz, 4 H) 1.07- | | | | |
| | | | | | 1.28 (m, 24 H) 0.98 (d, | | | | |
| | | | | | J = 6.45 Hz, 6 H) | | | | |
| 267 | 18-{[2-({[(17- | 7.97 Method: | 907 | | 1H NMR (300 MHz, DMSO-d6) | | | | |
| | carboxyheptadecanoyl)oxy]methyl} | 40-95% | | | δ ppm 7.45 (d, J = 2.93 Hz, 1 | | | | |
| | sulfanyl)-4-oxo-1-[2-(propan-2- | CAN | | | H) 6.47 (d, J = 3.52 Hz, 1 H) | | | | |
| | yloxy)ethyl]-1H,4H,5H-pyrrolo[3,2- | | | | 6.27 (s, 2 H) 5.79 (s, 2 H) | | | | |
| | d]pyrimidin-5-yl]methoxy}-18- | | | | 4.22 (br t, J = 4.98 Hz, 2 H) | | | | |
| | oxooctadecanoic acid | | | | 3.64 (br t, J = 5.27 Hz, 2 H) | | | | |
| | | | | | 2.03-2.35 (m, 8 H) 1.36- | | | | |
| | | | | | 1.57 (m, 9 H) 1.03-1.31 (m, | | | | |
| | | | | | 48 H) 0.94 (d, J = 6.45 Hz, 6 | | | | |
| | | | | | H) 0.77-0.87 (m, 2 H). | | | | |
| 268 | ({4-oxo-1-[2-(propan-2-yloxy)ethyl]- | | | | | | | | |
| | 1H,4H,5H-pyrrolo[3,2-d]pyrimidin-2- | | | | | | | | |
| | yl}sulfanyl)methyl 2,2-dimethylpent- | | | | | | | | |
| | 4-enoate | | | | | | | | |
| 269 | {4-oxo-1-[2-(propan-2-yloxy)ethyl]- | | | | | | | | |
| | 2-sulfanylidene-1H,2H,3H,4H,5H- | | | | | | | | |
| | pyrrolo[3,2-d]pyrimidin-5-yl}methyl | | | | | | | | |
| | 2,2-dimethylpent-4-enoate | | | | | | | | |
| 270 | (3-{[(2,2-dimethylpent-4- | | | | | | | | |
| | enoyl)oxy]methyl}-4-oxo-1-[2- | | | | | | | | |
| | (propan-2-yloxy)ethyl]-2- | | | | | | | | |
| | sulfanylidene-1H,2H,3H,4H,5H- | | | | | | | | |
| | pyrrolo[3,2-d]pyrimidin-5-yl)methyl | | | | | | | | |
| | 2,2-dimethylpent-4-enoate | | | | | | | | |
| 271 | {4-oxo-1-[2-(propan-2-yloxy)ethyl]- | | | | | | | | |
| | 2-sulfanylidene-1H,2H,3H,4H,5H- | | | | | | | | |
| | pyrrolo[3,2-d]pyrimidin-3-yl}methyl | | | | | | | | |
| | 2,2-dimethylpent-4-enoate | | | | | | | | |
| 272 | [(5-{[(2,2-dimethylpent-4- | | | | | | | | |
| | enoyl)oxy]methyl}-4-oxo-1-[2- | | | | | | | | |
| | (propan-2-yloxy)ethyl]-1H,4H,5H- | | | | | | | | |
| | pyrrolo[3,2-d]pyrimidin-2- | | | | | | | | |
| | yl)sulfanyl]methyl 2,2-dimethylpent- | | | | | | | | |
| | 4-enoate | | | | | | | | |

### Examples 273 and 274

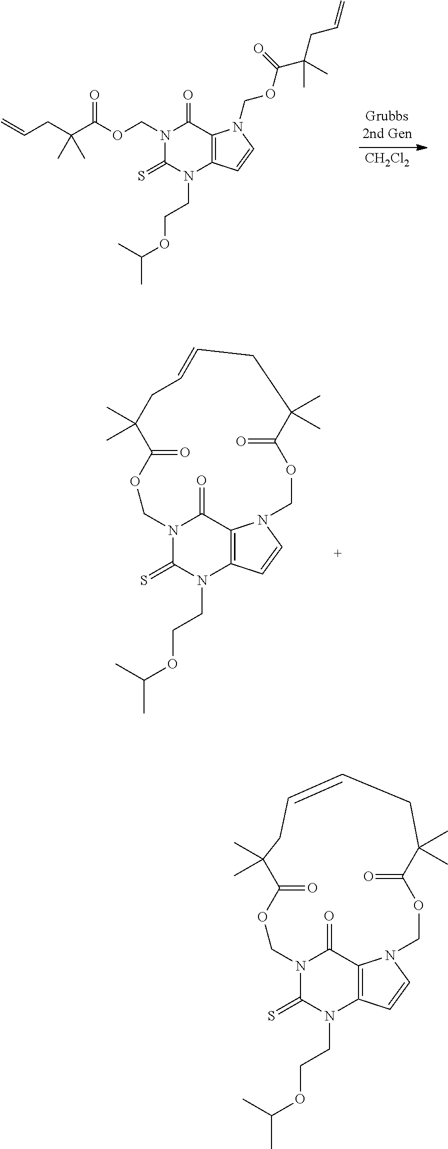

A solution of diene (60 mg, 0.112 mmol) in dichloromethane (100 mL) was degassed with N₂ for 10 min, followed by quick addition of Grubbs 2n d Gen (48 mg, 0.056 mmol) and degassing for 15 min. The mixture was heated at 40° C. for 24 h. The reaction was concentrated and the crude product mixture was purified twice by PLC eluted with 20% EtOAc/hexanes to get two pure isomer products as white solid. EXAMPLE 273 (28 mg, 50%), EXAMPLE 274 (16 mg, 28%). ¹H NMR (300 MHz, CHLOROFORM-d) δ 7.23 (d, J=2.93 Hz, 1H), 6.71 (s, 2H), 6.20 (d, J=2.93 Hz, 1H), 6.07 (br s, 2H), 5.16-5.33 (m, 1H), 4.74-4.92 (m, 1H), 4.60-4.67 (m, 2H), 3.88 (t, J=5.57 Hz, 2H), 3.48-3.59 (m, 1H), 2.07-2.35 (m, 4H), 1.19-1.29 (m, 6H), 1.08-1.19 (m, 6H), 1.04 (d, J=6.45 Hz, 6H). LC/MS Method A: Rₜ=6.48 mins., (M+H)⁺=507, purity >95%.

Additional compounds prepared by the method of EXAMPLES 273 and 274:

| | | | | | | | | | |
|---|---|---|---|---|---|---|---|---|---|
| | | LCMS | | | | Caco-2 | | | human |
| | | retention | | LCMS | | Pₐₚₚ | | | Plasma |
| Example | Name | time | M + H⁺ | method | NMR | (nm/s) | SGF | SIF | stability |

| | |
|---|---|
| 275 | (10Z)-8,8,13,13-tetramethyl-22-[2- |
| | (propan-2-yloxy)ethyl]-6,15-dioxa- |
| | 17-thia-4,19,22- |
| | triazatricyclo[16.3.1.0\hat{ }{4,21}]docosa- |
| | 1(21),2,10,18-tetraene-7,14,20- |
| | trione |
| 276 | (10E)-8,8,13,13-tetramethyl-22-[2- |
| | (propan-2-yloxy)ethyl]-6,15-dioxa- |
| | 17-thia-4,19,22- |
| | triazatricyclo[16.3.1.0\hat{ }{4,21}]docosa- |
| | 1(21),2,10,18-tetraene-7,14,20- |
| | trione |

### Examples 277-280

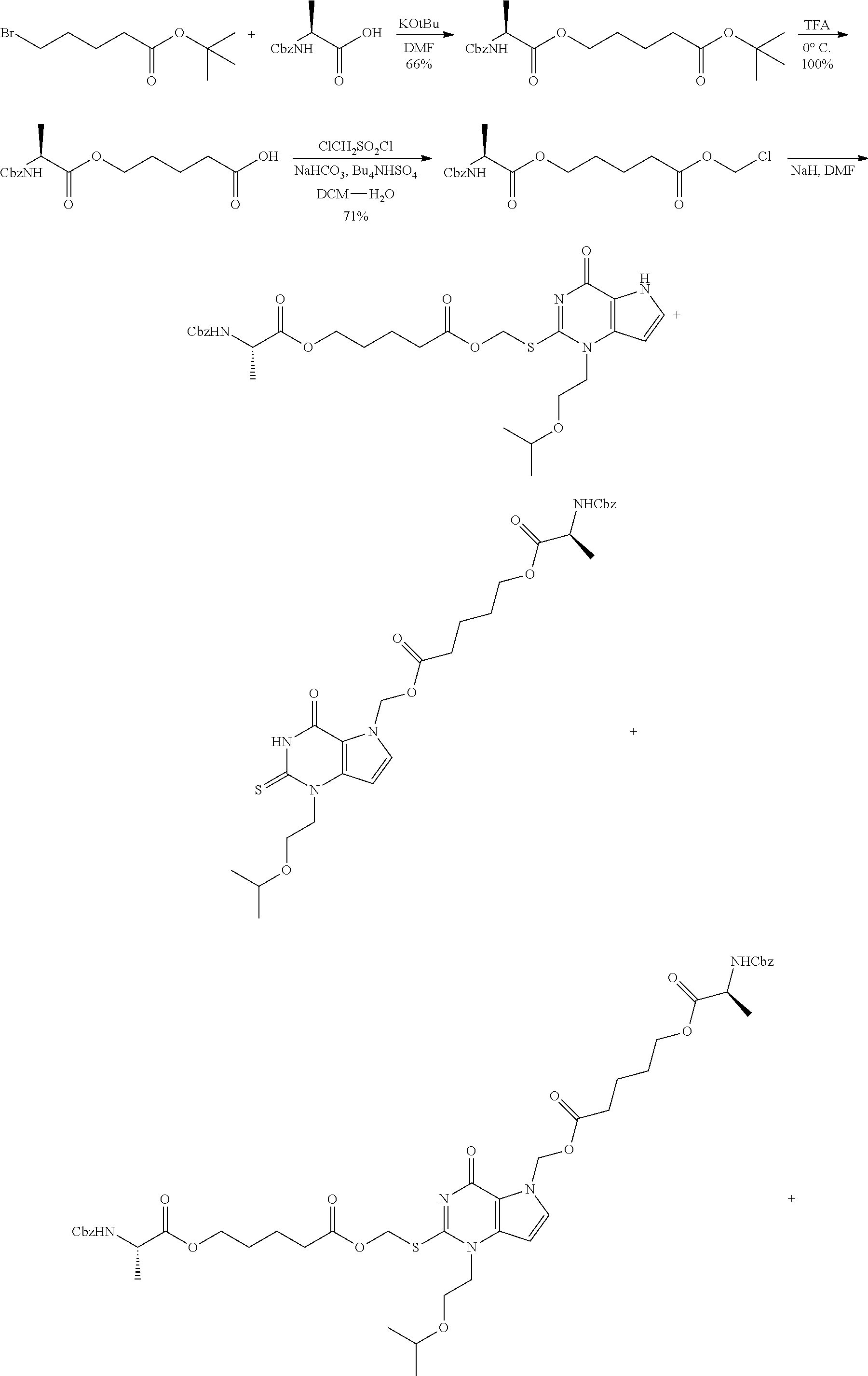

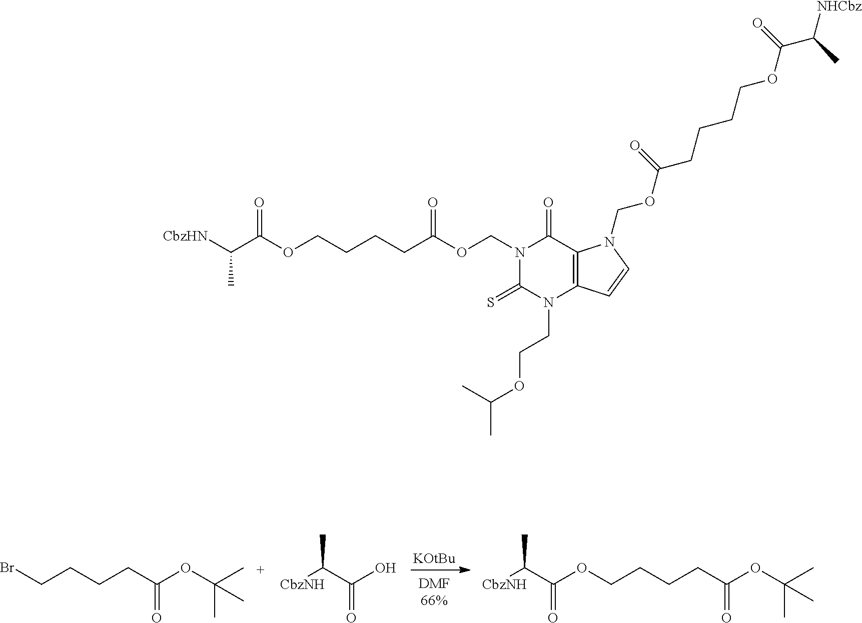

(S)-tert-Butyl 6-((2-(((benzyloxy)carbonyl)amino)-3-methylbutanoyl)oxy)pentanoate. To a solution of (S)-2-(((benzyloxy)carbonyl)amino)propanoic acid (1 g, 4.48 mmol) in DMF (15 mL) was added potassium tert-butoxide (0.55 g, 4.93 mmol) and the mixture was stirred at room temperature for 10 minutes. Tert-butyl 6-bromopentanoate (1.17 g, 4.93 mmol) was added and the reaction mixture was heated at 65° C. for 6 hours. After cooled to room temperature, the mixture was poured into saturated sodium bicarbonate aqueous solution and extracted by ethyl acetate (3×20 mL). The combined extracts were dried (Na₂SO₄), filtered and purified by silica chromatography eluted with 20% EtOAc/hexanes to get the product as a white solid (1.24 g, 66%).

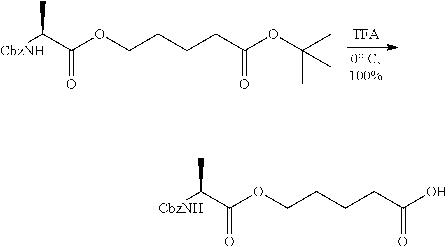

(S)-6-((2-(((benzyloxy)carbonyl)amino)-3-methyl butanoyl)oxy)pentanoic acid. (S)-tert-butyl 6-((2-(((benzyloxy)carbonyl)amino)-3-methylbutanoyl)oxy)pentanoate (1.24 g, 3.26 mmol) was added trifluoroacetic acid (5 mL) at 0° C. The solution was kept stirring at 0° C. for 2 hour and concentrated to give 0.95 g of the crude product as thick oil.

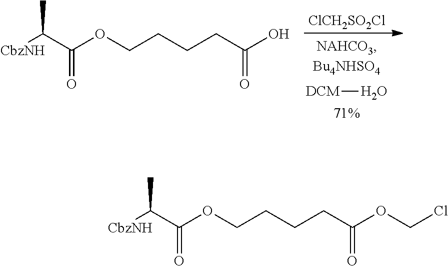

(S)-chloromethyl 6-((2-(((benzyloxy)carbonyl)amino)-3-methylbutanoyl)oxy)pentanoate. To a solution of (S)-6-((2-(((benzyloxy)carbonyl)amino)-3-methylbutanoyl)oxy)pentanoic acid (0.85 g, 2.64 mmol) in dichloromethane (8 mL) and water (8 mL) was added sodium bicarbonate (0.89 g, 10.6 mmol) and Bu₄HSO₄ (90 mg, 0.264 mmol) followed by dropwise addition of chloromethylchlorosulfonate (650 mg, 3.93 mmol). The mixture was stirred for 20 h, diluted with water (30 mL) and extracted with dichloromethane (50 mL). The organic layer was separated and washed with water (25 mL), dried (MgSO₄) and evaporated. The product mixture was purified by silica chromatography eluted with 20% EtOAc/hexanes to get the product as a white solid (500 mg, 51%).

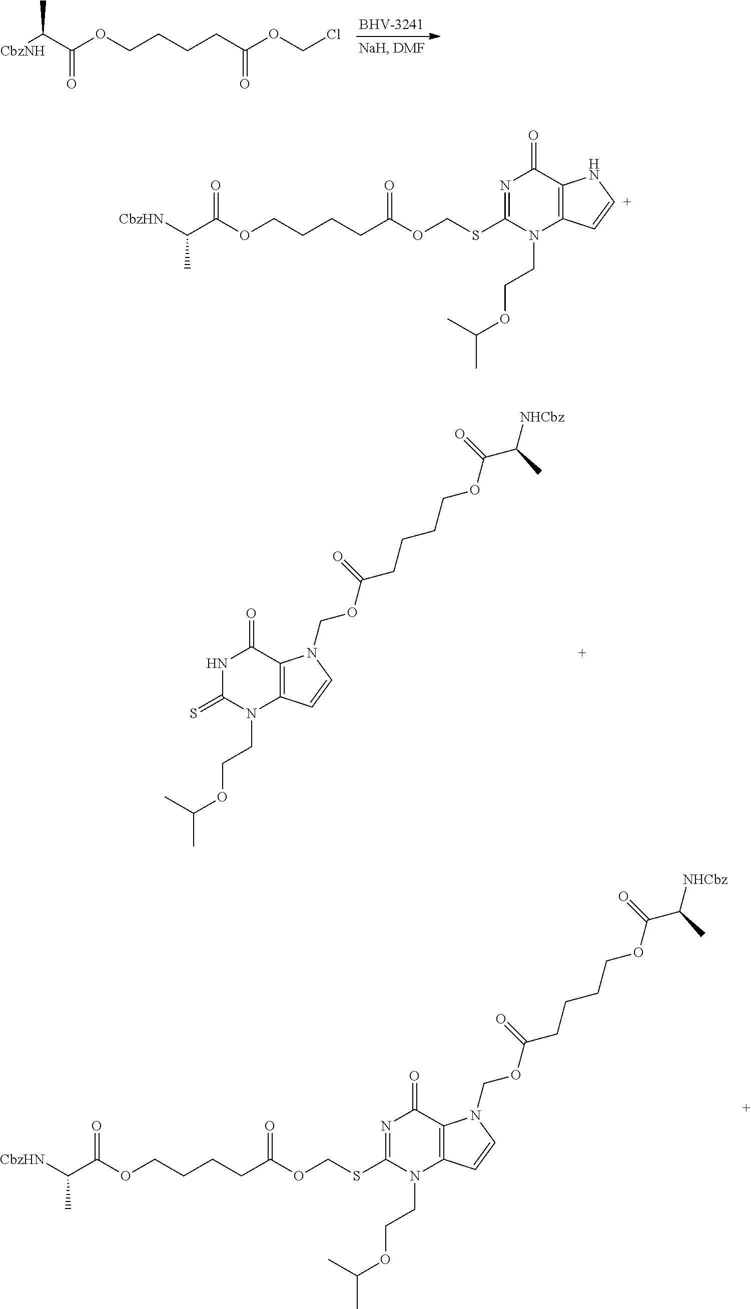

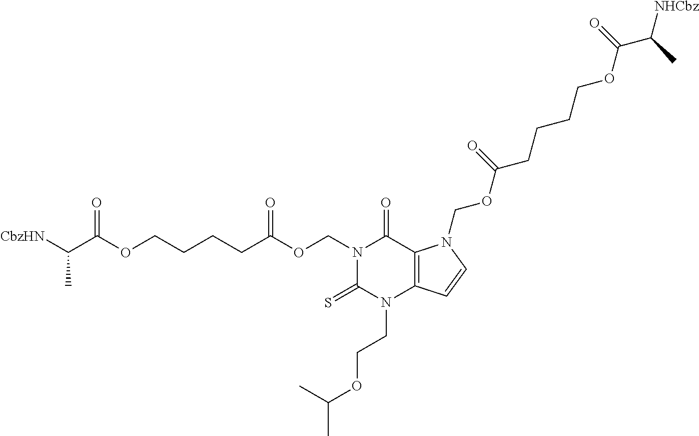 [SMILES: CC(C)OCCn1c(=S)n(COC(=O)CCCCOC(=O)[C@H](C)NC(=O)OCc2ccccc2)c(=O)c2c1ccn2COC(=O)CCCCOC(=O)[C@H](C)NC(=O)OCc1ccccc1]

A suspension of BHV-3241, 1-(2-isopropoxyethyl)-2-thioxo-2,3-dihydro-1H-pyrrolo[3,2-d]pyrimidin-4(5H)-one (100 mg, 0.395 mmol) in DMF (2 mL) was treated with sodium hydride (60% in mineral oil, 25.3 mg, 0.632 mmol) under N₂ and stirred for 30 minutes. (S)-Chloromethyl 6-((2-(((benzyloxy)carbonyl)amino)-3-methylbutanoyl)oxy)pentanoate (234 mg, 0.632 mmol) in DMF (0.5 mL) was added via syringe and the mixture was stirred for 18 h. The reaction was quenched with a few drops of saturated NH₄Cl, filtered with a syringe filter (45 μm) and the crude product mixture was purified by RP-HPLC (method B) to get the pure product separated from other isomers as a white solid (25 mg, 11%).

Additional compounds prepared similarly to EXAMPLES 277-280:

| | | | | | | | | | |
|---|---|---|---|---|---|---|---|---|---|
| | | LCMS | | | | Caco-2 | | | human |
| | | retention | | LCMS | | Pₐₚₚ | | | Plasma |
| Example | Name | time | M + H⁺ | method | NMR | (nm/s) | SGF | SIF | stability |

| | | | | | |
|---|---|---|---|---|---|
| 281 | {4-oxo-1-[2-(propan-2-yloxy)ethyl]- | 117 | 90% @ 1 h | 28% @ 1 h | 0% @ 1 h |
| | 2-sulfanylidene-1H,2H,3H,4H,5H- | | | | |
| | pyrrolo[3,2-d]pyrimidin-5-yl}methyl | | | | |
| | 2-{[(tert- | | | | |
| | butoxy)carbonyl]amino}acetate | | | | |
| 282 | [(5-{[(2-{[(tert- | | | | |
| | butoxy)carbonyl]amino}acetyl)oxy] | | | | |
| | methyl}-4-oxo-1-[2-(propan-2- | | | | |
| | yloxy)ethyl]-1H,4H,5H-pyrrolo[3,2- | | | | |
| | d]pyrimidin-2-yl) sulfanyl]methyl 2- | | | | |
| | {[(tert- | | | | |
| | butoxy)carbonyl]amino}acetate | | | | |
| 283 | (3-{[(2-{[(tert- | 57.8 | 82% @ 1 h | 1% @ 1 h | 26% @ 1 h |
| | butoxy)carbonyl]amino}acetyl)oxy] | | | | |
| | methyl}-4-oxo-1-[2-(propan-2- | | | | |
| | yloxy)ethyl]-2-sulfanylidene- | | | | |
| | 1H,2H,3H,4H,5H-pyrrolo[3,2- | | | | |
| | d]pyrimidin-5-yl)methyl 2-{[(tert- | | | | |
| | butoxy)carbonyl]amino}acetate | | | | |
| 284 | trifluoroacetic acid {4-oxo-1-[2- | 15.5 | 100% @ 1 h | 6% @ 1 h | 0% @ 1 h |
| | (propan-2-yloxy)ethyl]-2- | | | | |
| | sulfanylidene-1H,2H,3H,4H,5H- | | | | |
| | pyrrolo[3,2-d]pyrimidin-5-yl}methyl | | | | |
| | 2-aminoacetate | | | | |

### Examples 285-288

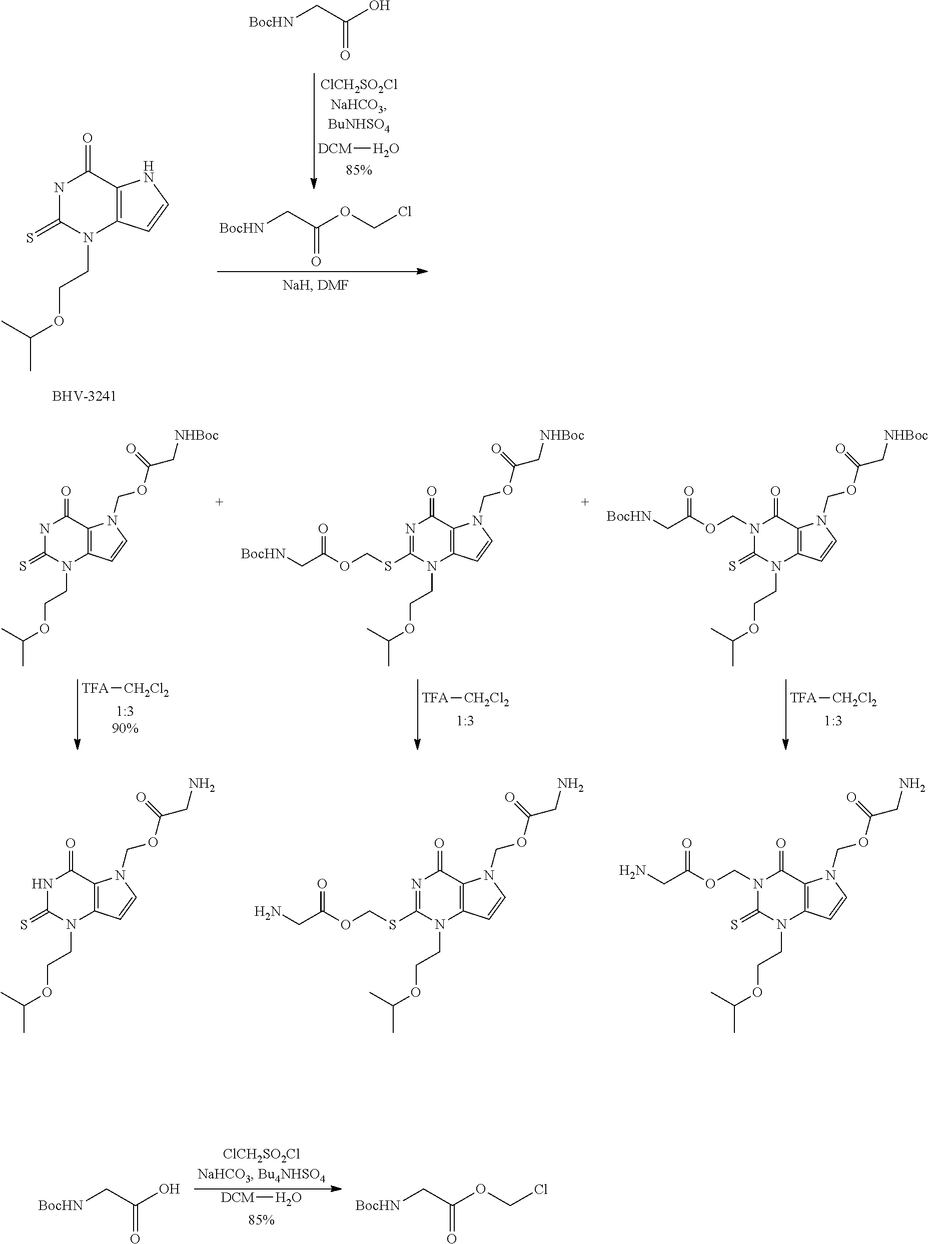

Chloromethyl 2-((tert-butoxycarbonyl)amino)acetate. To a solution of 2-((tert-butoxycarbonyl)amino)acetic acid (1.75 g, 10 mmol) in dichloromethane (20 mL) and water (20 mL) was added sodium bicarbonate (3.36 g, 40 mmol) and Bu₄HSO₄ (340 mg, 1 mmol) followed by dropwise addition of chloromethylchlorosulfonate (1.2 mL, 12 mmol). The mixture was stirred for 20 h, diluted with water (30 mL) and extracted with dichloromethane (50 mL). The organic layer was separated and washed with water (25 mL), dried (MgSO₄) and evaporated. The product mixture was purified by silica chromatography eluted with 30% EtOAc/hexanes to get the product as a white solid (1.9 g, 85%).

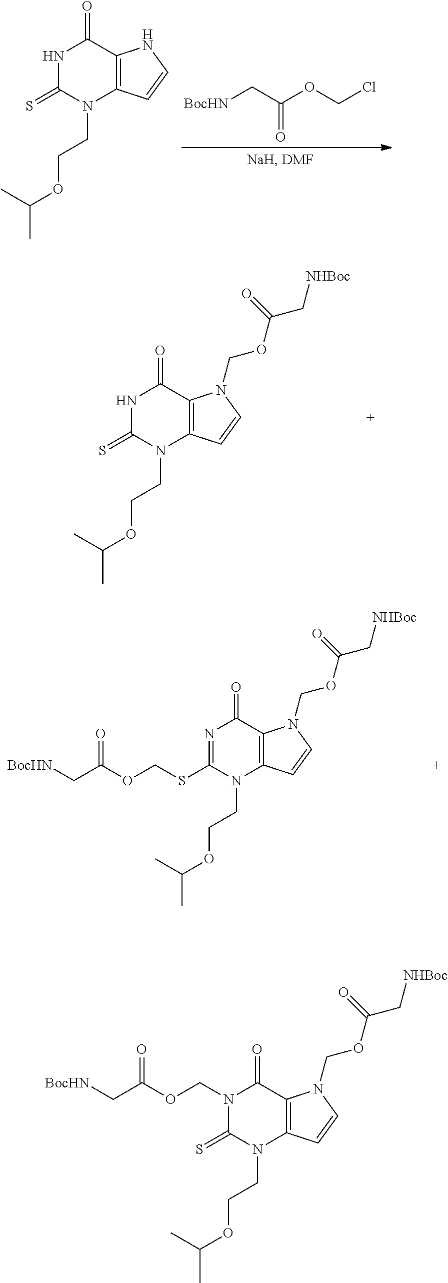 [SMILES: CC(C)OCCn1c(=S)[nH]c(=O)c2[nH]ccc21 ; CC(C)(C)OC(=O)NCC(=O)OCCl ; CC(C)OCCn1c(=S)[nH]c(=O)c2c1ccn2COC(=O)CNC(=O)OC(C)(C)C ; CC(C)OCCn1c(SCOC(=O)CNC(=O)OC(C)(C)C)nc(=O)c2c1ccn2COC(=O)CNC(=O)OC(C)(C)C ; CC(C)OCCn1c(=S)n(COC(=O)CNC(=O)OC(C)(C)C)c(=O)c2c1ccn2COC(=O)CNC(=O)OC(C)(C)C]

1-(2-isopropoxyethyl)-2-thioxo-2,3-dihydro-1H-pyrrolo[3,2-d]pyrimidin-4(5H)-one, by the same procedure as EXAMPLE 2, on 250 mg scale purified by Method B as white solid.

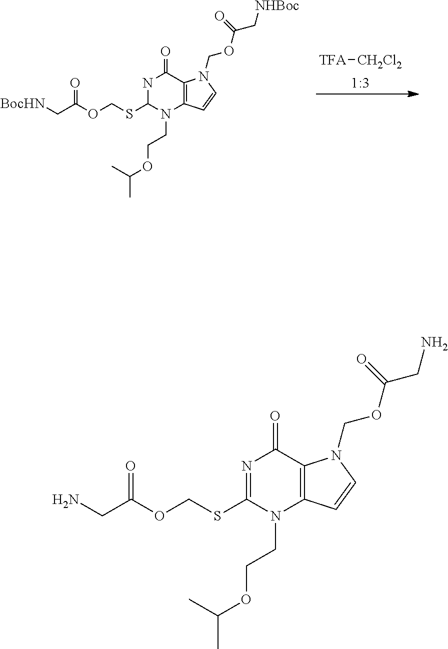

To a solution of (20 mg, 0.032 mmol) in dichloromethane (1.5 mL) was added trifluoroacetic acid (0.55 mL). The reaction mixture was complete upon LC-MS monitoring after stirring for 30 min. The solution was concentrated and the residue was purified by RP-HPLC (method B) to get the pure product as a white solid (10 mg, 59%).

Additional compounds prepared similarly to EXAMPLES 285-288:

| | | | | | | | | | |
|---|---|---|---|---|---|---|---|---|---|
| | | LCMS | | | | Caco-2 | | | human |
| | | retention | | LCMS | | Pₐₚₚ | | | Plasma |
| Example | Name | time | M + H⁺ | method | NMR | (nm/s) | SGF | SIF | stability |

| | | | | | | | |
|---|---|---|---|---|---|---|---|
| 289 | bis(trifluoroacetic acid) [3-({[(2S)-2- | | | <3.66 | 100% @ 1 h | 97% @ 1 h | 39% @ 1 h |
| | aminopropanoyl]oxy}methyl)-4-oxo- | | | | | | |
| | 1-[2-(propan-2-yloxy)ethyl]-2- | | | | | | |
| | sulfanylidene-1H,2H,3H,4H,5H- | | | | | | |
| | pyrrolo[3,2-d]pyrimidin-5-yl]methyl | | | | | | |
| | (2S)-2-aminopropanoate | | | | | | |
| 290 | trifluoroacetic acid [5- | | | unstable | 98% @ 1 h | 10% @ 1 h | |
| | (hydroxymethyl)-4-oxo-1-[2- | | | | | | |
| | (propan-2-yloxy)ethyl]-2- | | | | | | |
| | sulfanylidene-1H,2H,3H,4H,5H- | | | | | | |
| | pyrrolo[3,2-d]pyrimidin-3-yl]methyl | | | | | | |
| | (2S)-2-aminopropanoate | | | | | | |
| 291 | {4-oxo-1-[2-(propan-2-yloxy)ethyl]- | | | 219 | 66% @ 1 h | 30% @ 1 h | 0% @ 1 h |
| | 2-sulfanylidene-1H,2H,3H,4H,5H- | | | | | | |
| | pyrrolo[3,2-d]pyrimidin-5-yl}methyl | | | | | | |
| | (2R)-2-{[(tert- | | | | | | |
| | butoxy)carbonyl]amino}propanoate | | | | | | |
| 292 | trifluoroacetic acid {4-oxo-1-[2- | | | <10.6 | 99% @ 1 h | 7% @ 1 h | 20% @ 1 h |
| | (propan-2-yloxy)ethyl]-2- | | | | | | |
| | sulfanylidene-1H,2H,3H,4H,5H- | | | | | | |
| | pyrrolo[3,2-d]pyrimidin-5-yl}methyl | | | | | | |
| | (2R)-2-aminopropanoate | | | | | | |
| 293 | bis(trifluoroacetic acid) [3-({[(2R)-2- | | | <10.6 | 100% @ 1 h | 29% @ 1 h | 25% @ 1 h |
| | aminopropanoyl]oxy}methyl)-4-oxo- | | | | | | |
| | 1-[2-(propan-2-yloxy)ethyl]-2- | | | | | | |
| | sulfanylidene-1H,2H,3H,4H,5H- | | | | | | |
| | pyrrolo[3,2-d]pyrimidin-5-yl]methyl | | | | | | |
| | (2R)-2-aminopropanoate | | | | | | |
| 294 | {4-oxo-1-[2-(propan-2-yloxy)ethyl]- | | | 268 | 98% @ 1 h | 3% @ 1 h | 98% @ 1 h |
| | 2-sulfanylidene-1H,2H,3H,4H,5H- | | | | | | |
| | pyrrolo[3,2-d]pyrimidin-5-yl}methyl | | | | | | |
| | (2S)-2-{[(tert- | | | | | | |
| | butoxy)carbonyl]amino}-3- | | | | | | |
| | methylbutanoate | | | | | | |
| 295 | {[5-({[(2S)-2-{[(tert- | | | 170 | 58% @ 1 h | 0% @ 1 h | 81% @ 1 h |
| | butoxy) carbonyl]amino}-3- | | | | | | |
| | methylbutanoyl]oxy}methyl)-4-oxo- | | | | | | |
| | 1-[2-(propan-2-yloxy)ethyl]- | | | | | | |
| | 1H,4H,5H-pyrrolo[3,2-d]pyrimidin-2- | | | | | | |
| | yl]sulfanyl}methyl (2S)-2-{[(tert- | | | | | | |
| | butoxy)carbonyl]amino}-3- | | | | | | |
| | methylbutanoate | | | | | | |
| 296 | trifluoroacetic acid {4-oxo-1-[2- | | | 11.8 | 97% @ 1 h | 43% @ 1 h | 12% @ 1 h |
| | (propan-2-yloxy)ethyl]-2- | | | | | | |
| | sulfanylidene-1H,2H,3H,4H,5H- | | | | | | |
| | pyrrolo[3,2-d]pyrimidin-5-yl}methyl | | | | | | |
| | (2S)-2-amino-3-methylbutanoate | | | | | | |
| 297 | bis(trifluoroacetic acid) [3-({[(2S)-2- | | | 4.62 | 100% @ 1 h | 6% @ 1 h | 6% @ 1 h |
| | amino-3- | | | | | | |
| | methylbutanoyl]oxy}methyl)-4-oxo- | | | | | | |
| | 1-[2-(propan-2-yloxy)ethyl]-2- | | | | | | |
| | sulfanylidene-1H,2H,3H,4H,5H- | | | | | | |
| | pyrrolo[3,2-d]pyrimidin-5-yl]methyl | | | | | | |
| | (2S)-2-amino-3-methylbutanoate | | | | | | |
| 298 | {4-oxo-1-[2-(propan-2-yloxy)ethyl]- | | | 335 | 99% @ 1 h | 59% @ 1 h | 99% @ 1 h |
| | 2-sulfanylidene-1H,2H,3H,4H,5H- | | | | | | |
| | pyrrolo[3,2-d]pyrimidin-5-yl}methyl | | | | | | |
| | (2R)-2-{[(tert- | | | | | | |
| | butoxy)carbonyl]amino}-3- | | | | | | |
| | methylbutanoate | | | | | | |
| 299 | {[5-({[(2R)-2-{[(tert- | | | 158 | 54% @ 1 h | 27% @ 1 h | 94% @ 1 h |
| | butoxy)carbonyl]amino}-3- | | | | | | |
| | methylbutanoyl]oxy}methyl)-4-oxo- | | | | | | |
| | 1-[2-(propan-2-yloxy)ethyl]- | | | | | | |
| | 1H,4H,5H-pyrrolo[3,2-d]pyrimidin-2- | | | | | | |
| | yl]sulfanyl}methyl (2R)-2-{[(tert- | | | | | | |
| | butoxy)carbonyl]amino}-3- | | | | | | |
| | methylbutanoate | | | | | | |
| 300 | trifluoroacetic acid {4-oxo-1-[2- | | | 129 | 100% @ 1 h | 70% @ 1 h | 2% @ 1 h |
| | (propan-2-yloxy)ethyl]-2- | | | | | | |
| | sulfanylidene-1H,2H,3H,4H,5H- | | | | | | |
| | pyrrolo[3,2-d]pyrimidin-5-yl}methyl | | | | | | |
| | (2R)-2-amino-3-methylbutanoate | | | | | | |
| 301 | bis(trifluoroacetic acid) [3-({[(2R)- | | | 17.2 | 97% @ 1 h | 10% @ 1 h | 5% @ 1 h |
| | 2-amino-3- | | | | | | |
| | methylbutanoyl]oxy}methyl)-4-oxo- | | | | | | |
| | 1-[2-(propan-2-yloxy)ethyl]-2- | | | | | | |
| | sulfanylidene-1H,2H,3H,4H,5H- | | | | | | |
| | pyrrolo [3,2-d]pyrimidin-5-yl]methyl | | | | | | |
| | (2R)-2-amino-3-methylbutanoate | | | | | | |
| 302 | {4-oxo-1-[2-(propan-2-yloxy)ethyl]- | | | 44.8 | 97% @ 1 h | 5% @ 1 h | 67% @ 1 h |
| | 2-sulfanylidene-1H,2H,3H,4H,5H- | | | | | | |
| | pyrrolo[3,2-d]pyrimidin-5-yl}methyl | | | | | | |
| | (2S)-2-{[(tert- | | | | | | |
| | butoxy)carbonyl]amino}-3- | | | | | | |
| | phenylpropanoate | | | | | | |
| 303 | {[5-({[(2S)-2-{[(tert- | | | 55.6 | 46% @ 1 h | 1% @ 1 h | 28% @ 1 h |
| | butoxy)carbonyl]amino}-3- | | | | | | |
| | phenylpropanoyl]oxy}methyl)-4- | | | | | | |
| | oxo-1-[2-(propan-2-yloxy)ethyl]- | | | | | | |
| | 1H,4H,5H-pyrrolo[3,2-d]pyrimidin-2- | | | | | | |
| | yl]sulfanyl}methyl (2S)-2-{[(tert- | | | | | | |
| | butoxy)carbonyl]amino}-3- | | | | | | |
| | phenylpropanoate | | | | | | |
| 304 | [3-({[(2S)-2-{[(tert- | | | 3.4 | 44% @ 1 h | 67% @ 1 h | 99% @ 1 h |
| | butoxy)carbonyl]amino}-3- | | | | | | |
| | phenylpropanoyl]oxy}methyl)-4- | | | | | | |
| | oxo-1-[2-(propan-2-yloxy)ethyl]-2- | | | | | | |
| | sulfanylidene-1H,2H,3H,4H,5H- | | | | | | |
| | pyrrolo[3,2-d]pyrimidin-5-yl]methyl | | | | | | |
| | (2S)-2-{[(tert- | | | | | | |
| | butoxy)carbonyl]amino}-3- | | | | | | |
| | phenylpropanoate | | | | | | |
| 305 | {4-oxo-1-[2-(propan-2-yloxy)ethyl]- | | | 38.4 | 73% @ 1 h | 1% @ 1 h | 39% @ 1 h |
| | 2-sulfanylidene-1H,2H,3H,4H,5H- | | | | | | |
| | pyrrolo [3,2-d]pyrimidin-5-yl}methyl | | | | | | |
| | (2R)-2-{[(tert- | | | | | | |
| | butoxy)carbonyl]amino}-3- | | | | | | |
| | phenylpropanoate | | | | | | |
| 306 | {[5-({[(2R)-2-{[(tert- | | | 38 | 41% @ 1 h | 1% @ 1 h | 72% @ 1 h |
| | butoxy)carbonyl]amino}-3- | | | | | | |
| | phenylpropanoyl]oxy}methyl)-4- | | | | | | |
| | oxo-1-[2-(propan-2-yloxy)ethyl]- | | | | | | |
| | 1H,4H,5H-pyrrolo[3,2-d]pyrimidin-2- | | | | | | |
| | yl]sulfanyl}methyl (2R)-2-{[(tert- | | | | | | |
| | butoxy)carbonyl]amino}-3- | | | | | | |
| | phenylpropanoate | | | | | | |
| 307 | [3-({[(2R)-2-{[(tert- | | | 3.34 | 26% @ 1 h | 43% @ 1 h | 99% @ 1 h |
| | butoxy)carbonyl]amino}-3- | | | | | | |
| | phenylpropanoyl]oxy}methyl)-4- | | | | | | |
| | oxo-1-[2-(propan-2-yloxy)ethyl]-2- | | | | | | |
| | sulfanylidene-1H,2H,3H,4H,5H- | | | | | | |
| | pyrrolo[3,2-d]pyrimidin-5-yl]methyl | | | | | | |
| | (2R)-2-{[(tert- | | | | | | |
| | butoxy)carbonyl]amino}-3- | | | | | | |
| | phenylpropanoate | | | | | | |
| 308 | trifluoroacetic acid {4-oxo-1-[2- | | | <3.64 | 97% @ 1 h | 44% @ 1 h | 2.5% @ 1 h |
| | (propan-2-yloxy)ethyl]-2- | | | | | | |
| | sulfanylidene-1H,2H,3H,4H,5H- | | | | | | |
| | pyrrolo[3,2-d]pyrimidin-5-yl}methyl | | | | | | |
| | (2S)-2-amino-3-phenylpropanoate | | | | | | |
| 309 | bis(trifluoroacetic acid) {[5- | | | <1.23 | 98% @ 1 h | 86% @ 1 h | 5% @ 1 h |
| | ({[(2S)-2-amino-3- | | | | | | |
| | phenylpropanoyl]oxy}methyl)-4- | | | | | | |
| | oxo-1-[2-(propan-2-yloxy)ethyl]- | | | | | | |
| | 1H,4H,5H-pyrrolo[3,2-d]pyrimidin-2- | | | | | | |
| | yl]sulfanyl}methyl (2S)-2-amino-3- | | | | | | |
| | phenylpropanoate | | | | | | |
| 310 | trifluoroacetic acid ({4-oxo-1-[2- | | | | 99% @ 1 h | 32% @ 1 h | 4% @ 1 h |
| | (propan-2-yloxy)ethyl]-1H,4H,5H- | | | | | | |
| | pyrrolo[3,2-d]pyrimidin-2- | | | | | | |
| | yl}sulfanyl)methyl (2S)-2-amino-3- | | | | | | |
| | phenylpropanoate | | | | | | |
| 311 | trifluoroacetic acid {4-oxo-1-[2- | | | 54.4 | 98% @ 1 h | 7% @ 1 h | 4% @ 1 h |
| | (propan-2-yloxy)ethyl]-2- | | | | | | |
| | sulfanylidene-1H,2H,3H,4H,5H- | | | | | | |
| | pyrrolo[3,2-d]pyrimidin-5-yl}methyl | | | | | | |
| | (2R)-2-amino-3-phenylpropanoate | | | | | | |
| 312 | bis(trifluoroacetic acid) {[5- | | | >0.994 | 99% @ 1 h | 29% @ 1 h | 24% @ 1 h |
| | ({[(2R)-2-amino-3- | | | | | | |
| | phenylpropanoyl]oxy}methyl)-4- | | | | | | |
| | oxo-1-[2-(propan-2-yloxy)ethyl]- | | | | | | |
| | 1H,4H,5H-pyrrolo[3,2-d]pyrimidin-2- | | | | | | |
| | yl]sulfanyl} methyl (2R)-2-amino-3- | | | | | | |
| | phenylpropanoate | | | | | | |
| 313 | bis(trifluoroacetic acid) [3- | | | 4.9 | 88% @ 1 h | 51% @ 1 h | 99% @ 1 h |
| | ({[(2R)-2-amino-3- | | | | | | |
| | phenylpropanoyl]oxy}methyl)-4- | | | | | | |
| | oxo-1-[2-(propan-2-yloxy)ethyl]-2- | | | | | | |
| | sulfanylidene-1H,2H,3H,4H,5H- | | | | | | |
| | pyrrolo[3,2-d]pyrimidin-5-yl]methyl | | | | | | |
| | (2R)-2-amino-3-phenylpropanoate | | | | | | |
| 314 | {4-oxo-1-[2-(propan-2-yloxy)ethyl]- | | | | | | |
| | 2-sulfanylidene-1H,2H,3H,4H,5H- | | | | | | |
| | pyrrolo[3,2-d]pyrimidin-5-yl}methyl | | | | | | |
| | (2S)-2-{[(tert- | | | | | | |
| | butoxy)carbonyl]amino}-4- | | | | | | |
| | methylpentanoate | | | | | | |
| 315 | {[5-({[(2S)-2-{[ (tert- | | | 141 | 78%@ 1 h | 5%@ 1 h | 7%@ 1 h |
| | butoxy)carbonyl]amino}-4- | | | | | | |
| | methylpentanoyl]oxy}methyl)-4-oxo- | | | | | | |
| | 1-[2-(propan-2-yloxy)ethyl]- | | | | | | |
| | 1H,4H,5H-pyrrolo[3,2-d]pyrimidin-2- | | | | | | |
| | yl]sulfanyl} methyl (2S)-2-{[(tert- | | | | | | |
| | butoxy)carbonyl]amino}-4- | | | | | | |
| | methylpentanoate | | | | | | |
| 316 | [3-({[(2S)-2-{[(tert- | | | 76 | 99%@ 1 h | 6%@ 1 h | 37%@ 1 h |
| | butoxy)carbonyl]amino}-4- | | | | | | |
| | methylpentanoyl]oxy}methyl)-4-oxo- | | | | | | |
| | 1-[2-(propan-2-yloxy)ethyl]-2- | | | | | | |
| | sulfanylidene-1H,2H,3H,4H,5H- | | | | | | |
| | pyrrolo [3,2-d]pyrimidin-5-yl]methyl | | | | | | |
| | (2S)-2-{[(tert- | | | | | | |
| | butoxy)carbonyl]amino}-4- | | | | | | |
| | methylpentanoate | | | | | | |
| 317 | {4-oxo-1-[2-(propan-2-yloxy)ethyl]- | | | 0.93 | 56%@ 1 h | 30%@ 1 h | 99%@ 1 h |
| | 2-sulfanylidene-1H,2H,3H,4H,5H- | | | | | | |
| | pyrrolo[3,2-d]pyrimidin-5-yl}methyl | | | | | | |
| | (2R)-2-{[(tert- | | | | | | |
| | butoxy)carbonyl]amino}-4- | | | | | | |
| | methylpentanoate | | | | | | |
| 318 | {[5-({[(2R)-2-{[(tert- | | | 254 | 81%@ 1 h | 2%@ 1 h | 85%@ 1 h |
| | butoxy)carbonyl]amino}-4- | | | | | | |
| | methylpentanoyl]oxy}methyl)-4-oxo- | | | | | | |
| | 1-[2-(propan-2-yloxy)ethyl]- | | | | | | |
| | 1H,4H,5H-pyrrolo[3,2-d]pyrimidin-2- | | | | | | |
| | yl]sulfanyl}methyl (2R)-2-{[(tert- | | | | | | |
| | butoxy)carbonyl]amino}-4- | | | | | | |
| | methylpentanoate | | | | | | |
| 319 | [3-({[(2R)-2-{[(tert- | | | 105 | 34%@ 1 h | 0%@ 1 h | 42%@ 1 h |
| | butoxy)carbonyl]amino}-4- | | | | | | |
| | methylpentanoyl]oxy}methyl)-4-oxo- | | | | | | |
| | 1-[2-(propan-2-yloxy)ethyl]-2- | | | | | | |
| | sulfanylidene-1H,2H,3H,4H,5H- | | | | | | |
| | pyrrolo[3,2-d]pyrimidin-5-yl]methyl | | | | | | |
| | (2R)-2-{[(tert- | | | | | | |
| | butoxy)carbonyl]amino}-4- | | | | | | |
| | methylpentanoate | | | | | | |
| 320 | trifluoroacetic acid {4-oxo-1-[2- | | | <2.59 | 48%@ 1 h | 42%@ 1 h | 98%@ 1 h |
| | (propan-2-yloxy)ethyl]-2- | | | | | | |
| | sulfanylidene-1H,2H,3H,4H,5H- | | | | | | |
| | pyrrolo[3,2-d]pyrimidin-5-yl}methyl | | | | | | |
| | (2S)-2-amino-4-methylpentanoate | | | | | | |
| 321 | bis(trifluoroacetic acid) {[5- | | | <3.65 | 92%@ 1 h | 2%@ 1 h | 1%@ 1 h |
| | ({[(2S)-2-amino-4- | | | | | | |
| | methylpentanoyl]oxy}methyl)-4-oxo- | | | | | | |
| | 1-[2-(propan-2-yloxy)ethyl]- | | | | | | |
| | 1H,4H,5H-pyrrolo[3,2-d]pyrimidin-2- | | | | | | |
| | yl]sulfanyl}methyl (2S)-2-amino-4- | | | | | | |
| | methylpentanoate | | | | | | |
| 322 | bis(trifluoroacetic acid) [3- | | | <1.06 | 56%@ 1 h | 61%@ 1 h | 64%@ 1 h |
| | ({[(2S)-2-amino-4- | | | | | | |
| | methylpentanoyl]oxy}methyl)-4-oxo- | | | | | | |
| | 1-[2-(propan-2-yloxy)ethyl]-2- | | | | | | |
| | sulfanylidene-1H,2H,3H,4H,5H- | | | | | | |
| | pyrrolo[3,2-d]pyrimidin-5-yl]methyl | | | | | | |
| | (2S)-2-amino-4-methylpentanoate | | | | | | |
| 323 | trifluoroacetic acid [3- | | | <3.55 | 95%@ 1 h | 52%@ 1 h | 12%@ 1 h |
| | (hydroxymethyl)-4-oxo-1-[2- | | | | | | |
| | (propan-2-yloxy)ethyl]-2- | | | | | | |
| | sulfanylidene-1H,2H,3H,4H,5H- | | | | | | |
| | pyrrolo[3,2-d]pyrimidin-5-yl]methyl | | | | | | |
| | (2S)-2-amino-4-methylpentanoate | | | | | | |
| 324 | trifluoroacetic acid {4-oxo-1-[2- | | | | 98%@ 1 h | 24%@ 1 h | 1%@ 1 h |
| | (propan-2-yloxy)ethyl]-2- | | | | | | |
| | sulfanylidene-1H,2H,3H,4H,5H- | | | | | | |
| | pyrrolo[3,2-d]pyrimidin-5-yl}methyl | | | | | | |
| | (2R)-2-amino-4-methylpentanoate | | | | | | |
| 325 | bis(trifluoroacetic acid) {[5- | | | 9.08 | 99%@ 1 h | 3%@ 1 h | 1%@ 1 h |
| | ({[(2R)-2-amino-4- | | | | | | |
| | methylpentanoyl]oxy}methyl)-4-oxo- | | | | | | |
| | 1-[2-(propan-2-yloxy)ethyl]- | | | | | | |
| | 1H,4H,5H-pyrrolo[3,2-d]pyrimidin-2- | | | | | | |
| | yl]sulfanyl}methyl (2R)-2-amino-4- | | | | | | |
| | methylpentanoate | | | | | | |
| 326 | bis(trifluoroacetic acid) [3- | | | <3.38 | 97%@ 1 h | 9%@ 1 h | 17%@ 1 h |
| | ({[(2R)-2-amino-4- | | | | | | |
| | methylpentanoyl]oxy}methyl)-4-oxo- | | | | | | |
| | 1-[2-(propan-2-yloxy)ethyl]-2- | | | | | | |
| | sulfanylidene-1H,2H,3H,4H,5H- | | | | | | |
| | pyrrolo [3,2-d]pyrimidin-5-yl]methyl | | | | | | |
| | (2R)-2-amino-4-methylpentanoate | | | | | | |
| 327 | trifluoroacetic acid [3- | | | <10.3 | 100%@ 1 h | 49%@ 1 h | 21%@ 1 h |
| | (hydroxymethyl)-4-oxo-1-[2- | | | | | | |
| | (propan-2-yloxy)ethyl]-2- | | | | | | |
| | sulfanylidene-1H,2H,3H,4H,5H- | | | | | | |
| | pyrrolo[3,2-d]pyrimidin-5-yl]methyl | | | | | | |
| | (2R)-2-amino-4-methylpentanoate | | | | | | |
| 328 | {4-oxo-1-[2-(propan-2-yloxy)ethyl]- | | | | 97%@ 1 h | 6%@ 1 h | 11%@ 1 h |
| | 2-sulfanylidene-1H,2H,3H,4H,5H- | | | | | | |
| | pyrrolo[3,2-d]pyrimidin-5-yl}methyl | | | | | | |
| | 5-[(2-{[(tert- | | | | | | |
| | butoxy)carbonyl]amino}acetyl)oxy] | | | | | | |
| | pentanoate | | | | | | |
| 329 | ({5-[{{5-[(2-{[(tert- | | | 59.6 | 95% @ 1 h | 35% @ 1 h | 1% @ 1 h |
| | butoxy)carbonyl]amino}acetyl)oxy] | | | | | | |
| | pentanoyl}oxy)methyl]-4-oxo-1-[2- | | | | | | |
| | (propan-2-yloxy)ethyl]-1H,4H,5H- | | | | | | |
| | pyrrolo[3,2-d]pyrimidin-2- | | | | | | |
| | yl}sulfanyl)methyl 5-[(2-{[(tert- | | | | | | |
| | butoxy)carbonyl]amino}acetyl)oxy] | | | | | | |
| | pentanoate | | | | | | |
| 330 | trifluoroacetic acid {4-oxo-1-[2- | | | 6.41 | 84% @ 1 h | 7% @ 1 h | 2% @ 1 h |
| | (propan-2-yloxy)ethyl]-2- | | | | | | |
| | sulfanylidene-1H,2H,3H,4H,5H- | | | | | | |
| | pyrrolo[3,2-d]pyrimidin-5-yl}methyl | | | | | | |
| | 5-[(2-aminoacetyl)oxy]pentanoate | | | | | | |
| 331 | bis (trifluoroacetic acid) ({5- | | | 36.8 | 93% @ 1 h | 1% @ 1 h | 1% @ 1 h |
| | [{{5-[(2- | | | | | | |
| | aminoacetyl)oxy]pentanoyl}oxy)meth- | | | | | | |
| | yl]-4-oxo-1-[2-(propan-2- | | | | | | |
| | yloxy)ethyl]-1H,4H,5H-pyrrolo[3,2- | | | | | | |
| | d]pyrimidin-2-yl}sulfanyl)methyl 5- | | | | | | |
| | [(2-aminoacetyl)oxy]pentanoate | | | | | | |
| 332 | bis (trifluoroacetic acid) {3- | | | <0.73 | 91% @ 1 h | 4% @ 1 h | 4% @ 1 h |
| | [{{5-[(2- | | | | | | |
| | aminoacetyl)oxy]pentanoyl}oxy)meth- | | | | | | |
| | yl]-4-oxo-1-[2-(propan-2- | | | | | | |
| | yloxy)ethyl]-2-sulfanylidene- | | | | | | |
| | 1H,2H,3H,4H,5H-pyrrolo[3,2- | | | | | | |
| | d]pyrimidin-5-yl}methyl 5-[(2- | | | | | | |
| | aminoacetyl)oxy]pentanoate | | | | | | |
| 333 | {4-oxo-1-[2-(propan-2-yloxy)ethyl]- | | | <0.72 | 98% @ 1 h | 4% @ 1 h | 5% @ 1 h |
| | 2-sulfanylidene-1H,2H,3H,4H,5H- | | | | | | |
| | pyrrolo[3,2-d]pyrimidin-5-yl}methyl | | | | | | |
| | 5-{[(2S)-2-{[(tert- | | | | | | |
| | butoxy)carbonyl]amino}-3- | | | | | | |
| | methylbutanoyl]oxy}pentanoate | | | | | | |
| 334 | {4-oxo-1-[2-(propan-2-yloxy)ethyl]- | | | 62.6 | 90 | 2 | 58% remaining |
| | 2-sulfanylidene-1H,2H,3H,4H,5H- | | | | | | at 1 h |
| | pyrrolo[3,2-d]pyrimidin-5-yl}methyl | | | | | | |
| | 5-{[(2S)-2-{[(tert- | | | | | | |
| | butoxy)carbonyl]amino}-3- | | | | | | |
| | phenylpropanoyl]oxy}pentanoate | | | | | | |
| 335 | trifluoroacetic acid {4-oxo-1-[2- | | | 33.2 | 99 | 9 | 3% remaining |
| | (propan-2-yloxy)ethyl]-2- | | | | | | at 1 h |
| | sulfanylidene-1H,2H,3H,4H,5H- | | | | | | |
| | pyrrolo[3,2-d]pyrimidin-5-yl}methyl | | | | | | |
| | 5-{[(2S)-2-amino-3- | | | | | | |
| | methylbutanoyl]oxy}pentanoate | | | | | | |
| 336 | 2-[(hydroxymethyl)sulfanyl]-1-[2- | | | 18.2 | 93 | 6 | 73% remaining |
| | (propan-2-yloxy)ethyl]-1H,4H,5H- | | | | | | at 1 h |
| | pyrrolo[3,2-d]pyrimidin-4-one | | | | | | |
| 337 | bis(trifluoroacetic acid) (3-{[(5- | | | — | 98 | 30 | 60% remaining |
| | {[(2S)-2-amino-3- | | | | | | at 1 h |
| | methylbutanoyl]oxy}pentanoyl)oxy] | | | | | | |
| | methyl}-4-oxo-1-[2-(propan-2- | | | | | | |
| | yloxy)ethyl]-2-sulfanylidene- | | | | | | |
| | 1H,2H,3H,4H,5H-pyrrolo[3,2- | | | | | | |
| | d]pyrimidin-5-yl)methyl 5- | | | | | | |
| | {[(2S)-2-amino-3- | | | | | | |
| | methylbutanoyl]oxy}pentanoate | | | | | | |
| 338 | trifluoroacetic acid {4-oxo-1-[2- | | | | 99 | 3 | 4% remaining |
| | (propan-2-yloxy)ethyl]-2- | | | | | | at 1 h |
| | sulfanylidene-1H,2H,3H,4H,5H- | | | | | | |
| | pyrrolo[3,2-d]pyrimidin-5-yl}methyl | | | | | | |
| | 5-{[(2R)-2-amino-3- | | | | | | |
| | methylbutanoyl]oxy}pentanoate | | | | | | |
| 339 | bis(trifluoroacetic acid) (3-{[(5- | | | 38.6 | 99 | 72 | 44% remaining |
| | {[(2R)-2-amino-3- | | | | | | at 1 h |
| | methylbutanoyl]oxy}pentanoyl)oxy] | | | | | | |
| | methyl}-4-oxo-1-[2-(propan-2- | | | | | | |
| | yloxy)ethyl]-2-sulfanylidene- | | | | | | |
| | 1H,2H,3H,4H,5H-pyrrolo[3,2- | | | | | | |
| | d]pyrimidin-5-yl)methyl 5- | | | | | | |
| | {[(2R)-2-amino-3- | | | | | | |
| | methylbutanoyl]oxy}pentanoate | | | | | | |
| 340 | trifluoroacetic acid {4-oxo-1-[2- | | | 6.44 | 100 | 99 | 4% remaining |
| | (propan-2-yloxy)ethyl]-2- | | | | | | at 1 h |
| | sulfanylidene-1H,2H,3H,4H,5H- | | | | | | |
| | pyrrolo[3,2-d]pyrimidin-5-yl}methyl | | | | | | |
| | 5-{[(2S)-2-amino-3- | | | | | | |
| | phenylpropanoyl]oxy}pentanoate | | | | | | |
| 341 | trifluoroacetic acid {4-oxo-1-[2- | | | 2.59 | 99 | 33 | 1% remaining |
| | (propan-2-yloxy)ethyl]-2- | | | | | | at 1 h |
| | sulfanylidene-1H,2H,3H,4H,5H- | | | | | | |
| | pyrrolo[3,2-d]pyrimidin-5-yl}methyl | | | | | | |
| | 5-{[(2R)-2-amino-3- | | | | | | |
| | phenylpropanoyl]oxy}pentanoate | | | | | | |
| 342 | 5-oxo-5-({4-oxo-1-[2-(propan-2- | | | | | | |
| | yloxy)ethyl]-2-sulfanylidene- | | | | | | |
| | 1H,2H,3H,4H,5H-pyrrolo[3,2- | | | | | | |
| | d]pyrimidin-5-yl} methoxy)pentyl | | | | | | |
| | (2S)-2-{[(tert- | | | | | | |
| | butoxy)carbonyl]amino}-4- | | | | | | |
| | methylpentanoate | | | | | | |
| 343 | 5-{[2-({[(5-{[(2S)-2- | | | 46.7 | 82% remaining | 29% remaining | 1% remaining |
| | {[(tert-butoxy)carbonyl]amino}- | | | | at 1 h | at 1 h | at 1 h |
| | 4-methylpentanoyl]oxy}penta- | | | | | | |
| | noyl)oxy]methyl}sulfanyl)-4-oxo-1- | | | | | | |
| | [2-(propan-2-yloxy)ethyl]-1H,4H,5H- | | | | | | |
| | pyrrolo[3,2-d]pyrimidin-5- | | | | | | |
| | yl]methoxy}-5-oxopentyl (2S)-2- | | | | | | |
| | {[(tert-butoxy)carbonyl]amino}-4- | | | | | | |
| | methylpentanoate | | | | | | |
| 344 | 5-[(3-{[(5-{[(2S)-2-{[(tert- | | | 24.6 | 100% remaining | 26% remaining | 28% remaining |
| | butoxy)carbonyl]amino}-4- | | | | at 1 h | at 1 h | at 1 h |
| | methylpentanoyl]oxy}penta- | | | | | | |
| | noyl)oxy]methyl}- | | | | | | |
| | 4-oxo-1-[2-(propan-2- | | | | | | |
| | yloxy)ethyl]-2-sulfanylidene- | | | | | | |
| | 1H,2H,3H,4H,5H-pyrrolo[3,2- | | | | | | |
| | d]pyrimidin-5-yl)methoxy]-5- | | | | | | |
| | oxopentyl (2S)-2-{[(tert- | | | | | | |
| | butoxy)carbonyl]amino}-4- | | | | | | |
| | methylpentanoate | | | | | | |
| 345 | trifluoroacetic acid 5-oxo-5-({4-oxo- | | | <0.662 | 71% remaining | 2% remaining at | 77% remaining |
| | 1-[2-(propan-2-yloxy)ethyl]-2- | | | | at 1 h | 1 h | at 1 h |
| | sulfanylidene-1H,2H,3H,4H,5H- | | | | | | |
| | pyrrolo[3,2-d]pyrimidin-5- | | | | | | |
| | yl}methoxy)pentyl (2S)-2-amino-4- | | | | | | |
| | methylpentanoate | | | | | | |
| 346 | bis (trifluoroacetic acid) 5-{[2- | | | <0.623 | 97% remaining | 7% remaining | 98% remaining |
| | ({[(5-{[(2S)-2-amino-4- | | | | at 1 h | at 1 h | at 1 h |
| | methylpentanoyl]oxy}penta- | | | | | | |
| | noyl)oxy]methyl}sulfanyl)-4-oxo-1-[2- | | | | | | |
| | (propan-2-yloxy)ethyl]-1H,4H,5H- | | | | | | |
| | pyrrolo[3,2-d]pyrimidin-5- | | | | | | |
| | yl]methoxy}-5-oxopentyl (2S)-2- | | | | | | |
| | amino-4-methylpentanoate | | | | | | |
| 347 | 5-oxo-5-({4-oxo-1-[2-(propan-2- | | | 10.6 | 98% remaining | 4% remaining | 16% remaining |
| | yloxy)ethyl]-2-sulfanylidene- | | | | at 1 h | at 1 h | at 1 h |
| | 1H,2H,3H,4H,5H-pyrrolo[3,2- | | | | | | |
| | d]pyrimidin-5-yl}methoxy)pentyl | | | | | | |
| | (2R)-2-{[(tert- | | | | | | |
| | butoxy)carbonyl]amino}-4- | | | | | | |
| | methylpentanoate | | | | | | |
| 348 | 5-{[2-({[(5-{[(2R)-2- | | | <0.965 | 88% remaining | 20% remaining | 33% remaining |
| | {[(tert-butoxy)carbonyl]amino}-4- | | | | at 1 h | at 1 h | at 1 h |
| | methylpentanoyl]oxy}penta- | | | | | | |
| | noyl)oxy]methyl}sulfanyl)-4-oxo-1-[2- | | | | | | |
| | (propan-2-yloxy)ethyl]-1H,4H,5H- | | | | | | |
| | pyrrolo[3,2-d]pyrimidin-5- | | | | | | |
| | yl]methoxy}-5-oxopentyl (2R)-2- | | | | | | |
| | {[(tert-butoxy)carbonyl]amino}-4- | | | | | | |
| | methylpentanoate | | | | | | |
| 349 | 5-[(3-{[(5-{[(2R)-2-{[(tert- | | | 49.9 | 99% remaining | 8% remaining | 6% remaining |
| | butoxy)carbonyl]amino}-4- | | | | at 1 h | at 1 h | at 1 h |
| | methylpentanoyl]oxy}penta- | | | | | | |
| | noyl)oxy]methyl}-4-oxo-1-[2-(propan- | | | | | | |
| | 2-yloxy)ethyl]-2-sulfanylidene- | | | | | | |
| | 1H,2H,3H,4H,5H-pyrrolo[3,2- | | | | | | |
| | d]pyrimidin-5-yl)methoxy]-5- | | | | | | |
| | oxopentyl (2R)-2-{[(tert- | | | | | | |
| | butoxy)carbonyl]amino}-4- | | | | | | |
| | methylpentanoate | | | | | | |
| 350 | trifluoroacetic acid 5-oxo-5-({4-oxo- | | | 0.782 | 29% remaining | 52% remaining | 44% remaining |
| | 1-[2-(propan-2-yloxy)ethyl]-2- | | | | at 1 h | at 1 h | at 1 h |
| | sulfanylidene-1H, 2H,3H,4H,5H- | | | | | | |
| | pyrrolo[3,2-d]pyrimidin-5- | | | | | | |
| | yl}methoxy)pentyl (2R)-2-amino-4- | | | | | | |
| | methylpentanoate | | | | | | |
| 351 | bis(trifluoroacetic acid) 5-{[2- | | | | 69% remaining | 14% remaining | 99% remaining |
| | ({[(5-{[(2R)-2-amino-4- | | | | at 1 h | at 1 h | at 1 h |
| | methylpentanoyl]oxy}penta- | | | | | | |
| | noyl)oxy]methyl}sulfanyl)-4-oxo-1-[2- | | | | | | |
| | (propan-2-yloxy)ethyl]-1H,4H,5H- | | | | | | |
| | pyrrolo[3,2-d]pyrimidin-5- | | | | | | |
| | yl]methoxy}-5-oxopentyl (2R)-2- | | | | | | |
| | amino-4-methylpentanoate | | | | | | |
| 352 | {4-oxo-1-[2-(propan-2-yloxy)ethyl]- | | | 20.3 | 92% remaining | 9% remaining | 9% remaining |
| | 2-sulfanylidene-1H,2H,3H,4H,5H- | | | | at 1 h | at 1 h | at 1 h |
| | pyrrolo[3,2-d]pyrimidin-5-yl}methyl | | | | | | |
| | 5-{[(2S)-2-{[(tert- | | | | | | |
| | butoxy)carbonyl]amino}-4- | | | | | | |
| | (methylsulfanyl)butanoyl]oxy}pentanoate | | | | | | |
| 353 | trifluoroacetic acid {4-oxo-1-[2- | | | 5.49 | 33% remaining | 66% remaining | 3% remaining |
| | (propan-2-yloxy)ethyl]-2- | | | | at 1 h | at 1 h | at 1 h |
| | sulfanylidene-1H,2H,3H,4H,5H- | | | | | | |
| | pyrrolo[3,2-d]pyrimidin-5-yl}methyl | | | | | | |
| | 5-{[(2S)-2-amino-4- | | | | | | |
| | (methylsulfanyl)butanoyl]oxy}pentanoate | | | | | | |
| 354 | trifluoroacetic acid 2-(2- | | | | 99% remaining | 3% remaining | 1% remaining |
| | aminoethoxy)ethyl {4-oxo-1-[2- | | | | at 1 h | at 1 h | at 1 h |
| | (propan-2-yloxy)ethyl]-2- | | | | | | |
| | sulfanylidene-1H,2H,3H,4H,5H- | | | | | | |
| | pyrrolo[3,2-d]pyrimidin-5-yl}methyl | | | | | | |
| | carbonate | | | | | | |
| 355 | 4-oxo-4-({4-oxo-1-[2-(propan-2- | | | 21.6 | 99% remaining | 100% remaining | 43% remaining |
| | yloxy)ethyl]-2-sulfanylidene- | | | | at 1 h | at 1 h | at 1 h |
| | 1H,2H,3H,4H,5H-pyrrolo[3,2- | | | | | | |
| | d]pyrimidin-5-yl}methoxy)butyl (2R)- | | | | | | |
| | 2-{[(tert-butoxy)carbonyl]amino}-3- | | | | | | |
| | methylbutanoate | | | | | | |
| 356 | trifluoroacetic acid 4-oxo-4-({4-oxo- | | | 113 | 99% remaining | 25% remaining | 5% remaining |
| | 1-[2-(propan-2-yloxy)ethyl]-2- | | | | at 1 h | at 1 h | at 1 h |
| | sulfanylidene-1H,2H,3H,4H,5H- | | | | | | |
| | pyrrolo[3,2-d]pyrimidin-5- | | | | | | |
| | yl}methoxy)butyl (2R)-2-amino-3- | | | | | | |
| | methylbutanoate | | | | | | |
| 357 | 4-{[2-({[(4-{[(2R)-2- | | | 15 | 98% remaining | 14% remaining | 5% remaining |
| | {[(tert-butoxy)carbonyl]amino}-3- | | | | at 1 h | at 1 h | at 1 h |
| | methylbutanoyl]oxy}buta- | | | | | | |
| | noyl)oxy]methyl}sulfanyl)- | | | | | | |
| | 4-oxo-1-[2-(propan- | | | | | | |
| | 2-yloxy)ethyl]-1H,4H,5H-pyrrolo[3,2- | | | | | | |
| | d]pyrimidin-5-yl]methoxy}-4- | | | | | | |
| | oxobutyl (2R)-2-{[(tert- | | | | | | |
| | butoxy)carbonyl]amino}-3- | | | | | | |
| | methylbutanoate | | | | | | |
| 358 | bis(trifluoroacetic acid) 4-(((5-(((4- | | | 17.8 | 80% remaining | 48 | 12% remaining |
| | ((D-valyl)oxy)butanoyl)oxy)methyl)- | | | | at 1 h | | at 1 h |
| | 1-(2-isopropoxyethyl)-4-oxo-4,5- | | | | | | |
| | dihydro-1H-pyrrolo[3,2-d]pyrimidin- | | | | | | |
| | 2-yl)thio)methoxy)-4-oxobutyl D- | | | | | | |
| | valinate | | | | | | |
| 359 | (((1-(2-isopropoxyethyl)-4-oxo-2- | | | <2.97 | 99% remaining | 41% remaining | 8% remaining |
| | thioxo-1,4-dihydro-3H-pyrrolo[3,2- | | | | at 1 h | at 1 h | at 1 h |
| | d]pyrimidine-3,5(2H)- | | | | | | |
| | diyl)bis(methylene))bis(oxy))bis(4- | | | | | | |
| | oxobutane-4,1-diyl) (2R,2′R)-bis(2- | | | | | | |
| | ((tert-butoxycarbonyl)amino)-3- | | | | | | |
| | methylbutanoate) | | | | | | |
| 360 | bis(trifluoroacetic acid) 4-[(3- | | | <6.28 | 46% remaining | 56% remaining | 3% remaining |
| | {[(4-{[(2R)-2-amino-3- | | | | at 1 h | at 1 h | at 1 h |
| | methylbutanoyl]oxy}buta- | | | | | | |
| | noyl)oxy]methyl}-4-oxo-1-[2-(propan- | | | | | | |
| | 2-yloxy)ethyl]-2-sulfanylidene- | | | | | | |
| | 1H,2H,3H,4H,5H-pyrrolo[3,2- | | | | | | |
| | d]pyrimidin-5-yl)methoxy]-4- | | | | | | |
| | oxobutyl (2R)-2-amino-3- | | | | | | |
| | methylbutanoate | | | | | | |
| 361 | {4-oxo-1-[2-(propan-2-yloxy)ethyl]- | | | <3.10 | 95% remaining | 9% remaining | 8% remaining |
| | 2-sulfanylidene-1H,2H,3H,4H,5H- | | | | at 1 h | at 1 h | at 1h |
| | pyrrolo[3,2-d]pyrimidin-5-yl}methyl | | | | | | |
| | 4-{[(2R)-2-{[(tert- | | | | | | |
| | butoxy)carbonyl]amino}-3- | | | | | | |
| | phenylpropanoyl]oxy}butanoate | | | | | | |
| 362 | trifluoroacetic acid {4-oxo-1-[2- | | | 51.2 | 90% remaining | 2% remaining | 3% remaining |
| | (propan-2-yloxy)ethyl]-2- | | | | at 1 h | at 1 h | at 1 h |
| | sulfanylidene-1H,2H,3H,4H,5H- | | | | | | |
| | pyrrolo[3,2-d]pyrimidin-5-yl}methyl | | | | | | |
| | 4-{[(2R)-2-amino-3- | | | | | | |
| | phenylpropanoyl]oxy}butanoate | | | | | | |
| 363 | [(5-{[(4-{[(2R)-2-{[(tert- | | | 16.3 | 97% remaining | 18% remaining | 42% remaining |
| | butoxy)carbonyl]amino}-3- | | | | at 1 h | at 1 h | at 1 h |
| | phenyl propanoyl]oxy}butanoyl)oxy] | | | | | | |
| | methyl}-4-oxo-1-[2-(propan-2- | | | | | | |
| | yloxy)ethyl]-1H,4H,5H-pyrrolo[3,2- | | | | | | |
| | d]pyrimidin-2-yl) sulfanyl]methyl 4- | | | | | | |
| | {[(2R)-2-{[(tert- | | | | | | |
| | butoxy)carbonyl]amino}-3- | | | | | | |
| | phenylpropanoyl ]oxy}butanoate | | | | | | |
| 364 | bis(trifluoroacetic acid) [(5-{[(4- | | | 2.77 | 47% remaining | 1% remaining | 31% remaining |
| | {[(2R)-2-amino-3- | | | | at 1 h | at 1 h | at 1 h |
| | phenyl propanoyl]oxy}butanoyl)oxy] | | | | | | |
| | methyl}-4-oxo-1-[2-(propan-2- | | | | | | |
| | yloxy)ethyl]-1H,4H,5H-pyrrolo[3,2- | | | | | | |
| | d]pyrimidin-2-yl)sulfanyl]methyl 4- | | | | | | |
| | {[(2R)-2-amino-3- | | | | | | |
| | phenylpropanoyl]oxy}butanoate | | | | | | |
| 365 | (3-{[(4-{[(2R)-2-{[(tert- | | | <2.63 | 85% remaining | 5% remaining | 11% remaining |
| | butoxy)carbonyl]amino}-3- | | | | at 1 h | at 1 h | at 1 h |
| | phenylpropanoyl]oxy}butanoyl)oxy] | | | | | | |
| | methyl}-4-oxo-1-[2-(propan-2- | | | | | | |
| | yloxy)ethyl]-2-sulfanylidene- | | | | | | |
| | 1H,2H,3H,4H,5H-pyrrolo[3,2- | | | | | | |
| | d]pyrimidin-5-yl)methyl 4-{[(2R)-2- | | | | | | |
| | {[(tert-butoxy)carbonyl]amino}-3- | | | | | | |
| | phenylpropanoyl]oxy}butanoate | | | | | | |
| 366 | trifluoroacetic acid 3-oxo-3-({4-oxo- | | | <7.6 | 76% remaining | 2% remaining | 74% remaining |
| | 1-[2-(propan-2-yloxy)ethyl]-2- | | | | at 1 h | at 1 h | at 1 h |
| | sulfanylidene-1H,2H,3H,4H,5H- | | | | | | |
| | pyrrolo[3,2-d]pyrimidin-5- | | | | | | |
| | yl}methoxy)propyl (2R)-2-amino-4- | | | | | | |
| | (methylsulfanyl)butanoate | | | | | | |
| 367 | trifluoroacetic acid 3-oxo-3-({4-oxo- | | | <1.08 | 100% remaining | 0% remaining | 1% remaining |
| | 1-[2-(propan-2-yloxy)ethyl]-2- | | | | at 1 h | at 1 h | at 1 h |
| | sulfanylidene-1H,2H,3H,4H,5H- | | | | | | |
| | pyrrolo [3,2-d]pyrimidin-5- | | | | | | |
| | yl}methoxy)propyl (2S)-2-amino-4- | | | | | | |
| | methylpentanoate | | | | | | |
| 368 | trifluoroacetic acid 3-oxo-3-({4-oxo- | | | 1.2 | 100% remaining | 1% remaining | 1% remaining |
| | 1-[2-(propan-2-yloxy)ethyl]-2- | | | | at 1 h | at 1 h | at 1 h |
| | sulfanylidene-1H,2H,3H,4H,5H- | | | | | | |
| | pyrrolo[3,2-d]pyrimidin-5- | | | | | | |
| | yl}methoxy)propyl (2R)-2-amino-4- | | | | | | |
| | methylpentanoate | | | | | | |
| 369 | 2-{[{{[2-(2-{[(tert- | | | <1.07 | 89% remaining | 1% remaining | 1% remaining |
| | butoxy)carbonyl]amino}ethoxy)eth- | | | | at 1 h | at 1 h | at 1 h |
| | oxy]carbonyl}oxy)methyl]sulfanyl}-1- | | | | | | |
| | [2-(propan-2-yloxy)ethyl]-1H,4H,5H- | | | | | | |
| | pyrrolo[3,2-d]pyrimidin-4-one | | | | | | |
| 370 | 5-{[({2-[2-(2-{[(tert- | 516 | H NMR (300 MHz, | 15.4 | 99% remaining | 35% remaining | 1% remaining |
| | butoxy)carbonyl]amino}ethoxy)eth- | | CHLOROFORM-d6) δ 10.68 | | at 1 h | at 1 h | at 1 h |
| | oxy]ethoxy}carbonyl)oxy]methyl}-1- | | (br s, 1 H), 7.55 (t, J = 3.13 Hz, | | | | |
| | [2-(propan-2-yloxy)ethyl]-2- | | 1 H), 6.28-6.42 (m, 1 H), 6.04 (s, | | | | |
| | sulfanylidene-1H,2H,3H,4H,5H- | | 2 H), 4.38-4.49 (m, 2 H), 4.23 | | | | |
| | pyrrolo[3,2-d]pyrimidin-4-one | | 4.41 (m, 2 H), 3.70-3.83 (m, 2 | | | | |
| | | | H), 3.65-3.70 (m, 2 H), 3.45- | | | | |
| | | | 3.65 (m, 3 H), 3.28 (br s, 2 H), | | | | |
| | | | 1.41 (s, 9 H), 0.91-1.16 (m, 6 | | | | |
| | | | H). | | | | |
| 371 | trifluoroacetic acid 2-[2-(2- | 416 | 1H NMR (300 MHz, | | | | |
| | aminoethoxy)ethoxy]ethyl {4-oxo-1- | | CHLOROFORM-d6) δ 10.68 | | | | |
| | [2-(propan-2-yloxy)ethyl]-2- | | (br s, 1 H), 8.11 (br s, 2 H), 7.15- | | | | |
| | sulfanylidene-1H,2H,3H,4H,5H- | | 7.34 (m, 2 H), 6.26 (d, J = 3.52 | | | | |
| | pyrrolo[3,2-d]pyrimidin-5-yl}methyl | | Hz, 1 H), 6.20 (s, 2 H), 4.51 (t, | | | | |
| | carbonate | | J = 5.57 Hz, 2 H), 4.23-4.41 (m, | | | | |
| | | | 2 H), 3.65-3.93 (m, 6 H), 3.45- | | | | |
| | | | 3.65 (m, 1 H), 3.22 (br s, 2 H), | | | | |
| | | | 1.07 (d, J = 6.45 Hz, 6 H). | | | | |
| 372 | tert-butyl N-[2-(2-{[{{4-oxo- | 560 | 1H NMR (300 MHz, | 117 | 85% remaining | 60% remaining | 0% remaining |
| | 1-[2-(propan-2-yloxy)ethyl]-2- | | CHLOROFORM-d6) δ 9.55 (br | | at 1 h | at 1 h | at 1 h |
| | sulfanylidene-1H,2H,3H,4H,5H- | | s, 1 H), 7.28 (d, J = 2.93 Hz, 1 | | | | |
| | pyrrolo[3,2-d]pyrimidin-5- | | H), 6.15-6.34 (m, 3 H), 4.52 (t, | | | | |
| | yl}methoxy)carbonyl]amino}ethoxy) | | J = 5.57 Hz, 2 H), 4.21-4.35 (m, | | | | |
| | ethyl]carbamate | | 2 H), 3.83 (t, J = 5.57 Hz, 2 H), | | | | |
| | | | 3.64-3.76 (m, 2 H), 3.41-3.64 | | | | |
| | | | (m, 8 H), 3.28 (br s, 2 H), 1.42 | | | | |
| | | | (s, 9 H), 1.06 (d, J = 5.86 Hz, 6 | | | | |
| | | | H). | | | | |
| 373 | trifluoroacetic acid {4-oxo-1-[2- | 459 | 1H NMR (300 MHz, | 1.23 | 90% remaining | 72% remaining | 68% remaining |
| | (propan-2-yloxy)ethyl]-2- | | CHLOROFORM-d6) δ 10.78 | | at 1 h | at 1 h | at 1 h |
| | sulfanylidene-1H,2H,3H,4H,5H- | | (s, 1 H), 7.97 (br s, 2 H), 7.31 | | | | |
| | pyrrolo[3,2-d]pyrimidin-5-yl}methyl | | (d, J = 2.93 Hz, 1 H), 6.24 (d, | | | | |
| | N-[2-(2- | | J = 2.93 Hz, 1 H), 6.19 (s, 2 H), | | | | |
| | aminoethoxy)ethyl]carbamate | | 4.50 (t, J = 5.57 Hz, 2 H), 4.28 | | | | |
| | | | (dt, J = 3.81, 2.20 Hz, 2 H), 3.44 | | | | |
| | | | 3.83 (m, 11 H), 3.22 (br s, 2 H), | | | | |
| | | | 1.05 (d, J = 5.86 Hz, 6 H). | | | | |
| 374 | trifluoroacetic acid ({4-oxo-1-[2- | 515 | 1H NMR (300 MHz, | 90.6 | 75% remaining | 94% remaining | 82% remaining |
| | (propan-2-yloxy)ethyl]-1H,4H,5H- | | CHLOROFORM-d6) δ 9.64 (br | | at 1 h | at 1 h | at 1 h |
| | pyrrolo[3,2-d]pyrimidin-2- | | s, 1 H), 7.36 (d, J = 2.93 Hz, 1 | | | | |
| | yl}sulfanyl)methyl N-[2-(2- | | H), 6.13-6.28 (m, 3 H), 5.56 | | | | |
| | aminoethoxy)ethyl]carbamate | | (br s, 1 H), 4.52 (t, J = 5.86 Hz, 2 | | | | |
| | | | H), 3.82 (t, J = 5.86 Hz, 2 H), | | | | |
| | | | 3.39-3.64 (m, 5 H), 3.11-3.39 | | | | |
| | | | (m, 4 H), 1.42 (s, 9 H), 0.96- | | | | |
| | | | 1.18 (m, 6 H). | | | | |
| 375 | tert-butyl N-[2-(2-{[(1-{4-oxo- | 415 | 1H NMR (300 MHz, | <1.75 | 89% remaining | 99% remaining | 99% remaining |
| | 1-[2-(propan-2-yloxy)ethyl]-2- | | CHLOROFORM-d6) δ 7.33 (d, | | at 1 h | at 1 h | at 1 h |
| | sulfanylidene-1H,2H,3H,4H,5H- | | J = 3.52 Hz, 1 H), 6.20 (d, | | | | |
| | pyrrolo[3,2-d]pyrimidin-5- | | J = 2.93 Hz, 1 H), 6.09 (s, 2 H), | | | | |
| | yl}ethoxy)carbonyl]amino}eth- | | 4.49 (t, J = 5.86 Hz, 2 H), 3.79 (t, | | | | |
| | oxy)ethyl]carbamate | | J = 5.57 Hz, 2 H), 3.40-3.69 (m, | | | | |
| | | | 5 H), 3.29 (t, J = 4.69 Hz, 2 H), | | | | |
| | | | 3.09 (t, J = 4.69 Hz, 2 H), 1.04 | | | | |
| | | | (d, J = 5.86 Hz, 6 H). | | | | |
| 376 | N-[2-(2-aminoethoxy)ethyl]-4-oxo-1- | 415 | 1H NMR (300 MHz, | | 98% remaining | 99% remaining | 95% remaining |
| | [2-(propan-2-yloxy)ethyl]-2- | | CHLOROFORM-d6) δ 11.85 | | at 1 h | at 1 h | at 1 h |
| | sulfanylidene-1H, 2H,3H,4H,5H- | | (br s, 1 H), 8.20 (brs, 2 H), 7.10- | | | | |
| | pyrrolo[3,2-d]pyrimidine-5- | | 7.20 (m, 1 H), 6.08-6.20 (m, | | | | |
| | carboxamide; trifluoroacetic acid | | 1 H), 5.74 (s, 2 H), 4.22 (t, | | | | |
| | | | J = 5.27 Hz, 2 H), 3.59-3.88 (m, | | | | |
| | | | 5 H), 3.44-3.59 (m, 2 H), 3.25- | | | | |
| | | | 3.44 (m, 2 H), 2.96-3.24 (m, 2 | | | | |
| | | | H), 1.06 (d, J = 5.86 Hz, 6 H). | | | | |
| 377 | (2R)-2-{[(tert- | 528 | 1H NMR (300 MHz, | | 83 | 11 | 8% remaining |
| | butoxy)carbonyl]amino}-3-methyl-N- | | CHLOROFORM-d6) δ 10.99 | | | | at 1 h |
| | {2-[2-(2-{[{{4-oxo- | | (br s, 1 H), 8.15 (d, J = 3.52 Hz, | | | | |
| | 1-[2-(propan-2-yloxy)ethyl]-2- | | 1 H), 6.42 (d, J = 3.52 Hz, 1 H), | | | | |
| | sulfanylidene-1H,2H,3H,4H,5H-pyrrolo[3,2- | | 6.18 (brs, 1 H), 4.57 (t, J = 5.27 | | | | |
| | d]pyrimidin-5- | | Hz, 2 H), 3.85 (t, J = 5.27 Hz, 2 | | | | |
| | yl}methoxy)carbonyl]oxy}ethoxy)eth- | | H), 3.46-3.72 (m, 7 H), 3.38- | | | | |
| | oxy]ethyl}butanamide | | 3.40 (m, 2 H), 1.50 (s, 9 H), | | | | |
| | | | 1.05 (d, J = 5.86 Hz, 6 H). | | | | |
| 378 | tert-butyl N-[(1R)-2-methyl-1-({2-[2- | 385 | 1H NMR (300 MHz, | 0.903 | 98 | 96 | 93% remaining |
| | ({4-oxo-1-[2-(propan-2-yloxy)ethyl]- | | CHLOROFORM-d6) δ 12.00 | | | | at 1 h |
| | 2-sulfanylidene-1H,2H,3H,4H,5H- | | (br s, 1 H), 11.03 (t, J = 4.40 Hz, | | | | |
| | pyrrolo[3,2-d]pyrimidine-5- | | 1 H), 8.24 (br s, 3 H), 8.09 (d, | | | | |
| | carbonyl}amino)ethoxy]ethyl}carba- | | J = 3.52 Hz, 1 H), 6.29 (d, | | | | |
| | moyl)propyl]carbamate | | J = 3.52 Hz, 1 H), 4.33 (t, J = 5.28 | | | | |
| | | | Hz, 2 H), 3.58-3.91 (m, 8 H), | | | | |
| | | | 3.43-3.51 (m, 1 H), 3.22- | | | | |
| | | | 3.31 (m, 2 H), 1.01 (d, J = 5.86 | | | | |
| | | | Hz, 7 H). | | | | |
| 379 | tert-butyl N-(2-{2- | 659 | 1H NMR (300 MHz, | | 93% remaining | 69% remaining | 0% remaining |
| | [methyl({[{{4-oxo- | | CHLOROFORM-d6) δ 9.68 (br | | at 1 h | at 1 h | at 1 h |
| | 1-[2-(propan-2-yloxy)ethyl]- | | s, 1 H), 7.29 (d, J = 2.93 Hz, 1 | | | | |
| | 1H,4H,5H-pyrrolo[3,2-d]pyrimidin-2- | | H), 6.15-6.33 (m, 3 H), 4.53 (t, | | | | |
| | yl}sulfanyl)methoxy]carbonyl})ami- | | J = 5.86 Hz, 2 H), 4.22-4.37 (m, | | | | |
| | no]ethoxy}ethyl)carbamate | | 2 H), 3.89-4.02 (m, 1 H), 3.83 | | | | |
| | | | (t, J = 5.57 Hz, 2 H), 3.64-3.76 | | | | |
| | | | (m, 2 H), 3.26-3.64 (m, 9 H), | | | | |
| | | | 1.93-2.21 (m, 1 H), 1.43 (s, 9 | | | | |
| | | | H), 0.99-1.14 (m, 6 H), 0.72- | | | | |
| | | | 0.99 (m, 6 H). | | | | |
| 380 | trifluoroacetic acid ({4-oxo-1-[2- | 584 | 1H NMR (300 MHz, | | 100% remaining | 99% remaining | 85% remaining |
| | (propan-2-yloxy)ethyl]-1H,4H,5H- | | CHLOROFORM-d6) δ 12.02 | | at 1 h | at 1 h | at 1 h |
| | pyrrolo[3,2-d]pyrimidin-2- | | (s, 1 H), 11.00 (br s, 1 H), 8.16 | | | | |
| | yl}sulfanyl) methyl N-[2-(2- | | (d, J = 3.52 Hz, 1 H), 7.57 (br s, | | | | |
| | aminoethoxy)ethyl]-N- | | 1 H), 6.42 (d, J = 4.10 Hz, 1 H), | | | | |
| | methylcarbamate | | 5.38 (d, J = 9.96 Hz, 1 H), 4.44- | | | | |
| | | | 4.71 (m, 2 H), 4.04-4.31 (m, 2 | | | | |
| | | | H), 3.86 (t, J = 5.27 Hz, 2 H), | | | | |
| | | | 3.41-3.80 (m, 5 H), 3.22-3.41 | | | | |
| | | | (m, 1 H), 2.93 (br s, 2 H), 1.91 | | | | |
| | | | (dt, J = 8.65, 6.52 Hz, 1 H), 1.50 | | | | |
| | | | (s, 9 H), 1.05 (d, J = 5.86 Hz, 6 | | | | |
| | | | H), 0.76-1.00 (m, 6 H). | | | | |
| 381 | butoxy)carbonyl]amino}ethoxy)eth- | 529 | 1H NMR (300 MHz, | | 82% remaining | 97% remaining | 97% remaining |
| | yl](methyl)carbamoyl}oxy)methyl]sul- | | CHLOROFORM-d6) δ 12.58- | | at 1 h | at 1 h | at 1 h |
| | fanyl}-4-oxo-1-[2-(propan-2- | | 12.78 (m, 1 H), 9.90 (br s, 1 H), | | | | |
| | yloxy)ethyl]-1H,4H,5H-pyrrolo[3,2- | | 7.43 (t, J = 2.64 Hz, 1 H), 6.12- | | | | |
| | d]pyrimidin-5- | | 6.33 (m, 1 H), 5.96 (d, J = 7.62 | | | | |
| | yl)methoxy]carbonyl}(methyl)ami- | | Hz, 2 H), 4.31 (t, J = 5.57 Hz, 2 | | | | |
| | no)ethoxy]ethyl}carbamate | | H), 3.72 (t, J = 5.86 Hz, 2 H), | | | | |
| | | | 3.27-3.60 (m, 7 H), 3.05- | | | | |
| | | | 3.25 (m, 2 H), 2.90 (d, J = 17.58 | | | | |
| | | | Hz, 3 H), 1.35 (d, J = 2.34 Hz, 9 | | | | |
| | | | H), 0.98 (d, J = 6.45 Hz, 6 H). | | | | |
| 382 | bis(trifluoroacetic acid) (2-{[({[2-(2- | 429 | 1H NMR (300 MHz, | | 100% remaining | 99% remaining | 98% remaining |
| | aminoethoxy)ethyl](methyl)carba- | | CHLOROFORM-d6) δ 12.29 | | at 1 h | at 1 h | at 1 h |
| | moyl}oxy)methyl]sulfanyl}-4-oxo-1-[2- | | (br s, 1 H), 8.23 (br s, 2 H), 7.39- | | | | |
| | (propan-2-yloxy)ethyl]-1H,4H,5H- | | 7.41 (m, 1 H), 6.28-6.30 (m, | | | | |
| | pyrrolo[3,2-d]pyrimidin-5-yl)methyl | | 1 H), 5.82-5.90 (m, 3 H), 4.34 | | | | |
| | N-[2-(2-aminoethoxy)ethyl]-N- | | (t, J = 4.98 Hz, 2 H), 3.73-3.85 | | | | |
| | methylcarbamate | | (m, 2 H), 3.26-3.60 (m, 7 H), | | | | |
| | | | 3.10-3.23 (m, 2 H), 2.73- | | | | |
| | | | 2.98 (m, 3 H), 1.03 (d, J = 6.45 | | | | |
| | | | Hz, 6 H). | | | | |
| 383 | (2R)-2-{[(tert- | 803 | 1H NMR (300 MHz, | | | | |
| | butoxy)carbonyl]amino}-3-methyl-N- | | CHLOROFORM-d6) δ 7.82 (d, | | | | |
| | [2-(2-{[{{4-oxo-1-[2-(propan-2- | | J = 7.62 Hz, 1 H), 7.43-7.56 (m, | | | | |
| | yloxy)ethyl]-2-sulfanylidene- | | 1 H), 6.36 (d, J = 9.38 Hz, 2 H), | | | | |
| | 1H,2H,3H,4H,5H-pyrrolo[3,2- | | 6.27 (d, J = 2.93 Hz, 1 H), 5.85- | | | | |
| | d]pyrimidin-5- | | 6.02 (m, 2 H), 4.66 (br s, 2 H), | | | | |
| | yl}methoxy)carbonyl]oxy}ethoxy)eth- | | 4.28 (t, J = 5.57 Hz, 2 H), 3.67- | | | | |
| | yl]butanamide | | 3.81 (m, 2 H), 3.34-3.67 (m, | | | | |
| | | | 13 H), 3.09-3.34 (m, 4 H), | | | | |
| | | | 2.81-3.09 (m, 6 H), 1.28-1.50 | | | | |
| | | | (m, 18 H), 0.98-1.12 (m, 6 H). | | | | |
| 384 | (2R)-2-amino-3-methyl-N-[2-(2-{[{{4 | 603 | 1H NMR (300 MHz, | | | | |
| | oxo-1-[2-(propan-2-yloxy)ethyl]-2- | | CHLOROFORM-d6) δ 7.30- | | | | |
| | sulfanylidene-1H,2H,3H,4H,5H- | | 7.42 (m, 1 H), 6.20-6.30 (m, 3 | | | | |
| | pyrrolo[3,2-d]pyrimidin-5- | | H), 5.82 (br s, 2 H), 4.28 (t, | | | | |
| | yl}methoxy)carbonyl]oxy}ethoxy)eth- | | J = 5.57 Hz, 2 H), 3.67-3.81 (m, | | | | |
| | yl]butanamide; trifluoroacetic acid | | 4 H), 3.45-3.67 (m, 15 H), | | | | |
| | | | 2.79-3.09 (m, 6 H), 1.08 (d, | | | | |
| | | | J = 5.86 Hz, 6 H). | | | | |
| 385 | (2R)-2-{[(tert- | 637 | 1H NMR (300 MHz, | 38.7 | 94% remaining | 84% remaining | 1% remaining |
| | butoxy)carbonyl]amino}-4-methyl-N- | | CHLOROFORM-d6) δ 9.84 (br | | after 1 h | after 1 h | after 1 h |
| | [2-(2-{[{{4-oxo-1-[2- | | s, 1 H), 7.20-7.33 (m, 1 H), | | | | |
| | (propan-2-yloxy)ethyl]-2-sulfanylidene- | | 6.76 (br s, 1 H), 6.08-6.40 (m, | | | | |
| | 1H,2H,3H,4H,5H-pyrrolo[3,2- | | 3 H), 4.53 (t, J = 5.57 Hz, 2 H), | | | | |
| | d]pyrimidin-5- | | 4.28-4.35 (m, 2 H), 3.95 (t, | | | | |
| | yl}methoxy)carbonyl]oxy}ethoxy)eth- | | J = 5.56 Hz, 1 H), 3.83 (t, J = 5.57 | | | | |
| | yl]pentanamide | | Hz, 2 H), 3.61-3.77 (m, 2 H), | | | | |
| | | | 3.47-3.61 (m, 3 H), 2.87- | | | | |
| | | | 2.98 (m, 2 H), 2.01-2.10 (m, 1 | | | | |
| | | | H), 1.42 (s, 9 H), 1.07 (d, | | | | |
| | | | J = 6.45 Hz, 6 H), 0.75-0.99 (m, | | | | |
| | | | 6 H). | | | | |
| 386 | (2R)-2-amino-4-methyl-N-[2-(2-{[{{4- | 515 | 1H NMR (300 MHz, | 0.77 | 93% remaining | 86% remaining | 23% remaining |
| | oxo-1-[2-(propan-2-yloxy)ethyl]-2- | | CHLOROFORM-d6) δ 10.75 | | after 1 h | after 1 h | after 1 h |
| | sulfanylidene-1H,2H,3H,4H,5H- | | (br s, 1 H), 7.99 (br s, 1 H), 7.31 | | | | |
| | pyrrolo[3,2-d]pyrimidin-5- | | (d, J = 2.93 Hz, 1 H), 6.12-6.36 | | | | |
| | yl}methoxy)carbonyl]oxy}ethoxy)eth- | | (m, 3 H), 4.51 (br t, J = 5.57 Hz, | | | | |
| | yl]pentanamide; trifluoroacetic | | 2 H), 4.19-4.38 (m, 2 H), 3.95- | | | | |
| | acid | | 4.00 (m, 1 H), 3.81 (t, J = 5.57 | | | | |
| | | | Hz, 2 H), 3.62-3.70 (m, 2 H), | | | | |
| | | | 3.48-3.62 (m, 3 H), 3.40- | | | | |
| | | | 3.50 (m, 2 H), 2.05-2.34 (m, 1 | | | | |
| | | | H), 0.86-1.14 (m, 12 H). | | | | |
| 387 | (2R)-2-{[(tert- | (M + Na)⁺ = 651 | 1H NMR (300 MHz, | 45 | 92% remaining | 75% remaining | 1% remaining |
| | butoxy)carbonyl]amino}-N-[2-(2- | | CHLOROFORM-d6) δ 9.80 (br | | after 1 h | after 1 h | after 1 h |
| | {[{{4-oxo-1-[2-(propan-2- | | s, 1 H), 7.26-7.33 (m, 1 H), | | | | |
| | yloxy)ethyl]-2-sulfanylidene- | | 6.76 (br s, 1 H), 6.08-6.40 (m, | | | | |
| | 1H,2H,3H,4H,5H-pyrrolo[3,2- | | 3 H), 4.53 (t, J = 5.57 Hz, 2 H), | | | | |
| | d]pyrimidin-5- | | 4.22-4.32 (m, 2 H), 3.83 (t, | | | | |
| | yl}methoxy)carbonyl]oxy}ethoxy)eth- | | J = 5.57 Hz, 2 H), 3.61-3.77 (m, | | | | |
| | yl]-3-phenylpropanamide | | 2 H), 3.47-3.61 (m, 3 H), 3.40- | | | | |
| | | | 3.47 (m, 1 H), 3.01-3.23 (m, | | | | |
| | | | 2 H), 1.55-1.78 (m, 1 H), 1.31- | | | | |
| | | | 1.55 (m, 11 H), 1.06 (d, J = 5.86 | | | | |
| | | | Hz, 6 H), 0.81-0.98 (m, 6 H). | | | | |
| 388 | (2R)-2-amino-N-[2-(2-{[{{4- | 529 | 1H NMR (300 MHz, | <19.9 | 97% remaining | 73% remaining | 1% remaining |
| | oxo-1-[2-(propan-2-yloxy)ethyl]-2- | | CHLOROFORM-d6) δ 10.84 | | after 1 h | after 1 h | after 1 h |
| | sulfanylidene-1H,2H,3H,4H,5H- | | (br s, 1 H), 8.11 (br s, 2 H), 7.30 | | | | |
| | pyrrolo[3,2-d]pyrimidin-5- | | (d, J = 2.93 Hz, 1 H), 6.05-6.32 | | | | |
| | yl}methoxy)carbonyl]oxy}ethoxy)eth- | | (m, 3 H), 4.37-4.61 (m, 2 H), | | | | |
| | yl]-3-phenylpropanamide; | | 4.23-4.50 (m, 2 H), 4.05- | | | | |
| | trifluoroacetic acid | | 4.18 (m, 1 H), 3.81 (t, J = 5.57 | | | | |
| | | | Hz, 2 H), 3.62-3.70 (m, 2 H), | | | | |
| | | | 3.44-3.62 (m, 4 H), 3.31-3.44 | | | | |
| | | | (m, 1 H), 1.44-1.81 (m, 3 H), | | | | |
| | | | 1.06 (d, J = 6.45 Hz, 6 H), 0.89 | | | | |
| | | | (br t, J = 4.69 Hz, 6 H). | | | | |
| 389 | (2R)-2-{[(tert- | 663 | 1H NMR (300 MHz, | 59.4 | 98% remaining | 60% remaining | 1% remaining |
| | butoxy)carbonyl]amino}-4- | | CHLOROFORM-d6) δ 9.73 (br | | after 1 h | after 1 h | after 1 h |
| | (methylsulfanyl)-N-[2-(2-{[{{4-oxo- | | s, 1 H), 7.11-7.34 (m, 6 H), | | | | |
| | 1-[2-(propan-2-yloxy)ethyl]-2- | | 6.15-6.35 (m, 3 H), 4.53 (t, | | | | |
| | sulfanylidene-1H,2H,3H,4H,5H- | | J = 5.57 Hz, 2 H), 4.23 (t, J = 4.40 | | | | |
| | pyrrolo[3,2-d]pyrimidin-5- | | Hz, 2 H), 3.78-3.89 (m, 2 H), | | | | |
| | yl}methoxy)carbonyl]oxy}ethoxy)eth- | | 3.49-3.63 (m, 2 H), 3.23-3.48 | | | | |
| | yl]butanamide | | (m, 2 H), 2.98-3.05 (m, 2 H), | | | | |
| | | | 2.51-2.65 (m, 4 H), 1.39 (s, 9 | | | | |
| | | | H), 1.07 (d, J = 6.45 Hz, 6 H). | | | | |
| 390 | (2R)-2-amino-4-(methylsulfanyl)-N- | 563 | 1H NMR (300 MHz, | 4.9 | 100% remaining | 64% remaining | 0% remaining |
| | [2-(2-{[{{4-oxo-1-[2-(propan- | | CHLOROFORM-d6) δ 10.69 | | after 1 h | after 1 h | after 1 h |
| | 2-yloxy)ethyl]-2-sulfanylidene- | | (br s, 1 H), 7.62 (br s, 1 H), 7.04- | | | | |
| | 1H,2H,3H,4H,5H-pyrrolo[3,2- | | 7.33 (m, 6 H), 6.24 (d, J = 2.93 | | | | |
| | d]pyrimidin-5- | | Hz, 1 H), 6.03-6.21 (m, 2 H), | | | | |
| | yl}methoxy)carbonyl]oxy}ethoxy)eth- | | 4.50 (t, J = 5.57 Hz, 2 H), 4.27- | | | | |
| | yl]butanamide; trifluoroacetic acid | | 4.44 (m, 1 H), 4.18-4.21 (m, 2 | | | | |
| | | | H), 3.81 (t, J = 5.57 Hz, 2 H), | | | | |
| | | | 3.41-3.69 (m, 4 H), 3.09-3.41 | | | | |
| | | | (m, 5 H), 1.06 (d, J = 5.86 Hz, 6 | | | | |
| | | | H). | | | | |
| 391 | trifluoroacetic acid ({4-oxo-1-[2- | 647 | 1H NMR (300 MHz, | 36.5 | 96% remaining | 70% remaining | 1% remaining |
| | (propan-2-yloxy)ethyl]-1H,4H,5H- | | CHLOROFORM-d6) δ 9.76 (br | | after 1 h | after 1 h | after 1 h |
| | pyrrolo[3,2-d]pyrimidin-2- | | s, 1 H), 7.17-7.36 (m, 1 H), | | | | |
| | yl}sulfanyl) methyl N-(2-{2-[(2R)-2- | | 6.73 (br s, 1 H), 6.15-6.35 (m, | | | | |
| | amino-3- | | 3 H), 4.53 (t, J = 5.57 Hz, 2 H), | | | | |
| | methylbutanamido]ethoxy}ethyl)-N- | | 4.27-4.32 (m, 3 H), 3.84 (t, | | | | |
| | methylcarbamate | | J = 5.57 Hz, 2 H), 3.66 (t, J = 4.40 | | | | |
| | | | Hz, 2 H), 3.31-3.61 (m, 5 H), | | | | |
| | | | 2.50-2.60 (m, 2 H), 2.09 (s, 3 | | | | |
| | | | H), 1.80-1.98 (m, 2 H), 1.43 | | | | |
| | | | (s, 9 H), 0.97-1.19 (m, 6 H). | | | | |
| 392 | tert-butyl 3-(2-{[{{4-oxo-1- | 547 | 1H NMR (300 MHz, | 1.2 | 95% remaining | 71% remaining | 1% remaining |
| | [2-(propan-2-yloxy)ethyl]-2- | | CHLOROFORM-d6) δ 10.86 | | after 1 h | after 1 h | after 1 h |
| | sulfanylidene-1H,2H,3H,4H,5H- | | (br s, 1 H), 7.95 (br s, 1 H), 7.31 | | | | |
| | pyrrolo[3,2-d]pyrimidin-5- | | (d, J = 2.93 Hz, 1 H), 6.08-6.33 | | | | |
| | yl}methoxy)carbonyl]oxy}eth- | | (m, 3 H), 4.49-4.54 (m, 2 H), | | | | |
| | oxy)propanoate | | 4.24-4.32 (m, 3 H), 3.81 (t, | | | | |
| | | | J = 5.57 Hz, 2 H), 3.61-3.74 (m, | | | | |
| | | | 2 H), 3.21-3.60 (m, 5 H), 2.45 | | | | |
| | | | 2.70 (m, 2 H), 2.04-2.25 (m, 2 | | | | |
| | | | H), 2.02 (s, 3H), 0.92-1.12 (m, | | | | |
| | | | 7 H). | | | | |
| 393 | 1-tert-butyl 2-{4-oxo-1-[2-(propan-2- | 528 | 1H NMR (300 MHz, | 1.1 | 99% remaining | 94% remaining | 92% remaining |
| | yloxy)ethyl]-2-sulfanylidene- | | CHLOROFORM-d6) δ 12.00 | | after 1 h | after 1 h | after 1 h |
| | 1H,2H,3H,4H,5H-pyrrolo[3,2- | | (br s, 1 H), 8.52 (br s, 2 H), 8.17 | | | | |
| | d]pyrimidin-5-yl}methyl (2R)- | | (br s, 1 H), 7.40 (t, J = 2.64 Hz, 1 | | | | |
| | pyrrolidine-1,2-dicarboxylate | | H), 6.25-6.30 (m, 1 H), 6.13 (d, | | | | |
| | | | J = 11.14 Hz, 1 H), 5.57 (d, | | | | |
| | | | J = 11.14 Hz, 1 H), 4.30-4.40 | | | | |
| | | | (m, 2 H), 4.00-4.16 (m, 2 H), | | | | |
| | | | 3.77 (t, J = 5.57 Hz, 2 H), 3.41- | | | | |
| | | | 3.57 (m, 2 H), 3.02-3.42 (m, 6 | | | | |
| | | | H), 2.95 (s, 2 H), 2.84 (s, 1 H), | | | | |
| | | | 2.20-2.30 (m, 1 H), 0.92- | | | | |
| | | | 1.15 (m, 12 H). | | | | |
| 394 | trifluoroacetic acid {4-oxo-1-[2- | | Ref Joe's first batch | | | | |
| | (propan-2-yloxy)ethyl]-2- | | | | | | |
| | sulfanylidene-1H,2H,3H,4H,5H- | | | | | | |
| | pyrrolo[3,2-d]pyrimidin-5-yl}methyl | | | | | | |
| | (2R)-pyrrolidine-2-carboxylate | | | | | | |
| 395 | {4-oxo-1-[2-(propan-2-yloxy)ethyl]- | 501 | 1H NMR (300 MHz, | 144 | 97% remaining | 23% remaining | 1% remaining |
| | 2-sulfanylidene-1H,2H,3H,4H,5H- | | CHLOROFORM-d6) δ 7.28 (d, | | after 1 h | after 1 h | after 1 h |
| | pyrrolo[3,2-d]pyrimidin-5-yl}methyl | | J = 2.93 Hz, 1 H), 6.25-6.26 (m, | | | | |
| | 2-{[(tert- | | 3 H), 4.51 (s, 2 H), 4.24-4.27 | | | | |
| | butoxy)carbonyl](methyl)amino}acetate | | (m, 2 H), 3.82 (t, J = 5.57 Hz, 2 | | | | |
| | | | H), 3.62-3.67 (m, 4 H), 3.55 (s, | | | | |
| | | | 1 H), 2.43 (t, , J = 6.01 Hz, 1 H), | | | | |
| | | | 1.42 (s, 9 H), 1.05 (d, J = 5.86 | | | | |
| | | | Hz, 6 H). | | | | |
| 396 | trifluoroacetic acid {4-oxo-1-[2- | (M + Na)⁺ = 504 | 1H NMR (300 MHz, | | | | |
| | (propan-2-yloxy)ethyl]-2- | | CHLOROFORM-d6) δ 9.13- | | | | |
| | sulfanylidene-1H,2H,3H,4H,5H- | | 9.34 (m, 1 H), 7.25-7.33 (m, 1 | | | | |
| | pyrrolo[3,2-d]pyrimidin-5-yl}methyl | | H), 6.08-6.43 (m, 3 H), 4.42- | | | | |
| | 2-(methylamino)acetate | | 4.64 (m, 2 H), 4.15-4.36 (m, 1 | | | | |
| | | | H), 3.83 (t, J = 5.57 Hz, 2 H), | | | | |
| | | | 3.30-3.59 (m, 3 H), 2.05- | | | | |
| | | | 2.15 (m, 1 H), 1.69-1.99 (m, 3 | | | | |
| | | | H), 1.25 (s, 9 H), 1.05 (d, | | | | |
| | | | J = 5.86 Hz, 6 H). | | | | |
| 397 | 3-(2-{[{{4-oxo-1-[2-(propan-2- | 381 | 1H NMR (300 MHz, | | | | |
| | yloxy)ethyl]-2-sulfanylidene- | | CHLOROFORM-d6) δ 10.82 | | | | |
| | 1H,2H,3H,4H,5H-pyrrolo[3,2- | | (br s, 1 H), 7.13-7.43 (m, 1 H), | | | | |
| | d]pyrimidin-5- | | 6.18-6.42 (m, 3 H), 4.45-4.60 | | | | |
| | yl}methoxy)carbonyl]oxy}eth- | | (m, 3 H), 3.84 (t, J = 5.57 Hz, 2 | | | | |
| | oxy)propanoic acid | | H), 3.33-3.60 (m, 2 H), 2.30- | | | | |
| | | | 2.43 (m, 2 H), 1.94-2.26 (m, 3 | | | | |
| | | | H), 1.06 (d, J = 5.86 Hz, 6 H). | | | | |
| 398 | {4-oxo-1-[2-(propan-2-yloxy)ethyl]- | (M + Na)⁺ = 478 | 1H NMR (300 MHz, | | | | |
| | 2-sulfanylidene-1H,2H,3H,4H,5H- | | CHLOROFORM-d) δ 9.48 (br | | | | |
| | pyrrolo [3,2-d]pyrimidin-5-yl}methyl | | d, J = 13.48 Hz, 1 H), 7.21-7.36 | | | | |
| | (2R)-1-[2- | | (m, 1 H), 6.17-6.37 (m, 3 H), | | | | |
| | (acetyloxy)acetyl]pyrrolidine-2- | | 4.52 (t, J = 5.57 Hz, 2 H), 3.76- | | | | |
| | carboxylate | | 3.94 (m, 4 H), 3.50-3.60 (m, 1 | | | | |
| | | | H), 2.88 (d, J = 4.69 Hz, 3 H), | | | | |
| | | | 1.43 (s, 9 H), 1.06 (d, J = 5.86 | | | | |
| | | | Hz, 6 H). | | | | |
| 399 | {4-oxo-1-[2-(propan-2-yloxy)ethyl]- | 356 | 1H NMR (300 MHz, | | | | |
| | 2-sulfanylidene-1H,2H,3H,4H,5H- | | CHLOROFORM-d6) δ 11.21 (s, | | | | |
| | pyrrolo[3,2-d]pyrimidin-5-yl}methyl | | 1 H), 7.16-7.36 (m, 1 H), 6.18- | | | | |
| | (2R)-1-[(2S)-2-(benzoyloxy)-3- | | 6.42 (m, 3 H), 4.55 (t, J = 5.57 | | | | |
| | methylbutanoyl]pyrrolidine-2- | | Hz, 2 H), 3.95 (s, 2 H), 3.84 (t, | | | | |
| | carboxylate | | J = 5.27 Hz, 2 H), 3.25-3.60 | | | | |
| | | | (m, 1 H), 2.83 (s, 3 H), 1.06 (d, | | | | |
| | | | J = 5.86 Hz, 6 H). | | | | |
| 400 | {4-oxo-1-[2-(propan-2-yloxy)ethyl]- | 467 | 1H NMR (300 MHz, | 2.42 | 99% remaining | 26% remaining | 89% remaining |
| | 2-sulfanylidene-1H,2H,3H,4H,5H- | | CHLOROFORM-d6) δ 10.28 (s, | | after 1 h | after 1 h | after 1 h |
| | pyrrolo[3,2-d]pyrimidin-5-yl}methyl | | 1 H), 7.30 (d, J = 3.52 Hz, 1 H), | | | | |
| | 2-[2-(acetyloxy)-N- | | 6.11-6.37 (m, 3 H), 4.53 (t, | | | | |
| | methylacetamido]acetate | | J = 5.57 Hz, 2 H), 4.20-4.37 (m, | | | | |
| | | | 2 H), 3.52-3.93 (m, 7 H), 2.59 | | | | |
| | | | (t, J = 6.15 Hz, 2 H), 0.99-1.16 | | | | |
| | | | (m, 6 H). | | | | |
| 401 | {4-oxo-1-[2-(propan-2-yloxy)ethyl]- | 482 | 1H NMR (300 MHz, | 4.3 | 90% remaining | 8% remaining | 0% remaining |
| | 2-sulfanylidene-1H,2H,3H,4H,5H- | | CHLOROFORM-d6) δ 9.32 (s, | | after 1 h | after 1 h | after 1 h |
| | pyrrolo [3,2-d]pyrimidin-5-yl}methyl | | 1 H), 7.19-7.36 (m, 1 H), 6.24- | | | | |
| | 2-[(2S)-2-(benzoyloxy)-N,3- | | 6.38 (m, 3 H), 4.46-4.85 (m, 4 | | | | |
| | dimethylbutanamido]acetate | | H), 3.72-3.92 (m, 2 H), 3.43- | | | | |
| | | | 3.67 (m, 2 H), 2.03-2.24 (m, 5 | | | | |
| | | | H), 1.85-1.92 (m, 4 H), 1.06 | | | | |
| | | | (d, J = 5.86 Hz, 6 H). | | | | |
| 402 | 5-{({2-[2- | 586 | 1H NMR (300 MHz, | 246 | 99% remaining | 89% remaining | 99% remaining |
| | (carbamoyloxy)ethoxy]ethoxy}car- | | CHLOROFORM-d6) δ 9.39 (s, | | after 1 h | after 1 h | after 1 h |
| | bonyl)oxy]methyl}-1-[2-(propan-2- | | 1 H), 7.97-8.14 (m, 2 H), 7.52- | | | | |
| | yloxy)ethyl]-2-sulfanylidene- | | 7.63 (m, 1 H), 7.41-7.52 (m, 2 | | | | |
| | 1H,2H,3H,4H,5H-pyrrolo[3,2- | | H), 7.16-7.20 (m, 1 H), 6.12- | | | | |
| | d]pyrimidin-4-one | | 6.30 (m, 2 H), 6.09 (d, J = 2.93 | | | | |
| | | | Hz, 1 H), 5.05-5.13 (m, 1 H), | | | | |
| | | | 4.40-4.54 (m, 3 H), 4.01-4.10 | | | | |
| | | | (m, 1 H), 3.69-3.89 (m, 3 H), | | | | |
| | | | 3.40-3.62 (m, 2 H), 2.22-2.47 | | | | |
| | | | (m, 2 H), 1.84-2.14 (m, 2 H), | | | | |
| | | | 0.94-1.17 (m, 12 H). | | | | |
| 403 | 3-{[{{2-[2- | 457 | 1H NMR (300 MHz, | 4.77 | 98% remaining | 54% remaining | 0% remaining |
| | (carbamoyloxy)ethoxy]ethoxy}car- | | CHLOROFORM-d6) δ 9.24 | | after 1 h | after 1 h | after 1 h |
| | bonyl)oxy]methyl}-1-[2-(propan-2- | | (brs, 1 H), 7.13-7.23 (m, 1 H), | | | | |
| | yloxy)ethyl]-2-sulfanylidene- | | 6.17-6.31 (m, 3 H), 4.82 (s, 2 | | | | |
| | 1H,2H,3H,4H,5H-pyrrolo[3,2- | | H), 4.52 (t, J = 5.57 Hz, 2 H), | | | | |
| | d]pyrimidin-4-one | | 4.18 (s, 2 H), 3.84 (t, J = 5.57 | | | | |
| | | | Hz, 2 H), 3.44-3.63 (m, 1 H), | | | | |
| | | | 3.05 (s, 3 H), 2.23 (s, 3 H), 1.06 | | | | |
| | | | (d, J = 5.86 Hz, 6 H). | | | | |
| 404 | 17-[2-(propan-2-yloxy)ethyl]-16- | 560 | 1H NMR (300 MHz, | 50.8 | 92% remaining | 86% remaining | 77% remaining |
| | sulfanylidene-3,5,8,11,13- | | CHLOROFORM-d6) δ 9.46 (s, | | after 1 h | after 1 h | after 1 h |
| | pentaoxa-1,15,17- | | 1 H), 7.98-8.15 (m, 2 H), 7.51- | | | | |
| | triazatricyclo[13.5.2.0\hat{ }{18,21}]docosa- | | 7.69 (m, 1 H), 7.36-7.51 (m, 2 | | | | |
| | 18(21),19-diene-4,12,22-trione | | H), 7.18-7.34 (m, 1 H), 6.14- | | | | |
| | | | 6.37 (m, 3 H), 5.27 (d, J = 7.03 | | | | |
| | | | Hz, 2 H), 4.38-4.64 (m, 2 H), | | | | |
| | | | 3.70-3.92 (m, 3 H), 3.51- | | | | |
| | | | 3.61 (m, 1 H), 3.25 (s, 3 H), | | | | |
| | | | 2.22-2.44 (m, 1 H), 0.94-1.18 | | | | |
| | | | (m, 12 H). | | | | |
| 405 | {4-oxo-1-[2-(propan-2-yloxy)ethyl]- | 460 | 1H NMR (300 MHz, | | 83% remaining | 81% remaining | 1% remaining |
| | 2-sulfanylidene-1H,2H,3H,4H,5H- | | CHLOROFORM-d6) δ 9.43 (s, | | after 1 h | after 1 h | after 1 h |
| | pyrrolo[3,2-d]pyrimidin-5-yl}methyl | | 1 H), 7.26-7.33 (m, 1 H), 6.18- | | | | |
| | (2R)-1-[2-(2-{[(tert- | | 6.35 (m, 3 H), 4.78 (brs, 2 H), | | | | |
| | butoxy)carbonyl]amino}acetamido) | | 4.45-4.61 (m, 2 H), 4.24-4.42 | | | | |
| | acetyl]pyrrolidine-2-carboxylate | | (m, 2 H), 4.06-4.24 (m, 2 H), | | | | |
| | | | 3.76-3.87 (m, 2 H), 3.62-3.76 | | | | |
| | | | (m, 4 H), 3.41-3.62 (m, 1 H), | | | | |
| | | | 1.00-1.14 (m, 6 H). | | | | |
| 406 | trifluoroacetic acid {4-oxo-1-[2- | 460 | 1H NMR (300 MHz, | 34.2 | 88% remaining | 71% remaining | 0% remaining |
| | (propan-2-yloxy)ethyl]-2- | | CHLOROFORM-d6) δ 10.67 | | after 1 h | after 1 h | after 1 h |
| | sulfanylidene-1H,2H,3H,4H,5H- | | (br s, 1 H), 7.18-7.26 (m, 2 H), | | | | |
| | pyrrolo[3,2-d]pyrimidin-5-yl}methyl | | 6.66 (s, 2 H), 6.25 (t, J = 2.34 | | | | |
| | (2R)-1-[2-(2- | | Hz, 1 H), 5.08 (br s, 2 H), 4.64 | | | | |
| | aminoacetamido)acetyl]pyrrolidine- | | (t, J = 5.86 Hz, 2 H), 4.27-4.43 | | | | |
| | 2-carboxylate | | (m, 2 H), 4.10-4.27 (m, 2 H), | | | | |
| | | | 3.87 (t, J = 5.86 Hz, 2 H), 3.42- | | | | |
| | | | 3.79 (m, 5 H), 0.95-1.15 (m, 6 | | | | |
| | | | H). | | | | |
| 407 | {4-oxo-1-[2-(propan-2-yloxy)ethyl]- | 473 | 1H NMR (300 MHz, | 146 | 98% remaining | 1% remaining | 0% remaining |
| | 2-sulfanylidene-1H,2H,3H,4H,5H- | | CHLOROFORM-d6) δ 7.12 (d, | | after 1 h | after 1 h | after 1 h |
| | pyrrolo[3,2-d]pyrimidin-5-yl}methyl | | J = 2.93 Hz, 1 H), 6.83 (s, 2 H), | | | | |
| | 2-[2-(2-{[(tert- | | 6.25 (d, J = 2.93 Hz, 1 H), 6.14 | | | | |
| | butoxy)carbonyl]amino}acetamido)- | | (s, 2 H), 4.61 (t, J = 5.57 Hz, 2 | | | | |
| | N-methylacetamido]acetate | | H), 4.32-4.40 (m, 4 H), 3.76- | | | | |
| | | | 3.90 (m, 2 H), 3.64-3.76 (m, 4 | | | | |
| | | | H), 3.57 (dt, J = 12.02, 6.30 Hz, | | | | |
| | | | 1 H), 1.08 (d, J = 5.86 Hz, 6 H). | | | | |
| 408 | trifluoroacetic acid {4-oxo-1-[2- | 596 | 1H NMR (300 MHz, | | | | |
| | (propan-2-yloxy)ethyl]-2- | | CHLOROFORM-d6) δ 7.18- | | | | |
| | sulfanylidene-1H,2H,3H,4H,5H- | | 7.31 (m, 1 H), 6.07-6.41 (m, 3 | | | | |
| | pyrrolo[3,2-d]pyrimidin-5-yl}methyl | | H), 4.37-4.68 (m, 3 H), 4.08 | | | | |
| | 2-[2-(2-aminoacetamido)-N- | | (d, J = 5.86 Hz, 2 H), 3.80-3.90 | | | | |
| | methylacetamido]acetate | | (m, 3 H), 3.41-3.63 (m, 2 H), | | | | |
| | | | 2.65-2.72 (s, 2 H), 1.87-2.25 | | | | |
| | | | (m, 4 H), 1.45 (s, 9 H), 0.96- | | | | |
| | | | 1.19 (m, 6 H). | | | | |
| 409 | 15-[2-(propan-2-yloxy)ethyl]-14- | 496 | 1H NMR (300 MHz, | <1.07 | 94% remaining | 28% remaining | 4% remaining |
| | sulfanylidene-3,5,9,11-tetraoxa-1,13,15- | | CHLOROFORM-d6) δ 11.01 | | after 1 h | after 1 h | after 1 h |
| | triazatricyclo[11.5.2.0\hat{ }{16,19}]icosa | | (br s, 1 H), 8.57 (br s, 1 H), 8.11 | | | | |
| | 16(19),17-diene-4,10,20-trione | | (brs, 2 H), 7.25-7.34 (m, 1 H), | | | | |
| | | | 6.19-6.25 (m, 3 H), 4.40- | | | | |
| | | | 4.68 (m, 3 H), 3.75-4.10 (m, 5 | | | | |
| | | | H), 3.41-3.63 (m, 2 H), 2.75 (s, | | | | |
| | | | 2 H), 2.01-2.15 (m, 2 H), 1.72- | | | | |
| | | | 2.00 (m, 2 H), 1.05 (d, J = 6.45 | | | | |
| | | | Hz, 6 H). | | | | |
| 410 | 5-[{{[3- | 570 | 1H NMR (300 MHz, | | | | |
| | (carbamoyloxy)propoxy]carbon- | | CHLOROFORM-d6) δ 9.94 (br | | | | |
| | yl}oxy)methyl]-1-[2-(propan-2- | | s, 1 H), 7.20-7.35 (m, 1 H), | | | | |
| | yloxy)ethyl]-2-sulfanylidene- | | 6.18-6.39 (m, 3 H), 5.32 (br s, | | | | |
| | 1H,2H,3H,4H,5H-pyrrolo[3,2- | | 1 H), 4.53 (t, J = 5.57 Hz, 2 H), | | | | |
| | d]pyrimidin-4-one | | 4.08-4.24 (m, 3 H), 3.71-3.94 | | | | |
| | | | (m, 4 H), 3.50-3.61 (m, 1 H), | | | | |
| | | | 3.05 (s, 3 H), 2.87 (br s, 2 H), | | | | |
| | | | 1.42 (s, 9 H), 1.07 (d, J = 5.86 | | | | |
| | | | Hz, 6 H). | | | | |
| 411 | 5-[{[4- | 470 | 1H NMR (300 MHz, | <1.07 | 92% remaining | 22% remaining | 1% remaining |
| | (carbamoyloxy)butoxy]carbon- | | CHLOROFORM-d6) δ 11.06 | | after 1 h | after 1 h | after 1 h |
| | yl}oxy)methyl]-1-[2-(propan-2- | | (br s, 1 H), 8.33-8.67 (m, 1 H), | | | | |
| | yloxy)ethyl]-2-sulfanylidene- | | 7.84-8.13 (m, 2 H), 7.26-7.30 | | | | |
| | 1H,2H,3H,4H,5H-pyrrolo[3,2- | | (m, 1 H), 6.18-6.30 (m, 3 H), | | | | |
| | d]pyrimidin-4-one | | 4.34-4.49 (m, 2 H), 4.05-4.31 | | | | |
| | | | (m, 3 H), 3.65-3.85 (m, 3 H), | | | | |
| | | | 3.25-3.60 (m, 4 H), 3.09 (s, 3 | | | | |
| | | | H), 2.87 (br s, 2 H), 1.04 (d, | | | | |
| | | | J = 5.27 Hz, 6 H). | | | | |
| 412 | 3-[{[4- | 442 | 1H NMR (300 MHz, | | | | |
| | (carbamoyloxy)butoxy]carbon- | | CHLOROFORM-d6) δ 7.12 (d, | | | | |
| | yl}oxy)methyl]-1-[2-(propan-2- | | J = 2.93 Hz, 1 H), 6.90 (br s, 2 | | | | |
| | yloxy)ethyl]-2-sulfanylidene- | | H), 6.22-6.31 (m, 1 H), 6.13 | | | | |
| | 1H,2H,3H,4H,5H-pyrrolo[3,2- | | (br s, 2 H), 4.61 (t, J = 5.57 Hz, 2 | | | | |
| | d]pyrimidin-4-one | | H), 4.22-4.46 (m, 4 H), 3.84 (t, | | | | |
| | | | J = 5.57 Hz, 2 H), 3.50-3.62 | | | | |
| | | | (m, 1 H), 2.07-2.27 (m, 2 H), | | | | |
| | | | 1.02-1.16 (m, 6 H). | | | | |
| 413 | 16-[2-(propan-2-yloxy)ethyl]-15- | 429 | 1H NMR (300 MHz, | | | | |
| | sulfanylidene-3,5,10, 12-tetraoxa- | | CHLOROFORM-d6) δ 9.32 (br | | | | |
| | 1,14,16- | | s, 1 H), 7.28-7.35 (m, 2 H), | | | | |
| | triazatricyclo[12.5.2.0\hat{ }{17,20}]henicosa- | | 6.11-6.37 (m, 3 H), 4.64 (br s, | | | | |
| | 17(20),18-diene-4,11,21- | | 2 H), 4.53 (t, J = 5.86 Hz, 2 H), | | | | |
| | trione | | 4.25 (t, J = 6.45 Hz, 2 H), 4.03- | | | | |
| | | | 4.19 (m, 2 H), 3.84 (t, J = 5.57 | | | | |
| | | | Hz, 2 H), 3.50-3.60 (m, 1 H), | | | | |
| | | | 1.85-2.10 (m, 2 H), 1.07 (d, | | | | |
| | | | J = 5.86 Hz, 6 H). | | | | |

### Example 414

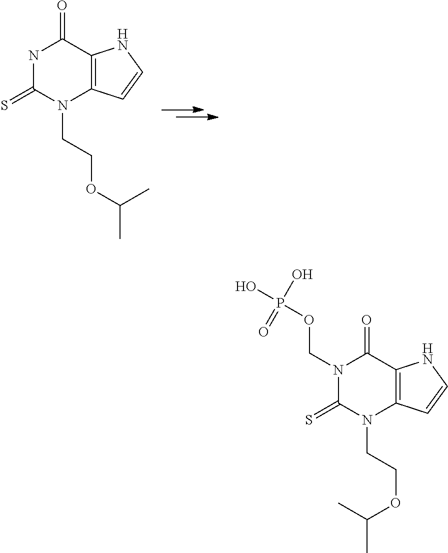 [SMILES: CC(C)OCCn1c(=S)[nH]c(=O)c2[nH]ccc21 ; CC(C)OCCn1c(=S)n(COP(=O)(O)O)c(=O)c2[nH]ccc21]

({4-oxo-1-[2-(propan-2-yloxy)ethyl]-2-sulfanylidene-1H,2H,3H,4H,5H-pyrrolo[3,2-d]pyrimidin-3-yl}methoxy)phosphonic acid. A solution of 1-[2-(propan-2-yloxy)ethyl]-2-sulfanylidene-1H,2H,3H,4H,5H-pyrrolo[3,2-d]pyrimidin-4-one (506 mg, 2.0 mmol) in DMF (8 mL) under nitrogen was treated with lithium hexamethyldisilylamide (1.0 M in THF, 1.8 mL, 1.8 mmol), and the reaction mixture was stirred for 15 minutes. Di-tert-butyl chloromethyl phosphate (520 mg, 2.0 mmol) was added via syringe with continued stirring of the reaction mixture overnight, followed by purification with RP-HPLC (Method D). The later fractions of similar retention time containing the desired product were combined and lyophilized to provide the di-tert-butyl protected intermediate (130 mg, 13.7%). Deprotection of tert-butyl protecting groups was carried out with glacial acetic acid and water (4:1 v/v) at 75° C. for 30 minutes followed by stirring at room temperature overnight. The reaction mixture was purified by RP-HPLC (Method D), and similar fractions were lyophilized to provide the title compound as a white solid (54 mg). ¹H NMR (300 MHz, DMSO-d₆) δ=12.50 (br s, 1H), 7.43 (t, J=2.9 Hz, 1H), 6.34 (t, J=2.6 Hz, 1H), 6.23 (d, J=4.7 Hz, 2H), 4.58 (t, J=6.4 Hz, 2H), 3.74 (t, J=6.2 Hz, 2H), 3.62-3.49 (m, 1H), 1.01 (d, J=6.1 Hz, 6H). LC/MS method A: Rₜ=3.02 mins. (M+H)⁺=364, purity=99%.

### Example 415

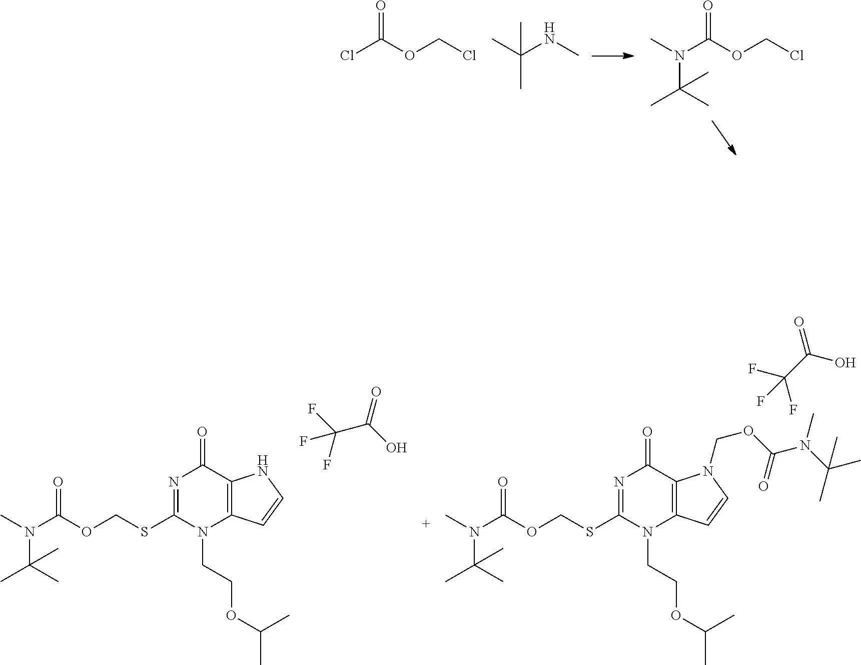

tert-Butyl-methyl-carbamic acid chloromethyl ester. To an ice cooled solution of t-butyl methyl amine (0.135 g, 1.55 mmole) in 2 mL of CH₂Cl₂ was added diisopropylethyl amine (0.67 mL, 2.36 mmole) and chloromethyl chloroformate (0.136 mL, 1.511 mmole) dropwise over 1 minute. After 18 hours, the reaction mixture was concentrated in vacuo and purified on a 40 g Agela column eluting with 0-60% EtOAc in hexanes. The desired fractions were concentrated in vacuo to yield the titled product (0.188 g, 67%). ¹H NMR (300 MHz, CHLOROFORM-d) δ=5.78 (s, 1H), 2.94 (s, 1H), 1.41 (s, 4H).

tert-Butyl-methyl-carbamic acid 1-(2-isopropoxy-ethyl)-4-oxo-4,5-dihydro-1H-pyrrolo[3,2-d]pyrimidin-2-ylsulfanylmethyl ester, trifluoroacetic acid salt. Sodium hydride (23 mg, 0.98 mmole) was added to a solution of 1-(2-Isopropoxy-ethyl)-2-thioxo-1,2,3,5-tetrahydro-pyrrolo[3,2-d]pyrimidin-4-one (0.10 g, 0.39 mmole) in 5 mL of THF. After gas evolution had ceased, tert-butyl-methyl-carbamic acid chloromethyl ester (0.156 g, 0.86 mmole) in 1 mL of THF was added. After 18 hours, the reaction was quenched with saturated aqueous NH 4 C1. The aqueous layer was extracted with 2×20 mL of EtOAc. The combined organic layers were washed with brine, dried over Na₂SO₄, filtered, and concentrated in vacuo. The product was purified by RP-HPLC 40-95% ACN in H₂O with 0.1% TFA. The desired fractions were lyophilized to yield tert-butyl-methyl-carbamic acid 1-(2-isopropoxy-ethyl)-4-oxo-4,5-dihydro-1H-pyrrolo[3,2-d]pyrimidin-2-ylsulfanylmethyl ester, trifluoroacetic acid salt (68.5 mg, 26%). ¹H NMR (300 MHz, CHLOROFORM-d) δ=12.76-12.39 (m, 1H), 7.60-7.41 (m, 1H), 6.35 (dd, J=1.9, 3.0 Hz, 1H), 5.99 (s, 2H), 4.39 (t, J=5.7 Hz, 3H), 3.89-3.67 (m, 2H), 3.50 (t, J=6.1 Hz, 1H), 2.89 (s, 3H), 1.37 (s, 9H), 1.04 (d, J=6.0 Hz, 6H). LC/MS: Rₜ=5.92 mins. (M+H)⁺=397, purity >95% and tert-butyl-methyl-carbamic acid 2-[(tert-butyl-methyl-carbamoyloxy)-methylsulfanyl]-1-(2-isopropoxy-ethyl)-4-oxo-1,4-dihydro-pyrrolo[3,2-d]pyrimidin-5-ylmethyl ester; trifluoro-acetic acid salt (69.0 mg, 27%). ¹H NMR (300 MHz, CHLOROFORM-d) δ=7.48 (d, J=3.3 Hz, 1H), 6.36 (s, 2H), 6.24 (d, J=3.3 Hz, 1H), 5.94 (s, 2H), 4.27 (t, J=5.8 Hz, 2H), 3.74 (br t, J=5.6 Hz, 7H), 3.56-3.44 (m, 1H), 2.89 (s, 3H), 2.87 (s, 3H), 1.37 (s, 9H), 1.35-1.32 (m, 9H), 1.04 (d, J=6.0 Hz, 6H). LC/MS: Rₜ=7.00 mins. (M+H)⁺=540, purity >95%.

### Example 416

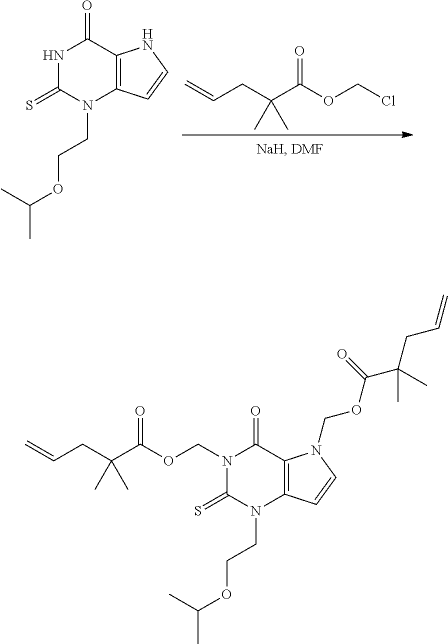

(1-(2-isopropoxyethyl)-4-oxo-2-thioxo-1H-pyrrolo[3,2-d]pyrimidine-3,5(2H,4H)-diyl)bis(methylene) bis(2,2-dimethylpent-4-enoate). A suspension of 1-(2-isopropoxyethyl)-2-thioxo-2,3-dihydro-1H-pyrrolo[3,2-d]pyrimidin-4(5H)-one (50 mg, 0.197 mmol) in DMF (1 mL) was treated with sodium hydride (60% in mineral oil, 12.6 mg, 0.315 mmol) under N₂ and stirred for 30 minutes. Chloromethyl 2,2-dimethylpent-4-enoate (69.5 mg, 0.394 mmol) in DMF (0.5 mL) was added via syringe and the mixture was stirred for 18 h. The reaction was quenched with a few drops of saturated aqueous NH₄Cl and purified by normal phase chromatography (20% EtOAc/hexanes) to get the pure product separated from other isomers as a white solid (12 mg, 15%). ¹H NMR (300 MHz, CHLOROFORM-d) δ=7.28 (d, J=3.3 Hz, 1H), 6.57 (s, 2H), 6.26-6.23 (m, 3H),), 5.71-5.85 (m, 1H),), 5.50-5.62 (m, 1H), 5.00-5.08 (m, 2H), 4.85-4.91 (m, 2H), 4.62 (t, J=5.6 Hz, 2H), 3.86 (t, J=5.6 Hz, 2H), 3.50-3.61 (m, 1H), 2.26-2.29 (m, 2H), 2.20-2.23 (m, 2H), 1.18 (s, 6H), 1.12 (s, 6H), 1.06 (d, J=6.4 Hz, 6H). LC/MS (method A) (M+H)⁺=535, purity >95%.

The above schemes and procedures are provided only to illustrate embodiments of the present invention. In some cases, the final product may be further modified, for example, by manipulation of substituents. These manipulations may include, but are not limited to, reduction, oxidation, alkylation, acylation, and hydrolysis reactions which are commonly known to those of ordinary skill in the art.

In some cases, the order of carrying out the reactions in the foregoing schemes may be varied to facilitate the reaction or to avoid unwanted reaction products. Additionally, various protecting group strategies may be employed to facilitate the reaction or to avoid unwanted reaction products. The following examples are provided so that the invention might be more fully understood. These examples are illustrative only and should not be construed as limiting the invention in any way.

### Compound Synthesis and Characterization

The compounds of the invention may be tested for biological activity as MPO inhibitors by methods known to those skilled in the art. In addition, methods such as, for example, a Caco-2 assay, may be used to test the bioavailability of the compounds of the invention. A description of a Caco-2 permeability assay is as follows:

### Caco-2 Permeability Assay

The Caco-2 monolayer is widely used across the pharmaceutical industry as an in vitro model of the human small intestinal mucosa to predict the absorption of orally administered drugs.¹⁻⁴
The Caco-2 cell model mimics processes such as transcellular transport, paracellular transport, and some aspects of efflux and active transport.
Readout: Pₐₚₚ (A→B), Pₐₚₚ (B→A), Efflux ratio, % Recovery
Controls: with and without Pgp inhibitor; Atenolol, Propanolol, Taxel-1

### Assay Description:

The Caco-2 cells are cultured to confluency, trypsinized and seeded onto a filter transwell insert at a density of ^{˜}32,000 cells/well in DMEM cell culture medium. Cells are grown in a humidified atmosphere of 5% CO₂ at 37° C. Following an overnight attachment period (24 h after seeding), the cell medium is replaced with fresh medium in both the apical and basolateral compartments every other day. The cell monolayers are used for transport studies 21 days post seeding after measuring the TEER values (>600 Ohms/cm²). The apical sides and basolateral sides are washed consecutively with HBSS 2.5% (v/v), HEPES (pH 7.4) or HBSS 2.5% (v/v), HEPES 10% (v/v), and Fetal Bovine Serum (pH 7.4) at 37° C. in an incubator under an atmosphere of 5% CO₂.

Donor working solution is prepared by dilution of DMSO stock of test article or positive control with transport media to 10 μM.

For A→B directional transport, the donor working solution (with test article or positive control, with or without Pgp inhibitor) is added to the apical (A) compartment and the transport media as receiver working solution is added to the basolateral (B) compartment. For B→A directional transport, the donor working solution (with positive control or test article, with or without Pgp inhibitor) is added to the basolateral (B) compartment and transport media as receiver working solution is added to the apical (A) compartment.

The cells are incubated in a humidified atmosphere of 5% CO₂ at 37° C. for 90 minutes.

At the end of the incubation, samples are taken from both donor and receiver compartments and transferred into 96-well assay plates containing internal standard solution (IS) in each well. After centrifugation, the supernatant solutions are transferred to clean 96 well plates and analyzed by LC-MS/MS. The MS detection is performed using a Sciex API 4000 instrument. Each compound is analyzed by reversed phase HPLC.

### Data Analysis:

The parameters Pₐₚₚ (apparent permeability) and efflux ratio are calculated as follows:

Pₐₚₚ=(dQ/dt)×(1/C₀)×(1/A)

Efflux ratio=Pₐₚₚ[B→A]/Pₐₚₚ[A→B]

where dQ/dt is the permeability rate, C₀ is the initial concentration in the donor compartment, and A is the surface area of the cell monolayer (0.33 cm²). The Papp value is a rate measured in cm/s. Calculated Pₐₚₚ is ranked as low (<1×10⁶ cm/s), moderate (1-10×10⁻⁶ cm/s), and high (>10×10⁶ cm/s).

Comparing the efflux ratios generated in the presence and absence of a Pgp inhibitor identifies whether the test article is a Pgp substrate. A compound is considered to be a Pgp substrate when the efflux ratio in the absence of inhibitor is >1.99 and is significantly reduced (≤1) in the presence of an inhibitor.

Recovery is calculated as follows:

% Recovery=(Total compound mass in donor and receiver compartments at the end of the incubation/Initial compound mass in the donor compartment)×100.

### Stability in Simulated Gastric Fluid (SGF) and Simulated Intestinal Fluid (SIF)

Simulated gastric fluid (SGF) and Simulated intestinal fluid (SIF) stability are in vitro metrics indicating the ability of a compound to pass through the gastrointestinal tract digested, partially digested or completely digested. An undigested or nearly undigested compound will usually survive gut transit long enough to be absorbed into the systemic circulation. Greater than 50% remaining intact compound at 1 hour indicates a relatively stable molecule that will likely be absorbed in the gut.

For each test compound, samples at a final concentration of 2 μg/mL are prepared in respective blank simulated gastric fluid with pepsin (pH 1.2) and simulated intestinal fluid with pancreatin (pH 6.8). All samples are incubated at 37° C. on a 150-rpm orbital shaker, and an aliquot is removed at predetermined time points (0 to 60 minutes). Samples are precipitated with three volumes of acetonitrile containing propranolol as internal standard, and centrifuged for 10 min at 2,000 g before LC-MS/MS analysis of the supernatant solutions. Percent parent compound remaining is determined relative to 0-minute incubation samples. If a sufficient number of time points are available, the degradation half-life is calculated based on the natural log of % compound remaining vs. time plot.

Plasma stability estimates the ability of a compound to remain intact in the blood. For prodrugs that are intended to be hydrolyzed by plasma enzymes a certain level of instability in plasma indicates the compound is probably breaking down to the parent drug. A stability range of 25-75% remaining parent compound after 1 hour in plasma indicates the compound is likely breaking down to the parent drug molecule and a patient will be exposed to this parent molecule.

### Plasma Stability

Assessment of plasma stability was carried out by individual incubations of drug candidates in fresh human plasma at a concentration of 1 μM for 1 hour at 37° C. After which, the samples were de-proteinized by addition of 2 volumes of acetonitrile containing 0.1% formic acid and internal standard, vortex mixed for 2 minutes and centrifuged at 4,000 rpm for 10 minutes to pellet precipitated protein. The resulting supernatant containing the drug candidates was diluted 5-fold with water containing 0.1% formic acid and submitted to LCMS/MS analysis. All determinations were done in triplicate. Plasma stability was expressed as percent of control remaining.

See Pelletier et al. ACS Med. Chem. Lett. 2018, 9, 752-756.

### Abbreviations

ACN Acetonitrile
DCM Dichloromethane
DMEM Dulbecco's Modified Eagle Medium
DMF Dimethylformamide
DMSO Dimethylsulfoxide
EtOAc Ethyl acetate
HBSS Hank's Buffered Salt Solution
HEPES 4-(2-Hydroxyethyl)-1-piperazineethanesulfonic acid
hPlasma Human plasma
HPLC High-performance liquid chromatography
MeOH Methanol
MPO Myeloperoxidase
LC/MS Liquid chromatography/mass spectrometry
Pgp P-glycoprotein 1, also known as multidrug resistance protein 1 (MDR1)
LC Liquid chromatography
LiHMDS Lithium bis(trimethylsilyl)amide
MS Mass spectrometry
NMR Nuclear magnetic resonance
RP-HPLC Reversed phase high-performance liquid chromatography
SIF Simulated intestinal fluid
SGF Simulated gastric fluid
TEER Transepithelial Resistance (in Ohm/cm²)
TFA Trifluoroacetic acid
THF Tetrahydrofuran
TMS Tetramethylsilane

### LITERATURE

1. Artursson, P.; Karlsson, J. “Correlation between oral drug absorption in humans and apparent drug permeability coefficients in human intestinal epithelial (Caco-2) cells”; Biochem. Biophys. Res. Commun. 175, 880, (1991).
2. Bohets, H.; Annaert, P.; Van Beijsterveldt, L.; Anciaux, K., Verboven, P.; Meuldermans, W., Lavrijsen, K. “Strategies for absorption screening in drug discovery and development”; 1, 367, (2001).
3. Artursson, P.; Palm, K.; Luthman, K. “Caco-2 monolayers in experimental and theoretical predictions of drug transport”; Adv. Drug Del. Rev. 64, 280, (2012).
4. Shah, P.; Jogani, V.; Bagchi, T.; Misra, A. “Role of Caco-2 cell monolayers in prediction of intestinal drug absorption”; Biotechnol. Progr. 22, 186, (2006).

Throughout this application, various publications are referenced by author name and date, or by patent number or patent publication number. The disclosures of these publications are hereby incorporated in their entireties by reference into this application in order to more fully describe the state of the art as known to those skilled therein as of the date of the invention described and claimed herein. However, the citation of a reference herein should not be construed as an acknowledgement that such reference is prior art to the present invention.

Those skilled in the art will recognize, or be able to ascertain using no more than routine experimentation, numerous equivalents to the specific procedures described herein. Such equivalents are considered to be within the scope of this invention and are covered by the following claims. For example, pharmaceutically acceptable salts other than those specifically disclosed in the description and Examples herein can be employed. Furthermore, it is intended that specific items within lists of items, or subset groups of items within larger groups of items, can be combined with other specific items, subset groups of items or larger groups of items whether or not there is a specific disclosure herein identifying such a combination.

What is claimed is:

1. A compound having Formula (I) or Formula (II): 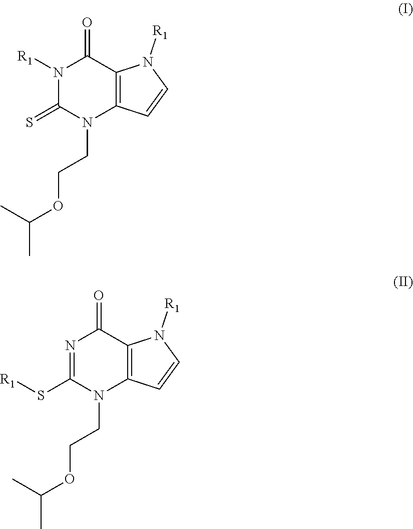
wherein, in Formulae (I) and (II):
each R₁ is independently H, 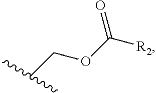
—CH₂OP(═O)(OH)₂, AA₁₋₃, C(═O)(CH₂)ₙ₁X₁AA₁₋₃, or C(═O)AA, provided that at least one R₁ is not H;
AA₁₋₃ is a moiety consisting of 1 to 3 natural amino acids linked through a peptide bond, wherein the natural amino acids are the same or different,
n1 is an integer of 1 to 5,
X₁ is S, O, or NH, and
R₂ is —[C(R₃)₂]ₙR₄, —NR₃R₄, or —OR₄, wherein each R₃ is independently hydrogen or C1-C10 alkyl wherein R₃ are optionally connected to form a ring, and R₄ is a substituted or unsubstituted C1-C20 alkyl group, a substituted or unsubstituted C2-C20 alkenyl group, a substituted or unsubstituted C2-C20 alkynyl group, a substituted or unsubstituted C1-C20 heteroalkyl group, a substituted or unsubstituted C2-C20 heteroalkenyl group, a substituted or unsubstituted C2-C20 heteroalkynyl group, a substituted or unsubstituted C3-C20 cycloalkyl group, a substituted or unsubstituted C3-C20 heterocycloalkyl group, a substituted or unsubstituted C6-C20 aryl group, or a substituted or unsubstituted C1-C20 heteroaryl group, and n is 0 or 1,
wherein, when each R₁ is 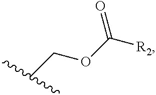
R₂ are optionally connected to form a ring.

2. The compound according to claim 2, wherein, in Formulae (I) and (II), each R₁ is

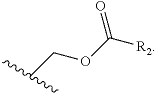

3. The compound according to claim 1, wherein at least one R₃ is not hydrogen.

4. The compound according to claim 2, wherein at least one R₃ is a methyl group.

5. The compound according to claim 2, wherein each R₃ is not hydrogen.

6. The compound according to claim 2, wherein each R₃ is a methyl group.

7. The compound according to claim 2, wherein R₄ is each independently a substituted or unsubstituted C1-C10 alkyl group, a substituted or unsubstituted C2-C10 alkenyl group, a substituted or unsubstituted C2-C10 alkynyl group, a substituted or unsubstituted C1-C10 heteroalkyl group, a substituted or unsubstituted C2-C10 heteroalkenyl group, a substituted or unsubstituted C2-C10 heteroalkynyl group, or a substituted or unsubstituted C6-C10 aryl group.

8. The compound according to claim 1, wherein Formula (I) is represented by one of Formulae (Ia), (Ib), and (Ic), and wherein Formula (II) is represented by one of Formulae (IIa) and (IIb): 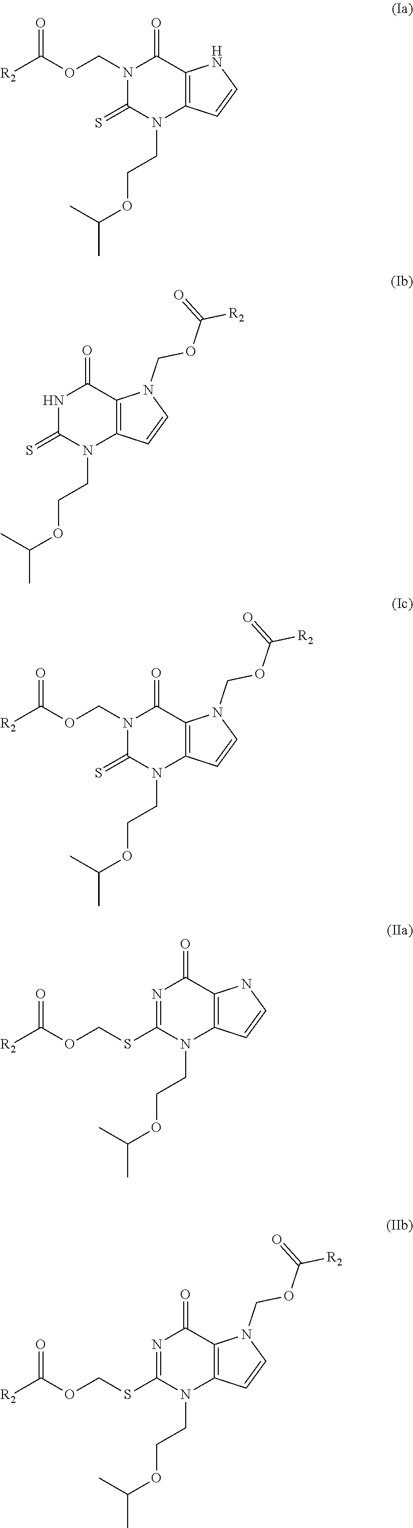
wherein, in Formulae (Ia), (Ib), (Ic), (IIa), and (IIb), R₂ is the same as that in claim 1.

9. The compound according to claim 8, wherein, in Formulae (Ic) and (IIb), R₂ are connected with a single bond to form a linking group L, as in Formulae (Id) and (IIc), respectively: 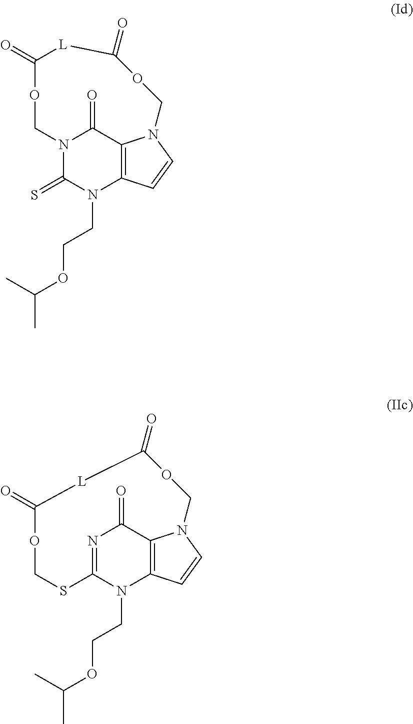
wherein, L is a substituted or unsubstituted C1-C10 alkylene group, a substituted or unsubstituted C2-C10 alkenylene group, a substituted or unsubstituted C2-C10 alkynylene group, a substituted or unsubstituted C1-C10 heteroalkylene group, a substituted or unsubstituted C2-C10 heteroalkenylene group, a substituted or unsubstituted C2-C10 heteroalkynylene group, a substituted or unsubstituted C3-C10 cycloalkylene group, a substituted or unsubstituted C3-C10 heterocycloalkylene group, a substituted or unsubstituted C6-C10 arylene group, or a substituted or unsubstituted C1-C10 heteroarylene group, or any combination thereof.

10. The compound according to claim 11, wherein the linking group L is: 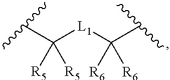
wherein
L₁ is a substituted or unsubstituted C1-C10 alkylene group, a substituted or unsubstituted C2-C10 alkenylene group, a substituted or unsubstituted C2-C10 alkynylene group, a substituted or unsubstituted C1-C10 heteroalkylene group, a substituted or unsubstituted C2-C10 heteroalkenylene group, a substituted or unsubstituted C2-C10 heteroalkynylene group, a substituted or unsubstituted C3-C10 cycloalkylene group, a substituted or unsubstituted C3-C10 heterocycloalkylene group, a substituted or unsubstituted C6-C10 arylene group, or a substituted or unsubstituted C1-C10 heteroarylene group, and
R₅ and R₆ are each independently hydrogen or C1-C10 alkyl, wherein two R₅ and two R₆ are optionally connected to form a ring.

11. The compound according to claim 10, wherein at least one R₅ is not hydrogen and at least one R₆ is not hydrogen.

12. The compound according to claim 10, wherein at least one R₅ is a methyl group and at least one R₆ is a methyl group.

13. The compound according to claim 10, wherein each R₅ is not hydrogen and each R₆ is not hydrogen.

14. The compound according to claim 10, wherein each R₅ is a methyl group and each R₆ is a methyl group.

15. The compound according to claim 1, wherein the compound is represented by Formula (III): 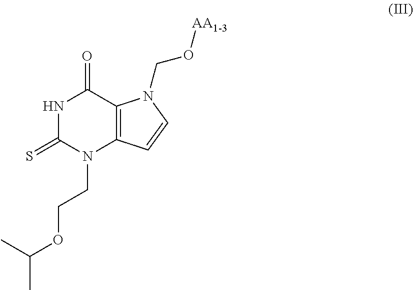
wherein, in Formula (V), AA₁₋₃ is a moiety consisting of 1 to 3 natural amino acids linked through a peptide bond.

16. The compound according to claim 1, wherein the compound is represented by Formula (IV): 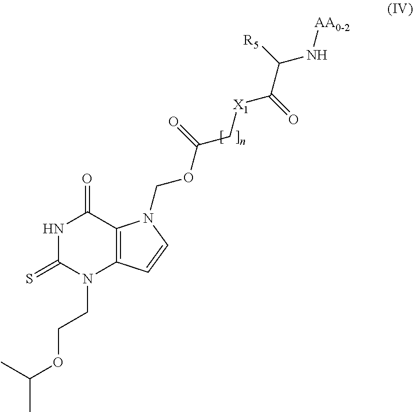
wherein, in Formula (IV), X₁ is S, O, or NH, R₅ is H or a side-chain group present in a natural amino acid, AA₀₋₂ is H or a moiety consisting of 1 or 2 natural amino acids linked through a peptide bond, and n is an integer of 1 to 5.

17. The compound according to claim 1, wherein the compound is represented by Formula (V): 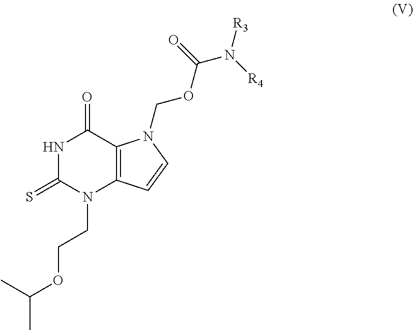
wherein, in Formula (V), R₃ and R₄ are each independently H, a substituted or unsubstituted C1-C10 alkyl group, a substituted or unsubstituted C2-C10 alkenyl group, a substituted or unsubstituted C2-C10 alkynyl group, a substituted or unsubstituted C1-C10 heteroalkyl group, a substituted or unsubstituted C2-C10 heteroalkenyl group, or a substituted or unsubstituted C2-C10 heteroalkynyl group.

18. The compound according to claim 1, wherein the compound is represented by Formula (VI): 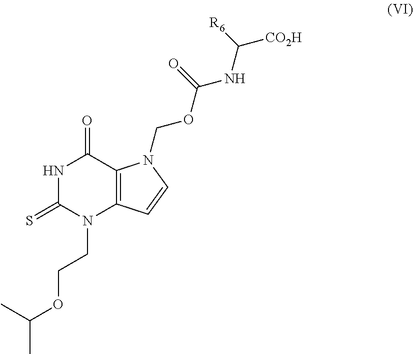
wherein, in Formula (VI), R₆ is H or a side-chain group present in a natural amino acid.

19. The compound according to claim 1, wherein the compound is represented by Formula (VII): 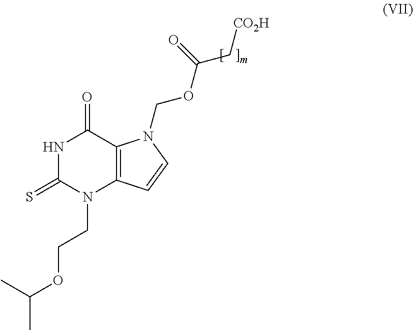
wherein, in Formula (VII), m is an integer of 1 to 25.

20. The compound according to claim 1, wherein the compound is represented by Formula (VIII): 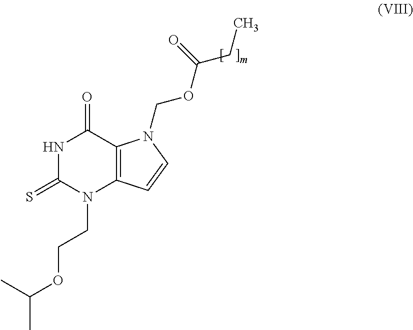
wherein, in Formula (VIII), m is an integer of 1 to 25.

21. The compound according to claim 1, wherein the compound is represented by Formula (IX): 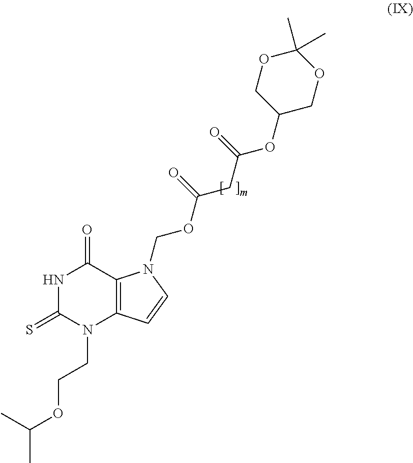
wherein, in Formula (IX), m is an integer of 1 to 25.

22. The compound according to claim 1, wherein the compound is represented by Formula (X): 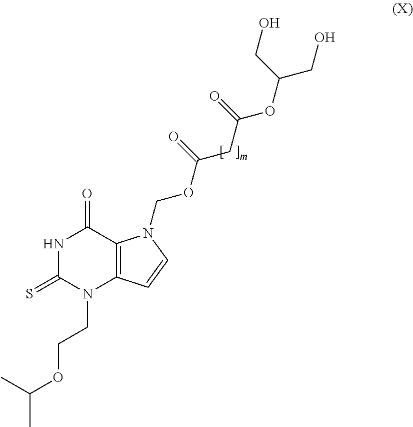
wherein, in Formula (X), m is an integer of 1 to 25.

23. The compound according to claim 1, wherein the compound is represented by Formula (XI): 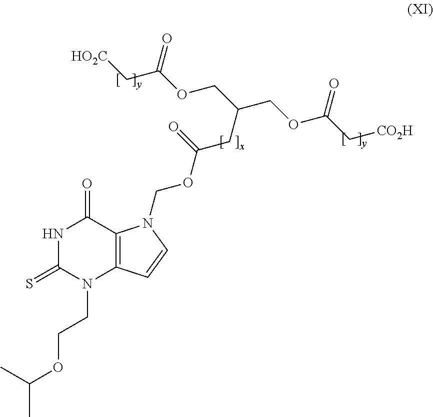
wherein, in Formula (XI), x and each y are independently an integer of 1 to 25.

24. The compound according to claim 1, wherein the compound is represented by Formula (XII): 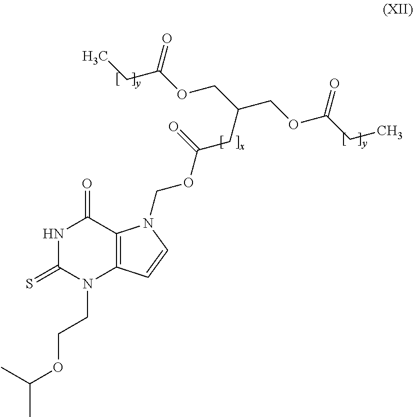
wherein, in Formula (XII), x and each y are independently an integer of 1 to 25.

25. The compound according to claim 1, wherein the compound is represented by Formula (XIII): 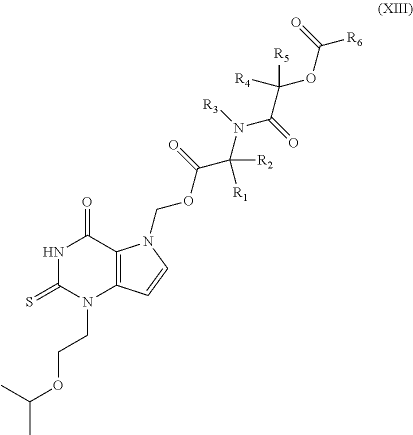
wherein, in Formula (XIII), R₁ to R₅ are each independently hydrogen or C1-C10 alkyl, and R₆ is hydrogen, C1-C10 alkyl, or C6-C10 aryl, wherein any of groups R₁ to R₅ are optionally connected to form a ring.

26. The compound according to claim 1, wherein the compound is represented by Formula (XIV): 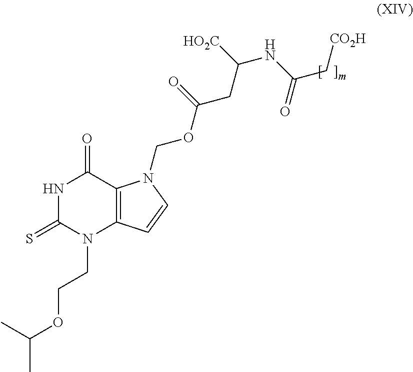
wherein, in Formula (XIV), m is an integer of 1 to 25.

27. The compound according to claim 1, wherein the compound is

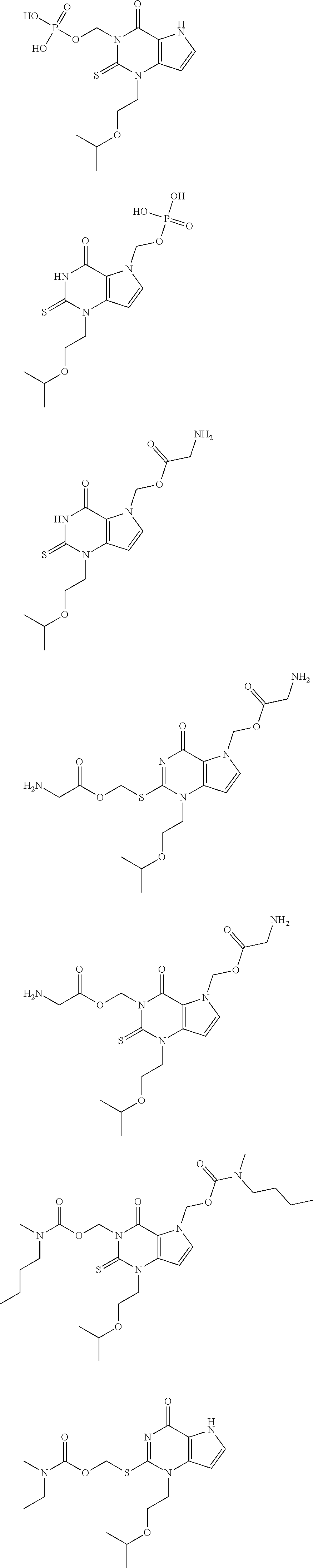

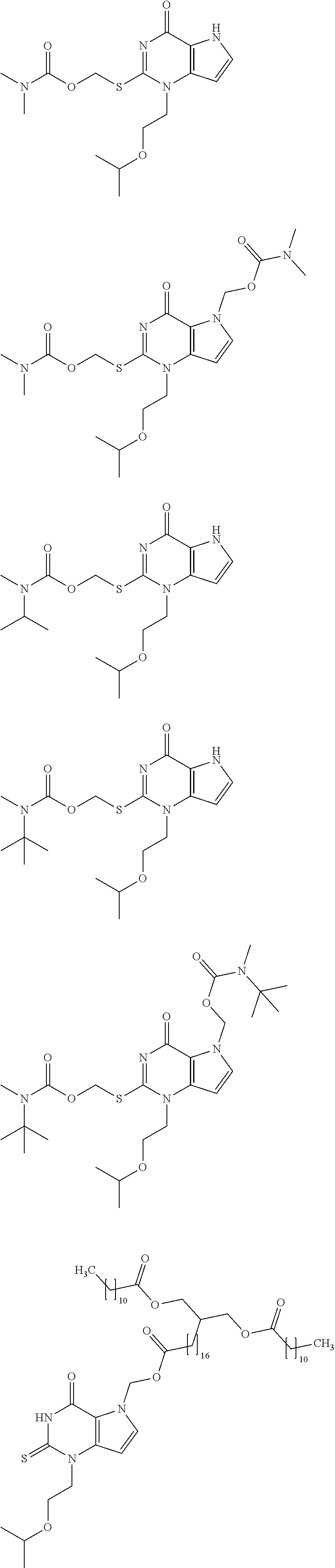

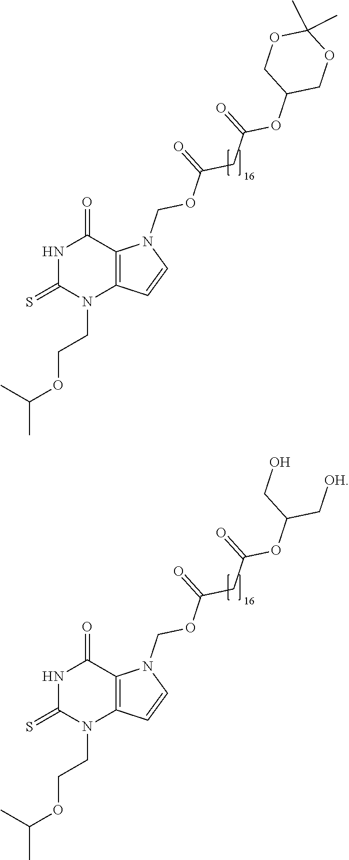

28. A compound selected from Compounds 1 to 416 described in the present application.

29. A pharmaceutical composition comprising a therapeutically effective amount of a compound in accordance with any one of claims 1-28.

30. The pharmaceutical composition of claim 29 in the form of a tablet.

31. A method of treating multiple system atrophy in a patient in need thereof, comprising administering to the patient a pharmaceutical composition comprising a therapeutically effective amount of a compound in accordance with any one of claims 1-28.

32. A method of treating amyotrophic lateral sclerosis in a patient in need thereof, comprising administering to the patient a pharmaceutical composition comprising a therapeutically effective amount of a compound in accordance with any one of claims 1-28.

33. A method of treating Huntington's disease in a patient in need thereof, comprising administering to the patient a pharmaceutical composition comprising a therapeutically effective amount of a compound in accordance with any one of claims 1-28.

34. A method for neuroprotection, comprising administering to the patient a pharmaceutical composition comprising a therapeutically effective amount of a compound in accordance with any one of claims 1-28.

35. A kit for treating multiple system atrophy in a patient, the kit comprising:
(a) a pharmaceutical composition comprising a therapeutically effective amount of a compound in accordance with any one of claims 1-28; and
(b) instructions for administering the pharmaceutical composition.

36. A kit for treating amyotrophic lateral sclerosis in a patient, the kit comprising:
(a) a pharmaceutical composition comprising a therapeutically effective amount of a compound in accordance with any one of claims 1-28; and
(b) instructions for administering the pharmaceutical composition.

37. A kit for treating Huntington's disease in a patient, the kit comprising:
(a) a pharmaceutical composition comprising a therapeutically effective amount of a compound in accordance with any one of claims 1-28; and
(b) instructions for administering the pharmaceutical composition.

38. A kit for neuroprotection, the kit comprising:
(a) a pharmaceutical composition comprising a therapeutically effective amount of a compound in accordance with any one of claims 1-28; and
(b) instructions for administering the pharmaceutical composition.
